(19) United States
(12) Patent Application Publication
(10) Pub. No.: US 2004/0106626 A1
(43) Pub. Date: Jun. 3, 2004

(54) 6-Membered unsaturated heterocyclic compounds useful for selective inhibition of the coagulation cascade

(75) Inventors: Michael S. South, St. Louis, MO (US); Ronald K. Webber, St. Charles, MO (US); Horng-Chih Huang, Chesterfield, MO (US); Mihaly V. Toth, St. Louis, MO (US); Alan E. Moormann, Weldon Springs, MO (US); Jeffery S. Snyder, Manchester, MO (US); Jeffrey A. Scholten, Chesterfield, MO (US); Danny J. Garland, Ballwin, MO (US); Melvin L. Rueppel, St. Louis, MO (US); William L. Neumann, St. Louis, MO (US); Scott Long, Ballwin, MO (US); Wei Huang, Wildwood, MO (US); John Trujillo, St. Peters, MO (US); John J. Parlow, Arnold, MO (US); Darin E. Jones, Ballwin, MO (US); Brenda Case, St. Louis, MO (US); Michael J. Hayes, St. Louis, MO (US); Qingping Zeng, Thousand Oaks, CA (US); Ricky L. Fenton, Collinsville, IL (US); Carrie L. Kusturin, Kirkwood, MO (US); Hayat K. Rahman, Belleville, IL (US); Zaheer Abbas, Chesterfield, MO (US); Kirby R. Sample, St. Louis, MO (US); Barbara A. Schweitzer, St. Louis, MO (US); Rhonda S. Wood, Rock Hill, MO (US); Jim Szalony, Hammond, IN (US); Osman D. Suleymanov, Arlington Heights, IL (US); Anita Salyers, Palatine, IL (US); Nancy S. Nicholson, Warrenville, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/263,525

(22) Filed: Oct. 3, 2002

Related U.S. Application Data
(60) Provisional application No. 60/326,721, filed on Oct. 3, 2001.
(60) Provisional application No. 60/338,623, filed on Oct. 24, 2001.
(60) Provisional application No. 60/332,857, filed on Nov. 6, 2001.
(60) Provisional application No. 60/333,292, filed on Nov. 14, 2001.
(60) Provisional application No. 60/332,107, filed on Nov. 21, 2001.
(60) Provisional application No. 60/332,014, filed on Nov. 21, 2001.

(51) Int. Cl. A61K 31/4965; C 07D 4 3/02

(52) U.S. Cl. 514/255.05; 514/255.06; 544/405; 544/407

(57) ABSTRACT

The present invention relates to compounds, and prodrugs thereof, composition and methods useful for preventing and treating thrombotic conditions in mammals. The compounds of the present invention, and prodrugs thereof, selectively inhibit certain proteases of the coagulation cascade.

### CROSS REFERENCE TO RELATED APPLICATIONS

This application claims priority from Provisional Application Serial No. 60/326,721 filed Oct. 3, 2001, No. 60/338,623 filed Oct. 24, 2001, No. 60/332,857 filed Nov. 6, 2001, No. 60/333,292 filed on Nov. 14, 2001, and No. 60/332,107 and 60/331,891 both filed on Nov. 21, 2001, which are all hereby incorporated by reference in their entirety.

### FIELD OF THE INVENTION

The present invention relates to compounds, compositions and methods for preventing and treating thrombotic conditions such as coronary artery and cerebrovascular disease. More particularly, the invention relates to compounds, and prodrugs thereof, that selectively inhibit serine proteases of the coagulation cascade.

### BACKGROUND OF THE INVENTION

Hemorrhage, intravascular thrombosis, and embolism are common clinical manifestations of many diseases (see R. I. Handin in Harrison's Principles of Internal Medicine (J. D. Wilson, et al. eds., 12th ed. 1991) New York, McGraw-Hill Book Co., pp. 348-351). The normal hemostatic system limits blood loss by precisely regulated interactions between components of the vessel wall, circulating blood platelets, and plasma proteins. However, unregulated activation of the hemostatic system may cause thrombosis, which can reduce blood flow to critical organs like the brain and myocardium.

Physiological systems control the fluidity of blood in mammals (see P. W. Majerus, et al. in Goodman & Gilman's The Pharmacological Basis of Therapeutics (J. G. Hardman & L. E. Limbird, eds., 9th ed. 1996) New York, McGraw-Hill Book Co., pp. 1341-1343). Blood must remain fluid within the vascular systems and yet quickly be able to undergo hemostasis. Hemostasis, or clotting, begins when platelets first adhere to macromolecules in subendothelian regions of injured and/or damaged blood vessels. These platelets aggregate to form the primary hemostatic plug and stimulate local activation of plasma coagulation factors leading to generation of a fibrin clot that reinforces the aggregated platelets. Plasma coagulation factors, also referred to as protease zymogens, include factors II, V, VII, VIII, IX, X, XI, and XII. These coagulation factors or protease zymogens are activated by serine proteases leading to coagulation in a so called “coagulation cascade” or chain reaction.

Coagulation or clotting occurs in two ways through different pathways. An intrinsic or contact pathway leads from XII to XIIa to XIa to IXa and to the conversion of X to Xa. Factor Xa in combination with factor Va converts prothrombin (II) to thrombin (IIa) leading to conversion of fibrinogen to fibrin. Polymerization of fibrin leads to a fibrin clot. An extrinsic pathway is initiated by the conversion of coagulation factor VII to VIIa by factor Xa. Factor VIIa, a plasma protease, is exposed to, and combines with its essential cofactor tissue factor (TF) which resides constitutively beneath the endothelium. The resulting factor VIIa/TF complex proteolytically activates its substrates, factors IX and X, triggering a cascade of reactions that leads to the generation of thrombin and a fibrin clot as described above.

While clotting as a result of an injury to a blood vessel is a critical physiological process for mammals, clotting can also lead to disease states. A pathological process called thrombosis results when platelet aggregation and/or a fibrin clot blocks (i.e., occludes) a blood vessel. Arterial thrombosis may result in ischemic necrosis of the tissue supplied by the artery. When the thrombosis occurs in a coronary artery, a myocardial infarction or heart attack can result. A thrombosis occurring in a vein may cause tissues drained by the vein to become edematous and inflamed. Thrombosis of a deep vein may be complicated by a pulmonary embolism. Preventing or treating clots in a blood vessel may be therapeutically useful by inhibiting formation of blood platelet aggregates, inhibiting formation of fibrin, inhibiting thrombus formation, inhibiting embolus formation, and for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels.

In order to treat such conditions, researchers have sought to discover chemical compounds that efficaciously and selectively control the clotting process. In addition, such compounds may provide a better understanding of the pathways involved in the coagulation process.

Thus far, many of the compounds that have been discovered possess a polar or basic functional group which is integrally responsible for the desired biological activity. Frequently, this polar functional group is a nitrogen atom of, for example, a guanidine, alkyl-amidine or aryl-amidine group. Because these functionalities are highly basic, they remain protonated at physiologically relevant pH's. The ionic nature of such protonated species hinders their permeability across lipophilic membranes, which can reduce bioavailability when the pharmaceutical agent is administered orally.

In order to circumvent such a problem, it is often advantageous to perform a derivatization or chemical modification of the polar functionality such that the pharmaceutical agent becomes neutrally charged and more lipophilic, thereby facilitating absorption of the drug. However, for the derivatization to be useful, the derivatization must be bioconvertable at the target site or sites of desired pharmacological activity and cleaved under normal physiological conditions to yield the biologically active drug. The term “prodrug” has been used to denote such a chemically modified intermediate.

### SUMMARY OF THE INVENTION

Among the various aspects of the present invention, therefore, is the provision of compounds useful for selective inhibition of certain enzymes that act upon the coagulation cascade thereby preventing and treating thrombotic conditions in mammals.

Another aspect of the present invention is the provision of prodrug compounds useful for selective inhibition of certain enzymes that act upon the coagulation cascade thereby preventing and treating thrombotic conditions in mammals. In general, these prodrug compounds undergo hydrolysis, oxidation, reduction or elimination at a derivatized amidine group to yield the active compound.

Briefly, therefore, the present invention is directed to a compound, per se, to a prodrug of the compound, to pharmaceutical compositions comprising the compound or prodrug and a pharmaceutically acceptable carrier, and to methods of use. The compound corresponds to formula (1):

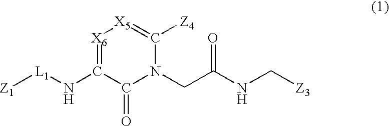

wherein:

X₁ and X₆ are members of an unsaturated heterocyclic ring, and are independently nitrogen, CH, C(F), C(Cl), or C(Br);

L₁ is a linker, linking Z₁ to the heterocyclic ring and optionally additionally containing a bond to X₆ to form a fused ring with the heterocyclic ring;

Z₁ is C₁-C₈ alkyl, C₂-C₈ alkenyl, or C₂-C₈ alkynyl, the alkyl, alkenyl, or alkynyl being optionally substituted with fluorine, hydroxy, carboxy, or alkoxycarbonyl;

Z₃ comprises a substituted phenyl, thienyl, or furanyl ring, the phenyl, thienyl, or furanyl ring being substituted with an amidine or a derivatized amidine group, and optionally substituted with fluorine, hydroxy, carboxy, alkoxycarbonyl, or hydrocarbyloxy;

Z₄ comprises a 5- or 6-membered heteroaryl or aryl ring, the ring atoms of Z₄ being Z₄₀, Z₄₁, Z₄₂₁ Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁/the substituent being R₄₃ when bonded to Z₄₃₁ the substituent being R₄₄ when bonded to Z₄₄, and the substituent being R₄₅ when bonded to Z₄₅;

R₄₂ is amino; and

R₄₁, R₄₃, R₄₄ and R₄, are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen.

Other aspects and features of this invention will be in part apparent and in part pointed out hereafter.

Abbreviations and Definitions

The term “elimination” as used herein is generally meant to encompass any one or more of the following reactions: (1) a reaction that results in a compound fragmenting into two or more compounds; and (2) a reaction that results in one or more groups being removed from a compounds without being replaced by other groups.

The term “oxidation” as used herein is generally meant to encompass any one or more of the following reactions: (1) a reaction that results in an increase in the oxidation number of an atom in a compound, whether the atom is uncharged or charged and whether free or covalently bound; (2) a reaction that results in the loss of hydrogen from a compound; (3) a reaction that results in the loss or removal of one or more electrons from a compound, with or without concomitant loss or removal of a proton or protons; (4) the action or process of reacting a compound with oxygen; and (5) a reaction that results in the addition of one or more oxygen atoms to a compound.

The term “reduction” as used herein is generally meant to encompass any one or more of the following reactions: (1) any reaction which results in a decrease in the oxidation number of an atom in a compound; and (2) any reaction that results in oxygen being withdrawn from, hydrogen being added to, or an electron being added to (with or without the addition of a proton) a compound.

The term “hydrolysis” as used herein is generally meant to encompass any one or more of the following reactions: (1) any reaction which results in the addition of a nucleophile to a compound to form a new bond with concurrent loss of a group from the compound; (2) any reaction which results in the addition of water to a compound; and (3) any reaction that results in the rupture of one or more chemical bonds by reaction with, and involving the addition of, the elements of water.

The term “physiological conditions” are those conditions characteristic to an organism's (to a human beings) healthy or normal functioning.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorus, boron, sulfur, or a halogen atom. Exemplary substituted hydrocarbyl moieties include, heterocyclo, alkoxyalkyl, alkenyloxyalkyl, alkynyloxyalkyl, aryloxyalkyl, hydroxyalkyl, protected hydroxyalkyl, keto, acyl, nitroalkyl, aminoalkyl, cyano, alkylthioalkyl, arylthioalkyl, ketals, acetals, amides, acids, esters and the like.

The term “heteroatom” shall mean atoms other than carbon and hydrogen.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, cycylopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furanyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furanyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “acetamidyl” as used herein describes a chemical moiety represented by the formula NR₁C(O)R₂.

The term “carboxamido” as used herein, describes a chemical moiety represented by the formula C(O)NR₁R₂.

The term “alkoxycarbonyl” as used herein describes a chemical moiety represented by the formula C(O)OR.

The term “sulfonamido” as used herein describes a chemical moiety represented by the formula SO₂NR₁R₂.

The term “alkylsulfonyl” as used herein describes a chemical moiety represented by the formula SO₂R.

The term “sulfonamidyl” as used herein describes a chemical moiety represented by the formula NRSO₂R.

As described herein for the terms “acetamidyl”, “carboxamido”, “alkocycarbonyl”, “sulfonamido”, “alkylsulfonyl”, and “sulfonamidyl”, R, R₁ and R₂ are independently hydrogen, alkyl, aryl, and arylakyl, optionally substituted with halogen, hydroxy or alkoxy.

### DESCRIPTION OF THE PREFERRED EMBODIMENT

One aspect of the invention embraces compounds that correspond to formula (1):

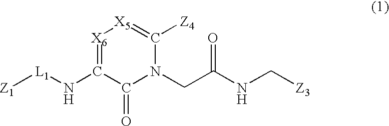

wherein:

X₅ and X₆ are members of an unsaturated heterocyclic ring, and are independently nitrogen, CH, C(F), C(Cl), or C(Br);

L₁ is a linker, linking Z₁ to the heterocyclic ring and optionally additionally containing a bond to X₆ to form a fused ring with the heterocyclic ring;

Z₁ is C₁-C₈ alkyl, C₂-C₈ alkenyl, or C₂-C₈ alkynyl, the alkyl, alkenyl, or alkynyl being optionally substituted with fluorine, hydroxy, carboxy, or alkoxycarbonyl;

Z₃ comprises a substituted phenyl, thienyl, or furanyl ring, the phenyl, thienyl, or furanyl ring being substituted with an amidine or a derivatized amidine group, and optionally substituted with fluorine, hydroxy, carboxy, alkoxycarbonyl, or hydrocarbyloxy;

Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄, when Z₄ is a 6-membered ring, Z₄₀, Z₄₁₁ Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄, each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅;

R₄₂ is amino; and

R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen.

In another embodiment of compounds of formula (1), Z₁ is C₁-C₈ alkyl, C₂-C₈ alkenyl, or C₂-C₈ alkynyl, the alkyl, alkenyl, or alkynyl being optionally substituted with fluorine;

Z₃ comprises a substituted phenyl or substituted thienyl ring, the phenyl or thienyl ring being substituted with an amidine or a derivatized amidine group, and optionally further substituted with fluorine or hydroxy;

R₄₄ is hydrocarbyl, substituted hydrocarbyl, heterocyclo, halogen, or a substituted or unsubstituted heteroatom selected from nitrogen, oxygen, sulfur and phosphorus; and

X₅, X₆, L₁, L₃, Z₄ and R₄₂ are as defined above.

In another embodiment of compounds corresponding to formula (1), X₅ is CH, X₆ is nitrogen and L₁, Z₁, Z₃ and Z₄ are as defined above. In an alternative embodiment of compounds of formula (1), X₅ and X₆ are CH and L₁, Z₁, Z₃ and Z₄ are as defined above. In another alternative embodiment of compounds of formula (1), X₅ is nitrogen, X₆ is CH and L₁, Z₁, Z₃ and Z₄ are as defined above. In yet another alternative embodiment of compounds of formula (1), X₅ and X₆ are nitrogen and L₁, Z₁, Z₃ and Z₄ are as defined above.

In one embodiment of compounds corresponding to formula (1), the L₁ linkage is a bond or an alkylene chain, (CH₂)ₘ wherein m is 0 to 5. In another embodiment, m is 0 to 2. A preferred L₁ linkage is a bond.

In one embodiment of compounds corresponding to formula (1), Z₁ is C₁-C₅ alkyl optionally substituted at any substitutable position with fluorine, hydroxy, carboxy or alkoxycarbonyl. Preferred C₁-C₅ alkyl groups include propyl, isopropyl, cyclopropyl, tert-butyl and cyclobutyl. In another alternative of this embodiment (i.e., when Z₁ is optionally substituted C₁-C₅ alkyl), Z₁ is other than isopropyl or cyclobutyl. In still another alternative of this embodiment, Z₁ is other than unsubstituted isopropyl or cyclobutyl. In yet still another alternative of this embodiment, Z₁ is isopropyl or cyclobutyl substituted with fluorine, hydroxy, carboxy, or alkocycarbonyl. In another alternative of this embodiment, Z₁ is trifluoroethyl or carboxymethyl.

In one embodiment of compounds corresponding to formula (1), Z₃ comprises a substituted phenyl, thienyl or furanyl ring, the phenyl, thienyl or furanyl ring being substituted with an amidine or a derivatized amidine group and optionally substituted at any substitutable position with fluorine, hydroxy, carboxy, alkoxycarbonyl or hydrocarbyloxy. In another alternative of this embodiment, Z₃ is subsituted with an amidine or a derivatized amidine group and at least one of hydroxy, carboxy, alkoxycarbonyl, or hydrocarbyloxy. In still another alternative of this embodiment, Z₃ is hydroxy or carboxy substituted. In yet another alternative of this embodiment, Z₃ corresponds to formula (a)

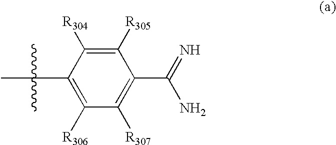

wherein

R₃₀₄ and R₃₀₆ are independently selected from the group consisting of hydrogen, fluorine, hydroxy, carboxy, hydrocarbyloxy, and alkoxycarbonyl; and

R₃₀₅ and R₃₀₇ are independently selected from the group consisting of hydrogen, fluorine, methoxy, hydroxy, and carboxy.

Preferred R₃₀₄, R₃₀₅, R₃₀₆, and R₃₀₇ include hydrogen, fluorine, hydroxy, carboxy and methoxy.

In still another alternative of this embodiment, Z₃ is other than 4-amidinobenzyl, 4-amidino-2-fluorobenzyl, and 4-amidino-3-fluorobenzyl.

In another embodiment, Z₃ is a phenyl, thienyl, or furanyl ring substituted with a derivatized amidine which, upon hydrolysis, oxidation, reduction, or elimination, or any combination thereof, under physiological conditions yields an amidine group, as discussed more fully below.

In one embodiment, Z₄ corresponds to formula (b)

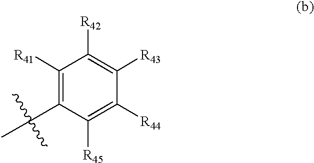

wherein

R₄₂ is amino;

R₄₄ is hydrocarbyl, substituted hydrocarbyl, halogen or an optionally substituted heteroatom selected from the group consisting of oxygen, nitrogen, and sulfur; and

R₄₁, R₄₃ and R₄₅ are independently hydrogen, hydrocarbyl, substituted hydrocarbyl, halogen or an optionally substituted heteroatom selected from the group consisting of oxygen, nitrogen and sulfur.

In another embodiment of compounds wherein Z₄ corresponds to formula (b) and R₄₂ is amino, R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₄ is selected from the group consisting of hydroxy, carboxy, carboxamido, alkoxy, alkylsulfonyl, sulfonamido, and alkoxycarbonyl. In yet another alternative of this embodiment, R₄₄ is selected from the group consisting of sec-butylamide, carboxy, ethoxycarbonyl, isopropoxycarbonyl, butoxycarbonyl, isopropylamide and hydroxy. In still another alternative of this embodiment R₄₁, R₄₃ and R₄₅ are independently selected from the group consisting of hydrogen, halogen, alkoxy, or hydroxy and R₄₄ is as defined in any of the alternative embodiments above. In yet another alternative of this embodiment, R₄₁, R₄₃ and R₄₅ are independently selected from the group consisting of hydrogen and halogen and R₄₄ is as defined in any of the alternative embodiments above. In still another alternative of this embodiment, Z₄₁, Z₄₃ or Z₄₅ is substituted with fluorine or chlorine. A preferred halogen is chlorine. A more preferred halogen is fluorine. A preferred alkoxy is methoxy.

In another embodiment of compounds wherein Z₄ corresponds to formula (b) and R₄₂ is amino, R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₅ is selected from the group consisting of hydroxy, carboxy, carboxamido, alkoxy, alkylsulfonyl, sulfonamido, and alkoxycarbonyl. In yet another alternative of this embodiment, R₄₅ is selected from the group consisting of sec-butylamide, carboxy, ethoxycarbonyl, isopropoxycarbonyl, butoxycarbonyl, isopropylamide and hydroxy. In still another alternative of this embodiment R₄₁, R₄₃ and R₄₄ are independently selected from the group consisting of hydrogen, halogen, alkoxy, or hydroxy and R₄₅ is as defined in any of the alternative embodiments above. In yet another alternative of this embodiment, R₄₁, R₄₃ and R₄₄ are independently selected from the group consisting of hydrogen and halogen and R₄₅ is as defined in any of the alternative embodiments above. In still another alternative of this embodiment, Z₄₁, Z₄₃ or Z₄₄ is substituted with fluorine or chlorine. A preferred halogen is chlorine. A more preferred halogen is fluorine. A preferred alkoxy is methoxy.

In yet another embodiment of compounds wherein Z₄ corresponds to formula (b) and R₄₂ is amino, R₄₃ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₃ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₃ is selected from the group consisting of hydroxy, carboxy, carboxamido, alkoxy, alkylsulfonyl, sulfonamido, and alkoxycarbonyl. In yet another alternative of this embodiment, R₄₃ is selected from the group consisting of sec-butylamide, carboxy, ethoxycarbonyl, isopropoxycarbonyl, butoxycarbonyl, isopropylamide and hydroxy. In still another alternative of this embodiment R₄₁, R₄₄ and R₄₅ are independently selected from the group consisting of hydrogen, halogen, alkoxy, or hydroxy and R₄₃ is as defined in any of the alternative embodiments above. In yet another alternative of this embodiment, R₄₁, R₄₄ and R₄₅ are independently selected from the group consisting of hydrogen and halogen and R₄₃ is as defined in any of the alternative embodiments above. In still another alternative of this embodiment, Z₄₁, Z₄₄ or Z₄₅ is substituted with fluorine or chlorine. A preferred halogen is chlorine. A more preferred halogen is fluorine. A preferred alkoxy is methoxy.

In still another embodiment of compounds wherein Z₄ corresponds to formula (b) and R₄₂ is amino, R₄₁ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₁ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₁ is selected from the group consisting of hydroxy, carboxy, carboxamido, alkoxy, alkylsulfonyl, sulfonamido, and alkoxycarbonyl. In yet another alternative of this embodiment, R₄₁ is selected from the group consisting of sec-butylamide, carboxy, ethoxycarbonyl, isopropoxycarbonyl, butoxycarbonyl, isopropylamide and hydroxy. In still another alternative of this embodiment R₄₃, R₄₄ and R₄₅ are independently selected from the group consisting of hydrogen, halogen, alkoxy, or hydroxy and R₄₁ is as defined in any of the alternative embodiments above. In yet another alternative of this embodiment, R₄₃, R₄₄ and R₄₅ are independently selected from the group consisting of hydrogen and halogen and R₄₁ is as defined in any of the alternative embodiments above. In still another alternative of this embodiment, Z₄₃, Z₄₄ or Z₄₅ is substituted with fluorine or chlorine. A preferred halogen is chlorine. A more preferred halogen is fluorine. A preferred alkoxy is methoxy.

In one embodiment of compounds corresponding to formula (1), L₁ is a bond. In one alternative of this embodiment (i.e., when L₁ is a bond), Z₁ is C₁-C₅ alkyl substituted at any substitutable position with fluorine, hydroxy, carboxy or alkoxycaronyl. In another alternative of this embodiment, Z₁ is an unsubstituted C₁-C₅ alkyl group. In another alternative of this embodiment, Z₃ is phenyl substituted with an amidine group and optionally substituted at any substitutable position with fluorine, hydroxy, carboxy, of hydrocarbyloxy. In another alternative of this embodiment, Z₃ is phenyl substituted with a derivatized amidine group which, upon hydrolysis, oxidation, reduction, or elimination, or any combination thereof, under physiological conditions yields an amidine group. In yet another alternative of this embodiment, Z₄ is phenyl substituted with R₄₂ and R₄₄ wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In still a further alternative of this embodiment, Z₄ is phenyl substituted with R₄₂ and R₄₅ wherein R₄₂ is amino and R₄₁ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative embodiment of this embodiment, Z₄ is phenyl substituted with R₄₂ and R₄₃ wherein R₄₂ is amino and R₄₃ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In still another alternative of this embodiment, Z₄ is phenyl substituted with R₄₂ and R₄₁ wherein R₄₂ is amino and R₄₁ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

In another embodiment of compounds corresponding to formula (1), Z₁ is C₁-C₅ alkyl optionally substituted at any substitutable position with fluorine, hydroxy, carboxy, or alkoxycarbonyl. In one alternative of this embodiment (i.e., when Z₁ is optionally substituted C₁-C₅ alkyl), L₁ is a bond, methylene or ethylene. In another alternative of this embodiment, Z₃ is phenyl substituted with an amidine group and optionally substituted at any substitutable position with fluorine, hydroxy, carboxy, alkoxycarbonyl, or hydrocarbyloxy. In another alternative of this embodiment, Z₃ is a phenyl substituted with a derivatized amidine group which, upon hydrolysis, oxidation, reduction, or elimination, or any combination thereof, under physiological conditions yields an amidine group. In still another alternative of this embodiment, Z₄ is phenyl substituted with R₄₂ and R₄₄ wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

In another embodiment of compounds corresponding to formula (1), Z₃ is phenyl substituted with an amidine group and optionally substituted at any substitutable position with fluorine, hydroxy, carboxy, alkoxycarbonyl, or hydrocarbyloxy. In one alternative of this embodiment (i.e., when Z₃ is substituted or unsubstituted benzamidine), L₁ is a bond, methylene or ethylene. In another alternative of this embodiment, Z₁ is C₁-C₅ alkyl optionally substituted at any substitutable position with fluorine, hydroxy, carboxy, or alkoxycarbonyl. In still another alternative of this embodiment, Z₄ is phenyl substituted with R₄₂ and R₄₄ wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

In yet another embodiment, Z₄ corresponds to formula (b) wherein R₄₂ is amino and R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen. In one alternative of this embodiment (i.e., when Z₄ is disubstituted phenyl), L₁ is a bond, methylene or ethylene. In another alternative of this embodiment, Z₁ is C₁-C₅ alkyl optionally substituted at any substitutable position with fluorine, hydroxy, carboxy or alkoxycarbonyl. In another alternative of this embodiment, Z₃ is phenyl substituted with an amidine group and optionally substituted at any substitutable position with fluorine, hydroxy, carboxy, alkoxycarbonyl, or hydrocarbyloxy. In another alternative of this embodiment, Z₃ is a phenyl substituted with a derivatized amidine group which, upon hydrolysis, oxidation, reduction, or elimination, or any combination thereof, under physiological conditions yields an amidine group.

In a particularly preferred embodiment of compounds corresponding to formula (1), L₁ is a bond, Z₁ is cyclopropyl or isopropyl optionally substituted with fluorine, hydroxy, carboxy, or alkoxycarbonyl, Z₃ is phenyl substituted with an amidine or derivatized amidine group and optionally further substituted at any substitutable position with fluorine, hydroxy, or carboxy and Z₄ is formula (b) wherein R₄₂ is amino and R₄₄ is selected from the group consisting of sec-butylamide, carboxy, ethoxycarbonyl, isopropoxycarbonyl, butoxycarbonyl, isopropylamide and hydroxy.

In another aspect of the invention, the heterocyclic ring forms a pyrazinone corresponding to formula (2):

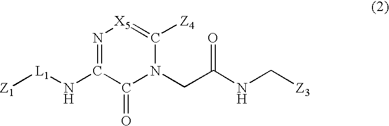

wherein X₅ is CH, C(Br), or C(F) and each of Z₁, Z₃, Z₄, L₁, L₃, R₄₂, and R₄₄ are as described above for formula (1). In one embodiment, Z₁ is other than isopropyl or cyclobutyl.

In one embodiment of compounds corresponding to formula (2), L₁ is a bond. In one alternative of this embodiment (i.e., when L₁ is a bond), X₅ is CH. In another alternative of this embodiment, one of the following conditions exist: (a) Z₁ is other than unsubstituted cyclobutyl when X₅ is CH; (b) Z₁ is other than unsubstituted isopropyl when (i) X₅ is CH and (ii) Z₄ is 3,5-diaminophenyl or 3-amino-5-(2,2,2-trifluoroacetamide)phenyl; or (c) Z₃ is other than 4-amidinobenzyl, 4-amidino-2-fluorobenzyl, or 4-amidino-3-fluorobenzyl. In still another alternative of this embodiment, Z₁ is isopropyl or cyclobutyl substituted with fluorine, hydroxy, carboxy, or alkoxycarbonyl.

In a preferred embodiment of compounds corresponding to formula (2), L₁ is a bond, Z₁ is selected from the group consisting of cyclopropyl, isopropyl, methyl, ethyl, cyclobutyl, isobutyl, tert-butyl, and sec-butyl optionally substituted at any substitutable position with fluorine, hydroxy, carboxy or alkoxycarbonyl, Z₃ is phenyl substituted with an amidine group and optionally substituted with fluorine, hydroxy, carboxy, alkoxycarbonyl, or hydrocarbyloxy, and Z₄ is formula (b) wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

In yet another preferred embodiment, compounds corresponding to formula (2) may be represented by formula (2-a)

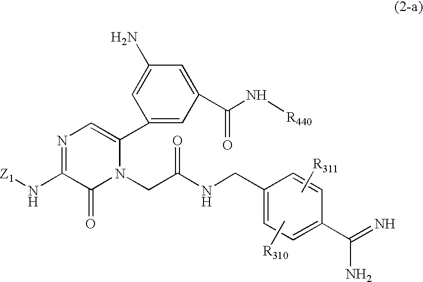

wherein

Z₁ is isopropyl or cyclopropyl optionally substituted at any substitutable position with fluorine, hydroxy, carboxy or alkoxycarbonyl;

R₄₄₀ is C₁-C₆ alkyl, aryl, aralkyl, carboxy, or carboxyalkyl, wherein said alkyl, aryl, aralkyl, carboxy, or carboxyalkyl is optionally further substituted with fluorine; and

R₃₁₀ and R₃₁₁ are independently selected from the group consisting of hydrogen, fluorine, hydroxy, alkoxy, and carboxy.

In another aspect of the invention, the heterocyclic ring forms a pyridone having the following formula (3):

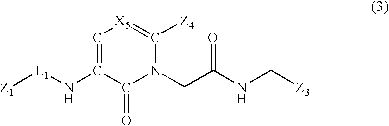

wherein X₅ is CH, C(Br), C(Cl), or C(F) and each of Z₁, Z₃, Z₄, L₁, R₄₂, and R₄₄ are as described above for formula (1). In one embodiment, Z₁ is other than isopropyl or cyclobutyl. In another embodiment, neither Z₄₁ nor Z₄₅ is sulfur when Z₄ is thienyl.

In one embodiment for compounds corresponding to formula (3), L₁ is a bond. In one alternative of this embodiment (i.e., when L₁ is a bond), X₅ is CH. In still another alternative of this embodiment, one of the following conditions exist: (a) Z₃ is other than 4-amidinobenzyl, 4-amidino-2-fluorobenzyl, and 4-amidino-3-fluorobenzyl; or (b) (i) Z₁ is other than unsubstituted cyclobutyl and unsubstituted isopropyl when X₅ is CH or C(Cl) and (ii) neither Z₄₁ nor Z₄₅ is sulfur when Z₄ is thienyl. In yet another alternative of this embodiment, Z₁ is isopropyl or cyclobutyl substituted with fluorine, hydroxy, carboxy, or alkoxycarbonyl.

In a preferred embodiment of compounds corresponding to formula (3), L₁ is a bond, Z₁ is selected from the group consisting of cyclopropyl, isopropyl, methyl, ethyl, cyclobutyl, isobutyl, tert-butyl, and sec-butyl optionally substituted at any substitutable position with fluorine, hydroxy, carboxy or alkoxycarbonyl, Z₃ is phenyl substituted with an amidine group and optionally substituted with fluorine, hydroxy, carboxy, alkoxycarbonyl, or hydrocarbyloxy, and Z₄ is formula (b) wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

In another aspect of the invention, the heterocyclic ring forms a pyrimidinone corresponding to formula (4):

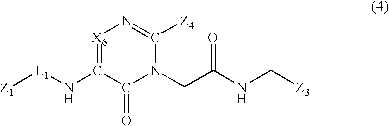

wherein X₆ is CH, C(Br), C(Cl), or C(F) and each of Z₁, Z₃, Z₄, L₁, R₄₂, and R₄₄ are as described above for formula (1) In one embodiment, Z₁ is other than isopropyl or cyclobutyl. In another embodiment neither Z₄₁ nor Z₄₅ is sulfur when Z₄ is thienyl. In a preferred embodiment, X₆ is CH.

In one embodiment of compounds corresponding to formula (4), L₁ is a bond. In one alternative of this embodiment (i.e., when L₁ is a bond), X₆ is CH. In another alternative of this embodiment, Z₁ is other than unsubstituted cyclobutyl or unsubstituted isopropyl and neither Z₄₁ nor Z₄₅ is sulfur when Z₄ is thienyl. In still another alternative of this embodiment, Z₁ is isopropyl or cyclobutyl substituted with fluorine, hydroxy, carboxy, or alkoxycarbonyl.

In a preferred embodiment of compounds corresponding to formula (4), L₁ is a bond, Z₁ is selected from the group consisting of cyclopropyl, methyl, ethyl, isobutyl, tert-butyl, and sec-butyl optionally substituted at any substitutable position with fluorine, hydroxy, carboxy or alkoxycarbonyl, Z₃ is phenyl substituted with an amidine group and optionally substituted by fluorine, hydroxy, carboxy, alkoxycarbonyl, or hydrocarbyloxy, and Z₄ corresponds to formula (b) wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

In another aspect of the invention, the heterocyclic ring forms a triazinone corresponding to formula (5):

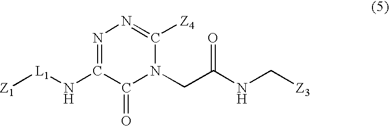

wherein each of Z₁, Z₃, Z₄, L₁, R₄₂, and R₄₄ are as described above for formula (1). In one embodiment, Z₁ is other than isopropyl or cyclobutyl. In another embodiment neither Z₄₁ nor Z₄₅ is sulfur when Z₄ is thienyl.

In one embodiment of compounds corresponding to formula (5), L₁ is a bond. In one alternative of this embodiment (i.e., when L₁ is a bond), Z₁ is other than unsubstituted cyclobutyl or unsubstituted isopropyl and neither Z₄₁ nor Z₄₅ is sulfur when Z₄ is thienyl. In another alternative of this embodiment, Z₁ is isopropyl or cyclobutyl substituted with fluorine, hydroxy, carboxy or alkoxycarbonyl.

In a preferred embodiment of compounds corresponding to formula (5), L₁ is a bond, Z₁ is selected from the group consisting of cyclopropyl, methyl, ethyl, isobutyl, tert-butyl, and sec-butyl optionally substituted at any substitutable position with fluorine, hydroxy, carboxy or alkoxycarbonyl, Z₃ is phenyl substituted with an amidine group and optionally substituted by fluorine, hydroxy, carboxy, alkoxycarbonyl, or hydrocarbyloxy, and Z₄ corresponds to formula (b) wherein R₄₂ is amino and R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

Another aspect of the invention embraces compounds which correspond to formula (1) having the fused ring formula (6):

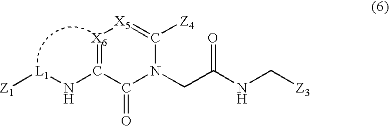

wherein Z₁, Z₄, Z₃, X₅, X₆ are as defined above for formula (1) and L₁ contains a bond directly to X₆ to form a fused ring with the heterocyclic ring. Exemplary linkages from L₁ to X₆ contain from one to six atoms forming an aryl, heteroaryl, heterocyclic or carbocyclic fused ring. Preferred exemplary linkages form a five or six membered aryl, heteroaryl, heterocyclic or carbocyclic fused ring.

In one embodiment, compounds corresponding to formula (6), may be represented by formula (7):

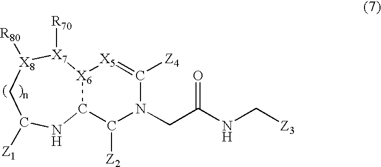

wherein

X₅ is nitrogen, CH, C(F), C(Cl), or C(Br);

X₆ is carbon or nitrogen, provided the dashed line represents a double bond when X₆ is carbon and the dashed line represents a single bond when X₆ is nitrogen;

X₇ and X₈ are independently carbon, nitrogen, oxygen or sulfur;

Z₂ is a hydrogen bond acceptor covalently bonded to the carbon gamma to X₅;

n is 0 to 2; and

Z₁, Z₃ and Z₄ are as defined for formula (1).

In one alternative of this embodiment (i.e., compounds corresponding to formula (7)), X₆ is carbon, thereby making the dashed lines represent a double bond. In another alternative of this embodiment, X₆ is nitrogen thereby making the dashed lines represent a single bond.

Generally, as used herein, hydrogen bond acceptors are heteroatoms that have a lone pair of electrons available for hydrogen bonding. When taken with the carbon to which Z₂ is attached, suitable hydrogen bond acceptors are selected from the group consisting of C(O), C(S), C(Cl), C(Br), C(F), C(OH), COCH₃, COR, C(SH), CSR, and CNR₁R₂ wherein R, R₁ and R₂ are independently hydrogen, alkyl, aryl, and arylakyl, optionally substituted with halogen, hydroxy or alkoxy.

In another aspect of the invention, compounds corresponding to any of formulas (1)-(7), have no zwitterionic effect. In one alternative of this embodiment (i.e., compounds having no zwitterionic effect) wherein Z₄ is formula (b) and R₄₂ is amino, R₄₄ is selected from other than carboxy. It is hypothesized that compounds lacking a zwitterionic effect have increased solubility over analogous compounds possessing such zwitterionic effect.

Another aspect of the invention embraces intermediate compounds having either of two formulae. Compounds corresponding to one of the formulas may be represented by formula (8):

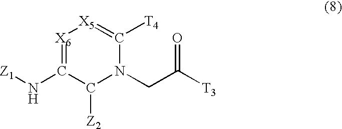

wherein

X₁ and X₆ are independently nitrogen, CH, C(F) or C(Br);

T₃ is hydroxy, alkoxy, substituted alkoxy, or substituted amino;

T₄ is Cl, Br, I, S(CH₃), or OSO₂ (CF₃);

Z₁ is C₁-C₈ alkyl, C₂-C₈ alkenyl, or C₂-C₈ alkynyl, the alkyl, alkenyl, or alkynyl being optionally substituted with fluorine, hydroxy, carboxy, or alkoxycarbonyl; and

Z₂ is a hydrogen bond acceptor covalently bonded to the carbon gamma to X₅.

Intermediate compounds represented by the other formula may be represented by formula (9):

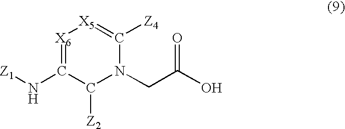

wherein

X₅ and X₆ are independently nitrogen, CH, C(F) or C(Br);

Z₁ is C₁-C₈ alkyl, C₂-C₈ alkenyl, or C₂-C₈ alkynyl, the alkyl, alkenyl, or alkynyl being optionally substituted with fluorine, hydroxy, carboxy, or alkoxycarbonyl;

Z₂ is a hydrogen bond acceptor covalently bonded to the carbon gamma to X₅; and

Z₄ is hydrocarbyl, substituted hydrocarbyl, or a 5- or 6-membered heterocyclic or carbocyclic ring, the ring atoms of the 5- or 6-membered heterocyclic or carboxylic ring of Z₄ being carbon, nitrogen, oxygen, or sulfur.

In one embodiment of compounds corresponding to either formula (8) or (9), hydrogen bond acceptors are as defined above.

Among the preferred embodiments, therefore, are compounds corresponding to any of formulas (1)-(7), wherein Z₁ is selected from the group consisting of cyclopropyl, isopropyl, methyl, ethyl, cyclobutyl, isobutyl, and sec-butyl optionally substituted with fluorine, hydroxy, carboxy, or alkoxycarbonyl, L₁ is a bond, Z₃ is phenyl, thienyl, or furanyl ring substituted with an amidine or a derivatized amidine group and optionally further substituted at any position with fluorine, hydroxy, carboxy, alkoxycarbonyl, or hydrocarbyloxy and Z₄ is a phenyl ring having two substituents, R₄₂ and R₄₄.

In another embodiment of compounds corresponding to any of formulas (1)-(7), Z₄ is phenyl ring having two substituents, R₄₂ and one of R₄₁, R₄₃₁ R₄₄, or R₄₅. Preferred substituents of Z₄ are R₄₂ and R₄₃. More preferred substituents of Z₄ are R₄₂ and R₄₁. The most preferred substituents of Z₄ are R₄₂ and R₄₄. Preferred R₄₂ and R₄₄ groups are as described above. Particularly preferred R₄₄ groups are sec-butylamide, carboxy, ethoxycarbonyl, isopropoxycarbonyl, butoxycarbonyl, isopropylamide and hydroxy.

In another embodiment of compounds corresponding to any of formulas (1)-(7), Z₄ is a 5-membered heteroaryl ring having two substituents, R₄₂ and R₄₄, provided neither Z₄₁ nor Z₄₅ is sulfur when Z₄ is thienyl. Preferred R₄₂ and R₄₄ groups are as described above. Particularly preferred R₄₄ groups are sec-butylamide, carboxy, ethoxycarbonyl, isopropoxycarbonyl, butoxycarbonyl, isopropylamide and hydroxy.

A further aspect of the invention embraces compounds that are prodrugs of any of the compounds corresponding to formulas (1)-(7). Generally speaking, any prodrug compound of the present invention having one or more prodrug moieties as part of the compound, can be converted under physiological conditions to the biologically active drug by a number of chemical and biological mechanisms. Typically, the prodrug compounds have phenyl or thienyl rings at position Z₃ substituted with a derivatized amidine which, upon hydrolysis, oxidation, reduction or elimination yields an amidine group. For illustrative purposes, the following paragraphs detail conversion of the prodrug to the biologically active compound when the prodrug moiety is covalently bonded to the amidine group on Z₃.

In one embodiment, conversion of the prodrug to the biologically active drug can be accomplished by hydrolysis of the prodrug moiety provided the prodrug moiety is chemically or enzymatically hydrolyzable with water. The reaction with water typically results in removal of the prodrug moiety and liberation of the biologically active drug. By way of example, a hydrolyzable prodrug derivative at the amidine group may be a carbonyl derivative such as N-acyl. Hydrolysis results in freeing the amidine group of the drug by removal of the acyl as carbon acid. Other suitable hydrolyzable prodrug derivatives include carbonyl, thiocarbonyl, imine, enamine, and oxygenated sulfur.

Yet another aspect of the invention provides conversion of the prodrug to the biologically active drug by reduction of the prodrug moiety. Typically in this embodiment, the prodrug moiety is reducible under physiological conditions in the presence of a reducing enzymatic process. The reduction preferably results in removal of the prodrug moiety and liberation of the biologically active drug. An example of a reducible prodrug derivative at the amidine group is an oxygen containing group in which an oxygen is directly attached to the amidine. Reduction results in freeing the amidine group of the drug by removal of oxygen as water or an alcohol. Generally speaking, other suitable reducible prodrug derivatives include a nitrogen containing group, and a sulfur containing group, provided both nitrogen and sulfur are each preferably in their most reduced state.

In another embodiment, conversion of the prodrug to the biologically active drug can also be accomplished by oxidation of the prodrug moiety. Typically in this embodiment, the prodrug moiety is oxidizable under physiological conditions in the presence of an oxidative enzymatic process. The oxidation preferably results in removal of the prodrug moiety and liberation of the biologically active drug. An example of an oxidizable prodrug derivative at the amidine group is a hydrocarbyl containing unsaturation in the carbon beta to the carbon directly connected to the amidine group. Oxidation results in forming an oxygenated intermediate that breaks down, thereby freeing the amidine group of the drug with concurrent hydrolysis of the oxygenated hydrocarbyl residue. Other suitable oxidizable prodrug derivatives of the amidine include saturated hydrocarbyl, unsaturated substituted hydrocarbyl, aryl, and aralkyl.

A further aspect of the invention encompasses conversion of the prodrug to the biologically active drug by elimination of the prodrug moiety. Generally speaking, in this embodiment the prodrug moiety is removed under physiological conditions with a chemical or biological reaction. The elimination results in removal of the prodrug moiety and liberation of the biologically active drug. By way of example, an eliminateable prodrug derivative at the amidine group is a hydrocarbyl containing an unsaturated electron withdrawing group bonded to the carbon beta to the carbon directly connected to the amidine. More specifically, for illustration purposes and exemplification, the hydrocarbyl group could have a cyano group beta to the carbon directly bonded to the amidino group. Elimination results in the freeing of the amidine group of the drug with concurrent removal of the unsaturated hydrocarbyl residue derived from the prodrug moiety. Other suitable eliminateable prodrug derivatives of the amidine include a hydrocarbyl substituted at the beta carbon with carbonyl, alkoxycarbonyl, amidocarbonyl, nitro, or sulfonyl or an alkyl group substituted with oxygen, nitrogen or sulfur at the carbon directly bonded to the amidine group.

Any prodrug compound of the present invention may undergo any combination of the above detailed mechanisms to convert the prodrug to the biologically active compound. For example, a particular compound may undergo hydrolysis, oxidation, elimination, and reduction to convert the prodrug to the biologically active compound. Equally, a particular compound may undergo only one of these mechanisms to convert the prodrug to the biologically active compound.

A further embodiment embraces compounds having any of formulas (1)-(7) wherein Z₃ is —R₃₀₀C(═NR₃₀₁)NR₃₀₂R₃₀₃, wherein R₃₀₀ is a 6-membered carbocyclic aromatic ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen. In another alternative of this embodiment, Z₃ is —R₃₀₀C(═NR₃₀₁)NR₃₀₂R₃₀₃, wherein R₃₀₀ is a 6-membered carbocyclic aromatic ring, and at least two of R₃₀₁, R₃₀₂, R₃₀₃ are ring atoms of a heterocyclic ring. In another alternative of this embodiment, Z₃ is —R₃₀₀C(═NR₃₀₁)NR₃₀₂R₃₀₃, wherein R₃₀₀ is a 6-membered carbocyclic aromatic ring, and at least one of R₃₀₁, R₃₀₂, R₃₀₃ are ring atoms of a heterocyclic ring fused to R₃₀₀.

Yet another embodiment encompasses compounds having any of formulas (1)-(7) wherein Z₃ is a benzamidine derivatized with one or more groups selected from carbonyl, thiocarbonyl, imino, enamino, phosphorus, and sulfur, where the benzamidine derivative hydrolyzes under physiological conditions to form benzamidine. In a further embodiment, Z₃ is a benzamidine derivatized with one or more groups selected from optionally substituted hydrocarbyl, provided that the carbon atom directly bonded to the amidine is sp³ hybridized and aryl, where the benzamidine derivative is oxidized under physiological conditions to form benzamidine. In yet another embodiment, Z₃ is a benzamidine derivatized with one or more heteroatoms selected from oxygen, nitrogen in its most reduced state, and sulfur in its most reduced state, where the benzamidine derivative is reduced under physiological conditions to form benzamidine. In still another embodiment, Z₃ is a benzamidine derivatized with one or more substituents selected from a hydrocarbyl substituted at the beta carbon with carbonyl, sulfonyl, sulfinyl, cyano, nitro and an alkyl, aryl, or heterocyclic group substituted with oxygen, nitrogen, or sulfur at the carbon directly bonded to the amidine group, where the benzamidine derivative undergoes elimination at physiological conditions to form benzamidine.

In a further embodiment for compounds having any of formulas (1)-(7), Z₃ corresponds to formula (c):

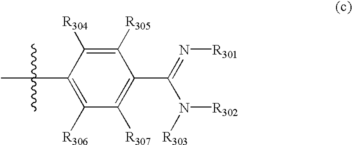

wherein:

R₃₀₁, R₃₀₂, and R₃₀₃ are independently selected from the group consisting of:

(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocylo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,

(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,

(iii) hydrogen, —OR_{b}, —SR_{b}, —NR_{b}, or —N(R_{b})₂, wherein each R_{b} is independently optionally substituted hydrocarbyl, and heterocylo, and

(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen;

R₃₀₄ is selected from the group consisting of halogen, hydrogen, hydroxyl, alkyl, sulfhydryl, alkoxy, and alkylthio;

R₃₀₅ is selected from the group consisting of oxygen, sulfur, halogen, hydrogen, hydroxyl, alkyl, sulfhydryl, alkoxy, and alkylthio;

R₃₀₆ is selected from the group consisting of halogen, hydrogen, hydroxyl, alkyl, sulfhydryl, alkoxy, and alkylthio; and

R₃₀₇ is selected from the group consisting of oxygen, sulfur, halogen, hydrogen, hydroxyl, alkyl, sulfhydryl, alkoxy, and alkylthio, wherein R₃₀₁, R₃₀₂, R₃₀₃, R₃₀₄, R₃₀₅, R₃₀₆ and R₃₀₇ are as defined below for each prodrug conversion mechanism.

In one embodiment, the benzamidine derivative is hydrolyzed under physiological conditions to form benzamidine when Z₃ is a benzamidine derivative having formula (c) and R₃₀₁, R₃₀₂, and R₃₀₃ are independently selected from hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocylo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl.

In a further embodiment, the benzamidine derivative is oxidized under physiological conditions to form benzamidine when Z₃ is a benzamidine derivative having formula (c) and R₃₀₁, R₃₀₂, and R₃₀₃ are independently selected from hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl.

In still another embodiment, the benzamidine derivative is reduced under physiological conditions to form benzamidine when Z₃ is a benzamidine derivative having formula (c) and R₃₀₁, R₃₀₂, and R₃₀₃ are independently selected from hydrogen, —OR_{b}, —SR_{b}, —NR_{b}, or —N(R_{b})₂, wherein each R_{b} is independently optionally substituted hydrocarbyl, and heterocylo.

In an alternative embodiment, the benzamidine derivative undergoes elimination at physiological conditions to form benzamidine when Z₃ is a benzamidine derivative having formula (c) and R₃₀₁, R₃₀₂, and R₃₀₃ are independently selected from hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group.

In a particularly preferred embodiment, the compound represented by any of formulas(1)-(7) is selected from the group of compounds illustrated in Table 1 below. Certain compounds listed in Table 1 are pharmaceutically acceptable salts of compounds having any of formulas (1)-(7). Some of the salts are depicted as the chemical formula with the respective compound. For example, compound 1 has 2 molecules of CF₃COOH salt per molecule of compound 1. Other salts are depicted as the structural formula with the respective compound. For example, compound 119 has 2.3 molecules of CF₃COOH salt per molecule of compound 119. For each compound listed in Table 1, the compound number corresponds to the example number.

TABLE 1

| Com- | |
|---|---|
| pound | |
| No. | Compound |
| 1. | 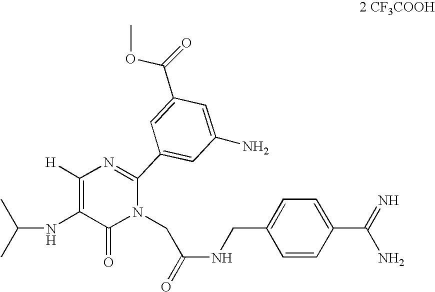 |
| 2. | 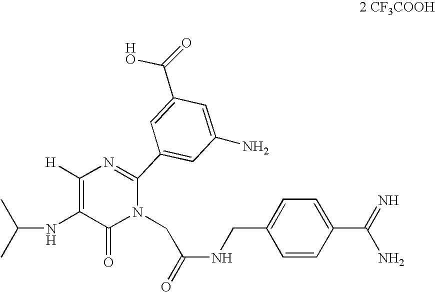 |
| 3. | 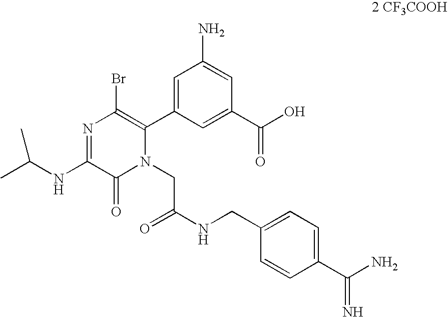 |
| 4. | 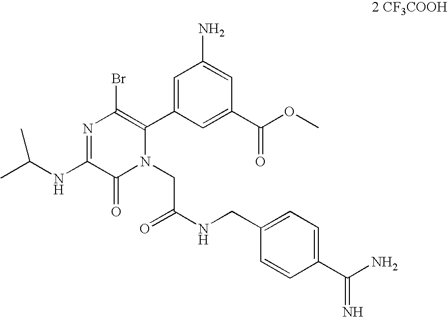 |
| 5. | 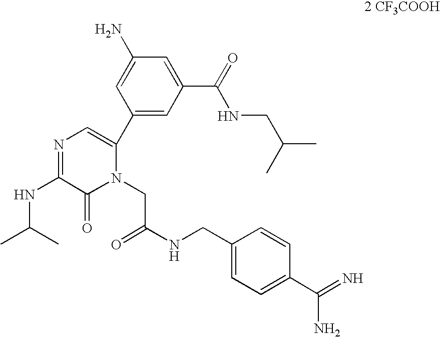 |
| 6. | 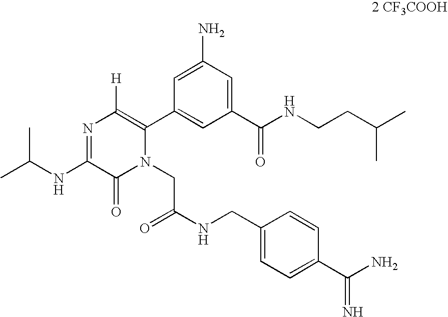 |
| 7. | 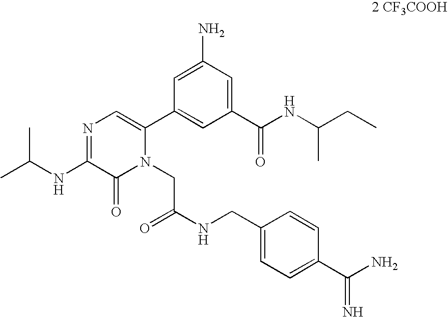 |
| 8. | 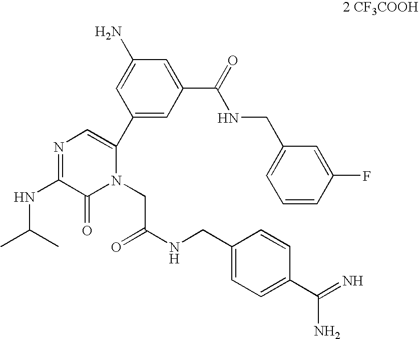 |
| 9. | 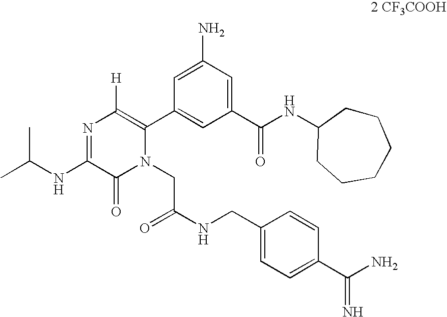 |
| 10. | 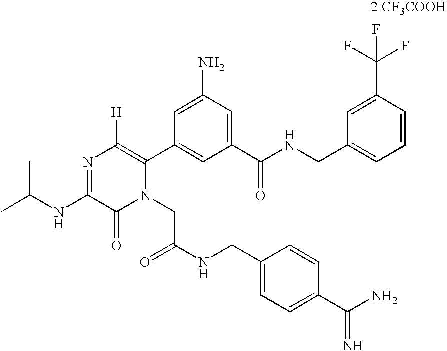 |
| 11. | 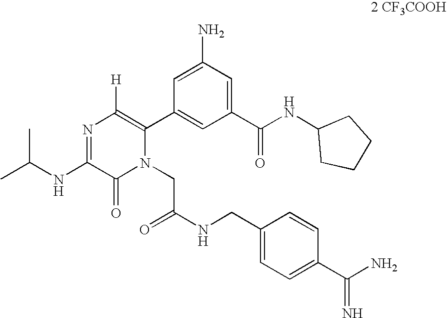 |
| 12. | 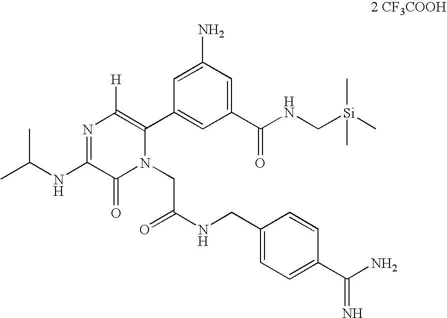 |
| 13. | 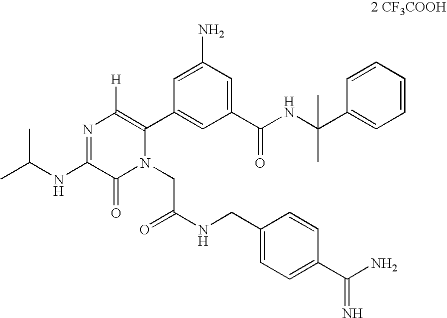 |
| 14. | 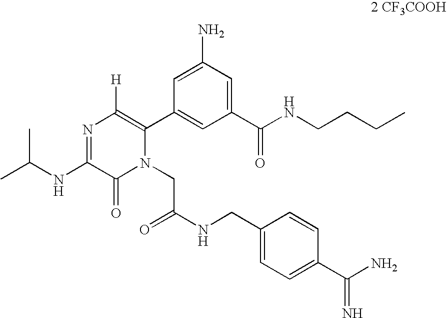 |
| 15. | 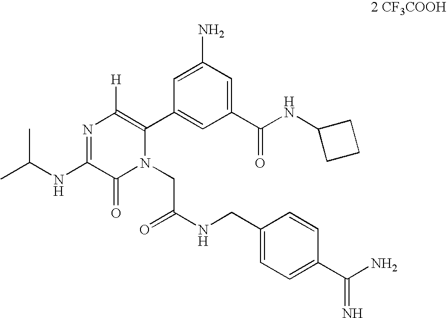 |
| 16. | 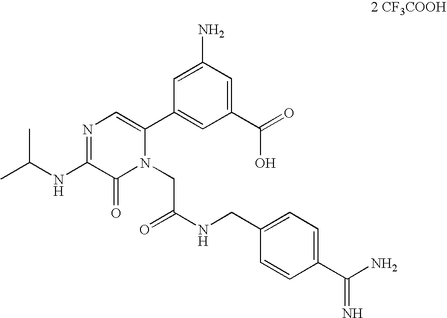 |
| 17. | 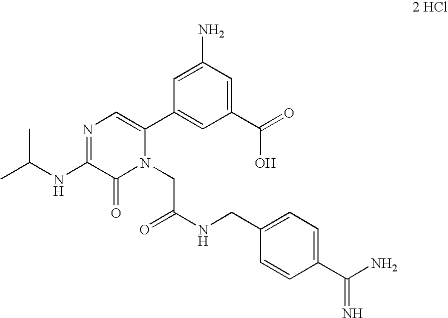 |
| 18. | 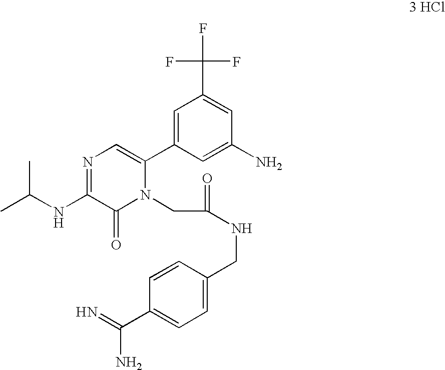 |
| 19. | 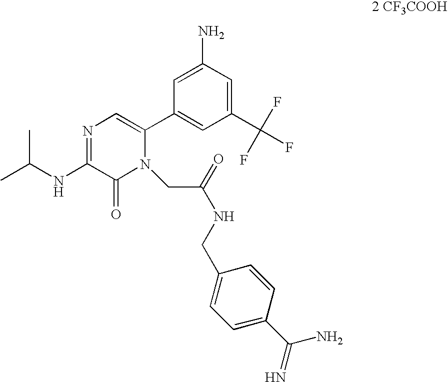 |
| 20. | 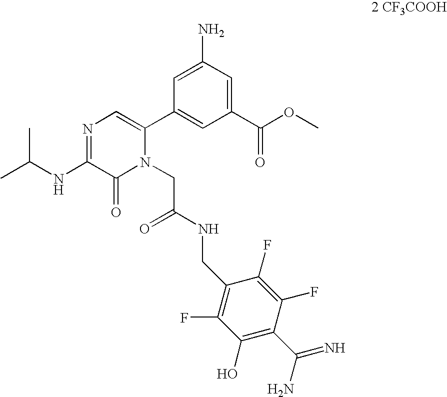 |
| 21. | 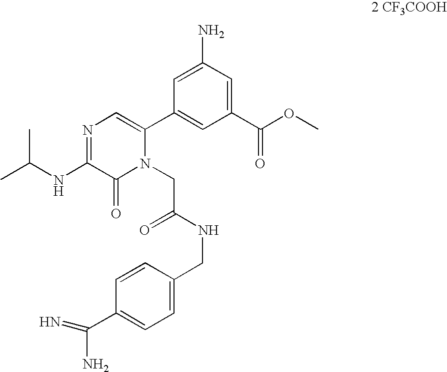 |
| 22. | 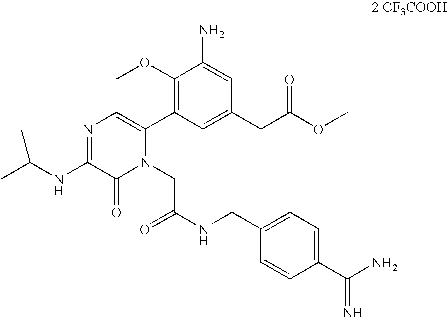 |
| 23. | 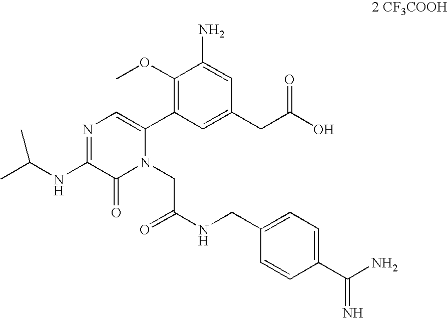 |
| 24. | 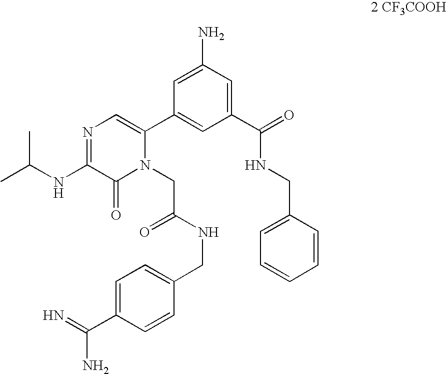 |
| 25. | 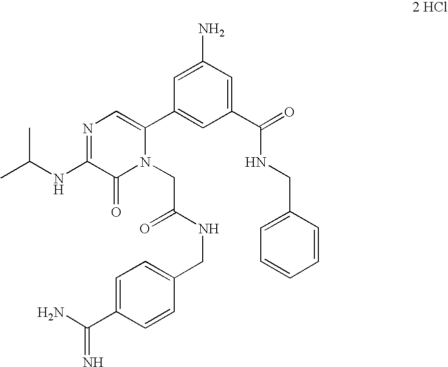 |
| 26. | 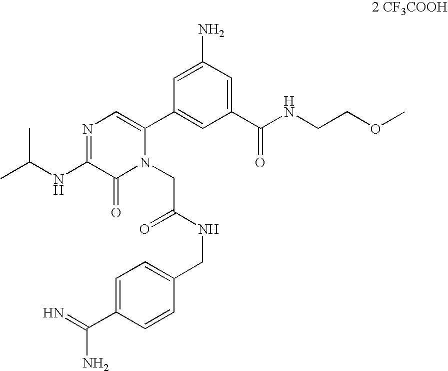 |
| 27. | 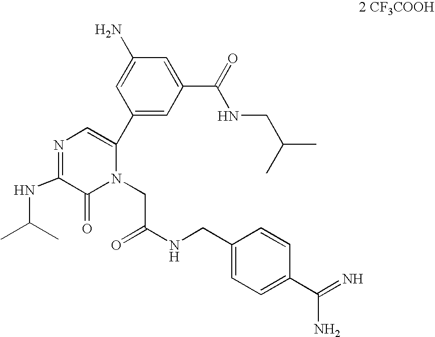 |
| 28. | 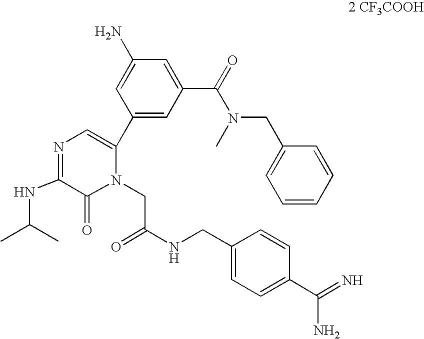 |
| 29. | 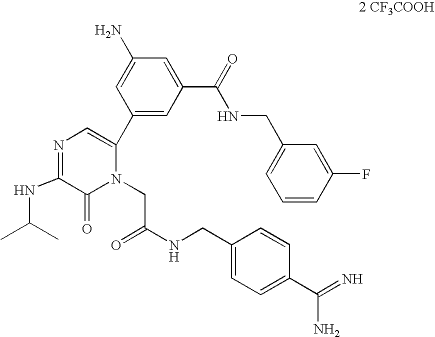 |
| 30. | 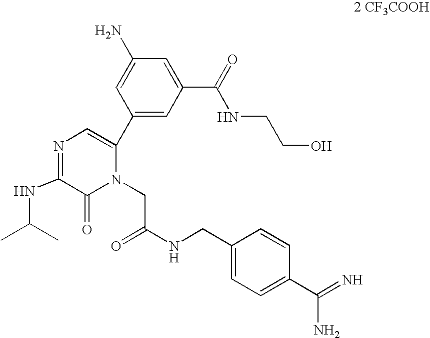 |
| 31. | 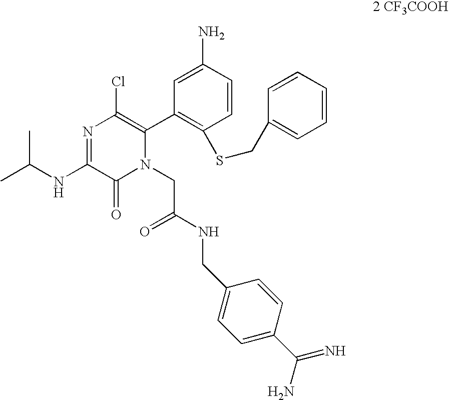 |
| 32. | 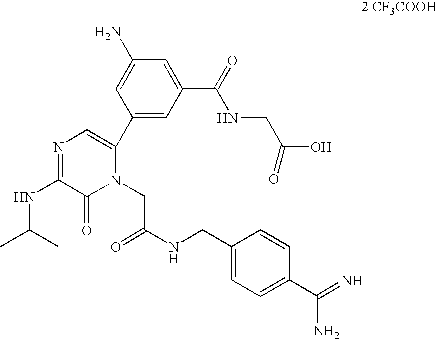 |
| 33. | 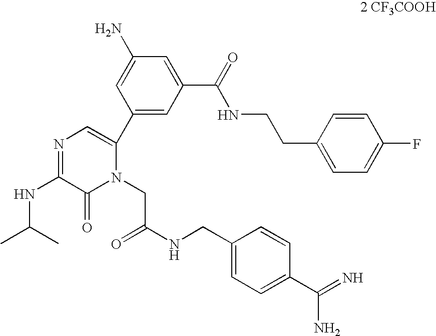 |
| 34. | 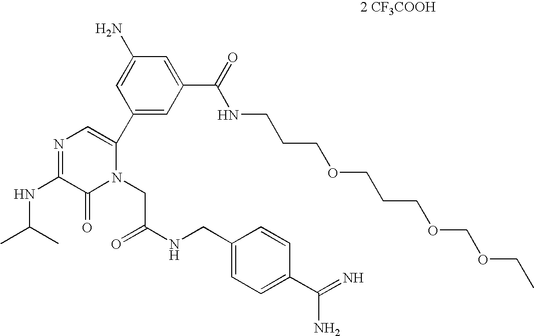 |
| 35. | 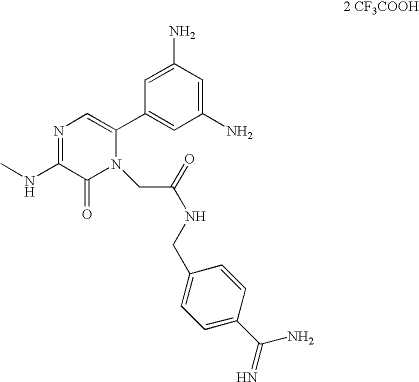 |
| 36. | 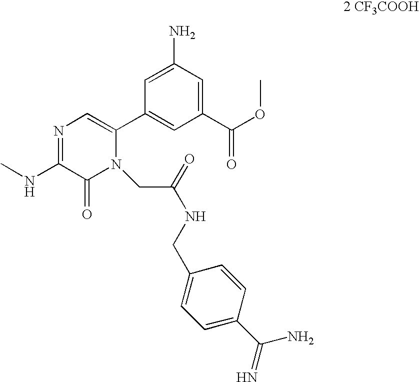 |
| 37. | 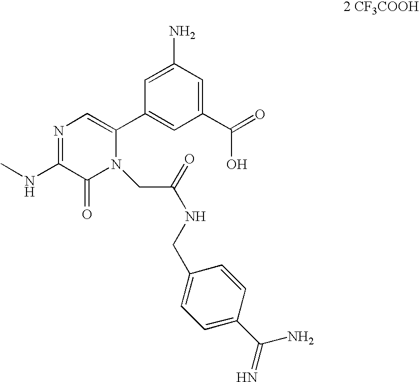 |
| 38. | 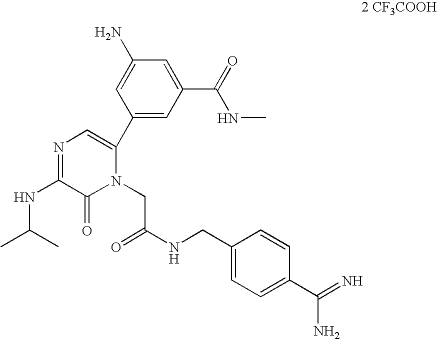 |
| 39. | 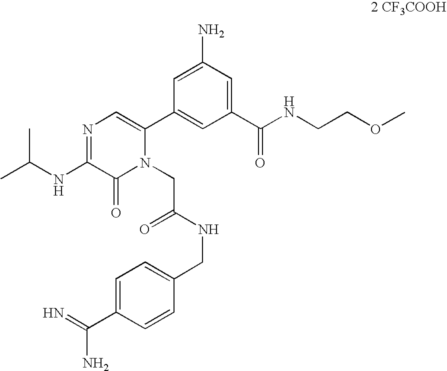 |
| 40. | 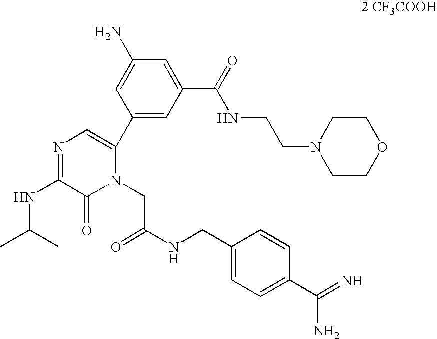 |
| 41. | 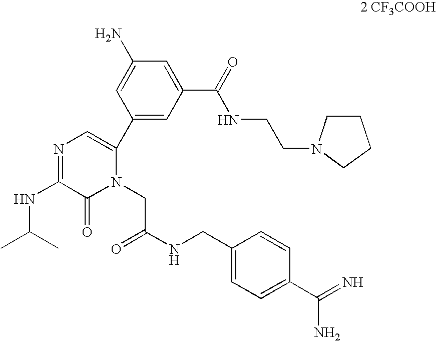 |
| 42. | 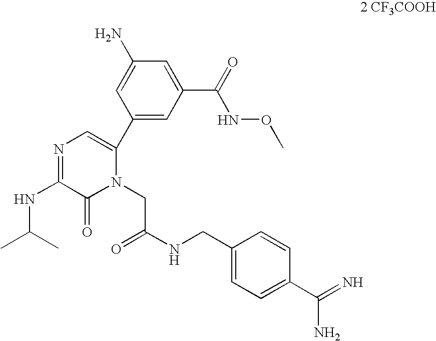 |
| 43. | 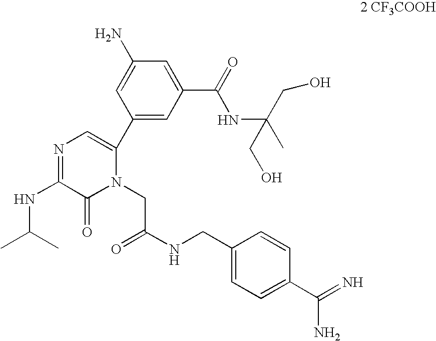 |
| 44. | 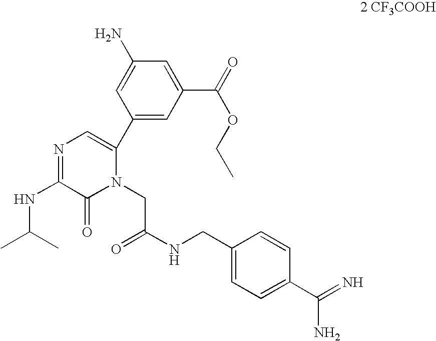 |
| 45. | 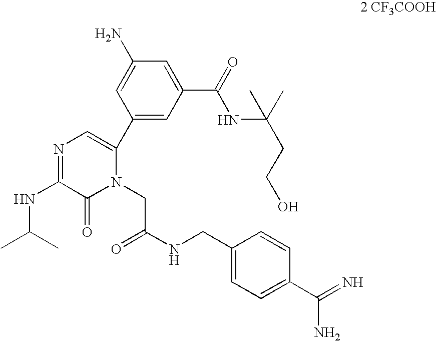 |
| 46. | 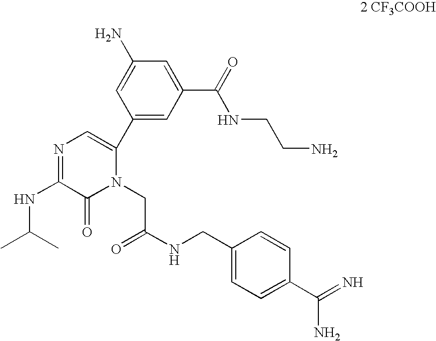 |
| 47. | 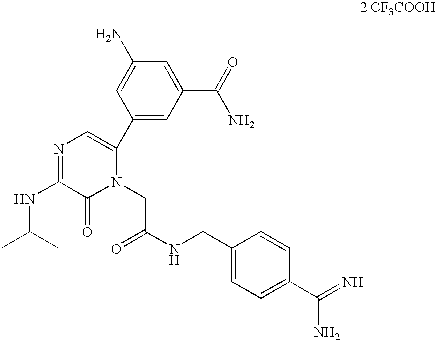 |
| 48. | 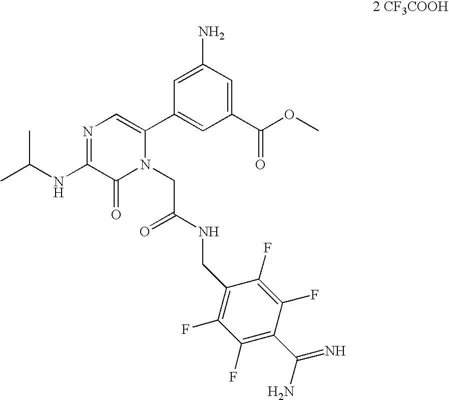 |
| 49. | 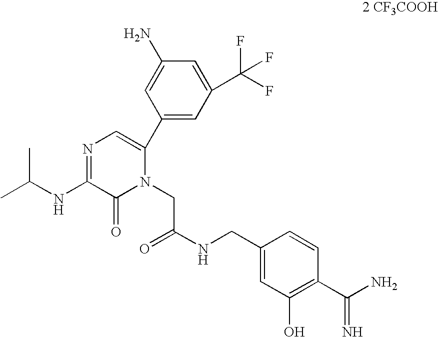 |
| 50. | 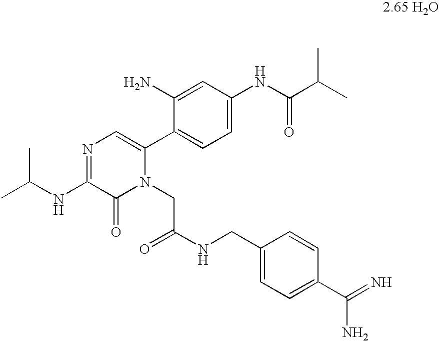 |
| 51. | 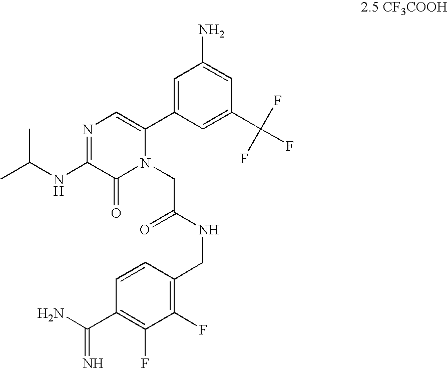 |
| 52. | 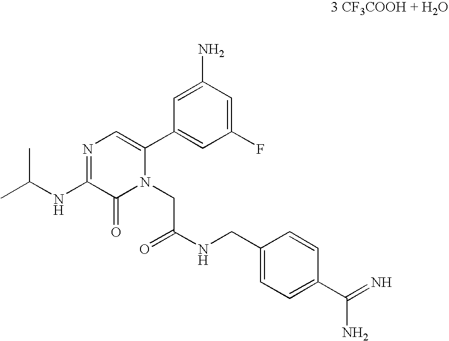 |
| 53. | 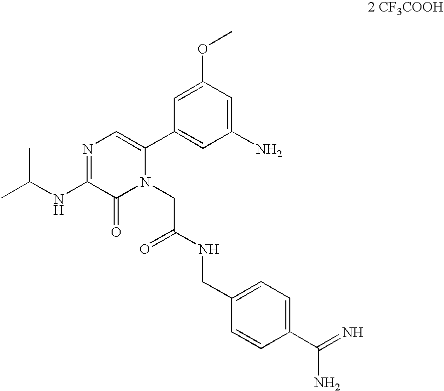 |
| 54. | 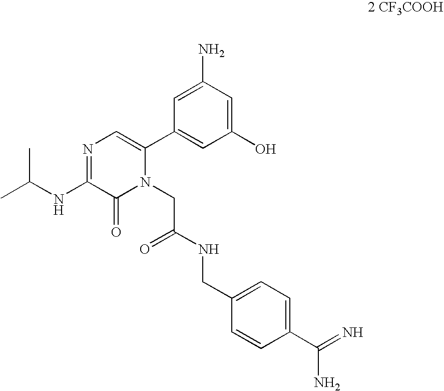 |
| 55. | 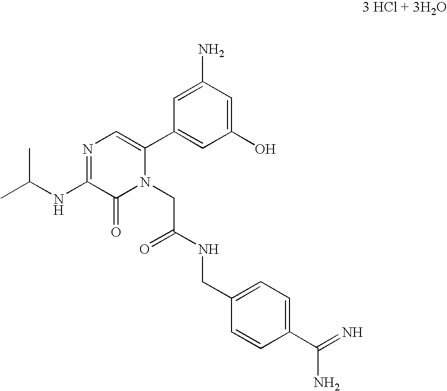 |
| 56. | 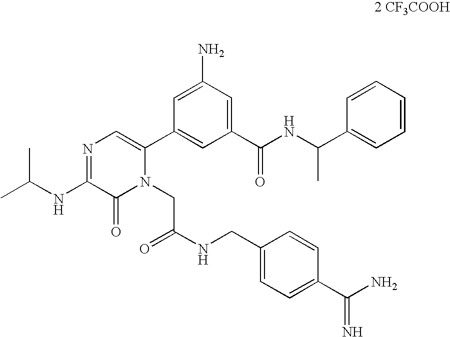 |
| 57. | 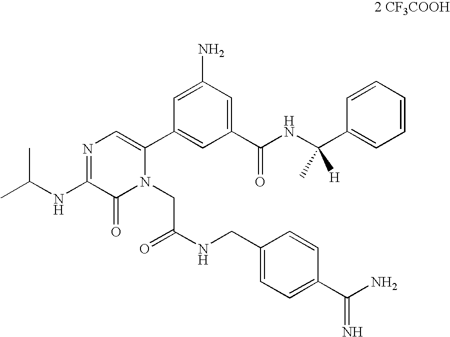 |
| 58. | 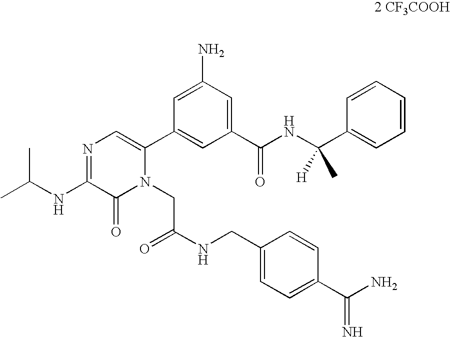 |
| 59. | 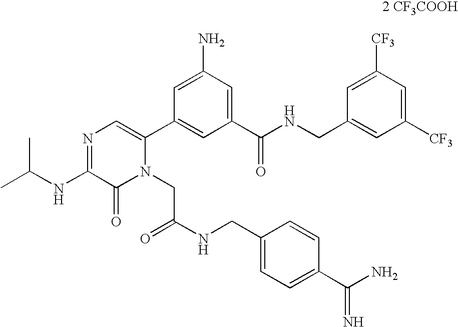 |
| 60. | 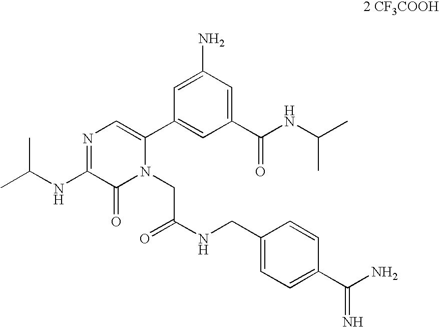 |
| 61. | 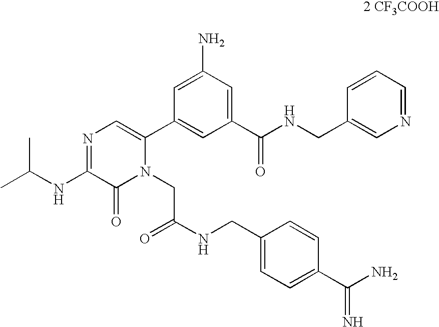 |
| 62. | 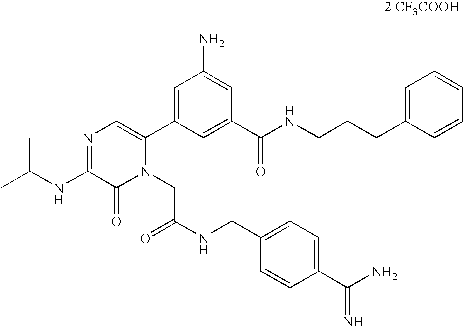 |
| 63. | 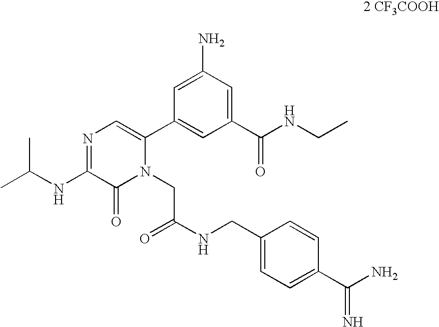 |
| 64. | 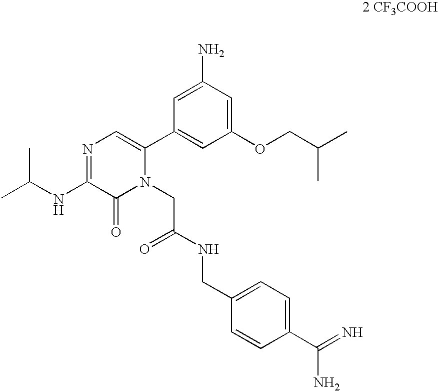 |
| 65. | 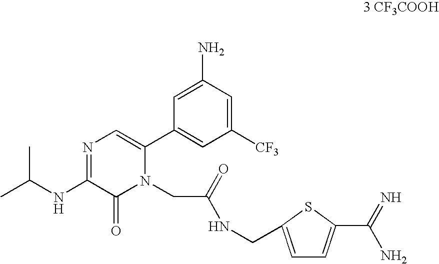 |
| 66. | 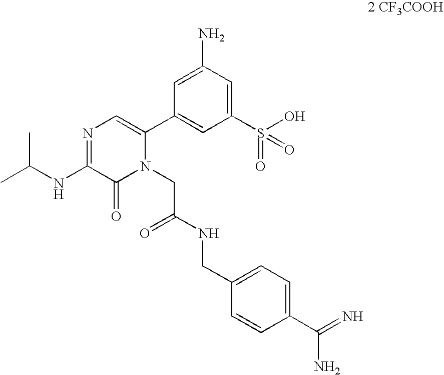 |
| 67. | 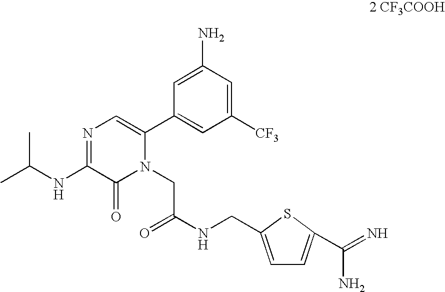 |
| 68. | 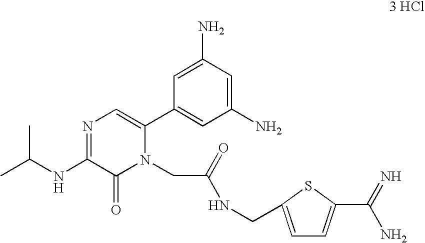 |
| 69. | 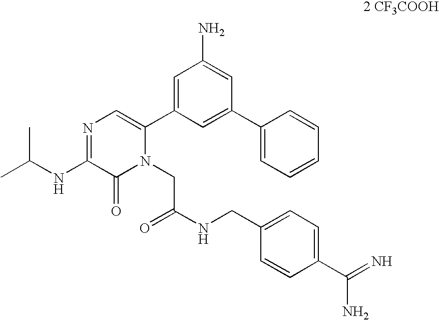 |
| 70. | 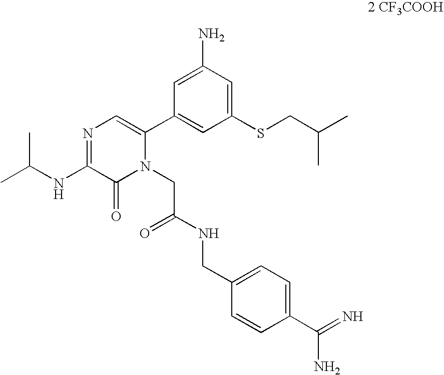 |
| 71. | 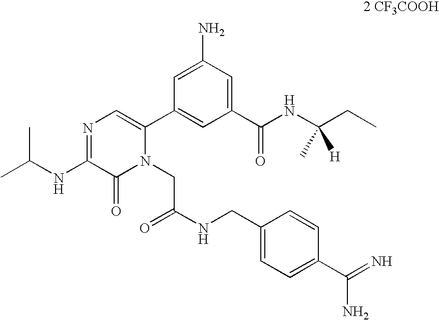 |
| 72. | 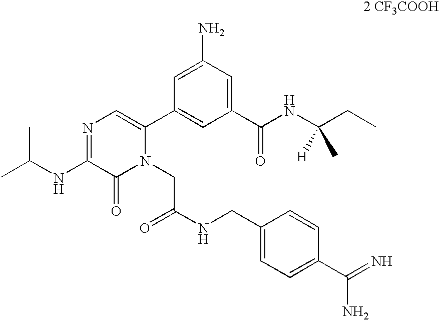 |
| 73. | 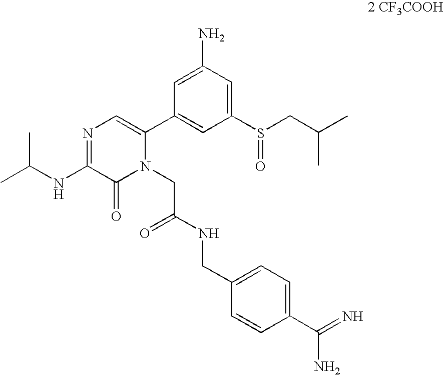 |
| 74. | 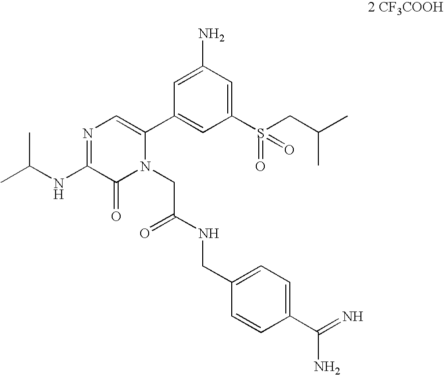 |
| 75. | 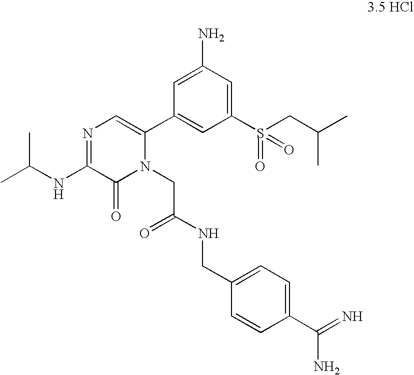 |
| 76. | 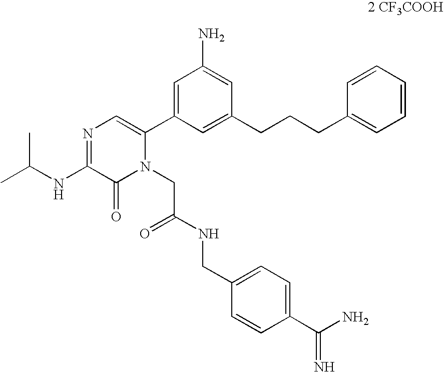 |
| 77. | 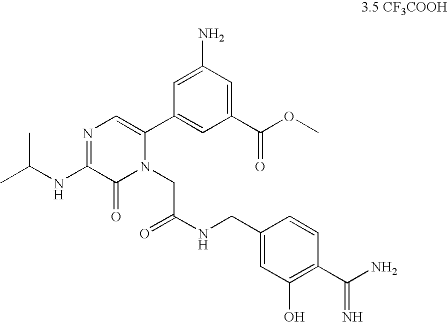 |
| 78. | 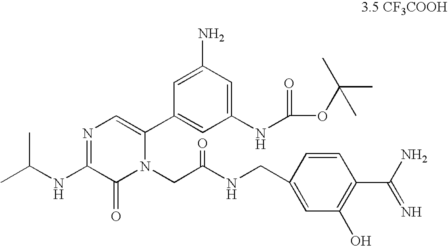 |
| 79. | 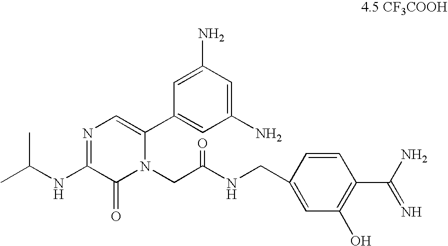 |
| 80. | 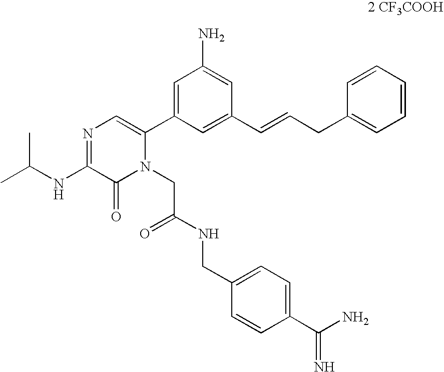 |
| 81. | 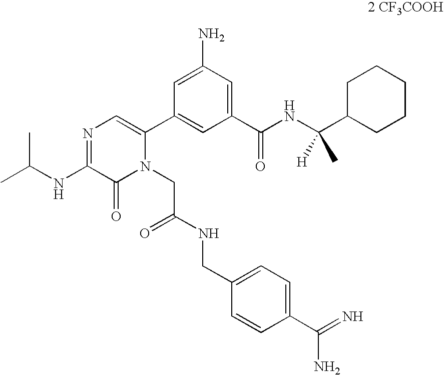 |
| 82. | 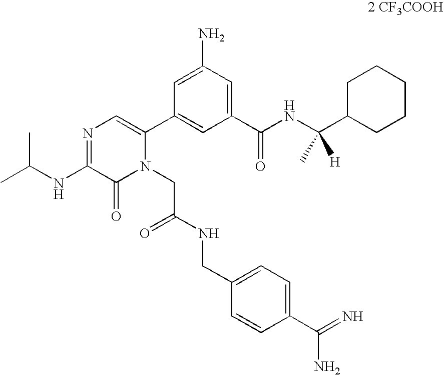 |
| 83. | 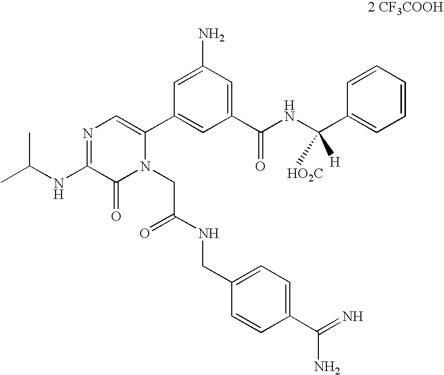 |
| 84. | 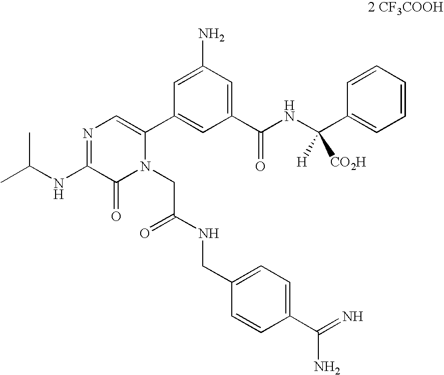 |
| 85. | 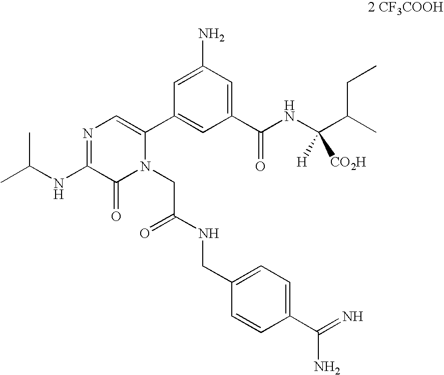 |
| 86. | 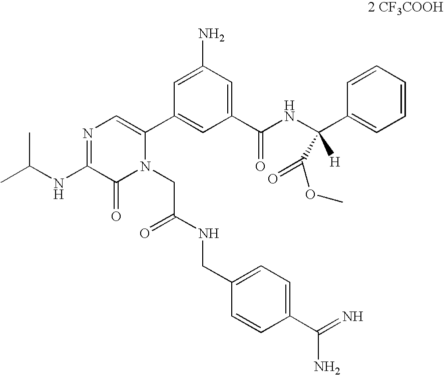 |
| 87. | 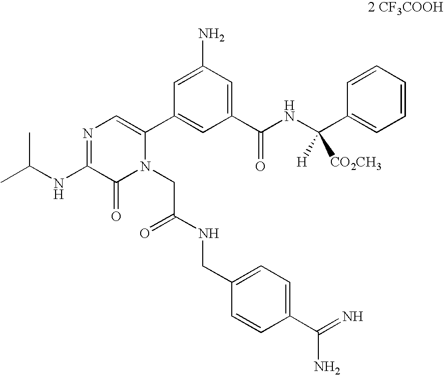 |
| 88. | 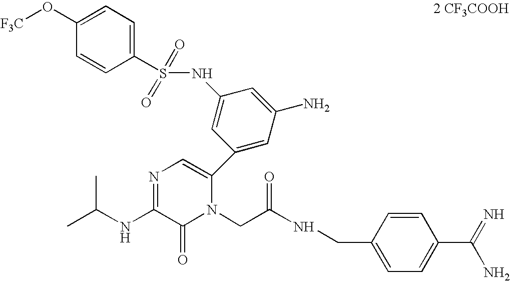 |
| 89. | 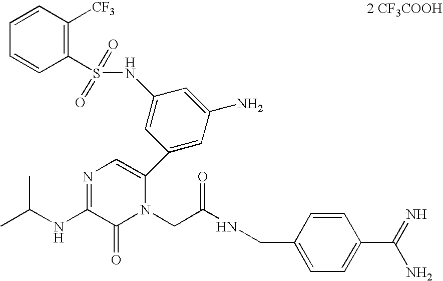 |
| 90. | 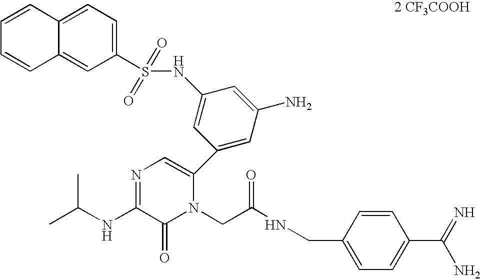 |
| 91. | 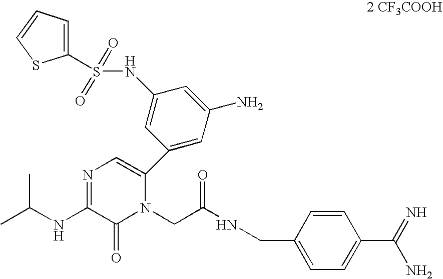 |
| 92. | 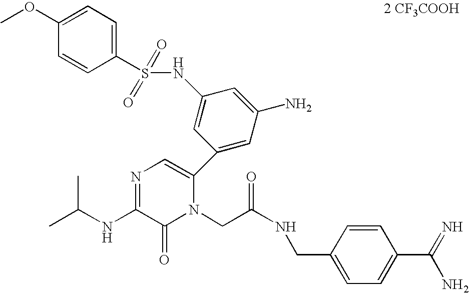 |
| 93. | 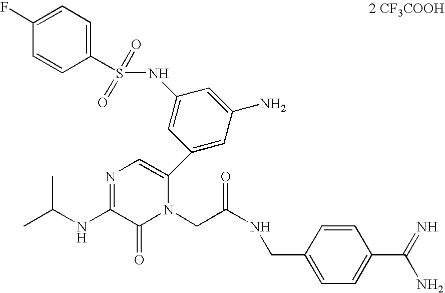 |
| 94. | 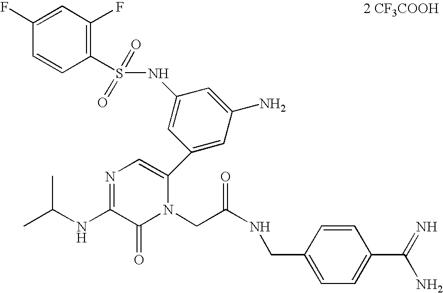 |
| 95. | 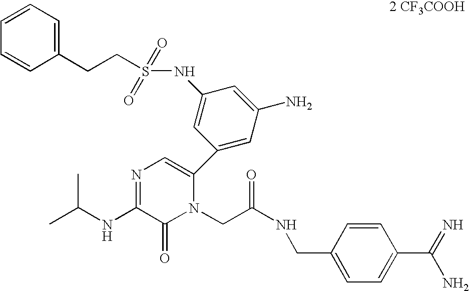 |
| 96. | 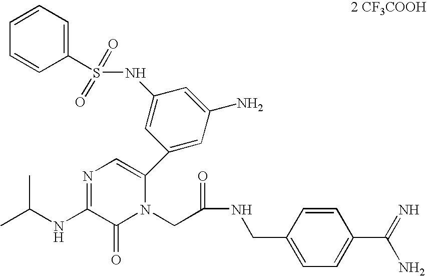 |
| 97. | 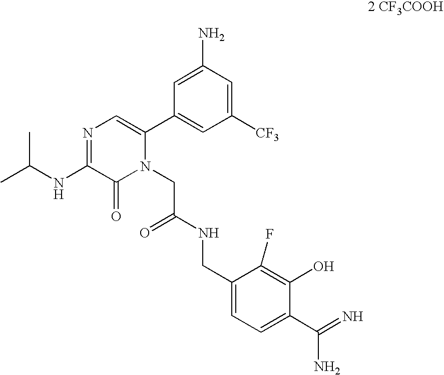 |
| 98. | 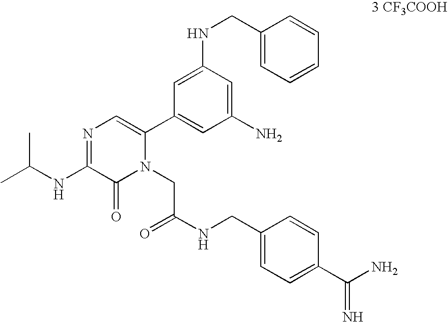 |
| 99. | 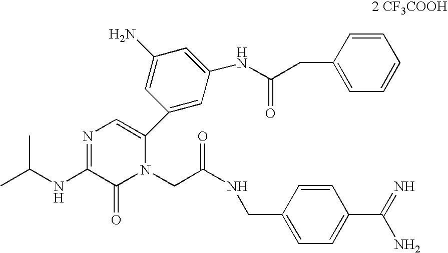 |
| 100. | 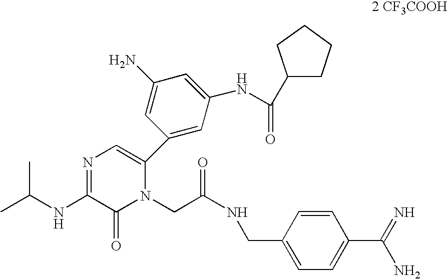 |
| 101. | 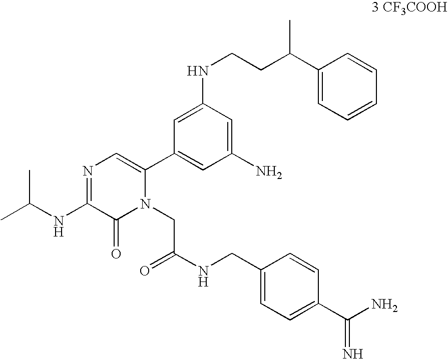 |
| 102. | 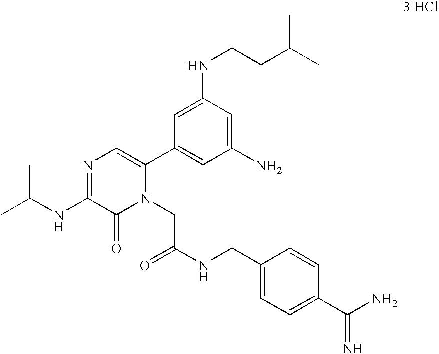 |
| 103. | 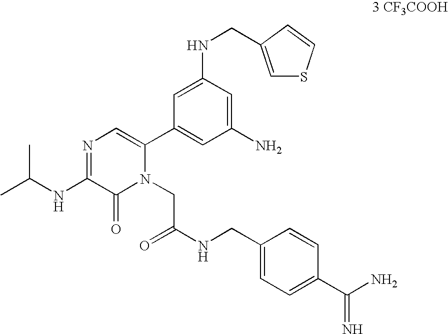 |
| 104. | 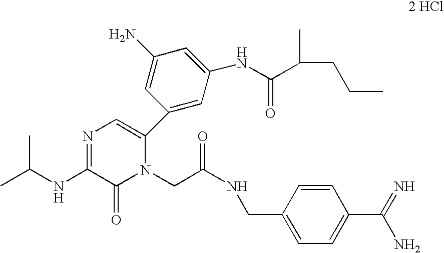 |
| 105. | 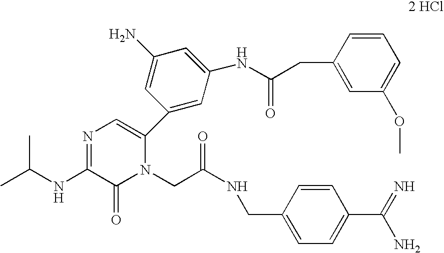 |
| 106. | 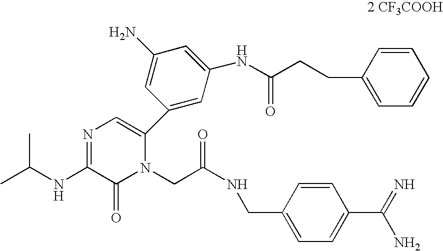 |
| 107. | 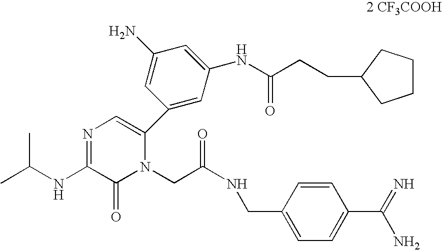 |
| 108. | 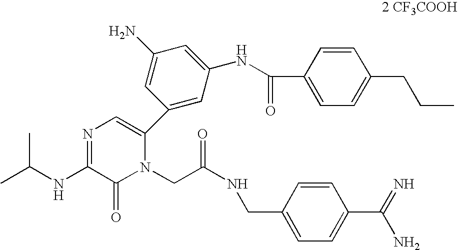 |
| 109. | 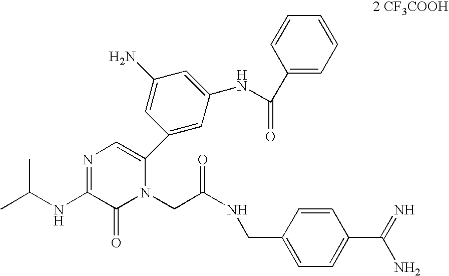 |
| 110. | 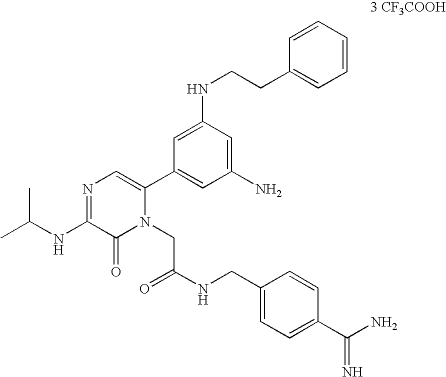 |
| 111. | 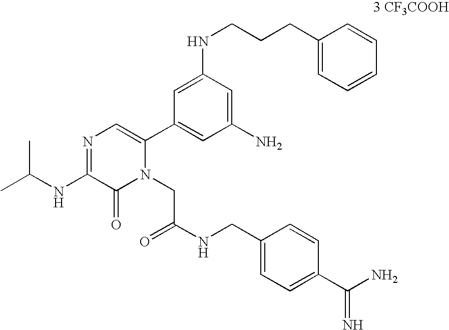 |
| 112. | 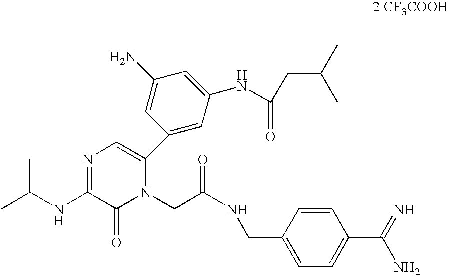 |
| 113. | 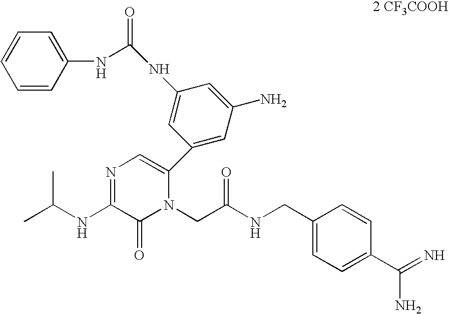 |
| 114. | 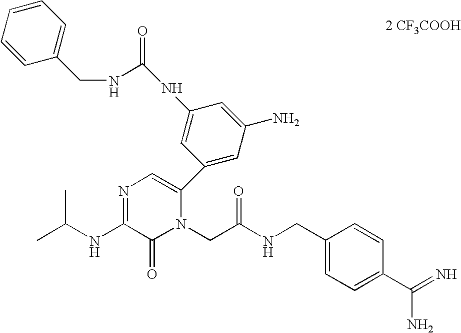 |
| 115. | 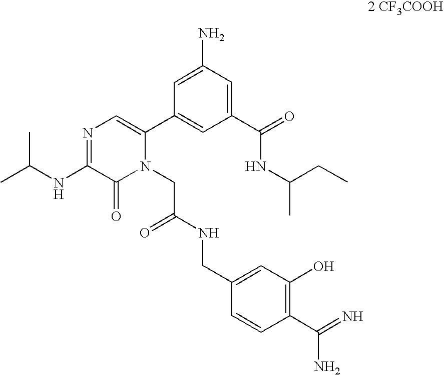 |
| 116. | 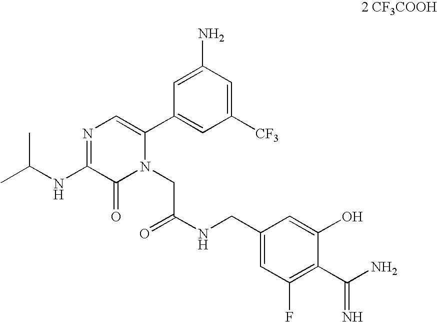 |
| 117. | 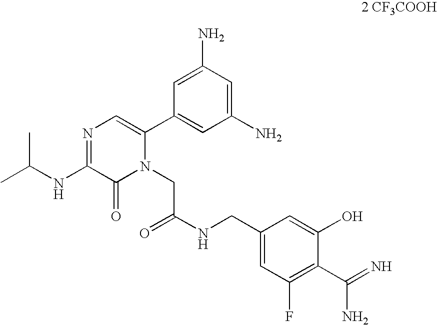 |
| 118. | 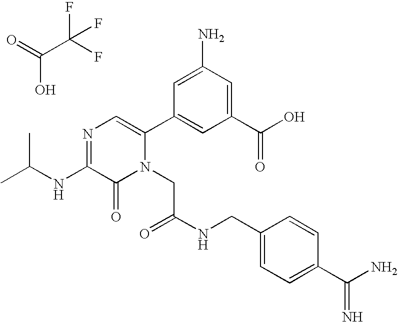 |
| 119. | 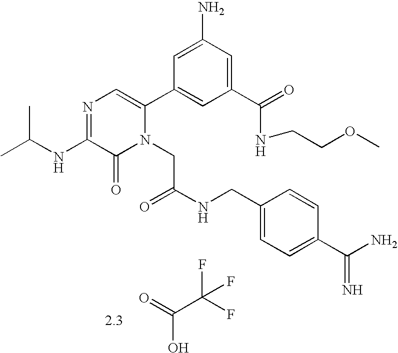 |
| 120. | 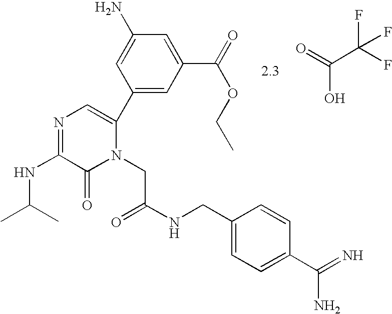 |
| 121. | 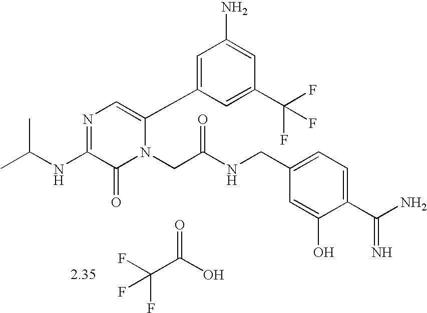 |
| 122. | 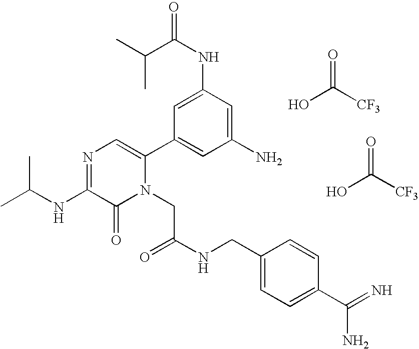 |
| 123. | 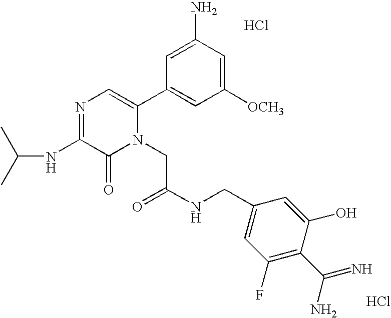 |
| 124. | 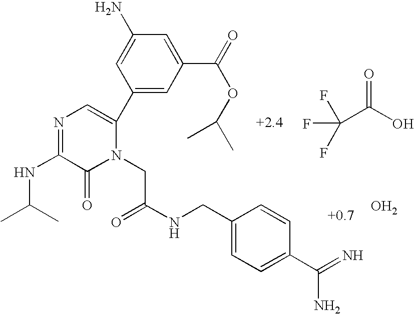 |
| 125. | 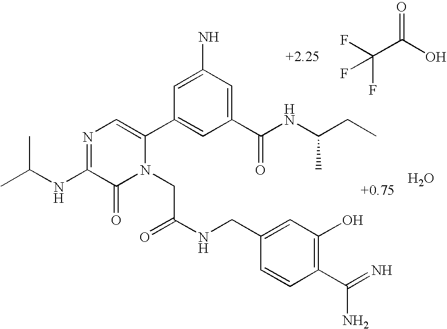 |
| 126. | 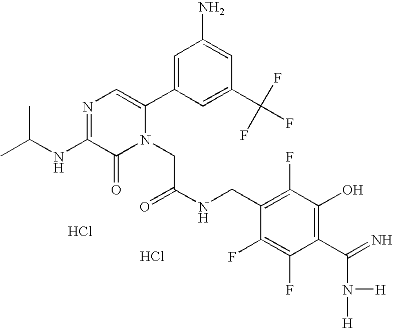 |
| 127. | 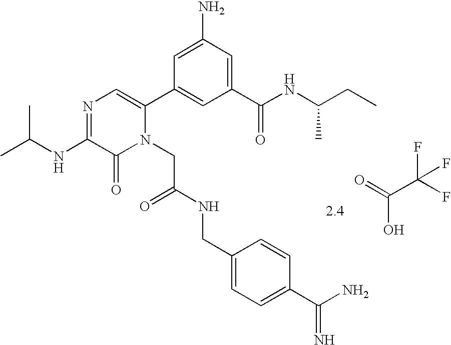 |
| 128. | 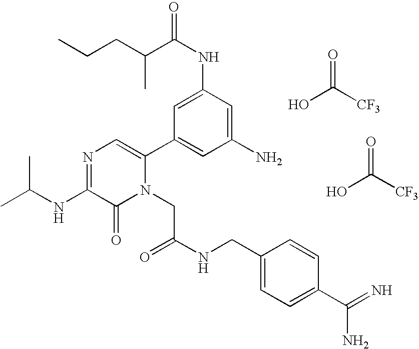 |
| 129. | 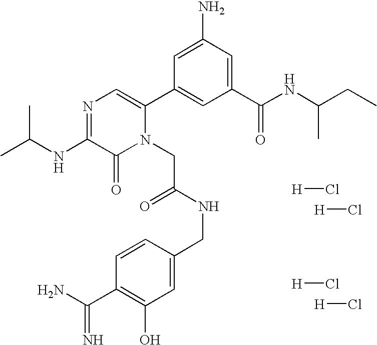 |
| 130. | 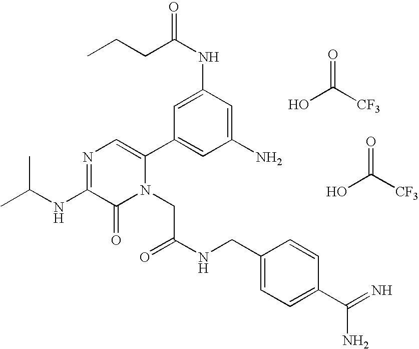 |
| 131. | 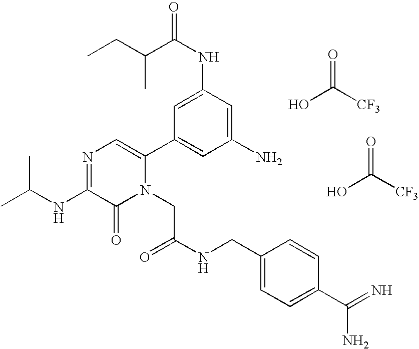 |
| 132. | 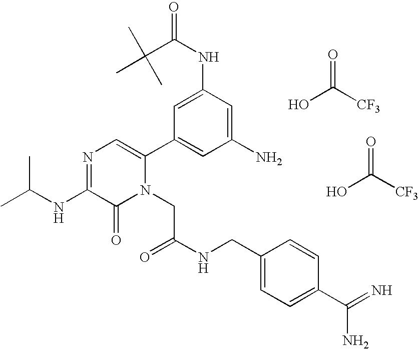 |
| 133. | 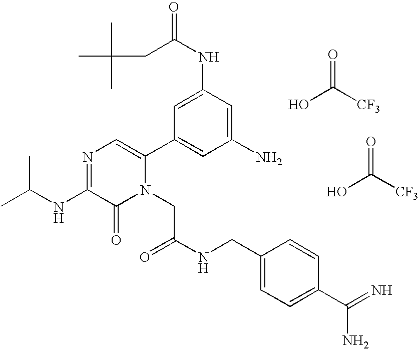 |
| 134. | 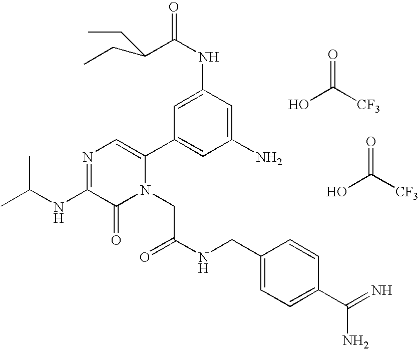 |
| 135. | 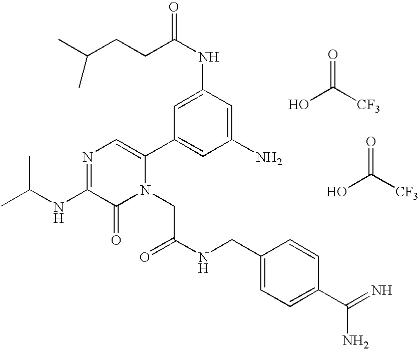 |
| 136. | 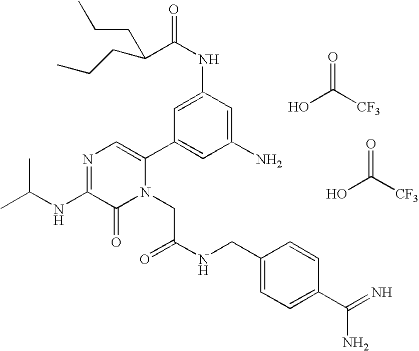 |
| 137. | 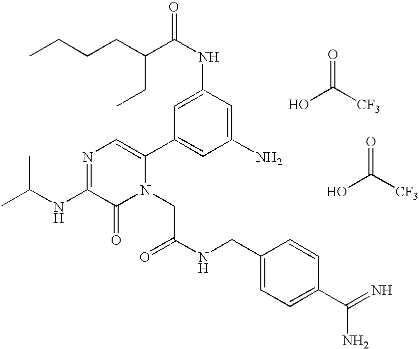 |
| 138. | 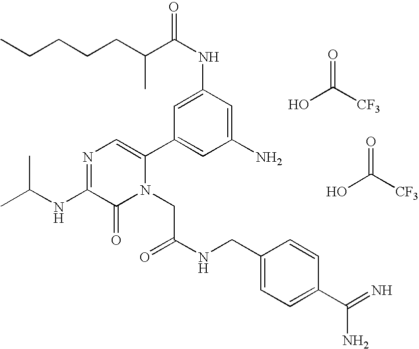 |
| 139. | 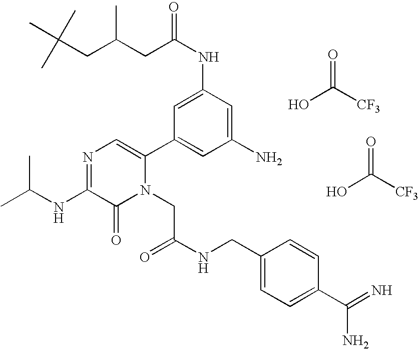 |
| 140. | 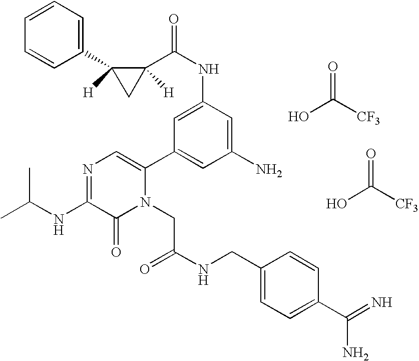 |
| 141. | 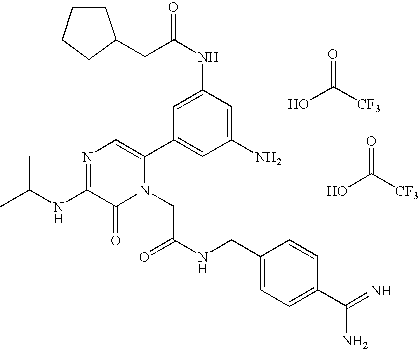 |
| 142. | 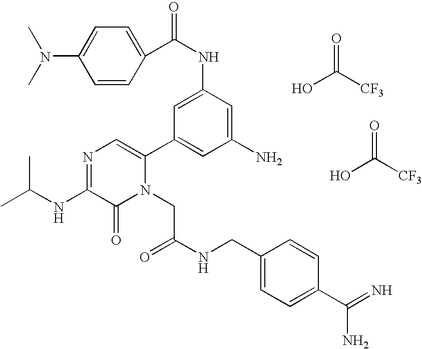 |
| 143. | 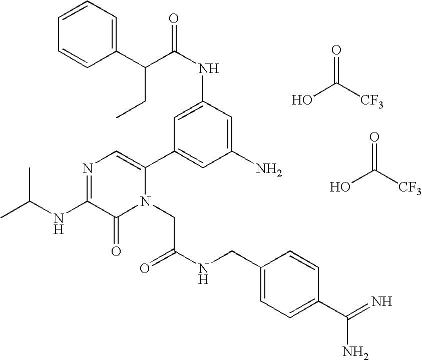 |
| 144. | 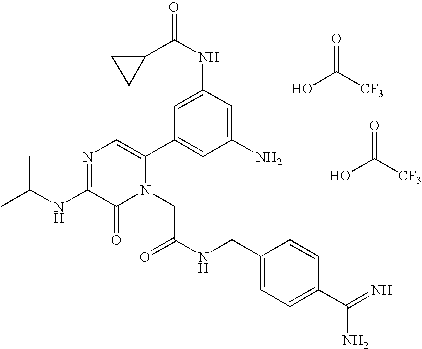 |
| 145. | 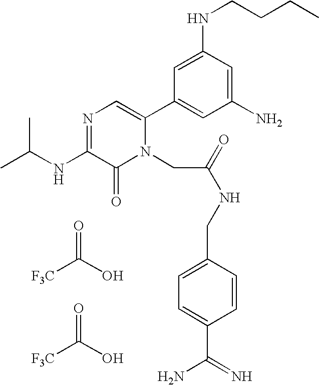 |
| 146. | 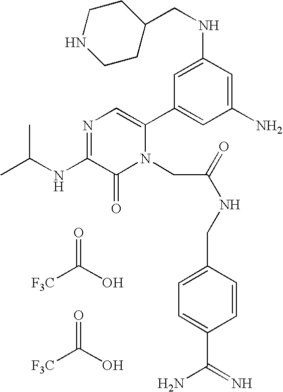 |
| 147. | 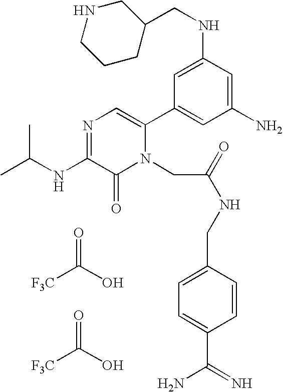 |
| 148. | 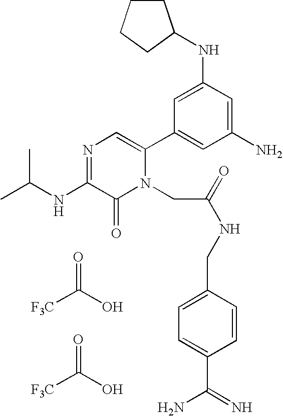 |
| 149. | 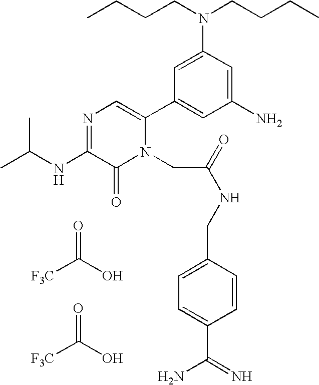 |
| 150. | 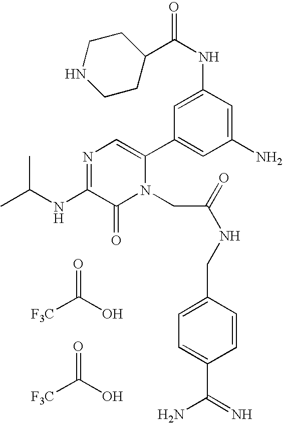 |
| 151. | 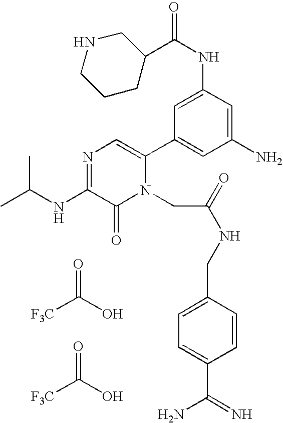 |
| 152. | 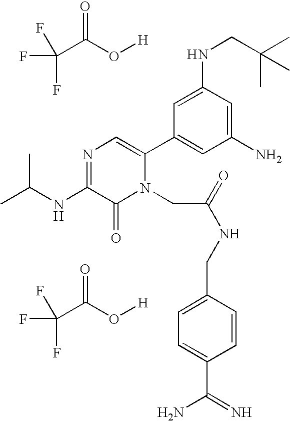 |
| 153. | 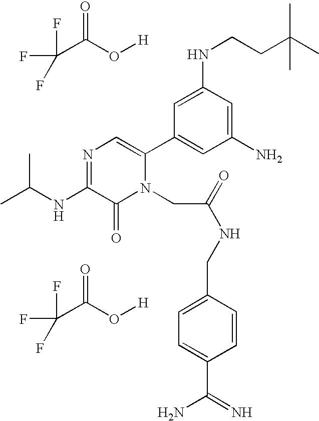 |
| 154. | 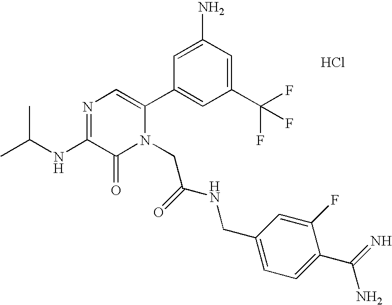 |
| 155. | 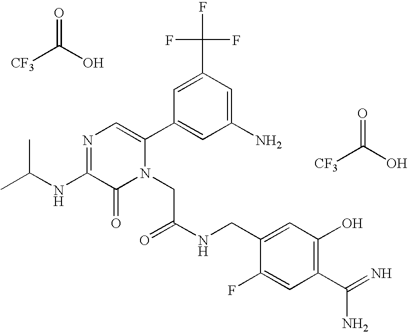 |
| 156. | 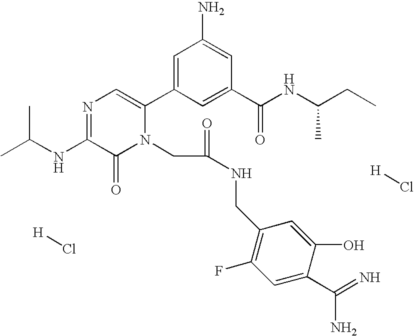 |
| 157. | 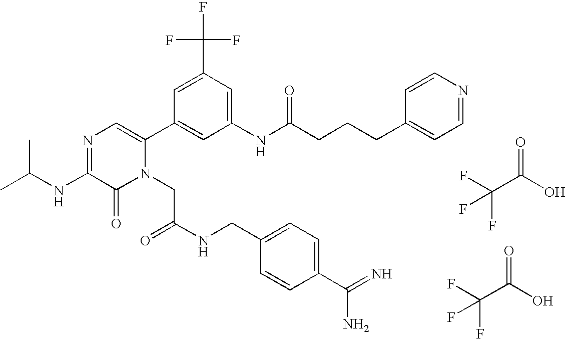 |
| 158. | 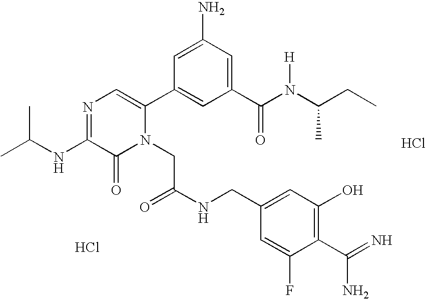 |
| 159. | 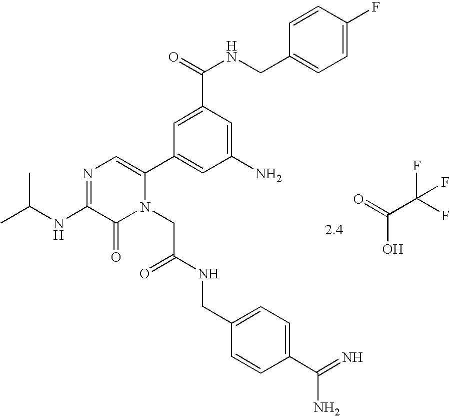 |
| 160. | 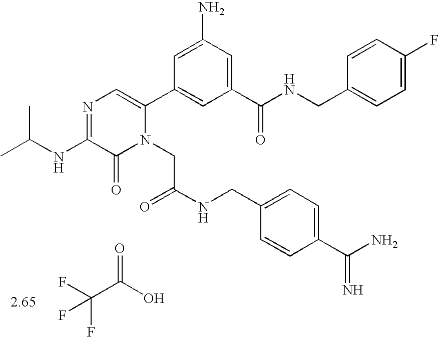 |
| 161. | 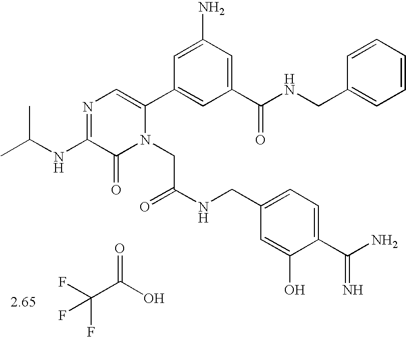 |
| 162. | 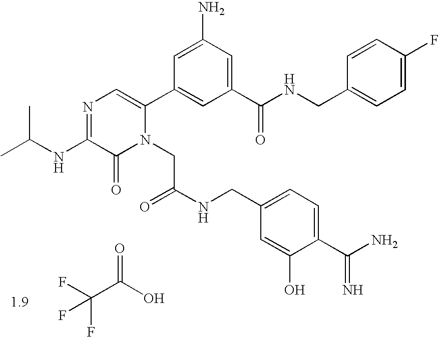 |
| 163. | 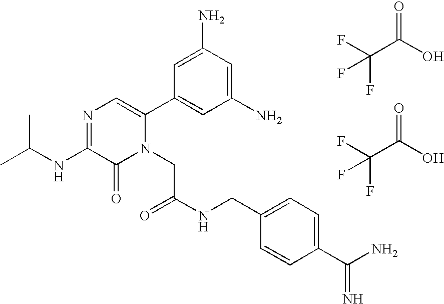 |
| 164. | 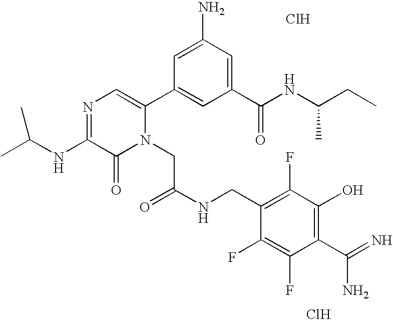 |
| 165. | 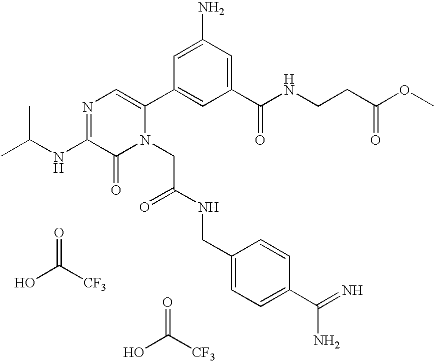 |
| 166. | 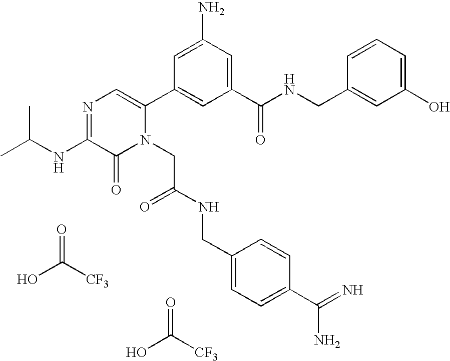 |
| 167. | 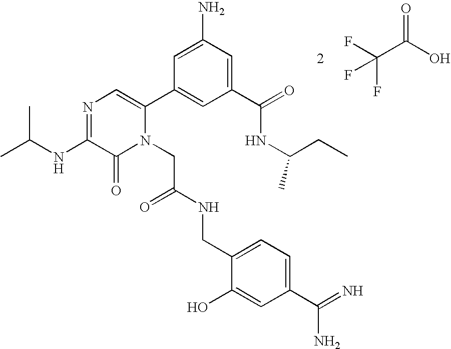 |
| 168. | 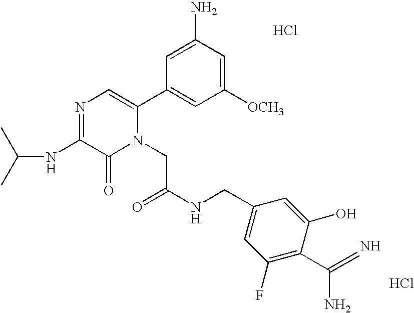 |
| 169. | 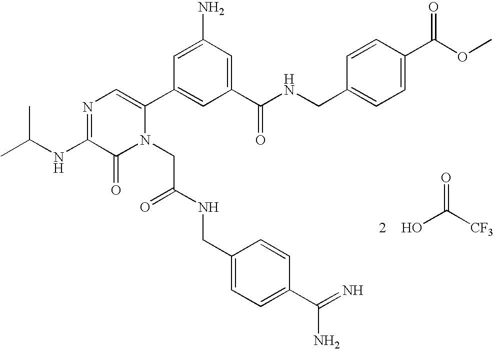 |
| 170. | 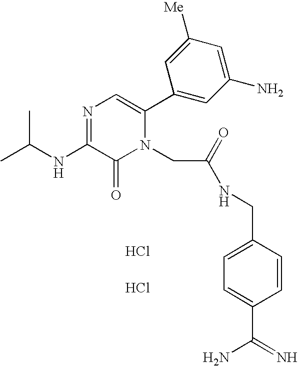 |
| 171. | 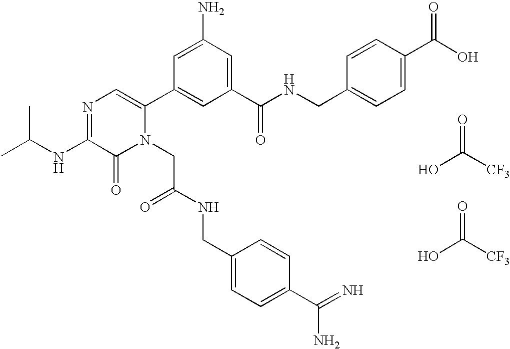 |
| 172. | 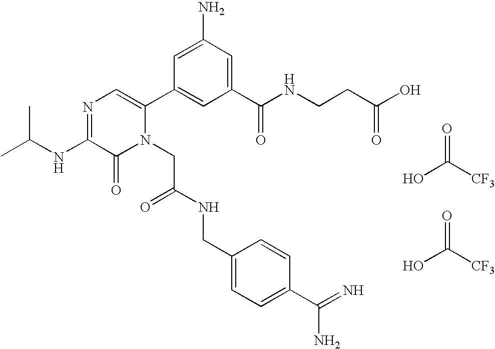 |
| 173. | 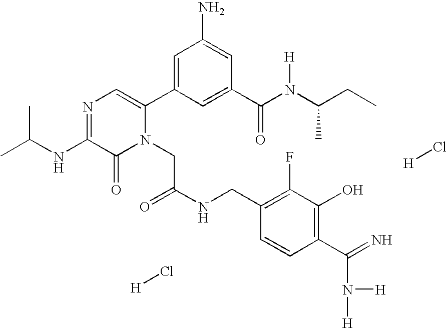 |
| 174. | 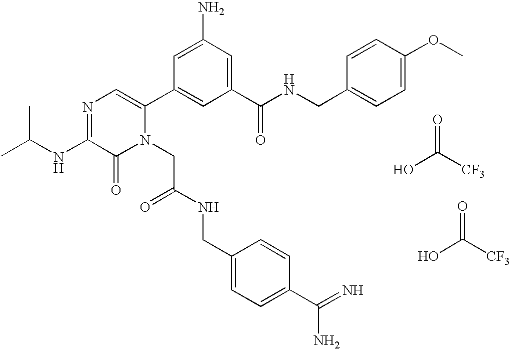 |
| 175. | 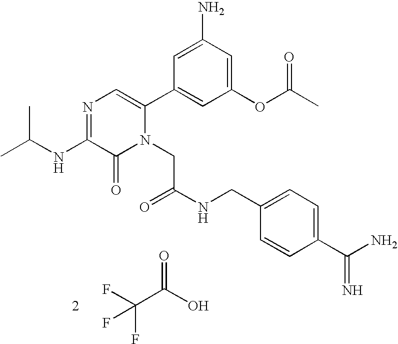 |
| 176. | 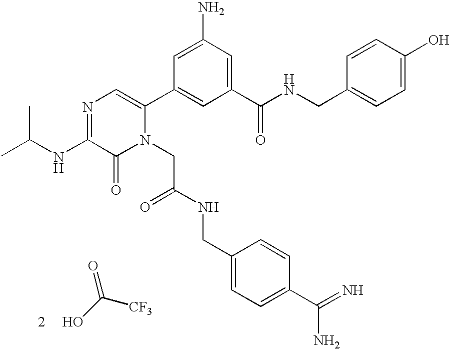 |
| 177. | 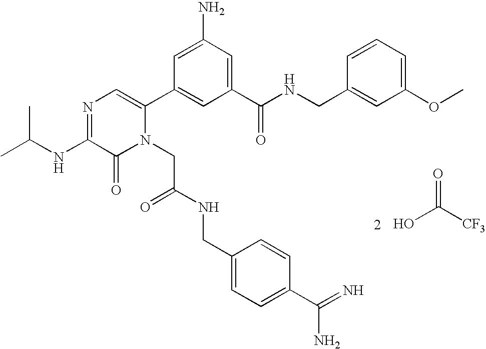 |
| 178. | 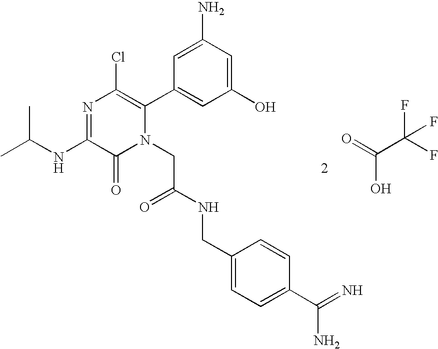 |
| 179. | 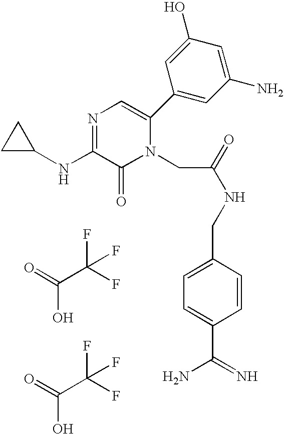 |
| 180. | 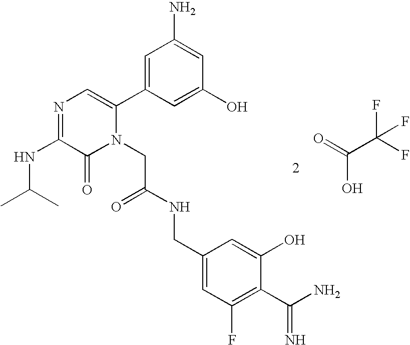 |
| 181. | 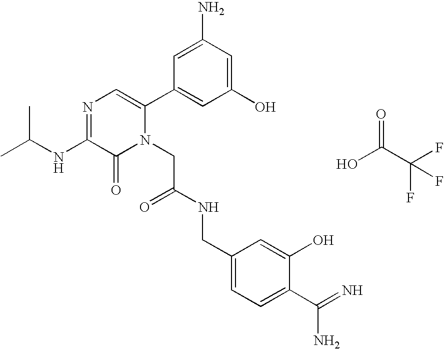 |
| 182. | 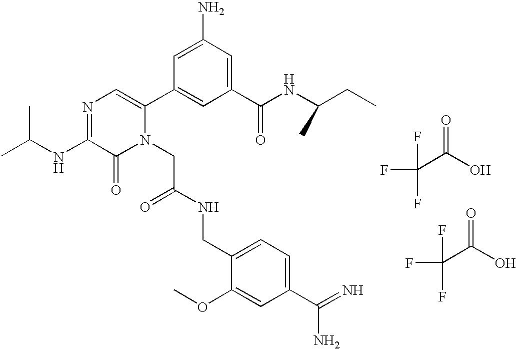 |
| 183. | 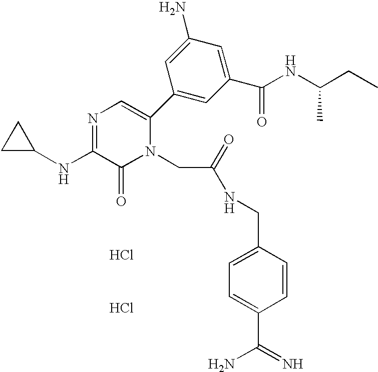 |
| 184. | 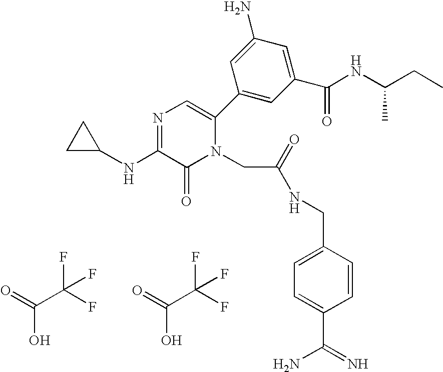 |
| 185. | 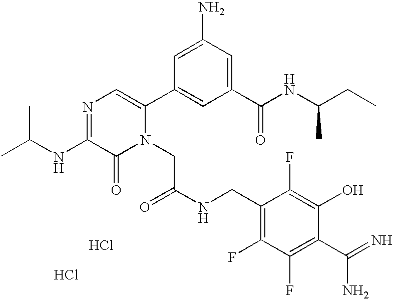 |
| 186. | 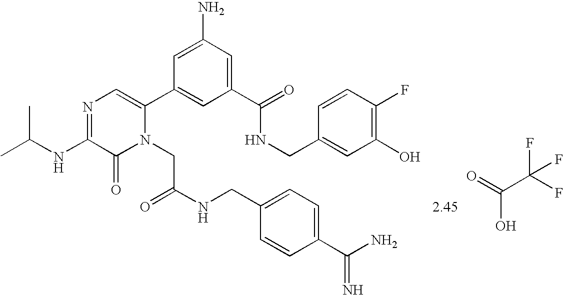 |
| 187. | 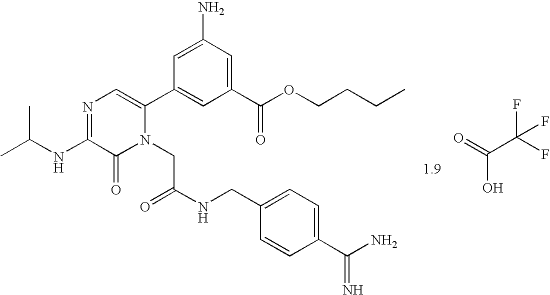 |
| 188. | 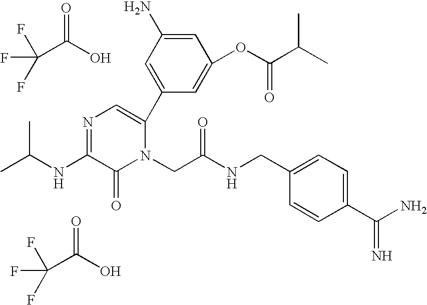 |
| 189. | 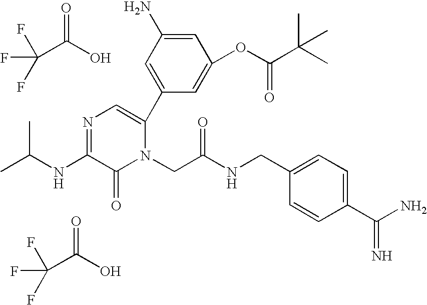 |
| 190. | 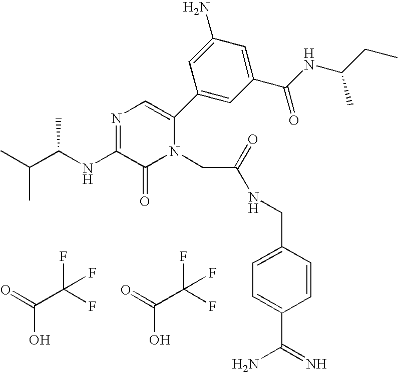 |
| 191. | 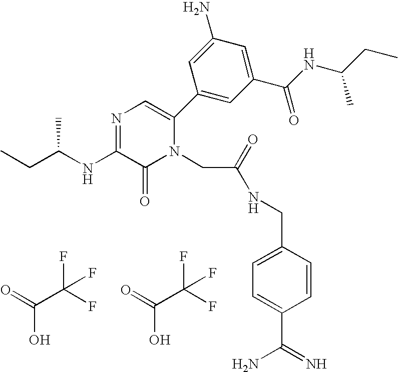 |
| 192. | 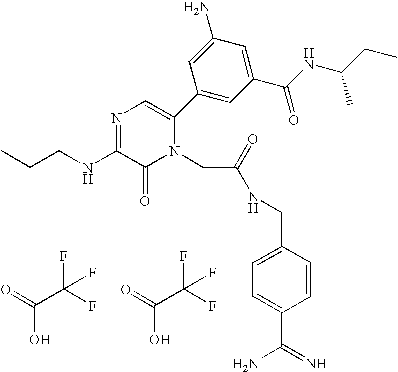 |
| 193. | 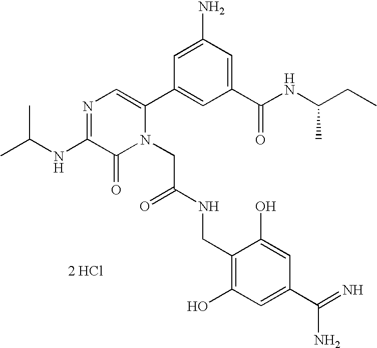 |
| 194. | 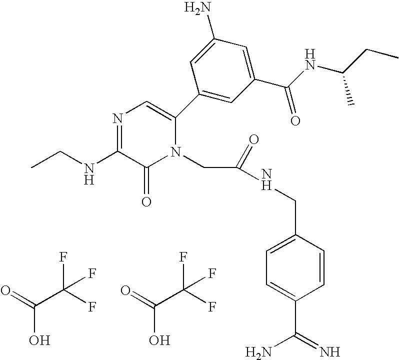 |
| 195. | 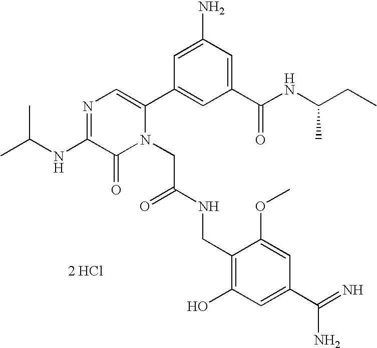 |
| 196. | 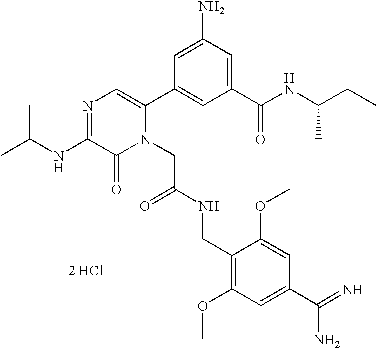 |
| 197. | 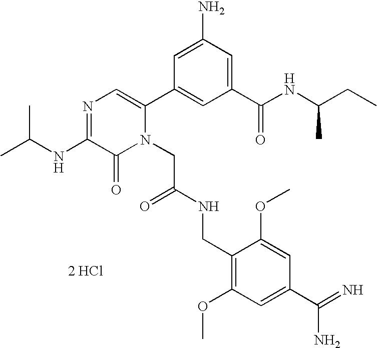 |
| 198. | 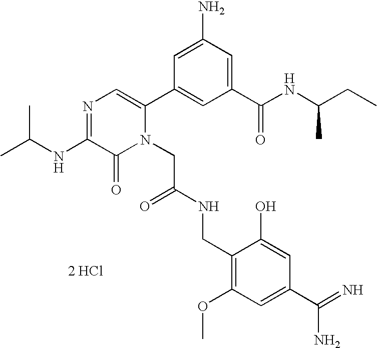 |
| 199. | 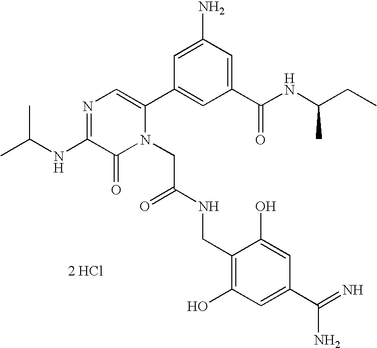 |
| 200. | 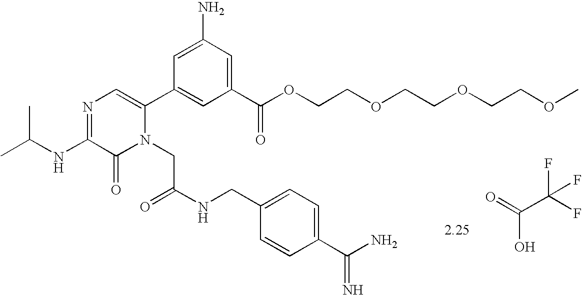 |
| 201. | 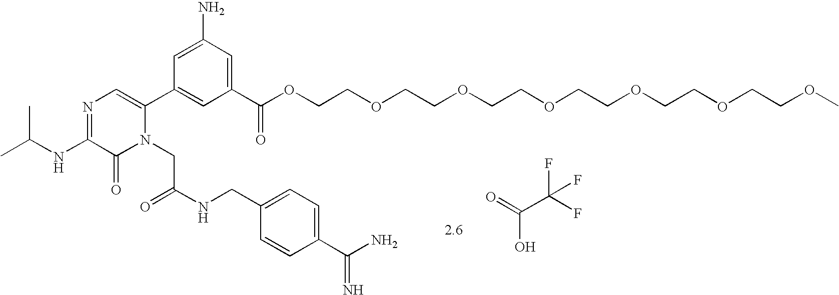 |
| 202. | 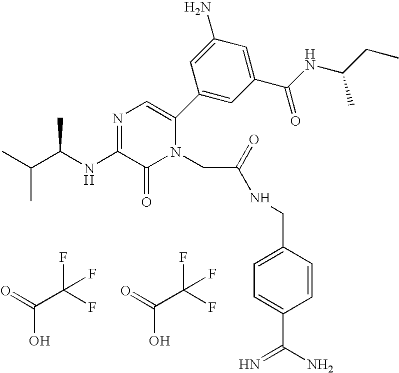 |
| 203. | 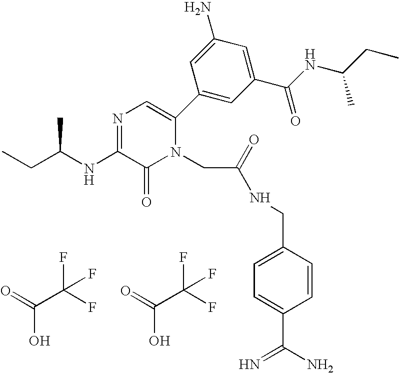 |
| 204. | 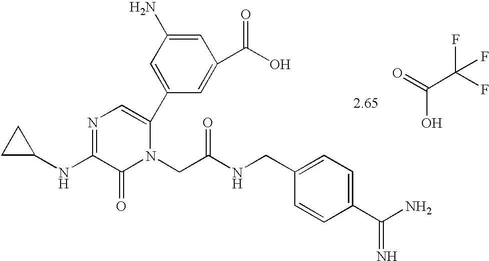 |
| 205. | 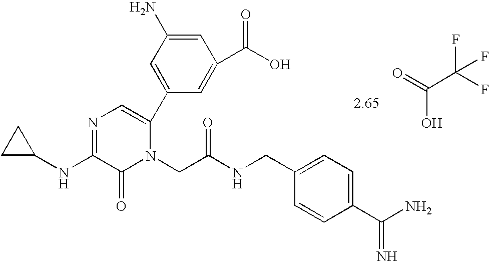 |
| 206. | 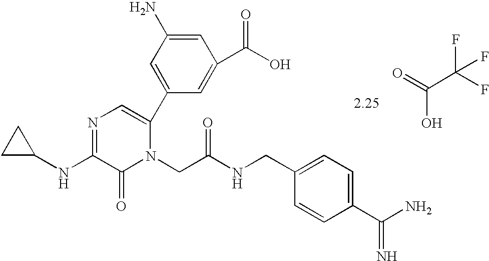 |
| 207. | 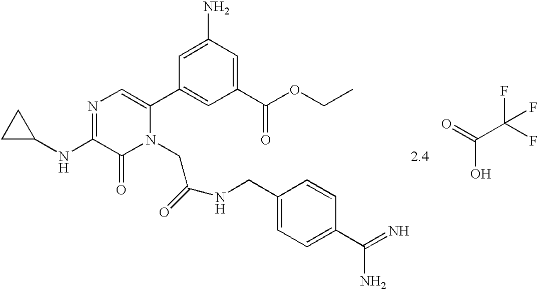 |
| 208. | 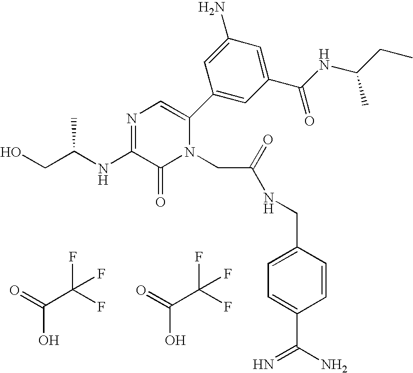 |
| 209. | 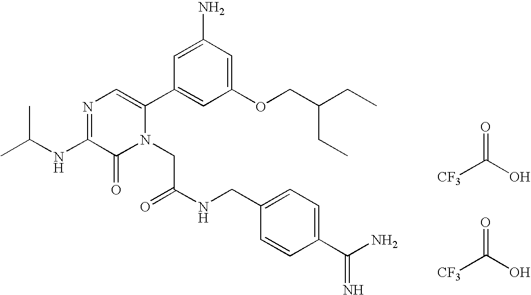 |
| 210. | 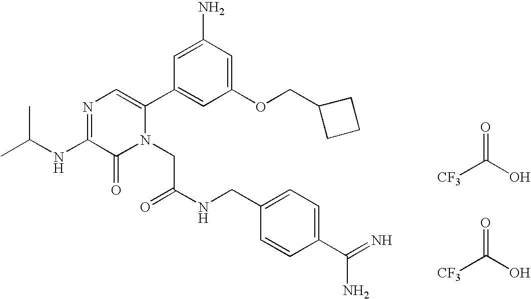 |
| 211. | 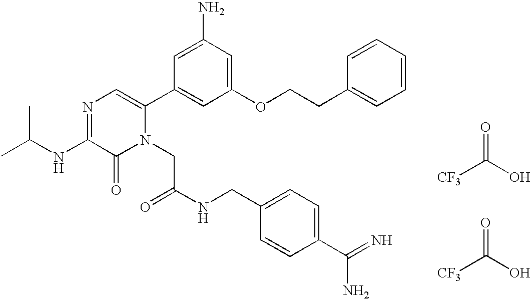 |
| 212. | 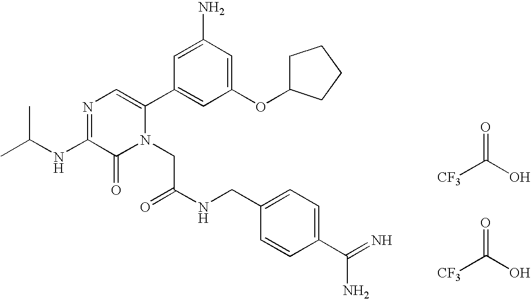 |
| 213. | 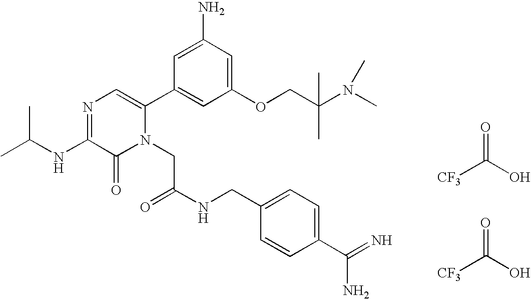 |
| 214. | 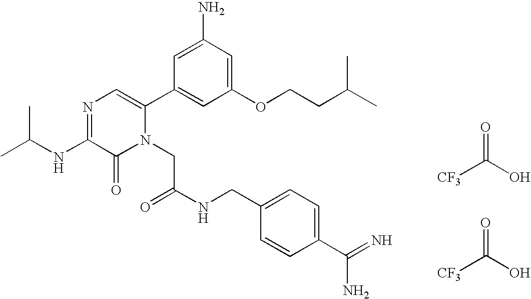 |
| 215. | 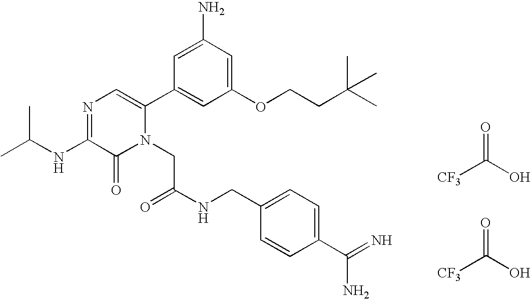 |
| 216. | 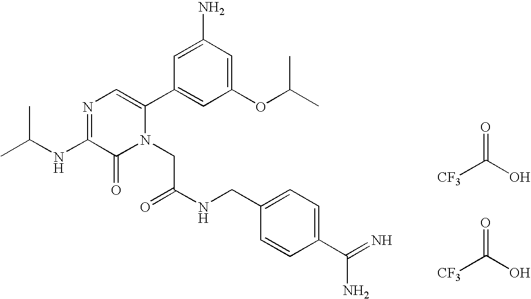 |
| 217. | 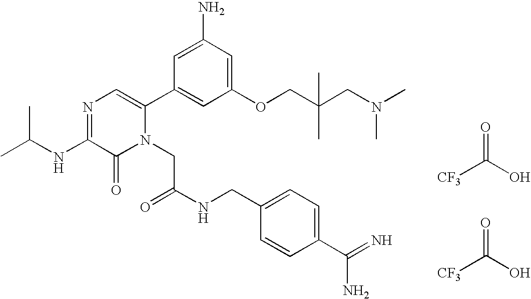 |
| 218. | 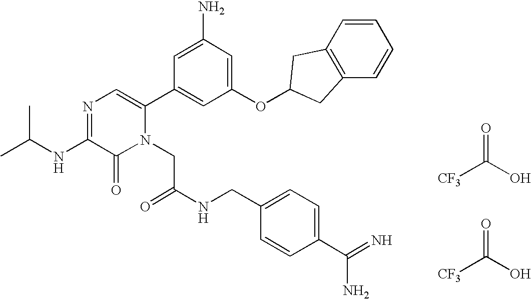 |
| 219. | 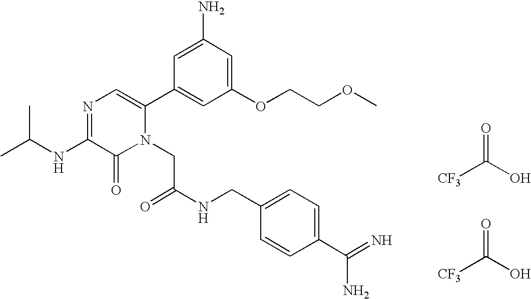 |
| 220. | 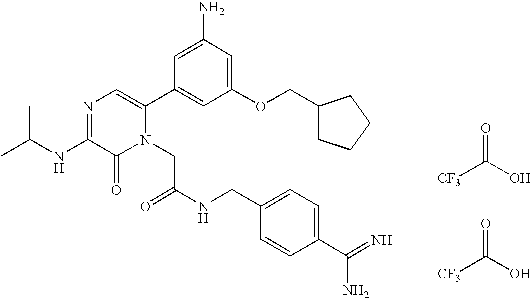 |
| 221. | 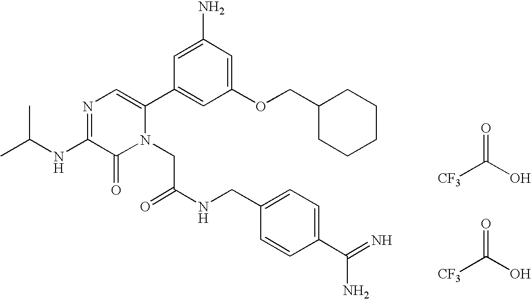 |
| 222. | 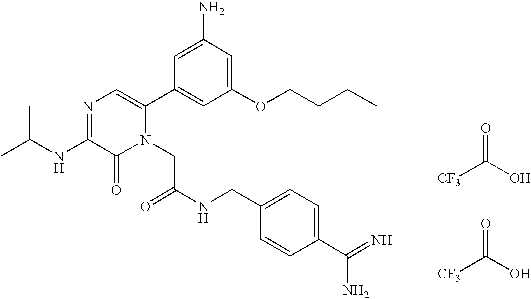 |
| 223. | 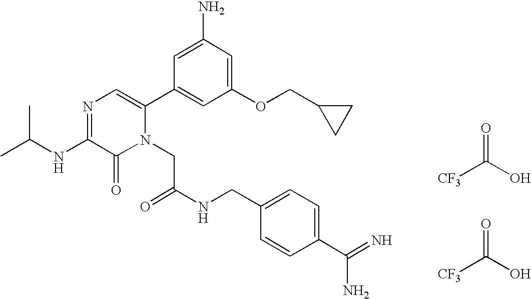 |
| 224. | 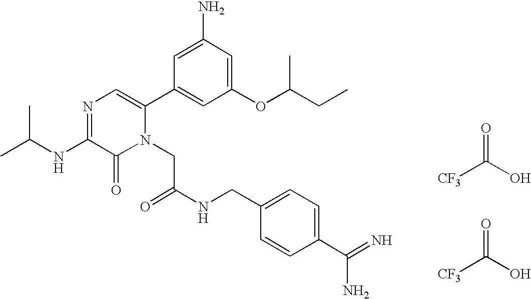 |
| 225. | 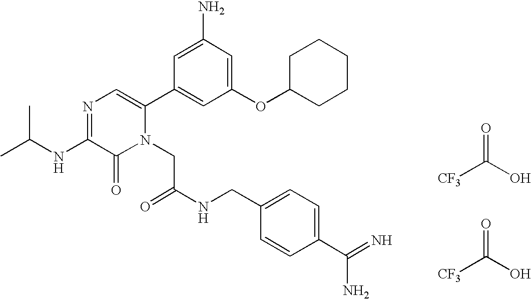 |
| 226. | 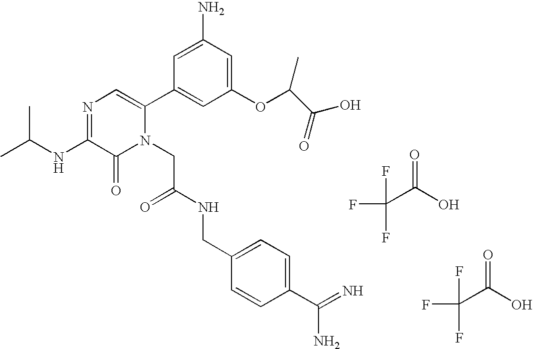 |
| 227. | 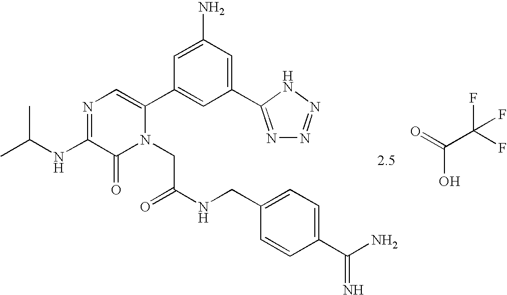 |
| 228. | 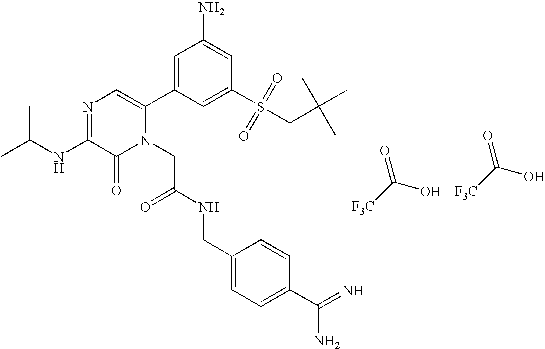 |
| 229. | 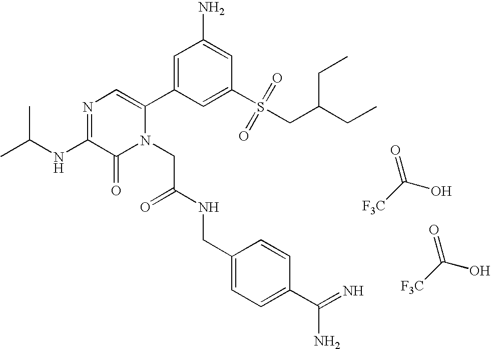 |
| 230. | 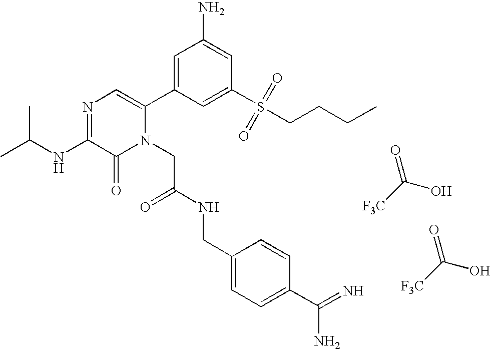 |
| 231. | 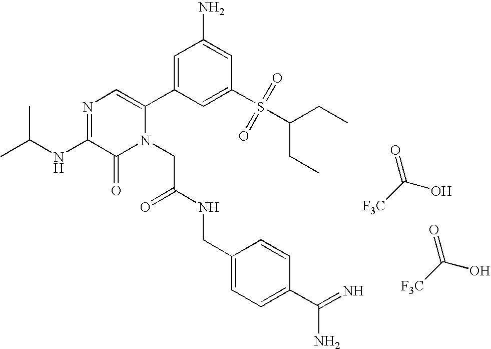 |
| 232. | 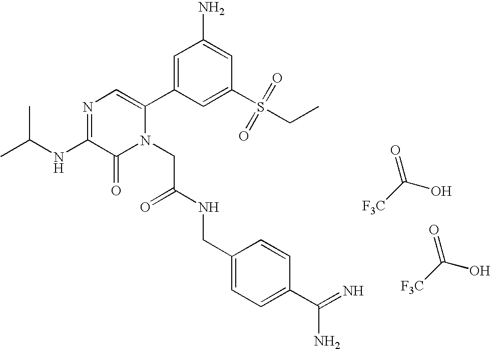 |
| 233. | 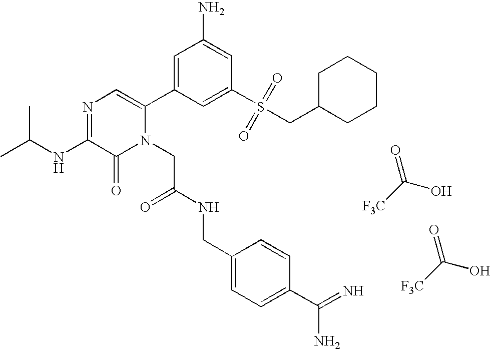 |
| 234. | 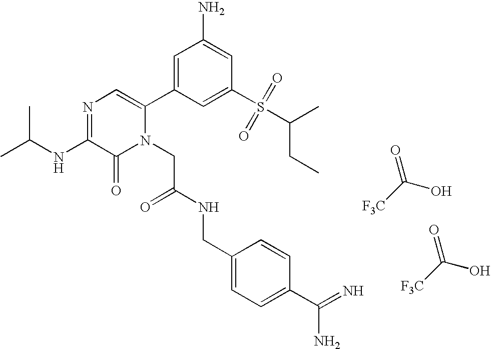 |
| 235. | 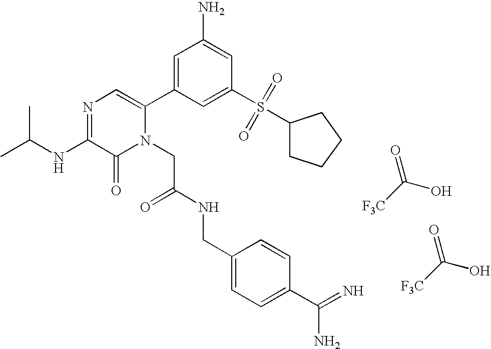 |
| 236. | 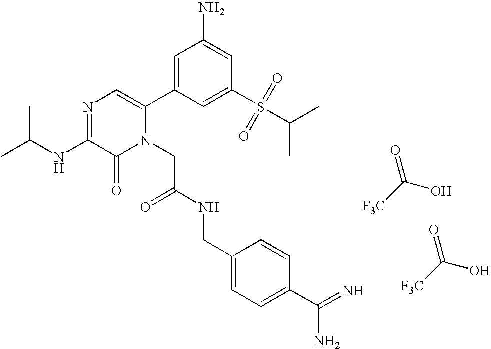 |
| 237. | 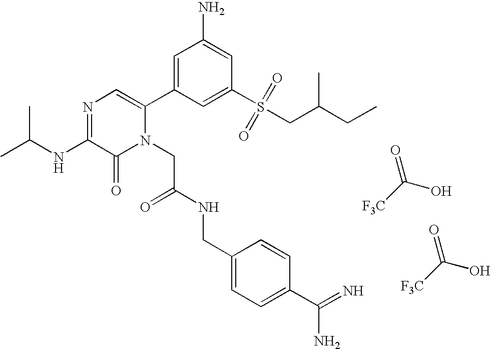 |
| 238. | 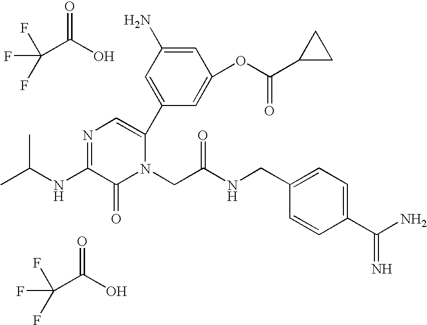 |
| 239. | 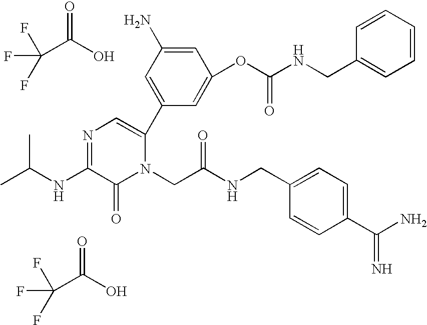 |
| 240. | 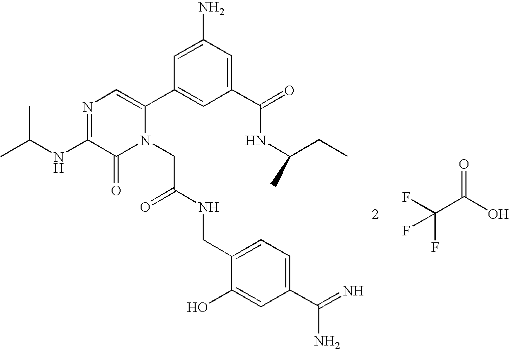 |
| 241. | 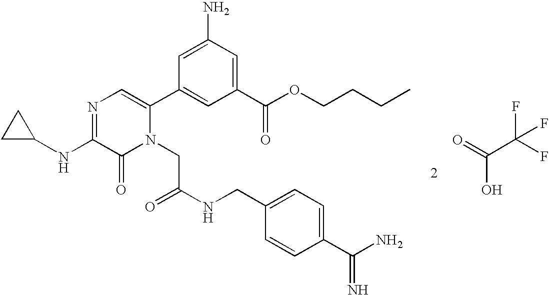 |
| 242. | 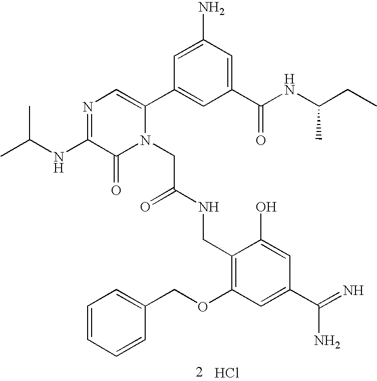 |
| 243. | 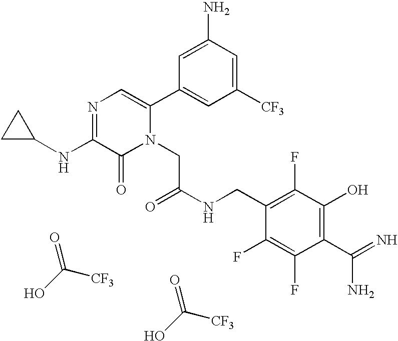 |
| 244. | 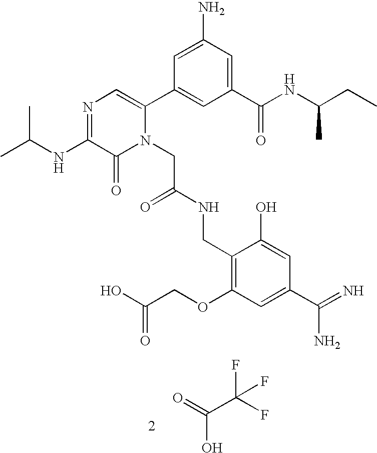 |
| 245. | 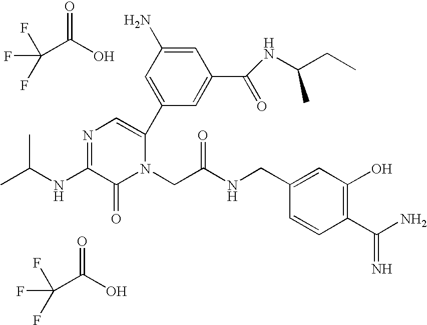 |
| 246. | 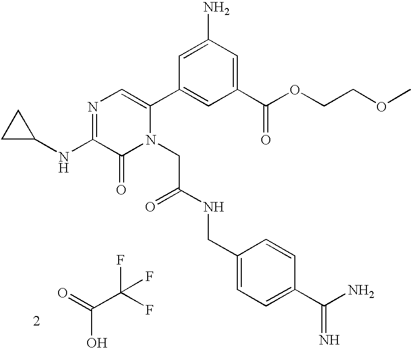 |
| 247. | 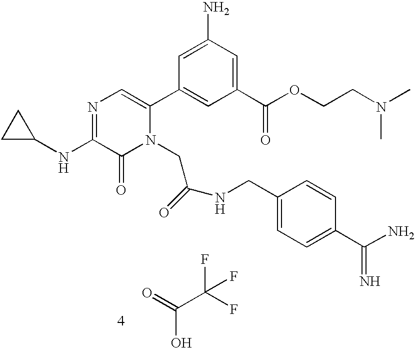 |
| 248. | 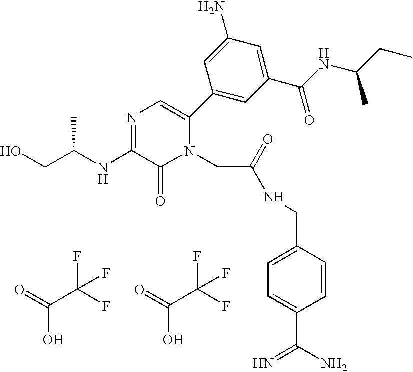 |
| 249. | 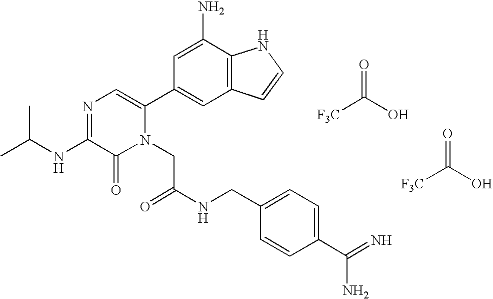 |
| 250. | 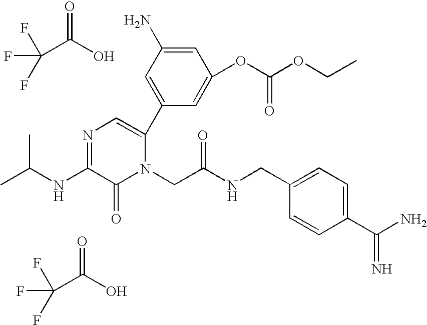 |
| 251. | 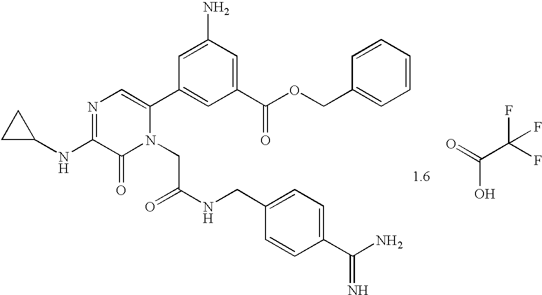 |
| 252. | 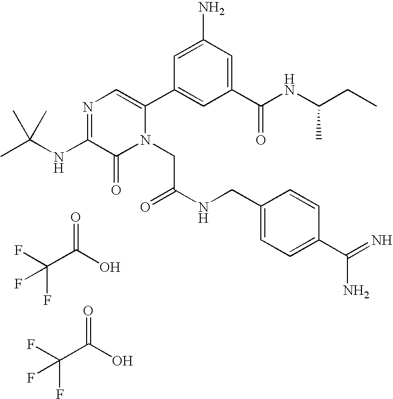 |
| 253. | 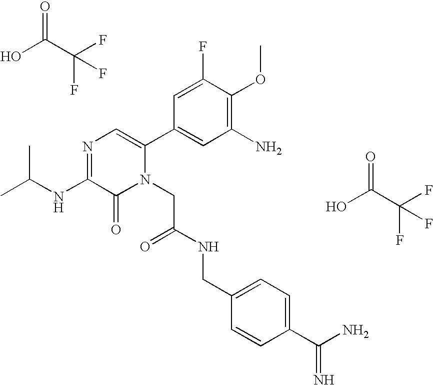 |
| 254. | 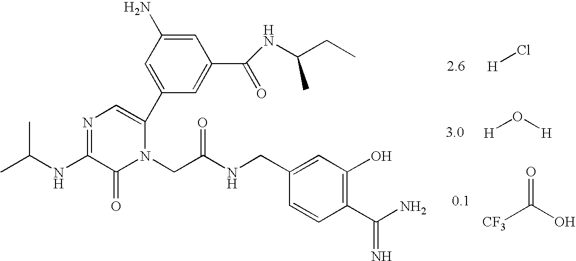 |
| 255. | 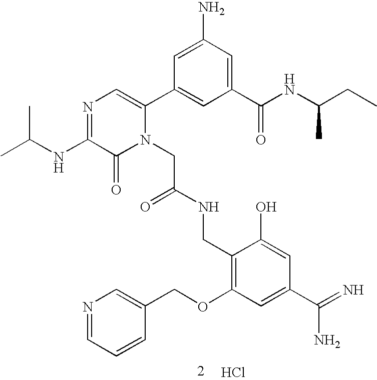 |
| 256. | 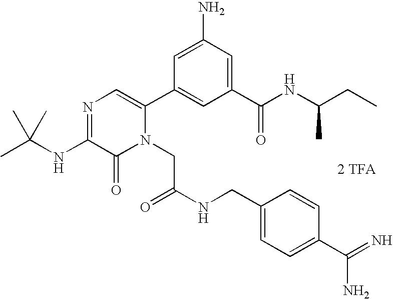 |
| 257. | 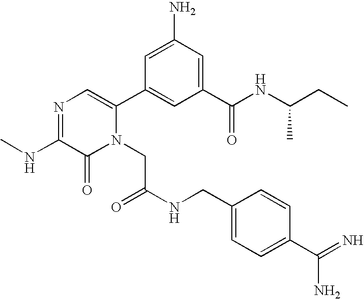 |
| 258. | 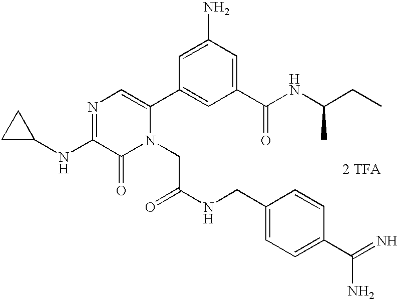 |

Following the processes described in the Schemes, Examples or elsewhere herein, compounds corresponding to each of formulae A, B, C, and D and having any of the combinations of substituents identified in Table 2 may be prepared.

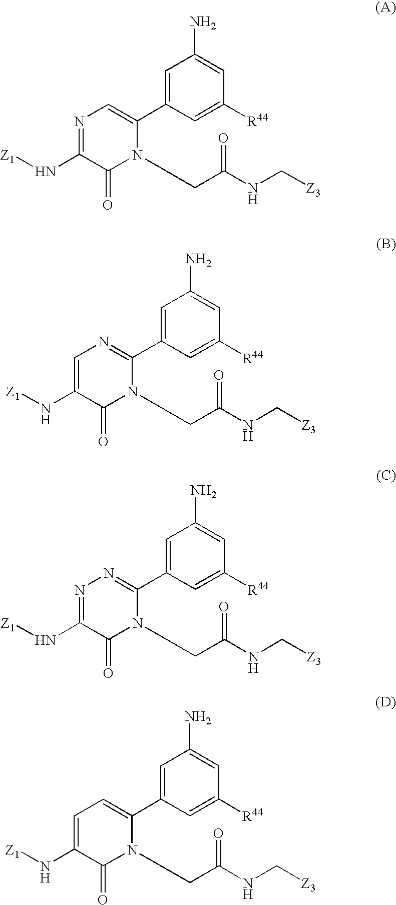

TABLE 2

| Z₁ | R⁴⁴ | Z₃ |
|---|---|---|
| methyl, ethyl, propyl, | hydroxy | benzamidine-4-yl |
| butyl, isopropyl, | | |
| cyclopropyl, secbutyl, | | |
| or cyclobutyl | | |
| methyl, ethyl, propyl, | hydroxy | 3-hydroxy- |
| butyl, isopropyl, | | benzamidine-4-yl |
| cyclopropyl, secbutyl, | | |
| or cyclobutyl | | |
| methyl, ethyl, propyl, | hydroxy | 3,5-dihydroxy- |
| butyl, isopropyl, | | benzamidine-4-yl |
| cyclopropyl, secbutyl, | | |
| or cyclobutyl | | |
| methyl, ethyl, propyl, | hydroxy | 2-hydroxy- |
| butyl, isopropyl, | | benzamidine-4-yl |
| cyclopropyl, secbutyl, | | |
| or cyclobutyl | | |
| methyl, ethyl, propyl, | hydroxy | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | hydroxy | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | isobutyl- | benzamidine-4-yl |
| butyl, isopropyl, | sulfonyl | |
| cyclopropyl, secbutyl, | | |
| or cyclobutyl | | |
| methyl, ethyl, propyl, | isobutyl- | 3-hydroxy- |
| butyl, isopropyl, | sulfonyl | benzamidine-4-yl |
| cyclopropyl, secbutyl, | | |
| or cyclobutyl | | |
| methyl, ethyl, propyl, | isobutyl- | 3,5-dihydroxy- |
| butyl, isopropyl, | sulfonyl | benzamidine-4-yl |
| cyclopropyl, secbutyl, | | |
| or cyclobutyl | | |
| methyl, ethyl, propyl, | isobutyl- | 2-hydroxy- |
| butyl, isopropyl, | sulfonyl | benzamidine-4-yl |
| cyclopropyl, secbutyl, | | |
| or cyclobutyl | | |
| methyl, ethyl, propyl, | isobutyl- | 3,5,6-trifluoro- |
| butyl, isopropyl, | sulfonyl | 2-hydroxy- |
| cyclopropyl, secbutyl, | | benzamide-4-yl |
| or cyclobutyl | | |
| methyl, ethyl, propyl, | isobutyl- | 2,5,6-trifluoro- |
| butyl, isopropyl, | sulfonyl | 3-hydroxy- |
| cyclopropyl, secbutyl, | | benzamidine-4-yl |
| or cyclobutyl | | |
| methyl, ethyl, propyl, | trifluoro- | benzamidine-4-yl |
| butyl, isopropyl, | methyl | |
| cyclopropyl, secbutyl, | | |
| or cyclobutyl | | |
| methyl, ethyl, propyl, | trifluoro- | 3-hydroxy- |
| butyl, isopropyl, | methyl | benzamidine-4-yl |
| cyclopropyl, secbutyl, | | |
| or cyclobutyl | | |
| methyl, ethyl, propyl, | trifluoro- | 3,5-dihydroxy- |
| butyl, isopropyl, | methyl | benzamidine-4-yl |
| cyclopropyl, secbutyl, | | |
| or cyclobutyl | | |
| methyl, ethyl, propyl, | trifluoro- | 2-hydroxy- |
| butyl, isopropyl, | methyl | benzamidine-4-yl |
| cyclopropyl, secbutyl, | | |
| or cyclobutyl | | |
| methyl, ethyl, propyl, | trifluoro- | 3,5,6-trifluoro- |
| butyl, isopropyl, | methyl | 2-hydroxy- |
| cyclopropyl, secbutyl, | | benzamide-4-yl |
| or cyclobutyl | | |
| methyl, ethyl, propyl, | trifluoro- | 2,5,6-trifluoro- |
| butyl, isopropyl, | methyl | 3-hydroxy- |
| cyclopropyl, secbutyl, | | benzamidine-4-yl |
| or cyclobutyl | | |
| methyl, ethyl, propyl, | carboxamido- | benzamidine-4-yl |
| butyl, isopropyl, | benzyl | |
| cyclopropyl, secbutyl, | | |
| or cyclobutyl | | |
| methyl, ethyl, propyl, | carboxamido- | 3-hydroxy- |
| butyl, isopropyl, | benzyl | benzamidine-4-yl |
| cyclopropyl, secbutyl, | | |
| or cyclobutyl | | |
| methyl, ethyl, propyl, | carboxamido- | 3,5-dihydroxy- |
| butyl, isopropyl, | benzyl | benzamidine-4-yl |
| cyclopropyl, secbutyl, | | |
| or cyclobutyl | | |
| methyl, ethyl, propyl, | carboxamido- | 2-hydroxy- |
| butyl, isopropyl, | benzyl | benzamidine-4-yl |
| cyclopropyl, secbutyl, | | |
| or cyclobutyl | | |
| methyl, ethyl, propyl, | carboxamido- | 3,5,6-trifluoro- |
| butyl, isopropyl, | benzyl | 2-hydroxy- |
| cyclopropyl, secbutyl, | | benzamide-4-yl |
| or cyclobutyl | | |
| methyl, ethyl, propyl, | carboxamido- | 2,5,6-trifluoro- |
| butyl, isopropyl, | benzyl | 3-hydroxy- |
| cyclopropyl, secbutyl, | | benzamidine-4-yl |
| or cyclobutyl | | |
| methyl, ethyl, propyl, | carboxamido- | benzamidine-4-yl |
| butyl, isopropyl, | butyl-2-yl | |
| cyclopropyl, secbutyl, | | |
| or cyclobutyl | | |
| methyl, ethyl, propyl, | carboxamido- | 3-hydroxy- |
| butyl, isopropyl, | butyl-2-yl | benzamidine-4-yl |
| cyclopropyl, secbutyl, | | |
| or cyclobutyl | | |
| methyl, ethyl, propyl, | carboxamido- | 3,5-dihydroxy- |
| butyl, isopropyl, | butyl-2-yl | benzamidine-4-yl |
| cyclopropyl, secbutyl, | | |
| or cyclobutyl | | |
| methyl, ethyl, propyl, | carboxamido- | 2-hydroxy- |
| butyl, isopropyl, | butyl-2-yl | benzamidine-4-yl |
| cyclopropyl, secbutyl, | | |
| or cyclobutyl | | |
| methyl, ethyl, propyl, | carboxamido- | 3,5,6-trifluoro- |
| butyl, isopropyl, | butyl-2-yl | 2-hydroxy- |
| cyclopropyl, secbutyl, | | benzamide-4-yl |
| or cyclobutyl | | |
| methyl, ethyl, propyl, | isobutyramido | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | isobutyramido | benzamidine-4-yl |
| butyl, isopropyl, | | |
| cyclopropyl, secbutyl, | | |
| or cyclobutyl | | |
| methyl, ethyl, propyl, | isobutyramido | 3-hydroxy- |
| butyl, isopropyl, | | benzamidine-4-yl |
| cyclopropyl, secbutyl, | | |
| or cyclobutyl | | |
| methyl, ethyl, propyl, | isobutyramido | 3,5-dihydroxy- |
| butyl, isopropyl, | | benzamidine-4-yl |
| cyclopropyl, secbutyl, | | |
| or cyclobutyl | | |
| methyl, ethyl, propyl, | isobutyramido | 2-hydroxy- |
| butyl, isopropyl, | | benzamidine-4-yl |
| cyclopropyl, secbutyl, | | |
| or cyclobutyl | | |
| methyl, ethyl, propyl, | isobutyramido | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | isobutyramido | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | isobutoxy | benzamidine-4-yl |
| butyl, isopropyl, | | |
| cyclopropyl, secbutyl, | | |
| or cyclobutyl | | |
| methyl, ethyl, propyl, | isobutoxy | 3-hydroxy- |
| butyl, isopropyl, | | benzamidine-4-yl |
| cyclopropyl, secbutyl, | | |
| or cyclobutyl | | |
| methyl, ethyl, propyl, | isobutoxy | 3,5-dihydroxy- |
| butyl, isopropyl, | | benzamidine-4-yl |
| cyclopropyl, secbutyl, | | |
| or cyclobutyl | | |
| methyl, ethyl, propyl, | isobutoxy | 2-hydroxy- |
| butyl, isopropyl, | | benzamidine-4-yl |
| cyclopropyl, secbutyl, | | |
| or cyclobutyl | | |
| methyl, ethyl, propyl, | isobutoxy | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | isobutoxy | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | carboethoxy | benzamidine-4-yl |
| butyl, isopropyl, | | |
| cyclopropyl, secbutyl, | | |
| or cyclobutyl | | |
| methyl, ethyl, propyl, | carboethoxy | 3-hydroxy- |
| butyl, isopropyl, | | benzamidine-4-yl |
| cyclopropyl, secbutyl, | | |
| or cyclobutyl | | |
| methyl, ethyl, propyl, | carboethoxy | 3,5-dihydroxy- |
| butyl, isopropyl, | | benzamidine-4-yl |
| cyclopropyl, secbutyl, | | |
| or cyclobutyl | | |
| methyl, ethyl, propyl, | carboethoxy | 2-hydroxy- |
| butyl, isopropyl, | | benzamidine-4-yl |
| cyclopropyl, secbutyl, | | |
| or cyclobutyl | | |
| methyl, ethyl, propyl, | carboethoxy | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | carboethoxy | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | carboxyl | benzamidine-4-yl |
| butyl, isopropyl, | | |
| cyclopropyl, secbutyl, | | |
| or cyclobutyl | | |
| methyl, ethyl, propyl, | carboxyl | 3-hydroxy- |
| butyl, isopropyl, | | benzamidine-4-yl |
| cyclopropyl, secbutyl, | | |
| or cyclobutyl | | |
| methyl, ethyl, propyl, | carboxyl | 3,5-dihydroxy- |
| butyl, isopropyl, | | benzamidine-4-yl |
| cyclopropyl, secbutyl, | | |
| or cyclobutyl | | |
| methyl, ethyl, propyl, | carboxyl | 2-hydroxy- |
| butyl, isopropyl, | | benzamidine-4-yl |
| cyclopropyl, secbutyl, | | |
| or cyclobutyl | | |
| methyl, ethyl, propyl, | carboxyl | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | carboxyl | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | amino | benzamidine-4-yl |
| butyl, isopropyl, | | |
| cyclopropyl, secbutyl, | | |
| or cyclobutyl | | |
| methyl, ethyl, propyl, | amino | 3-hydroxy- |
| butyl, isopropyl, | | benzamidine-4-yl |
| cyclopropyl, secbutyl, | | |
| or cyclobutyl | | |
| methyl, ethyl, propyl, | amino | 3,5-dihydroxy- |
| butyl, isopropyl, | | benzamidine-4-yl |
| cyclopropyl, secbutyl, | | |
| or cyclobutyl | | |
| methyl, ethyl, propyl, | amino | 2-hydroxy- |
| butyl, isopropyl, | | benzamidine-4-yl |
| cyclopropyl, secbutyl, | | |
| or cyclobutyl | | |
| methyl, ethyl, propyl, | amino | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | amino | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | 3- | benzamidine-4-yl |
| butyl, isopropyl, | aminomethyl- | |
| cyclopropyl, secbutyl, | thiphene | |
| or cyclobutyl | | |
| methyl, ethyl, propyl, | 3- | 3-hydroxy- |
| butyl, isopropyl, | aminomethyl- | benzamidine-4-yl |
| cyclopropyl, secbutyl, | thiphene | |
| or cyclobutyl | | |
| methyl, ethyl, propyl, | 3- | 3,5-dihydroxy- |
| butyl, isopropyl, | aminomethyl- | benzamidine-4-yl |
| cyclopropyl, secbutyl, | thiphene | |
| or cyclobutyl | | |
| methyl, ethyl, propyl, | 3- | 2-hydroxy- |
| butyl, isopropyl, | aminomethyl- | benzamidine-4-yl |
| cyclopropyl, secbutyl, | thiphene | |
| or cyclobutyl | | |
| methyl, ethyl, propyl, | 3- | 3,5,6-trifluoro- |
| butyl, isopropyl, | aminomethyl- | 2-hydroxy- |
| cyclopropyl, secbutyl, | thiphene | benzamide-4-yl |
| or cyclobutyl | | |
| methyl, ethyl, propyl, | 3- | 2,5,6-trifluoro- |
| butyl, isopropyl, | aminomethyl- | 3-hydroxy- |
| cyclopropyl, secbutyl, | thiphene | benzamidine-4-yl |
| or cyclobutyl | | |
| methyl, ethyl, propyl, | benzylamine | benzamidine-4-yl |
| butyl, isopropyl, | | |
| cyclopropyl, secbutyl, | | |
| or cyclobutyl | | |
| methyl, ethyl, propyl, | benzylamine | 3-hydroxy- |
| butyl, isopropyl, | | benzamidine-4-yl |
| cyclopropyl, secbutyl, | | |
| or cyclobutyl | | |
| methyl, ethyl, propyl, | benzylamine | 3,5-dihydroxy- |
| butyl, isopropyl, | | benzamidine-4-yl |
| cyclopropyl, secbutyl, | | |
| or cyclobutyl | | |
| methyl, ethyl, propyl, | benzylamine | 2-hydroxy- |
| butyl, isopropyl, | | benzamidine-4-yl |
| cyclopropyl, secbutyl, | | |
| or cyclobutyl | | |
| methyl, ethyl, propyl, | benzylamine | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | benzylamine | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | phenethylamine | benzamidine-4-yl |
| butyl, isopropyl, | | |
| cyclopropyl, secbutyl, | | |
| or cyclobutyl | | |
| methyl, ethyl, propyl, | phenethylamine | 3-hydroxy- |
| butyl, isopropyl, | | benzamidine-4-yl |
| cyclopropyl, secbutyl, | | |
| or cyclobutyl | | |
| methyl, ethyl, propyl, | phenethylamine | 3,5-dihydroxy- |
| butyl, isopropyl, | | benzamidine-4-yl |
| cyclopropyl, secbutyl, | | |
| or cyclobutyl | | |
| methyl, ethyl, propyl, | phenethylamine | 2-hydroxy- |
| butyl, isopropyl, | | benzamidine-4-yl |
| cyclopropyl, secbutyl, | | |
| or cyclobutyl | | |
| methyl, ethyl, propyl, | phenethylamine | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | phenethylamine | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | isobutylamine | benzamidine-4-yl |
| butyl, isopropyl, | | |
| cyclopropyl, secbutyl, | | |
| or cyclobutyl | | |
| methyl, ethyl, propyl, | isobutylamine | 3-hydroxy- |
| butyl, isopropyl, | | benzamidine-4-yl |
| cyclopropyl, secbutyl, | | |
| or cyclobutyl | | |
| methyl, ethyl, propyl, | isobutylamine | 3,5-dihydroxy- |
| butyl, isopropyl, | | benzamidine-4-yl |
| cyclopropyl, secbutyl, | | |
| or cyclobutyl | | |
| methyl, ethyl, propyl, | isobutylamine | 2-hydroxy- |
| butyl, isopropyl, | | benzamidine-4-yl |
| cyclopropyl, secbutyl, | | |
| or cyclobutyl | | |
| methyl, ethyl, propyl, | isobutylamine | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | isobutylamine | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | methoxy- | benzamidine-4-yl |
| butyl, isopropyl, | methylamide | |
| cyclopropyl, secbutyl, | | |
| or cyclobutyl | | |
| methyl, ethyl, propyl, | methoxy- | 3-hydroxy- |
| butyl, isopropyl, | methylamide | benzamidine-4-yl |
| cyclopropyl, secbutyl, | | |
| or cyclobutyl | | |
| methyl, ethyl, propyl, | methoxy- | 3,5-dihydroxy- |
| butyl, isopropyl, | methylamide | benzamidine-4-yl |
| cyclopropyl, secbutyl, | | |
| or cyclobutyl | | |
| methyl, ethyl, propyl, | methoxy- | 2-hydroxy- |
| butyl, isopropyl, | methylamide | benzamidine-4-yl |
| cyclopropyl, secbutyl, | | |
| or cyclobutyl | | |
| methyl, ethyl, propyl, | methoxy- | 3,5,6-trifluoro- |
| butyl, isopropyl, | methylamide | 2-hydroxy- |
| cyclopropyl, secbutyl, | | benzamide-4-yl |
| or cyclobutyl | | |
| methyl, ethyl, propyl, | methoxy- | 2,5,6-trifluoro- |
| butyl, isopropyl, | methylamide | 3-hydroxy- |
| cyclopropyl, secbutyl, | | benzamidine-4-yl |
| or cyclobutyl | | |
| methyl, ethyl, propyl, | 1-carboxyl- | benzamidine-4-yl |
| butyl, isopropyl, | benzylamide | |
| cyclopropyl, secbutyl, | | |
| or cyclobutyl | | |
| methyl, ethyl, propyl, | 1-carboxyl- | 3-hydroxy- |
| butyl, isopropyl, | benzylamide | benzamidine-4-yl |
| cyclopropyl, secbutyl, | | |
| or cyclobutyl | | |
| methyl, ethyl, propyl, | 1-carboxyl- | 3,5-dihydroxy- |
| butyl, isopropyl, | benzylamide | benzamidine-4-yl |
| cyclopropyl, secbutyl, | | |
| or cyclobutyl | | |
| methyl, ethyl, propyl, | 1-carboxyl- | 2-hydroxy- |
| butyl, isopropyl, | benzylamide | benzamidine-4-yl |
| cyclopropyl, secbutyl, | | |
| or cyclobutyl | | |
| methyl, ethyl, propyl, | 1-carboxyl- | 3,5,6-trifluoro- |
| butyl, isopropyl, | benzylamide | 2-hydroxy- |
| cyclopropyl, secbutyl, | | benzamide-4-yl |
| or cyclobutyl | | |
| methyl, ethyl, propyl, | 1-carboxyl- | 2,5,6-trifluoro- |
| butyl, isopropyl, | benzylamide | 3-hydroxy- |
| cyclopropyl, secbutyl, | | benzamidine-4-yl |
| or cyclobutyl | | |
| methyl, ethyl, propyl, | p-fluoro- | benzamidine-4-yl |
| butyl, isopropyl, | benzylamide | |
| cyclopropyl, secbutyl, | | |
| or cyclobutyl | | |
| methyl, ethyl, propyl, | p-fluoro- | 3-hydroxy- |
| butyl, isopropyl, | benzylamide | benzamidine-4-yl |
| cyclopropyl, secbutyl, | | |
| or cyclobutyl | | |
| methyl, ethyl, propyl, | p-fluoro- | 3,5-dihydroxy- |
| butyl, isopropyl, | benzylamide | benzamidine-4-yl |
| cyclopropyl, secbutyl, | | |
| or cyclobutyl | | |
| methyl, ethyl, propyl, | p-fluoro- | 2-hydroxy- |
| butyl, isopropyl, | benzylamide | benzamidine-4-yl |
| cyclopropyl, secbutyl, | | |
| or cyclobutyl | | |
| methyl, ethyl, propyl, | p-fluoro- | 3,5,6-trifluoro- |
| butyl, isopropyl, | benzylamide | 2-hydroxy- |
| cyclopropyl, secbutyl, | | benzamide-4-yl |
| or cyclobutyl | | |
| methyl, ethyl, propyl, | p-fluoro- | 2,5,6-trifluoro- |
| butyl, isopropyl, | benzylamide | 3-hydroxy- |
| cyclopropyl, secbutyl, | | benzamidine-4-yl |
| or cyclobutyl | | |
| methyl, ethyl, propyl, | cyclobutyl- | benzamidine-4-yl |
| butyl, isopropyl, | amide | |
| cyclopropyl, secbutyl, | | |
| or cyclobutyl | | |
| methyl, ethyl, propyl, | cyclobutyl- | 3-hydroxy- |
| butyl, isopropyl, | amide | benzamidine-4-yl |
| cyclopropyl, secbutyl, | | |
| or cyclobutyl | | |
| methyl, ethyl, propyl, | cyclobutyl- | 3,5-dihydroxy- |
| butyl, isopropyl, | amide | benzamidine-4-yl |
| cyclopropyl, secbutyl, | | |
| or cyclobutyl | | |
| methyl, ethyl, propyl, | cyclobutyl- | 2-hydroxy- |
| butyl, isopropyl, | amide | benzamidine-4-yl |
| cyclopropyl, secbutyl, | | |
| or cyclobutyl | | |
| methyl, ethyl, propyl, | cyclobutyl- | 3,5,6-trifluoro- |
| butyl, isopropyl, | amide | 2-hydroxy- |
| cyclopropyl, secbutyl, | | benzamide-4-yl |
| or cyclobutyl | | |
| methyl, ethyl, propyl, | cyclobutyl- | 2,5,6-trifluoro- |
| butyl, isopropyl, | amide | 3-hydroxy- |
| cyclopropyl, secbutyl, | | benzamidine-4-yl |
| or cyclobutyl | | |
| methyl, ethyl, propyl, | m-fluoro- | benzamidine-4-yl |
| butyl, isopropyl, | benzylamide | |
| cyclopropyl, secbutyl, | | |
| or cyclobutyl | | |
| methyl, ethyl, propyl, | m-fluoro- | 3-hydroxy- |
| butyl, isopropyl, | benzylamide | benzamidine-4-yl |
| cyclopropyl, secbutyl, | | |
| or cyclobutyl | | |
| methyl, ethyl, propyl, | m-fluoro- | 3,5-dihydroxy- |
| butyl, isopropyl, | benzylamide | benzamidine-4-yl |
| cyclopropyl, secbutyl, | | |
| or cyclobutyl | | |
| methyl, ethyl, propyl, | m-fluoro- | 2-hydroxy- |
| butyl, isopropyl, | benzylamide | benzamidine-4-yl |
| cyclopropyl, secbutyl, | | |
| or cyclobutyl | | |
| methyl, ethyl, propyl, | m-fluoro- | 3,5,6-trifluoro- |
| butyl, isopropyl, | benzylamide | 2-hydroxy- |
| cyclopropyl, secbutyl, | | benzamide-4-yl |
| or cyclobutyl | | |
| methyl, ethyl, propyl, | m-fluoro- | 2,5,6-trifluoro- |
| butyl, isopropyl, | benzylamide | 3-hydroxy- |
| cyclopropyl, secbutyl, | | benzamidine-4-yl |
| or cyclobutyl | | |
| methyl, ethyl, propyl, | 1-methyl- | benzamidine-4-yl |
| butyl, isopropyl, | benzylamide | |
| cyclopropyl, secbutyl, | | |
| or cyclobutyl | | |
| methyl, ethyl, propyl, | 1-methyl- | 3-hydroxy- |
| butyl, isopropyl, | benzylamide | benzamidine-4-yl |
| cyclopropyl, secbutyl, | | |
| or cyclobutyl | | |
| methyl, ethyl, propyl, | 1-methyl- | 3,5-dihydroxy- |
| butyl, isopropyl, | benzylamide | benzamidine-4-yl |
| cyclopropyl, secbutyl, | | |
| or cyclobutyl | | |
| methyl, ethyl, propyl, | 1-methyl- | 2-hydroxy- |
| butyl, isopropyl, | benzylamide | benzamidine-4-yl |
| cyclopropyl, secbutyl, | | |
| or cyclobutyl | | |
| methyl, ethyl, propyl, | 1-methyl- | 3,5,6-trifluoro- |
| butyl, isopropyl, | benzylamide | 2-hydroxy- |
| cyclopropyl, secbutyl, | | benzamide-4-yl |
| or cyclobutyl | | |
| methyl, ethyl, propyl, | 1-methyl- | 2,5,6-trifluoro- |
| butyl, isopropyl, | benzylamide | 3-hydroxy- |
| cyclopropyl, secbutyl, | | benzamidine-4-yl |
| or cyclobutyl | | |
| methyl, ethyl, propyl, | secbutyl, | benzamidine-4-yl |
| butyl, isopropyl, | butylamide | |
| cyclopropyl, secbutyl, | | |
| or cyclobutyl | | |
| methyl, ethyl, propyl, | secbutyl, | 3-hydroxy- |
| butyl, isopropyl, | butylamide | benzamidine-4-yl |
| cyclopropyl, secbutyl, | | |
| or cyclobutyl | | |
| methyl, ethyl, propyl, | secbutyl, | 3,5-dihydroxy- |
| butyl, isopropyl, | butylamide | benzamidine-4-yl |
| cyclopropyl, secbutyl, | | |
| or cyclobutyl | | |
| methyl, ethyl, propyl, | secbutyl, | 2-hydroxy- |
| butyl, isopropyl, | butylamide | benzamidine-4-yl |
| cyclopropyl, secbutyl, | | |
| or cyclobutyl | | |
| methyl, ethyl, propyl, | secbutyl, | 3,5,6-trifluoro- |
| butyl, isopropyl, | butylamide | 2-hydroxy- |
| cyclopropyl, secbutyl, | | benzamide-4-yl |
| or cyclobutyl | | |
| methyl, ethyl, propyl, | secbutyl, | 2,5,6-trifluoro- |
| butyl, isopropyl, | butylamide | 3-hydroxy- |
| cyclopropyl, secbutyl, | | benzamidine-4-yl |
| or cyclobutyl | | |
| methyl, ethyl, propyl, | benzylacyl- | benzamidine-4-yl |
| butyl, isopropyl, | amine | |
| cyclopropyl, secbutyl, | | |
| or cyclobutyl | | |
| methyl, ethyl, propyl, | benzylacyl- | 3-hydroxy- |
| butyl, isopropyl, | amine | benzamidine-4-yl |
| cyclopropyl, secbutyl, | | |
| or cyclobutyl | | |
| methyl, ethyl, propyl, | benzylacyl- | 3,5-dihydroxy- |
| butyl, isopropyl, | amine | benzamidine-4-yl |
| cyclopropyl, secbutyl, | | |
| or cyclobutyl | | |
| methyl, ethyl, propyl, | benzylacyl- | 2-hydroxy- |
| butyl, isopropyl, | amine | benzamidine-4-yl |
| cyclopropyl, secbutyl, | | |
| or cyclobutyl | | |
| methyl, ethyl, propyl, | benzylacyl- | 3,5,6-trifluoro- |
| butyl, isopropyl, | amine | 2-hydroxy- |
| cyclopropyl, secbutyl, | | benzamide-4-yl |
| or cyclobutyl | | |
| methyl, ethyl, propyl, | benzylacyl- | 2,5,6-trifluoro- |
| butyl, isopropyl, | amine | 3-hydroxy- |
| cyclopropyl, secbutyl, | | benzamidine-4-yl |
| or cyclobutyl | | |
| methyl, ethyl, propyl, | isobutylamide | benzamidine-4-yl |
| butyl, isopropyl, | | |
| cyclopropyl, secbutyl, | | |
| or cyclobutyl | | |
| methyl, ethyl, propyl, | isobutylamide | 3-hydroxy- |
| butyl, isopropyl, | | benzamidine-4-yl |
| cyclopropyl, secbutyl, | | |
| or cyclobutyl | | |
| methyl, ethyl, propyl, | isobutylamide | 3,5-dihydroxy- |
| butyl, isopropyl, | | benzamidine-4-yl |
| cyclopropyl, secbutyl, | | |
| or cyclobutyl | | |
| methyl, ethyl, propyl, | isobutylamide | 2-hydroxy- |
| butyl, isopropyl, | | benzamidine-4-yl |
| cyclopropyl, secbutyl, | | |
| or cyclobutyl | | |
| methyl, ethyl, propyl, | isobutylamide | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | isobutylamide | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | secbutyl, | benzamidine-4-yl |
| butyl, isopropyl, | pentylamine | |
| cyclopropyl, secbutyl, | | |
| or cyclobutyl | | |
| methyl, ethyl, propyl, | secbutyl, | 3-hydroxy- |
| butyl, isopropyl, | pentylamine | benzamidine-4-yl |
| cyclopropyl, secbutyl, | | |
| or cyclobutyl | | |
| methyl, ethyl, propyl, | secbutyl, | 3,5-dihydroxy- |
| butyl, isopropyl, | pentylamine | benzamidine-4-yl |
| cyclopropyl, secbutyl, | | |
| or cyclobutyl | | |
| methyl, ethyl, propyl, | secbutyl, | 2-hydroxy- |
| butyl, isopropyl, | pentylamine | benzamidine-4-yl |
| cyclopropyl, secbutyl, | | |
| or cyclobutyl | | |
| methyl, ethyl, propyl, | secbutyl, | 3,5,6-trifluoro- |
| butyl, isopropyl, | pentylamine | 2-hydroxy- |
| cyclopropyl, secbutyl, | | benzamide-4-yl |
| or cyclobutyl | | |
| methyl, ethyl, propyl, | secbutyl, | 2,5,6-trifluoro- |
| butyl, isopropyl, | pentylamine | 3-hydroxy- |
| cyclopropyl, secbutyl, | | benzamidine-4-yl |
| or cyclobutyl | | |
| methyl, ethyl, propyl, | cyclopentyl- | benzamidine-4-yl |
| butyl, isopropyl, | acylamine | |
| cyclopropyl, secbutyl, | | |
| or cyclobutyl | | |
| methyl, ethyl, propyl, | cyclopentyl- | 3-hydroxy- |
| butyl, isopropyl, | acylamine | benzamidine-4-yl |
| cyclopropyl, secbutyl, | | |
| or cyclobutyl | | |
| methyl, ethyl, propyl, | cyclopentyl- | 3,5-dihydroxy- |
| butyl, isopropyl, | acylamine | benzamidine-4-yl |
| cyclopropyl, secbutyl, | | |
| or cyclobutyl | | |
| methyl, ethyl, propyl, | cyclopentyl- | 2-hydroxy- |
| butyl, isopropyl, | acylamine | benzamidine-4-yl |
| cyclopropyl, secbutyl, | | |
| or cyclobutyl | | |
| methyl, ethyl, propyl, | cyclopentyl- | 3,5,6-trifluoro- |
| butyl, isopropyl, | acylamine | 2-hydroxy- |
| cyclopropyl, secbutyl, | | benzamidine-4-yl |
| or cyclobutyl | | |
| methyl, ethyl, propyl, | cylopentyl- | 2,5,6-trifluoro- |
| butyl, isopropyl, | acylamine | 3-hydroxy- |
| cyclopropyl, secbutyl, | | benzamidine-4-yl |
| or cyclobutyl | | |
| methyl, ethyl, propyl, | 1-carboxyl, | benzamidine-4-yl |
| butyl, isopropyl, | 2-methyl- | |
| cyclopropyl, secbutyl, | butylamide | |
| or cyclobutyl | | |
| methyl, ethyl, propyl, | 1-carboxyl, | 3-hydroxy- |
| butyl, isopropyl, | 2-methyl- | benzamidine-4-yl |
| cyclopropyl, secbutyl, | butylamide | |
| or cyclobutyl | | |
| methyl, ethyl, propyl, | 1-carboxyl, | 3,5-dihydroxy- |
| butyl, isopropyl, | 2-methyl- | benzamidine-4-yl |
| cyclopropyl, secbutyl, | butylamide | |
| or cyclobutyl | | |
| methyl, ethyl, propyl, | 1-carboxyl, | 2-hydroxy- |
| butyl, isopropyl, | 2-methyl- | benzamidine-4-yl |
| cyclopropyl, secbutyl, | butylamide | |
| or cyclobutyl | | |
| methyl, ethyl, propyl, | 1-carboxyl, | 3,5,6-trifluoro- |
| butyl, isopropyl, | 2-methyl- | 2-hydroxy- |
| cyclopropyl, secbutyl, | butylamide | benzamide-4-yl |
| or cyclobutyl | | |
| methyl, ethyl, propyl, | 1-carboxyl, | 2,5,6-trifluoro- |
| butyl, isopropyl, | 2-methyl- | 3-hydroxy- |
| cyclopropyl, secbutyl, | butylamide | benzamidine-4-yl |
| or cyclobutyl | | |
| methyl, ethyl, propyl, | isobutyl- | benzamidine-4-yl |
| butyl, isopropyl, | acylamine | |
| cyclopropyl, secbutyl, | | |
| or cyclobutyl | | |
| methyl, ethyl, propyl, | isobutyl- | 3-hydroxy- |
| butyl, isopropyl, | acylamine | benzamidine-4-yl |
| cyclopropyl, secbutyl, | | |
| or cyclobutyl | | |
| methyl, ethyl, propyl, | isobutyl- | 3,5-dihydroxy- |
| butyl, isopropyl, | acylamine | benzamide-4-yl |
| cyclopropyl, secbutyl, | | |
| or cyclobutyl | | |
| methyl, ethyl, propyl, | isobutyl- | 2-hydroxy- |
| butyl, isopropyl, | acylamine | benzamidine-4-yl |
| cyclopropyl, secbutyl, | | |
| or cyclobutyl | | |
| methyl, ethyl, propyl, | isobutyl- | 3,5,6-trifluoro- |
| butyl, isopropyl, | acylamine | 2-hydroxy- |
| cyclopropyl, secbutyl, | | benzamide-4-yl |
| or cyclobutyl | | |
| methyl, ethyl, propyl, | isobutyl- | 2,5,6-trifluoro- |
| butyl, isopropyl, | acylamine | 3-hydroxy- |
| cyclopropyl, secbutyl, | | benzamidine-4-yl |
| or cyclobutyl | | |
| methyl, ethyl, propyl, | isobutyl- | benzamidine-4-yl |
| butyl, isopropyl, | sulfoxyl | |
| cyclopropyl, secbutyl, | | |
| or cyclobutyl | | |
| methyl, ethyl, propyl, | isobutyl- | 3-hydroxy- |
| butyl, isopropyl, | sulfoxyl | benzamidine-4-yl |
| cyclopropyl, secbutyl, | | |
| or cyclobutyl | | |
| methyl, ethyl, propyl, | isobutyl- | 3,5-dihydroxy- |
| butyl, isopropyl, | sulfoxyl | benzamidine-4-yl |
| cyclopropyl, secbutyl, | | |
| or cyclobutyl | | |
| methyl, ethyl, propyl, | isobutyl- | 2-hydroxy- |
| butyl, isopropyl, | sulfoxyl | benzamidine-4-yl |
| cyclopropyl, secbutyl, | | |
| or cyclobutyl | | |
| methyl, ethyl, propyl, | isobutyl- | 3,5,6-trifluoro- |
| butyl, isopropyl, | sulfoxyl | 2-hydroxy- |
| cyclopropyl, secbutyl, | | benzamide-4-yl |
| or cyclobutyl | | |
| methyl, ethyl, propyl, | isobutyl- | 2,5,6-trifluoro- |
| butyl, isopropyl, | sulfoxyl | 3-hydroxy- |
| cyclopropyl, secbutyl, | | benzamidine-4-yl |
| or cyclobutyl | | |
| methyl, ethyl, propyl, | 2-cyclohexylamide | benzamidine-4-yl |
| butyl, isopropyl, | | |
| cyclopropyl, secbutyl, | | |
| or cyclobutyl | | |
| methyl, ethyl, propyl, | 2-cyclohexylamide | 3-hydroxy- |
| butyl, isopropyl, | | benzamidine-4-yl |
| cyclopropyl, secbutyl, | | |
| or cyclobutyl | | |
| methyl, ethyl, propyl, | 2-cyclohexylamide | 3,5-dihydroxy- |
| butyl, isopropyl, | | benzamidine-4-yl |
| cyclopropyl, secbutyl, | | |
| or cyclobutyl | | |
| methyl, ethyl, propyl, | 2-cyclohexylamide | 2-hydroxy- |
| butyl, isopropyl, | | benzamidine-4-yl |
| cyclopropyl, secbutyl, | | |
| or cyclobutyl | | |
| methyl, ethyl, propyl, | 2-cyclohexylamide | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | 2-cyclohexylamide | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | methoxyl | benzamidine-4-yl |
| butyl, isopropyl, | | |
| cyclopropyl, secbutyl, | | |
| or cyclobutyl | | |
| methyl, ethyl, propyl, | methoxyl | 3-hydroxy- |
| butyl, isopropyl, | | benzamidine-4-yl |
| cyclopropyl, secbutyl, | | |
| or cyclobutyl | | |
| methyl, ethyl, propyl, | methoxyl | 3,5-dihydroxy- |
| butyl, isopropyl, | | benzamidine-4-yl |
| cyclopropyl, secbutyl, | | |
| or cyclobutyl | | |
| methyl, ethyl, propyl, | methoxyl | 2-hydroxy- |
| butyl, isopropyl, | | benzamidine-4-yl |
| cyclopropyl, secbutyl, | | |
| or cyclobutyl | | |
| methyl, ethyl, propyl, | methoxyl | 3,5,6-trifluoro- |
| butyl, isopropyl, | | 2-hydroxy- |
| cyclopropyl, secbutyl, | | benzamide-4-yl |
| or cyclobutyl | | |
| methyl, ethyl, propyl, | methoxyl | 2,5,6-trifluoro- |
| butyl, isopropyl, | | 3-hydroxy- |
| cyclopropyl, secbutyl, | | benzamidine-4-yl |
| or cyclobutyl | | |
| methyl, ethyl, propyl, | sulfonamide | benzamidine-4-yl |
| butyl, isopropyl, | | |
| cyclopropyl, secbutyl, | | |
| or cyclobutyl | | |
| methyl, ethyl, propyl, | sulfonamide | 3-hydroxy- |
| butyl, isopropyl, | | benzamidine-4-yl |
| cyclopropyl, secbutyl, | | |
| or cyclobutyl | | |
| methyl, ethyl, propyl, | sulfonamide | 3,5-dihydroxy- |
| butyl, isopropyl, | | benzamidine-4-yl |
| cyclopropyl, secbutyl, | | |
| or cyclobutyl | | |
| methyl, ethyl, propyl, | sulfonamide | 2-hydroxy- |
| butyl, isopropyl, | | benzamidine-4-yl |
| cyclopropyl, secbutyl, | | |
| or cyclobutyl | | |
| methyl, ethyl, propyl, | sulfonamide | 3,5,6-trifluoro- |
| butyl, isopropyl, | | 2-hydroxy- |
| cyclopropyl, secbutyl, | | benzamidine-4-yl |
| or cyclobutyl | | |
| methyl, ethyl, propyl, | sulfonamide | 2,5,6-trifluoro- |
| butyl, isopropyl, | | 3-hydroxy- |
| cyclopropyl, secbutyl, | | benzamide-4-yl |
| or cyclobutyl | | |
| methyl, ethyl, propyl, | isobutyl- | benzamidine-4-yl |
| butyl, isopropyl, | sulfonamide | |
| cyclopropyl, secbutyl, | | |
| or cyclobutyl | | |
| methyl, ethyl, propyl, | isobutyl- | 3-hydroxy- |
| butyl, isopropyl, | sulfonamide | benzamidine-4-yl |
| cyclopropyl, secbutyl, | | |
| or cyclobutyl | | |
| methyl, ethyl, propyl, | isobutyl- | 3,5-dihydroxy- |
| butyl, isopropyl, | sulfonamide | benzamidine-4-yl |
| cyclopropyl, secbutyl, | | |
| or cyclobutyl | | |
| methyl, ethyl, propyl, | isobutyl- | 2-hydroxy- |
| butyl, isopropyl, | sulfonamide | benzamidine-4-yl |
| cyclopropyl, secbutyl, | | |
| or cyclobutyl | | |
| methyl, ethyl, propyl, | isobutyl- | 3,5,6-trifluoro- |
| butyl, isopropyl, | sulfonamide | 2-hydroxy- |
| cyclopropyl, secbutyl, | | benzamide-4-yl |
| or cyclobutyl | | |
| methyl, ethyl, propyl, | isobutyl- | 2,5,6-trifluoro- |
| butyl, isopropyl, | sulfonamide | 3-hydroxy- |
| cyclopropyl, secbutyl, | | benzamidine-4-yl |
| or cyclobutyl | | |

For convenience, each of the substituents identified for R⁴⁴ and Z₃ in Table 2 is set forth below.

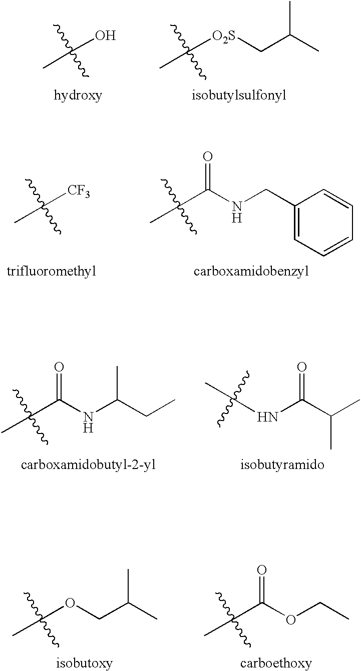

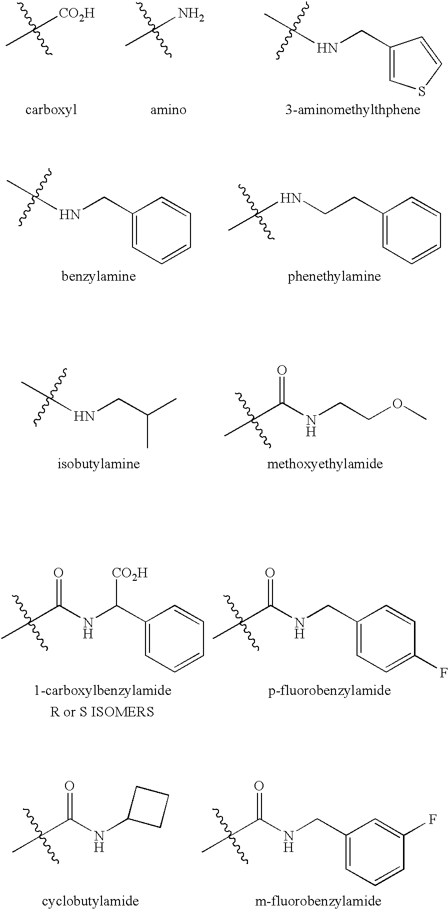

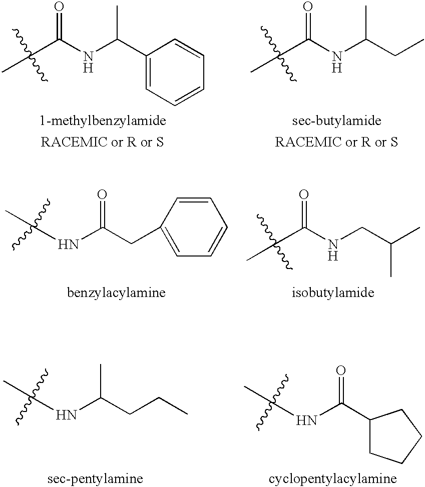

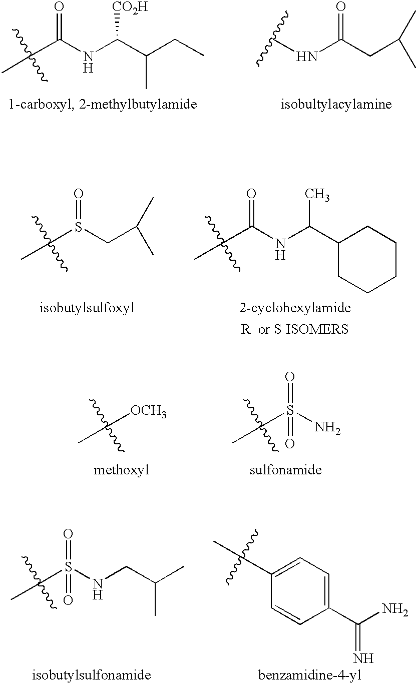

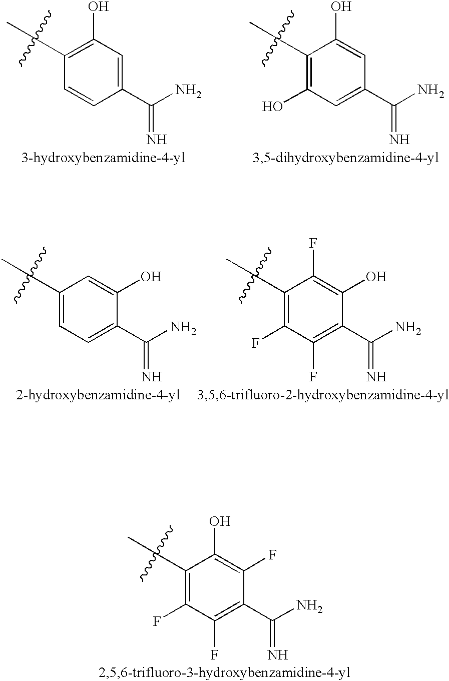

As a further embodiment, compounds of the present invention or a pharmaceutically-acceptable salt thereof, comprise a treatment and prophylaxis for thrombotic events resulting from coronary artery disease, cerebrovascular disease and other coagulation cascade related disorders in a subject, comprising administering to the subject having such disorder a therapeutically-effective amount of compounds the present invention or a pharmaceutically-acceptable salt thereof.

In another aspect of the invention, the compounds may also be used whenever inhibition of blood coagulation is required such as to prevent coagulation of stored whole blood and to prevent coagulation in other biological samples for testing or storage. Thus coagulation inhibitors of the present inhibition can be added to or contacted with stored whole blood and any medium containing or suspected of containing plasma coagulation factors and in which it is desired that blood coagulation be inhibited, e.g. when contacting the mammal's blood with material selected from the group consisting of vascular grafts, stents, orthopedic prothesis, cardiac prosthesis, and extracorporeal circulation systems.

Compounds of the invention are capable of inhibiting activity of serine proteases related to the coagulation cascade, and thus could be used in the manufacture of a medicament, a method for the prophylactic or therapeutic treatment of diseases mediated by coagulation cascade serine proteases, such as inhibiting the formation of blood platelet aggregates, inhibiting the formation of fibrin, inhibiting thrombus formation, and inhibiting embolus formation in a mammal, in blood, in blood products, and in mammalian organs. The compounds also can be used for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels in a mammal. The compounds also can be used to study the mechanism of action of coagulation cascade serine proteases to enable the design of better inhibitors and development of better assay methods. The compounds would be also useful in prevention of cerebral vascular accident (CVA) or stroke. Also included in the family of compounds are the pharmaceutically-acceptable salts thereof. The term “pharmaceutically-acceptable salt” embraces salts commonly used to form alkali metal salts and to form addition salts of free acids or free bases. The nature of the salt is not critical, provided that it is pharmaceutically acceptable. Suitable pharmaceutically-acceptable acid addition salts of compounds of formulas (1)-(7) may be prepared from inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, galacturonic acid. Suitable pharmaceutically-acceptable base addition salts of compounds of any of formulas (1)-(7) include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compound by reacting, for example, the appropriate acid or base with the compound of the present invention.

The present invention also comprises a pharmaceutical composition comprising a therapeutically-effective amount of the compound in association with at least one pharmaceutically-acceptable carrier, adjuvant or diluent. Pharmaceutical compositions of the present invention can comprise the active compounds in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended.

The active compounds and composition may, for example, be administered orally, intravascularly, intraperitoneally, subcutaneously, intramuscularly, oculary, or topically. For treating ocular build up of fibrin, the compounds may be administered intraocularly or topically as well as orally or parenterally.

The compounds can be administered in the form of a depot injection or implant preparation which may be formulated in such a manner as to permit a sustained release of the active ingredient. The active ingredient can be compressed into pellets or small cylinders and implanted subcutaneously or intramusculary as depot injections or implants. Implants may employ inert materials such as biodegradable polymers or synthetic silicones, for example, Silastic, silicone rubber or other silicon containing polymers.

The compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or ployethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphitpathic block copolymers of hydrogels.

For oral administration, the pharmaceutical composition may be in the form of, for example, tablets, capsules (each of which includes sustained release or timed release formulations), pills, powders, granules, elixers, tinctures, suspensions, liquids including syrups, and emulsions. The pharmaceutical composition is preferably made in the form of a dosage unit containing a particular amount of the active ingredient. Examples of such dosage units are tablets or capsules. The active ingredient may also be administered by injection as a composition wherein, for example, saline, dextrose or water may be used as a suitable carrier.

The amount of therapeutically active compounds which are administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely.

The pharmaceutical compositions may contain active ingredients in the range of about 0.1 to 2000 mg, and preferably in the range of about 0.5 to 500 mg. A daily dose of about 0.01 to 100 mg/kg body weight, and preferably between about 0.5 and about 20 mg/kg body weight, may be appropriate. The daily dose can be administered in one to four doses per day.

The compounds may be formulated in topical ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

For therapeutic purposes, the active compounds of the present invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Compounds of the present invention can exist in tautomeric, geometiric or stereoisomeric forms. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof, as falling within the scope of the invention. Pharmaceutically acceptable salts of such tautomeric, geometric or stereoisomeric forms are also included within the invention.

The terms “cis” and “trans”, as used herein, denote a form of geometric isomerism in which two carbon atoms connected by a double bond will each have a hydrogen atom on the same side of the double bond (“sis”) or on opposite sides of the double bond (“trans”).

Some of the compounds described contain alkenyl groups, and are meant to include both cis and trans or “E” and “Z” geometric forms.

Some of the compounds described contain one or more stereocenters and are meant to include R, S, and mixtures or R and S forms for each stereocenter present.

In addition to a compound having any of formulas (1)-(7), the composition of the invention may also comprise any agent, which when administered as part of a combination therapy with a compound having any of formulas (1)-(7), provides enhanced treatment options as compared to administration of either agent alone for the particular indication being treated. These indications are referred to herein as “thrombolytic conditions” and the agent administered with a compound having formula (1)-(7) is referred to as a “thrombolytic agent.” Generally speaking, the term “thrombolytic condition, “as used herein, encompasses vaso-occlusive events or related disorders including but not limited to, myocardial infarction, stroke, transient ischemic attacks including myocardial infarction and stroke, amaurosis fugax, aortic stenosis, cardiac stenosis, coronary stenosis and pulmonary stenosis. Stenosis is the narrowing or stricture of a duct or canal. Coronary stenosis is the narrowing or stricture of a coronary artery. Cardiac stenosis is a narrowing or diminution of any heart passage or cavity. Pulmonary stenosis is the narrowing of the opening between the pulmonary artery and the right ventricle. Aortic stenosis is narrowing of the aortic orifice of the heart or of the aorta itself. Moreover, the term “thrombolytic agent,” as used herein unless otherwise indicated, includes anti-platelet agents, anticoagulation agents, and cardiovascular therapeutic agents.

In one embodiment, when general thrombolytic condition are being treated, the thrombolytic agent may belong to a class of compounds that cause a reduction in platelet aggregation and thereby diminishes the size of or prevents the formation of a thrombus or occlusion. One such class of compounds are anti-platelet inhibitors that ameliorate prostaglandin synthesis. These agents include salicylates (e.g. aspirin) and other NSAIDS, ticlopidine, or clopidrogel. In a further embodiment, the anti-platelet inhibitor is a GP IIb/IIIa inhibitor.

In yet another embodiment, when indications such as unstable angina, thrombolytic occlusions or prevention of reocculsion after angioplasty and restenosis are being treated, the thrombolytic agent co-administered along with compounds having any of formulas (1)-(7) include fibrinogen receptor antagonists, anti-coagulants such as warfarin or heparins, and plasminogen activators, such as tissue plasminogen activator (tPA) or streptokinase. In still another embodiment, when the indication being treated is coronary artery disease or patients subjected to angioplasty procedures, suitable agents include antihypercholesterolemics (e.g. HMG CoA reductase inhibitors such as mevastatin, lovastatin, simvastatin, pravastatin, and fluvastatin, HMG CoA synthatase inhibitors, etc.), anti-diabetic drugs, or other cardiovascular agents (e.g. loop diuretics, thiazide type diuretics, nitrates, aldosterone antagonistics (e.g. spironolactone and epoxymexlerenone), angiotensin converting enzyme (e.g. ACE) inhibitors, angiotensin II receptor antagonists, beta-blockers, antiarrythmics, anti-hypertension agents, and calcium channel blockers to treat or prevent atheriosclerosis.

Generally speaking, the pharmacokinetics of the particular agent to be administered will dictate the most preferred method of administration and dosing regiment. For example, when the thrombolytic agent has a rapid plasma clearance time and a short half-life, a preferred mode of administration is as a bolus injection followed by an intravenous infusion. Alternatively, when the thrombolytic agent has a lower plasma clearance time and a longer half-life, a preferred mode of administration is as a single bolus injection.

Additionally typical doses of compounds of the present invention with other suitable thrombolytic agents may be the same as those doses of compounds having formula (1)-(7) without coadministration of the thrombolytic agent, or may be substantially less than those doses of compounds having formula (1)-(7) administered without coadministration of the thrombolytic agents and will vary depending on a subject's therapeutic needs. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The timing of the administration of the compound having formula (1)-(7) in relation to the administration of the thrombolytic agent may also vary from subject to subject and depend upon the thrombolytic condition being treated. In one embodiment of the invention, the compound of formula (1)-(7) and thrombolytic agent may be administered substantially simultaneously, meaning that both agents may be administered to the subject at approximately the same time. For example, the compound of formula (1)-(7) or pharmaceutically acceptable salt or prodrug thereof is administered during a continuous period beginning on the same day as the beginning of the thrombolytic agent and extending to a period after the end of the thrombolytic agent. Alternatively, the compound of formula (1)-(7) and thrombolytic agent may be administered sequentially, meaning that they are administered at separate times during separate treatments. In one embodiment, for example, the compound of formula (1)-(7) or a pharmaceutically acceptable salt or prodrug thereof is administered during a continuous period beginning prior to administration of the thrombolytic agent and ending after administration of the thrombolytic agent. Of course, it is also possible that the compound of formula (1)-(7) may be administered either more or less frequently than the thrombolytic agent. One skilled in the art can readily design suitable treatment regiments for a particular subject depending on the particular thrombolytic condition being treated. Moreover, it will be apparent to those skilled in the art that it is possible, and perhaps desirable, to combine various times and methods of administration in the practice of the present invention.

In some aspects, the invention provides treatment for subjects who are at risk of a thrombolytic condition. These subjects may or may not have had a previous thrombolytic condition. The invention embraces the treatment of subjects prior to a thrombolytic condition, at a time of a thrombolytic condition and following a thrombolytic condition. Thus, as used herein, the “treatment” of a subject is intended to embrace both prophylactic and therapeutic treatment, and can be used either to limit or to eliminate altogether the symptoms or the occurrence of a thrombolytic condition. In one embodiment, the subject may exhibit symptoms of a thrombolytic condition.

The invention also embraces the treatment of a subject that has an abnormally elevated risk of a thrombolytic condition. The subject may have vascular disease. The vascular disease may be selected from the group consisting of arteriosclerosis, cardiovascular disease, cerebrovascular disease, renovascular disease, mesenteric vascular disease, pulmonary vascular disease, ocular vascular disease or peripheral vascular disease.

In one embodiment, however, the subject has had a primary thrombolytic condition. The composition of the invention may be administered to a subject following such a primary thrombolytic condition. The method of the invention also embraces treatment of a subject to reduce the risk of a secondary thrombotic event or to inhibit the propagation of an existing thrombotic event. By way of example, the thrombotic event may be selected from the group consisting of arterial thrombosis, coronary thrombosis, heart valve thrombosis, coronary stenosis, stent thrombosis and graft thrombosis. The thrombolytic condition also includes disorders or conditions that may arise from a thrombotic event or a thromboembolic event and in this regard a thrombolytic condition includes, but is not limited to myocardial infarction, stroke and transient ischemic attack. In one embodiment, the thrombolytic condition is myocardial infarction. In yet another embodiment, the subject has had a myocardial infarction. A subject who has hypercholesterolemia, hypertension or atherosclerosis also can be treated by the methods of the invention.

In yet another embodiment, the subject is one who will undergo an elective surgical procedure. The composition of the invention may be administered to such a subject prior to the elective surgical procedure. The method of the invention can also be directed towards a subject who has undergone a surgical procedure. As used herein, a “surgical procedure” is meant to embrace those procedures that have been classically regarded as surgical procedures as well as interventional cardiology procedures such as arteriography, angiography, angioplasty and stenting. Thus, the surgical procedure, whether elective or not, can be selected from the group consisting of coronary angiography, coronary stent placement, coronary by-pass surgery, carotid artery procedure, peripheral stent placement, vascular grafting, thrombectomy, peripheral vascular surgery, vascular surgery, organ transplant, artificial heart transplant, vascular angioplasty, vascular laser therapy, vascular replacement, prosthetic valve replacement and vascular stenting.

The present novel methods preferably employ compounds which selectively inhibit human TF-VIIA over the inhibition of both human Thrombin II and human factor Xa. Preferably, the compounds have a human TF-VIIA IC₅₀ of less than 0.5 mM and also have a selectivity ratio of TF-VIIA inhibition over both human Thrombin II and human factor Xa inhibition of at least 10, and more preferably at least 100. Even more preferably, the compounds have a human TF-VIIA IC₅₀ of less than 0.1 mM and also have a selectivity ratio of TF-VIIA inhibition over both human Thrombin II and human factor Xa inhibition of at least 1000, and most preferably at least 10,000.

All mentioned references are incorporated by reference as if here written.

Although this invention has been described with respect to specific embodiments, the details of these embodiments are not to be construed as limitations. Without further elaboration, it is believed that one skilled in the art can, using the preceding descriptions, utilize the present invention to its fullest extent. Compounds containing multiple variations of the structural modifications illustrated in the Schemes are also contemplated. Those skilled in the art will readily understand that known variations of the conditions and processes of the following preparative procedures can be used to prepare these compounds.

General Synthetic Procedures and Specific Examples

The compounds of the present invention can be—synthesized, for example, according to the following procedures and Schemes given below.

Abbreviations used in the schemes and tables include: “AA” represents amino acids, “AcCN” represents acetonitrile, “AcOH” represents acetic acid, “BINAP” represents 2,2′-bis(diphenylphosphino)-1,1′-binaphthyl, “BnOH” represents benzyl alcohol, “BnCHO” represents 2-phenylethanal, “BnSO₂Cl” represents benzylsulfonyl chloride, “Boc” represents tert-butyloxycarbonyl, “BOP” represents benzotriazol-1-yl-oxy-tris-(dimethylamino), “bu” represents butyl, “dba” represents dibenzylidene-acetone, “DCC” represents 1,3-dicyclohexylcarbodiimide, “DCM” represents dichloromethane or methylene chloride, “DIBAH” or “DIBAL” represents diisobutylaluminum hydride, “DMF” represents dimethylformamide, “DMSO” represents dimethylsulfoxide, “DPPA” represents diphenylphosphoryl azide”, “EDC” represents 1-[3-(dimethylamino)propyl]-3-ethylcarbodiimide hydrochloride, “Ex. No.” represents Example Number, “Fmoc” represents 9-fluorenylmethoxycarbonyl, “HOBt” represents hydroxybenzoltriazole”, “LDA” represents lithium diisopropylamide, “MW” represents molecular weight, “NMM” represents N-methylmorpholine, “Ph” represents phenyl or aryl, “PHTH” represents a phthaloyl group, “pnZ” represents 4-nitrobenzyloxy-carbonyl, “PTC” represents a phase transfer catalyst, “py” represents pyridine, “RNH₂” represents a primary organic amine, “SEM” represents 2-(trimethylsilyl)ethoxy-methyl chloride, “p-TsOH” represents paratoluenesulfonic acid, “TBAF” represents tetrabutylammonium fluoride, “TBTU” represents 2-(1H-benzotriozole-1-yl)-1,1,3,3-tetramethyl uronium tetrafluoroborate, “TEA” represents triethylamine, “TFA” represents trifluoroacetic acid, “THF” represents tetrahydrofuran, “TMS” represents trimethylsilyl, “TMSCN” represents trimethylsilyl cyanide, and “Cbz” or “Z” represents benzyloxycarbonyl.

As used in the schemes and examples, L₁, Z₁, Z₃, Z₄, R₄₄, and R₈₀ along with any other variable depicted, encompasses every group described for each particular variable for each embodiment of compounds having any of the formulas detailed herein. Further, R^{4a} and R^{4b} are hydrogen, Z₁ and Z₆ are independently hydrogen or halogen, and L₆ is a bond.

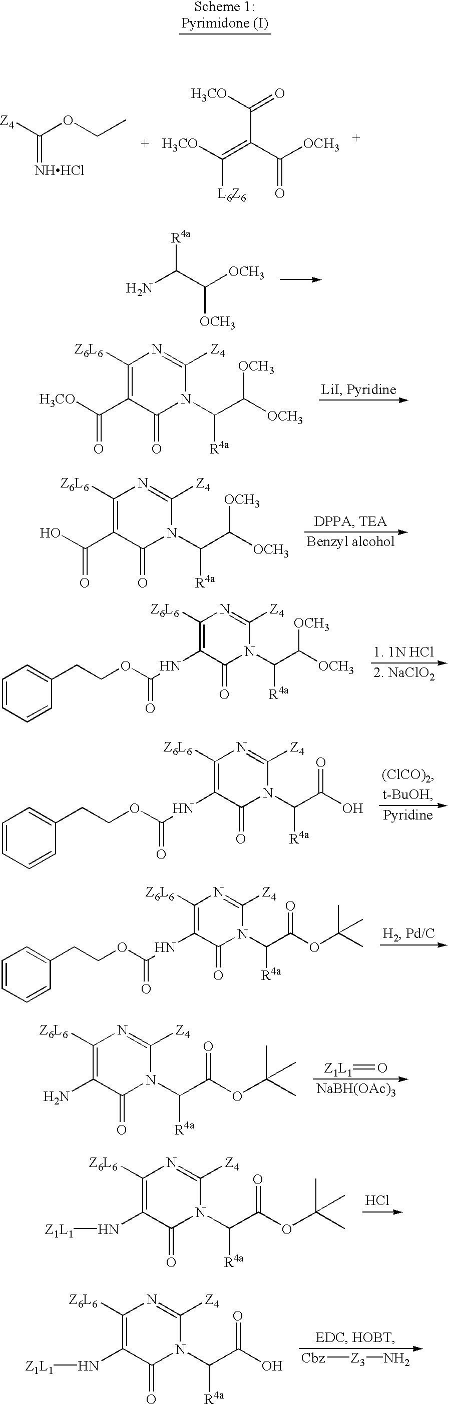

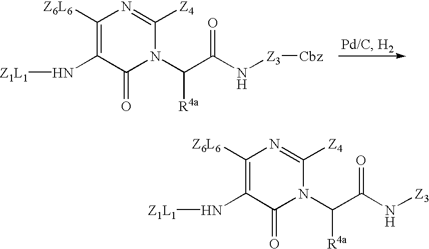

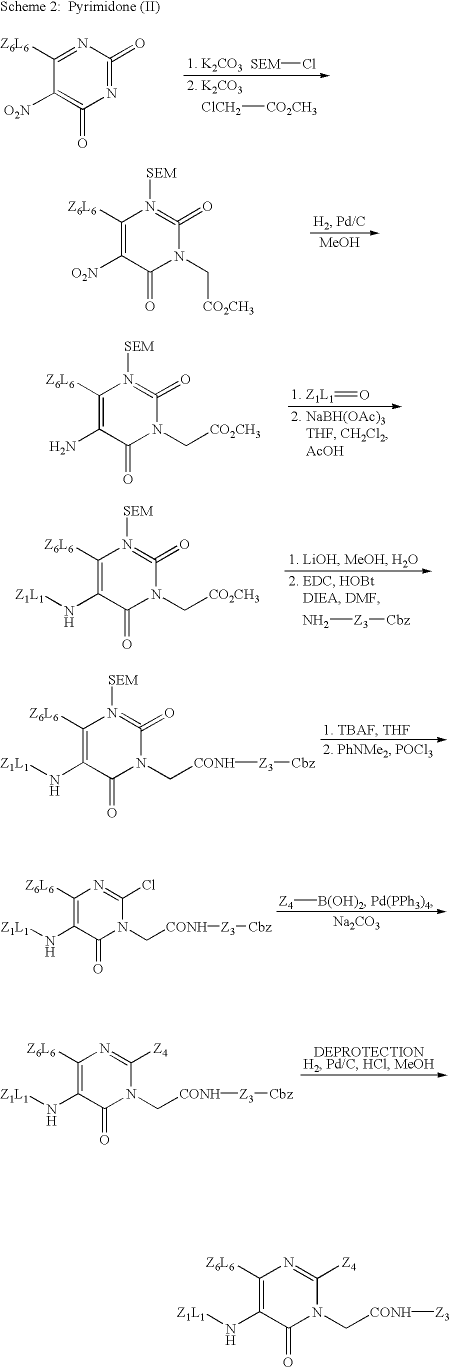

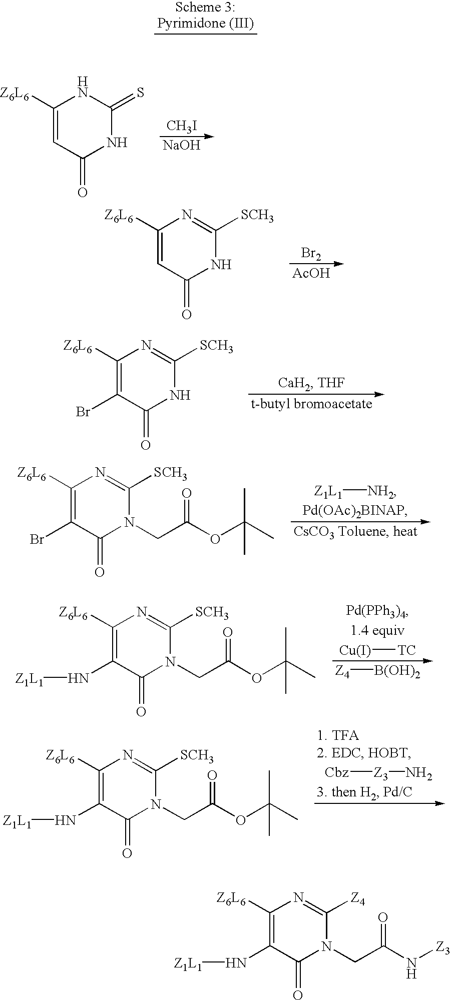

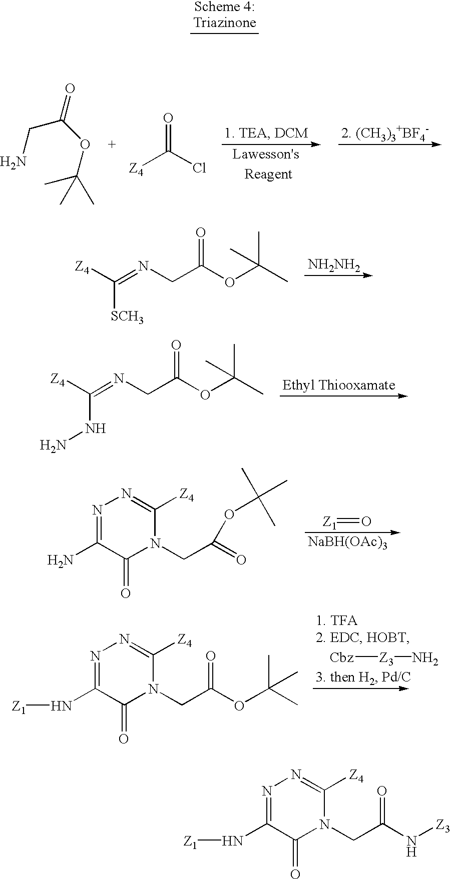

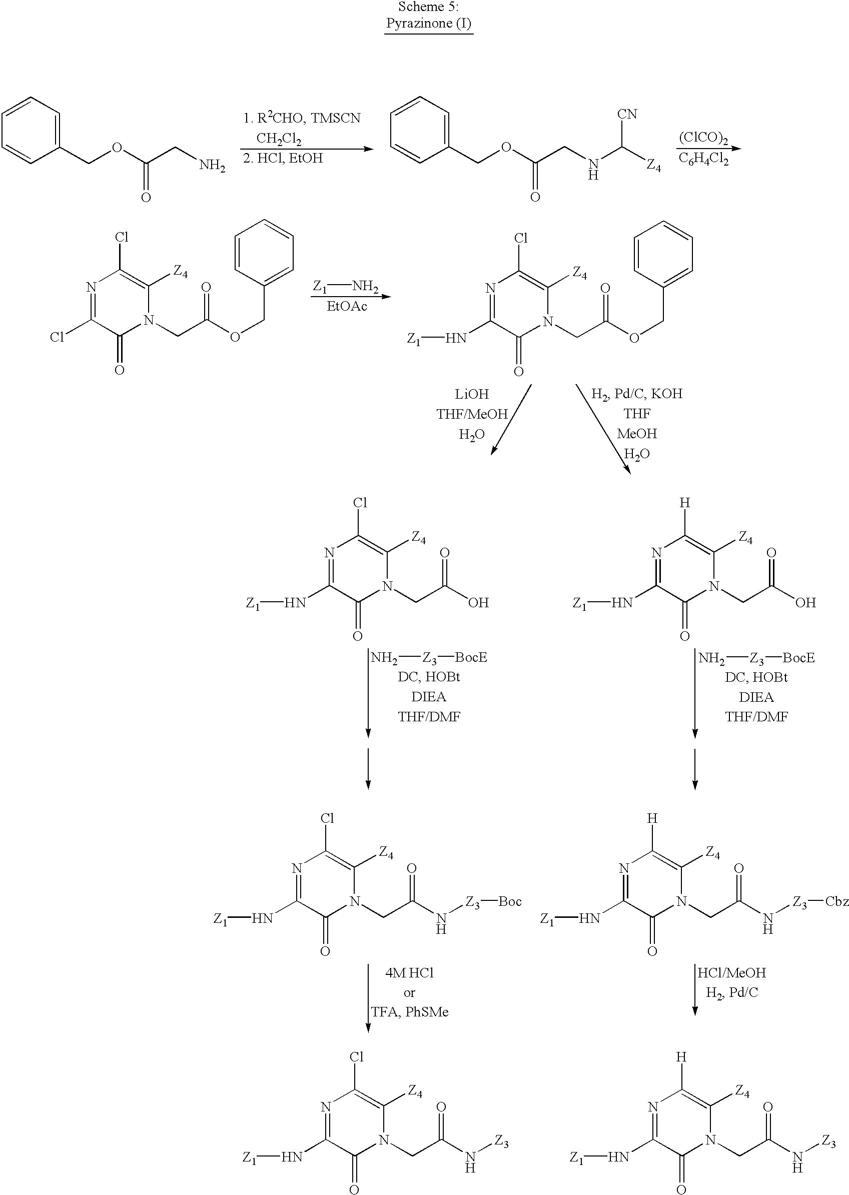

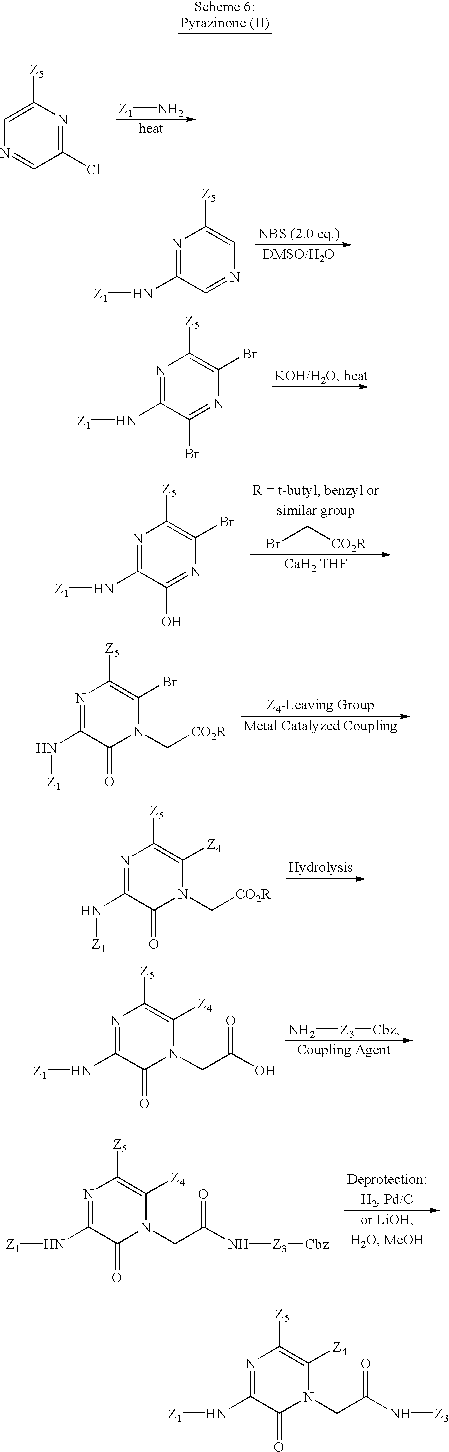

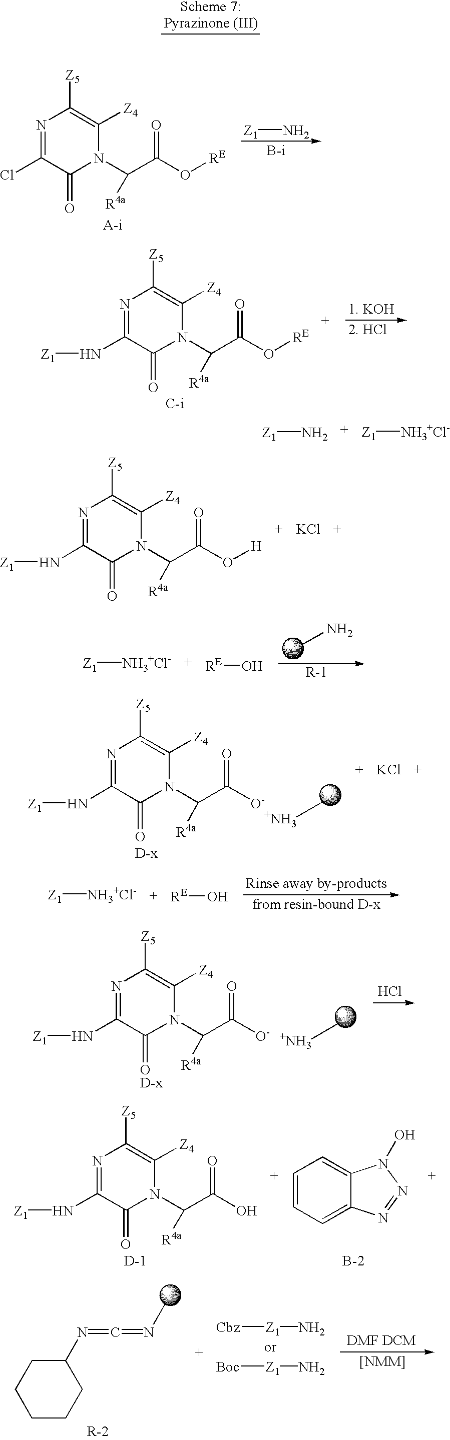

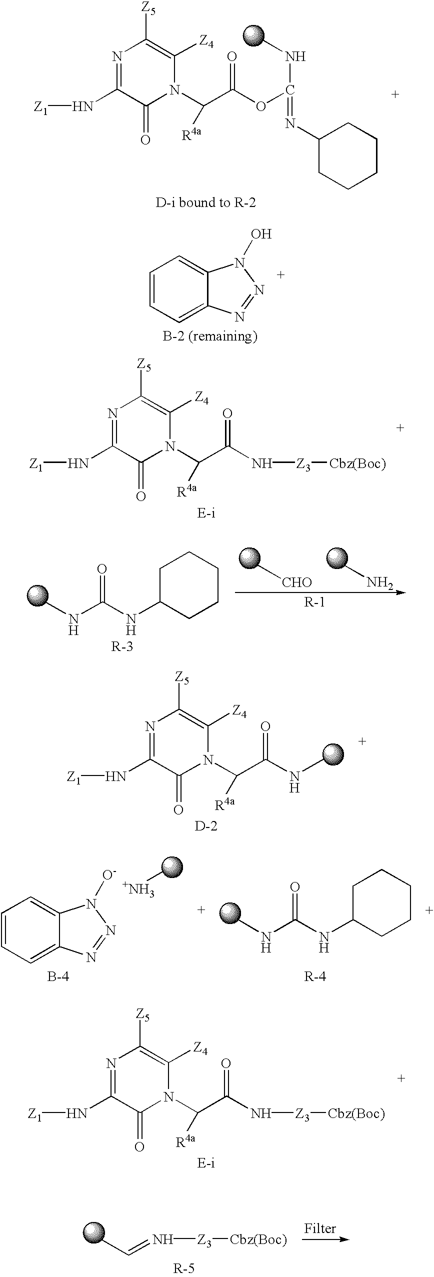

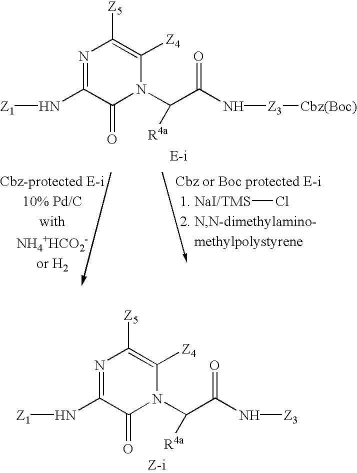

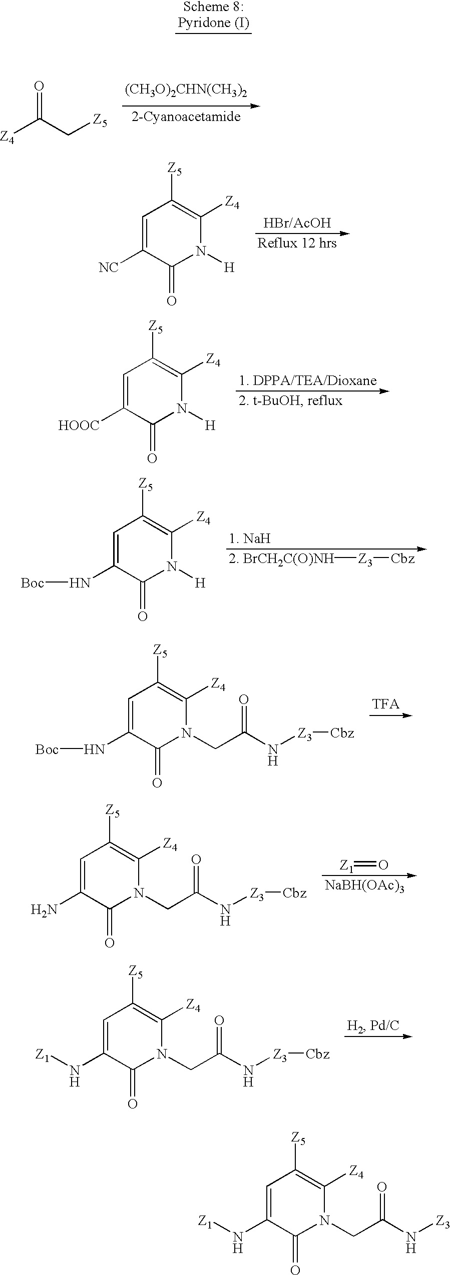

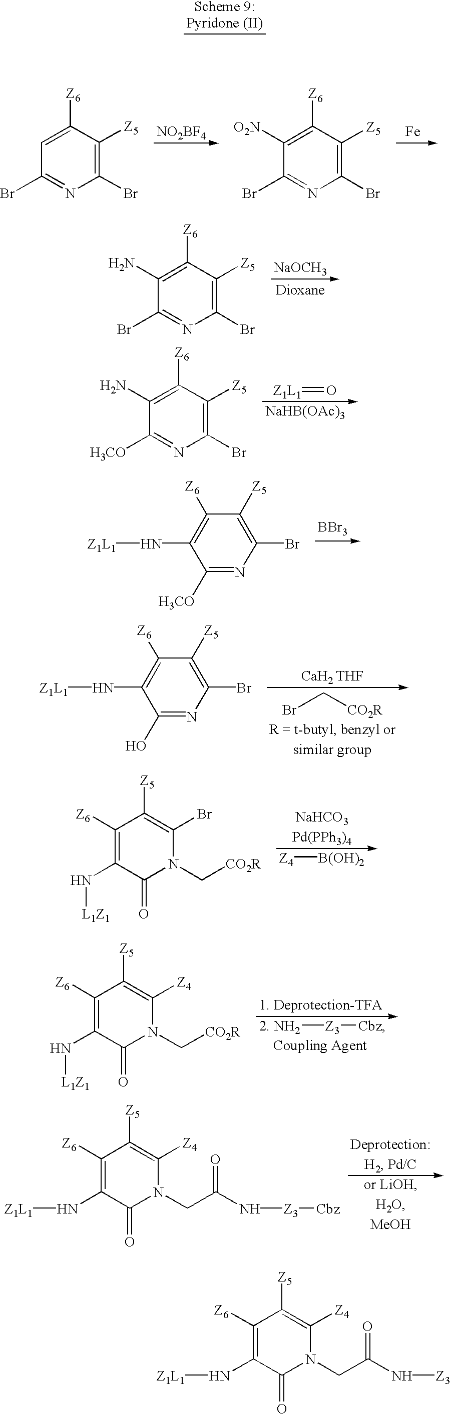

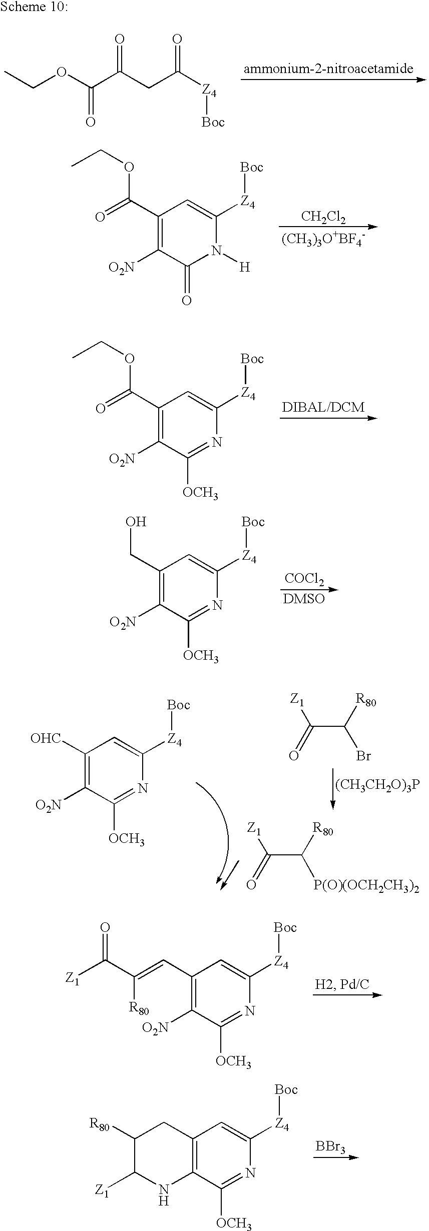

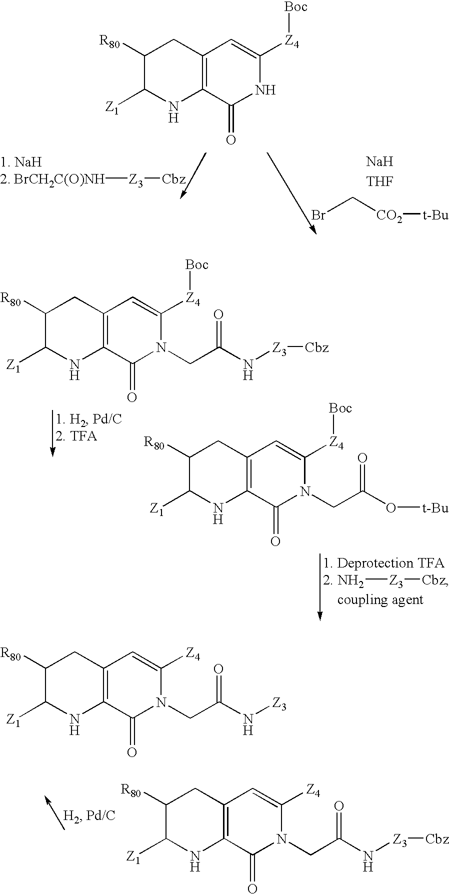

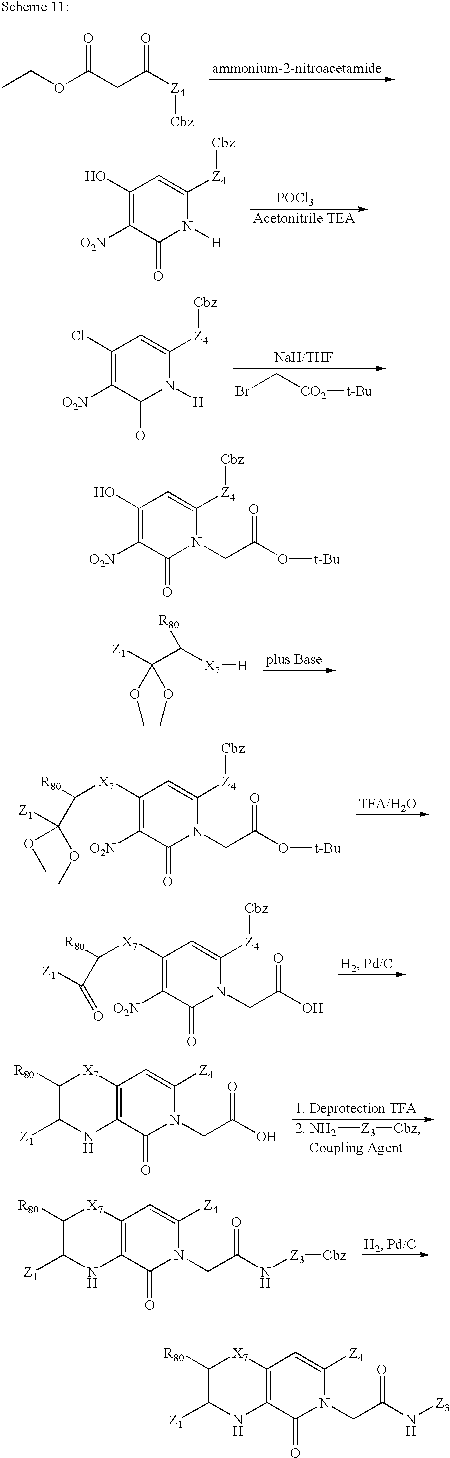

### Assays for Biological Activity

### TF-VIIa Assay

In this assay 100 nM recombinant soluble tissue factor and 2 nM recombinant human factor VIIa are added to a 96-well assay plate containing 0.4 mM of the substrate, N-methylsulfonyl-D-phe-gly-arg-p-nitroaniline and either inhibitor or buffer (5 mM CaCl₂, 50 mM Tris-HCl, pH 8.0, 100 mM NaCl, 0.1% BSA). The reaction, in a final volume of 100 ul is measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of TF-VIIa activity is calculated from OD405 nm value from the experimental and control sample.

### Xa Assay

Human factor Xa (0.3 nM) and 0.15 mM -a-Benzyloxycarbonyl-D-arginyl-L-glycyl-L-arginine-p-nitroaniline-dihydrochloride (S-2765) are added to a 96-well assay plate containing either inhibitor or buffer (50 mM Tris-HCl, pH 8.0, 100 mM NaCl, 0.1% BSA). The reaction, in a final volume of 100 ul is measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of Xa activity is calculated from OD405 nm value from the experimental and control sample.

### Thrombin Assay

Human thrombin (0.28 nM) and 0.06 mM H-D-Phenylalanyl-L-pipecolyl-L-arginine-p-nitroaniline dihydrochloride are added to a 96-well assay plate containing either inhibitor or buffer (50 mM Tris-HCl, pH 8.0, 100 mM NaCl, 0.1% BSA). The reaction, in a final volume of 100 ul is measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of thrombin activity is calculated from OD405 nm value from the experimental and control sample.

Trypsin Assay Trypsin (5 ug/ml; type IX from porcine pancreas) and 0.375 mM N-a-Benzoyl-L-arginine-p-nitroanilide (L-BAPNA) are added to a 96-well assay plate containing either inhibitor or buffer (50 mM Tris-HCl, pH 8.0, 100 mM NaCl, 0.1% BSA). The reactions, in a final volume of 100 ul are measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of trypsin activity is calculated from OD405 nm value from the experimental and control sample.

Recombinant soluble TF, consisting of amino acids 1-219 of the mature protein sequence was expressed in E. coli and purified using a Mono Q Sepharose FPLC. Recombinant human VIIa was purchased from American Diagnostica, Greenwich CT and chromogenic substrate N-methylsulfonyl-D-phe-gly-arg-p-nitroaniline was prepared by American Peptide Company, Inc., Sunnyvale, Calif. Factor Xa was obtained from Enzyme Research Laboratories, South Bend Ind., thrombin from Calbiochem, La Jolla, Calif., and trypsin and L-BAPNA from Sigma, St. Louis Mo. The chromogenic substrates S-2765 and S-2238 were purchased from Chromogenix, Sweden.

### Prothrombin Time Assay

The prothrombin time (PT) assay is a clotting assay that is used to determine deficiencies of clotting factor activity in the extrinsic pathway. The assay measures time to clotting after the addition of thromboplastin (human tissue factor). Normal human pooled plasma is incubated with 100 uM compound or saline in a coagulation machine. Thromboplastin is added and time to clotting is measured. Compounds with greater than 3×normal PT of 11.0 seconds are further analyzed by concentration response to determine at what concentration the prothrombin time is prolonged by 2×normal.

Using the bioassay procedures described herein, the biological activity of the compounds of Table 1 are summarized below in Table 3. While all of the compounds in Table 3 exhibit activity towards TF-VIIa, a compound selective for TF-VIIa preferably possesses an IC₅₀ value of less than 0.1 uM for TF-VIIa and greater than 30.0 uM against thrombin and/or factor Xa. Compounds meeting these criteria are at least 300×more selective against TF-VIIa than against other proteases along the extrinsic coagulation pathway. Additionally, in general, compounds that selectively inhibit TF-VIIa at a concentration of less than 0.1 uM effect a 2×prolongation of prothrombin time at a concentration of less than 50 uM. Accordingly, the data for each compound in Table 3 is reported as less than or greater than 0.1 uM for TF-VIIa, as less than or greater than 30 uM for both thrombin and factor Xa, and as less than or greater than 50 uM for prothrombin time. As used herein, the term “ND” is used to indicate that no data is available.

TABLE 3

| | | | Factor Xa | PT |
|---|---|---|---|---|
| Example | TF-VIIa | Thrombin (II) | (II) | (Prothrombin time) |
| No. | IC₅₀ (uM) | IC₅₀ (uM) | IC₅₀ (uM) | 2xPT (uM) |
| 1 | <0.1 | >30.0 | >30.0 | ND |
| 2 | <0.1 | >30.0 | >30.0 | ND |
| 3 | <0.1 | >30.0 | >30.0 | <50.0 |
| 4 | <0.1 | >30.0 | >30.0 | >50.0 |
| 5 | <0.1 | <30.0 | >30.0 | ND |
| 6 | <0.1 | >30.0 | >30.0 | <50.0 |
| 7 | <0.1 | <30.0 | >30.0 | <50.0 |
| 8 | <0.1 | >30.0 | >30.0 | ND |
| 9 | <0.1 | <30.0 | >30.0 | ND |
| 10 | <0.1 | >30.0 | >30.0 | <50.0 |
| 11 | <0.1 | <30.0 | <30.0 | ND |
| 12 | <0.1 | >30.0 | >30.0 | ND |
| 13 | <0.1 | <30.0 | >30.0 | ND |
| 14 | ND | ND | ND | ND |
| 15 | <0.1 | <30.0 | >30.0 | <50.0 |
| 16 | <0.1 | >30.0 | >30.0 | <50.0 |
| 17 | <0.1 | >30.0 | >30.0 | <50.0 |
| 18 | <0.1 | >30.0 | >30.0 | <50.0 |
| 19 | ND | ND | ND | ND |
| 20 | >0.1 | >30.0 | >30.0 | ND |
| 21 | <0.1 | >30.0 | >30.0 | ND |
| 22 | >0.1 | >30.0 | >30.0 | ND |
| 23 | >0.1 | >30.0 | >30.0 | ND |
| 24 | <0.1 | >30.0 | >30.0 | <50.0 |
| 25 | <0.1 | <30.0 | >30.0 | ND |
| 26 | <0.1 | >30.0 | >30.0 | <50.0 |
| 27 | <0.1 | >30.0 | >30.0 | ND |
| 28 | <0.1 | >30.0 | >30.0 | <50.0 |
| 29 | <0.1 | >30.0 | >30.0 | <50.0 |
| 30 | >0.1 | >30.0 | >30.0 | ND |
| 31 | >0.1 | >30.0 | >30.0 | ND |
| 32 | >0.1 | >30.0 | >30.0 | ND |
| 33 | <0.1 | >30.0 | >30.0 | ND |
| 34 | ND | ND | ND | ND |
| 35 | <0.1 | >30.0 | >30.0 | <50.0 |
| 36 | >0.1 | >30.0 | >30.0 | ND |
| 37 | >0.1 | >30.0 | >30.0 | ND |
| 38 | <0.1 | ND | >30.0 | ND |
| 39 | <0.1 | ND | >30.0 | ND |
| 40 | <0.1 | ND | >30.0 | ND |
| 41 | <0.1 | ND | >30.0 | ND |
| 42 | <0.1 | ND | >30.0 | ND |
| 43 | <0.1 | ND | >30.0 | ND |
| 44 | <0.1 | ND | >30.0 | ND |
| 45 | <0.1 | ND | >30.0 | ND |
| 46 | <0.1 | ND | >30.0 | ND |
| 47 | <0.1 | ND | >30.0 | ND |
| 48 | >0.1 | >30.0 | >30.0 | ND |
| 49 | >0.1 | >30.0 | >30.0 | <50.0 |
| 50 | <0.1 | <30.0 | >30.0 | <50.0 |
| 51 | >0.1 | >30.0 | >30.0 | <50.0 |
| 52 | <0.1 | <30.0 | >30.0 | ND |
| 53 | <0.1 | >30.0 | >30.0 | <50.0 |
| 54 | <0.1 | >30.0 | >30.0 | <50.0 |
| 55 | <0.1 | >30.0 | >30.0 | ND |
| 56 | <0.1 | <30.0 | >30.0 | <50.0 |
| 57 | <0.1 | <30.0 | >30.0 | <50.0 |
| 58 | <0.1 | <30.0 | >30.0 | <50.0 |
| 59 | >0.1 | >30.0 | >30.0 | >50.0 |
| 60 | <0.1 | <30.0 | >30.0 | <50.0 |
| 61 | <0.1 | >30.0 | >30.0 | <50.0 |
| 62 | <0.1 | <30.0 | >30.0 | <50.0 |
| 63 | <0.1 | >30.0 | >30.0 | <50.0 |
| 64 | <0.1 | >30.0 | >30.0 | <50.0 |
| 65 | >0.1 | >30.0 | >30.0 | >50.0 |
| 66 | <0.1 | >30.0 | >30.0 | <50.0 |
| 67 | <0.1 | >30.0 | <30.0 | <50.0 |
| 68 | <0.1 | >30.0 | <30.0 | <50.0 |
| 69 | <0.1 | >30.0 | >30.0 | <50.0 |
| 70 | <0.1 | >30.0 | >30.0 | <50.0 |
| 71 | <0.1 | <30.0 | >30.0 | <50.0 |
| 72 | <0.1 | <30.0 | >30.0 | <50.0 |
| 73 | <0.1 | >30.0 | >30.0 | <50.0 |
| 74 | <0.1 | >30.0 | >30.0 | <50.0 |
| 75 | ND | ND | ND | ND |
| 76 | <0.1 | >30.0 | >30.0 | <50.0 |
| 77 | >0.1 | >30.0 | >30.0 | <50.0 |
| 78 | >0.1 | >30.0 | >30.0 | <50.0 |
| 79 | >0.1 | >30.0 | >30.0 | <50.0 |
| 80 | <0.1 | >30.0 | >30.0 | >50.0 |
| 81 | <0.1 | <30.0 | >30.0 | <50.0 |
| 82 | <0.1 | <30.0 | >30.0 | <50.0 |
| 83 | <0.1 | >30.0 | >30.0 | <50.0 |
| 84 | <0.1 | >30.0 | >30.0 | <50.0 |
| 85 | <0.1 | >30.0 | >30.0 | <50.0 |
| 86 | <0.1 | <30.0 | >30.0 | <50.0 |
| 87 | <0.1 | <30.0 | >30.0 | <50.0 |
| 88 | >0.1 | >30.0 | >30.0 | <50.0 |
| 89 | <0.1 | >30.0 | >30.0 | <50.0 |
| 90 | <0.1 | >30.0 | >30.0 | <50.0 |
| 91 | <0.1 | >30.0 | >30.0 | <50.0 |
| 92 | <0.1 | >30.0 | >30.0 | <50.0 |
| 93 | <0.1 | >30.0 | >30.0 | <50.0 |
| 94 | <0.1 | >30.0 | >30.0 | <50.0 |
| 95 | <0.1 | >30.0 | >30.0 | <50.0 |
| 96 | <0.1 | >30.0 | >30.0 | <50.0 |
| 97 | >0.1 | >30.0 | >30.0 | ND |
| 98 | <0.1 | >30.0 | >30.0 | <50.0 |
| 99 | <0.1 | >30.0 | >30.0 | <50.0 |
| 100 | <0.1 | <30.0 | >30.0 | <50.0 |
| 101 | <0.1 | <30.0 | >30.0 | <50.0 |
| 102 | <0.1 | <30.0 | >30.0 | <50.0 |
| 103 | <0.1 | >30.0 | >30.0 | <50.0 |
| 104 | <0.1 | <30.0 | >30.0 | <50.0 |
| 105 | <0.1 | >30.0 | >30.0 | <50.0 |
| 106 | <0.1 | >30.0 | >30.0 | <50.0 |
| 107 | <0.1 | >30.0 | >30.0 | <50.0 |
| 108 | <0.1 | <30.0 | >30.0 | <50.0 |
| 109 | <0.1 | <30.0 | >30.0 | <50.0 |
| 110 | <0.1 | >30.0 | >30.0 | <50.0 |
| 111 | <0.1 | >30.0 | >30.0 | <50.0 |
| 112 | <0.1 | <30.0 | >30.0 | <50.0 |
| 113 | <0.1 | >30.0 | >30.0 | <50.0 |
| 114 | <0.1 | >30.0 | >30.0 | <50.0 |
| 115 | <0.1 | ND | ND | <50.0 |
| 116 | >0.1 | >30.0 | <30.0 | <50.0 |
| 117 | ND | ND | ND | <50.0 |
| 118 | <0.1 | >30.0 | >30.0 | <50.0 |
| 119 | <0.1 | <30.0 | >30.0 | <50.0 |
| 120 | ND | ND | ND | ND |
| 121 | <0.1 | <30.0 | >30.0 | ND |
| 122 | ND | ND | ND | ND |
| 123 | <0.1 | >30.0 | >30.0 | <50.0 |
| 124 | <0.1 | >30.0 | >30.0 | <50.0 |
| 125 | ND | ND | ND | ND |
| 126 | <0.1 | <30.0 | >30.0 | ND |
| 127 | <0.1 | <30.0 | >30.0 | <50.0 |
| 128 | <0.1 | >30.0 | >30.0 | <50.0 |
| 129 | <0.1 | >30.0 | >30.0 | ND |
| 130 | <0.1 | <30.0 | >30.0 | <50.0 |
| 131 | <0.1 | <30.0 | >30.0 | <50.0 |
| 132 | <0.1 | <30.0 | >30.0 | ND |
| 133 | <0.1 | <30.0 | >30.0 | <50.0 |
| 134 | <0.1 | <30.0 | >30.0 | <50.0 |
| 135 | <0.1 | <30.0 | >30.0 | <50.0 |
| 136 | <0.1 | <30.0 | >30.0 | <50.0 |
| 137 | <0.1 | <30.0 | >30.0 | <50.0 |
| 138 | <0.1 | <30.0 | >30.0 | ND |
| 139 | <0.1 | <30.0 | >30.0 | <50.0 |
| 140 | <0.1 | <30.0 | >30.0 | <50.0 |
| 141 | <0.1 | <30.0 | >30.0 | <50.0 |
| 142 | <0.1 | >30.0 | >30.0 | <50.0 |
| 143 | <0.1 | <30.0 | >30.0 | <50.0 |
| 144 | <0.1 | >30.0 | >30.0 | <50.0 |
| 145 | <0.1 | ND | ND | <50.0 |
| 146 | <0.1 | ND | ND | <50.0 |
| 147 | <0.1 | ND | ND | <50.0 |
| 148 | <0.1 | ND | ND | <50.0 |
| 149 | >0.1 | ND | ND | ND |
| 150 | <0.1 | ND | ND | ND |
| 151 | <0.1 | ND | ND | ND |
| 152 | ND | ND | ND | <50.0 |
| 153 | ND | ND | ND | <50.0 |
| 154 | ND | ND | ND | >50.0 |
| 155 | ND | ND | ND | ND |
| 156 | ND | ND | ND | ND |
| 157 | ND | ND | ND | ND |
| 158 | ND | ND | ND | ND |
| 159 | ND | ND | ND | ND |
| 160 | ND | ND | ND | ND |
| 161 | <0.1 | >30.0 | >30.0 | <50.0 |
| 162 | <0.1 | >30.0 | >30.0 | <50.0 |
| 163 | >0.1 | >30.0 | >30.0 | ND |
| 164 | <0.1 | >30.0 | >30.0 | <50.0 |
| 165 | <0.1 | >30.0 | >30.0 | <50.0 |
| 166 | <0.1 | >30.0 | >30.0 | <50.0 |
| 167 | <0.1 | <30.0 | >30.0 | <50.0 |
| 168 | >0.1 | >30.0 | >30.0 | ND |
| 169 | <0.1 | <30.0 | >30.0 | <50.0 |
| 170 | <0.1 | >30.0 | >30.0 | <50.0 |
| 171 | <0.1 | >30.0 | >30.0 | <50.0 |
| 172 | <0.1 | >30.0 | >30.0 | <50.0 |
| 173 | <0.1 | >30.0 | >30.0 | ND |
| 174 | <0.1 | >30.0 | >30.0 | <50.0 |
| 175 | <0.1 | >30.0 | >30.0 | <50.0 |
| 176 | <0.1 | >30.0 | >30.0 | <50.0 |
| 177 | <0.1 | >30.0 | >30.0 | <50.0 |
| 178 | <0.1 | >30.0 | >30.0 | <50.0 |
| 179 | <0.1 | >30.0 | >30.0 | <50.0 |
| 180 | >0.1 | >30.0 | >30.0 | ND |
| 181 | >0.1 | >30.0 | >30.0 | >50.0 |
| 182 | <0.1 | <30.0 | >30.0 | <50.0 |
| 183 | <0.1 | <30.0 | >30.0 | ND |
| 184 | <0.1 | <30.0 | >30.0 | <50.0 |
| 185 | <0.1 | >30.0 | >30.0 | <50.0 |
| 186 | <0.1 | >30.0 | >30.0 | <50.0 |
| 187 | <0.1 | >30.0 | >30.0 | <50.0 |
| 188 | ND | ND | ND | ND |
| 189 | ND | ND | ND | ND |
| 190 | <0.1 | <30.0 | >30.0 | <50.0 |
| 191 | <0.1 | <30.0 | >30.0 | <50.0 |
| 192 | <0.1 | <30.0 | >30.0 | <50.0 |
| 193 | >0.1 | >30.0 | >30.0 | <50.0 |
| 194 | <0.1 | >30.0 | >30.0 | <50.0 |
| 195 | <0.1 | >30.0 | >30.0 | <50.0 |
| 196 | >0.1 | >30.0 | >30.0 | ND |
| 197 | >0.1 | >30.0 | >30.0 | ND |
| 198 | <0.1 | >30.0 | >30.0 | <50.0 |
| 199 | <0.1 | >30.0 | >30.0 | <50.0 |
| 200 | <0.1 | >30.0 | >30.0 | ND |
| 201 | <0.1 | >30.0 | >30.0 | ND |
| 202 | <0.1 | <30.0 | >30.0 | <50.0 |
| 203 | <0.1 | <30.0 | >30.0 | <50.0 |
| 204 | <0.1 | >30.0 | >30.0 | <50.0 |
| 205 | ND | ND | ND | ND |
| 206 | ND | ND | ND | ND |
| 207 | <0.1 | >30.0 | >30.0 | <50.0 |
| 208 | <0.1 | <30.0 | >30.0 | ND |
| 209 | <0.1 | >30.0 | >30.0 | 50 |
| 210 | <0.1 | >30.0 | >30.0 | <50.0 |
| 211 | <0.1 | >30.0 | >30.0 | <50.0 |
| 212 | <0.1 | >30.0 | >30.0 | <50.0 |
| 213 | <0.1 | >30.0 | >30.0 | <50.0 |
| 214 | <0.1 | >30.0 | >30.0 | <50.0 |
| 215 | <0.1 | >30.0 | >30.0 | <50.0 |
| 216 | <0.1 | >30.0 | >30.0 | <50.0 |
| 217 | <0.1 | <30.0 | >30.0 | <50.0 |
| 218 | <0.1 | >30.0 | >30.0 | <50.0 |
| 219 | <0.1 | >30.0 | >30.0 | <50.0 |
| 220 | <0.1 | >30.0 | >30.0 | <50.0 |
| 221 | <0.1 | >30.0 | >30.0 | >50.0 |
| 222 | <0.1 | >30.0 | >30.0 | <50.0 |
| 223 | <0.1 | >30.0 | >30.0 | <50.0 |
| 224 | <0.1 | >30.0 | >30.0 | <50.0 |
| 225 | <0.1 | >30.0 | >30.0 | >50.0 |
| 226 | <0.1 | >30.0 | >30.0 | ND |
| 227 | <0.1 | >30.0 | >30.0 | <50.0 |
| 228 | <0.1 | >30.0 | >30.0 | <50.0 |
| 229 | <0.1 | >30.0 | >30.0 | <50.0 |
| 230 | <0.1 | >30.0 | >30.0 | <50.0 |
| 231 | <0.1 | >30.0 | >30.0 | <50.0 |
| 232 | <0.1 | >30.0 | >30.0 | <50.0 |
| 233 | <0.1 | >30.0 | >30.0 | <50.0 |
| 234 | <0.1 | >30.0 | >30.0 | <50.0 |
| 235 | <0.1 | >30.0 | >30.0 | <50.0 |
| 236 | <0.1 | >30.0 | >30.0 | <50.0 |
| 237 | <0.1 | >30.0 | >30.0 | <50.0 |
| 238 | <0.1 | >30.0 | >30.0 | <50.0 |
| 239 | <0.1 | >30.0 | >30.0 | <50.0 |
| 240 | <0.1 | >30.0 | >30.0 | <50.0 |
| 241 | ND | ND | ND | ND |
| 242 | <0.1 | >30.0 | >30.0 | ND |
| 243 | >0.1 | >30.0 | ND | ND |
| 244 | >0.1 | >30.0 | ND | ND |
| 245 | ND | ND | ND | ND |
| 246 | ND | ND | ND | ND |
| 247 | ND | ND | ND | ND |
| 248 | <0.1 | <30.0 | ND | ND |
| 249 | <0.1 | >30.0 | ND | <50.0 |
| 250 | ND | ND | ND | ND |
| 251 | <0.1 | >30.0 | ND | <50.0 |
| 252 | ND | ND | ND | ND |
| 253 | >0.1 | <30.0 | ND | <50.0 |
| 254 | <0.1 | >30.0 | ND | <50.0 |
| 255 | >0.1 | >30.0 | ND | ND |
| 256 | <0.1 | <30.0 | >30.0 | ND |
| 257 | <0.1 | >30.0 | >30.0 | ND |
| 258 | <0.1 | <30.0 | >30.0 | ND |

### EXAMPLE 1

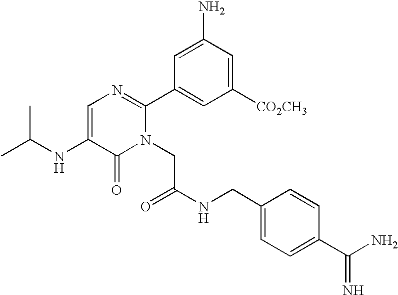

Prepared as described in the schemes above, Example 1 was obtained: ¹H NMR (300 MHz, CD₃OD) δ 8.76 (t, 1H), 7.75 (d, J=6.3 Hz, 2H), 7.49 (s, (2H), 7.47 (d, J=56.3 Hz, 2H), 7.41 (s, 1H), 7.14 (s, 1H), 7.01 (t, J=1.2 Hz, 1H), 4.69 (s, 2H), 4.49-4.51 (m, 2H), 3.86 (s, 3H), 3.62 (septet, J=4.5 Hz, 1H), 1.27 (d, J=4.5 Hz, 6H); LRMS (ESI) [M+H]⁺=492.

### EXAMPLE 2

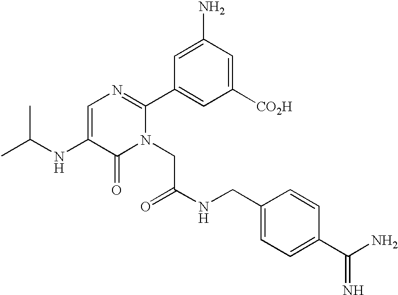

Using the product of Example 1, hydrolysis was used to obtain Example 2: ¹H NMR (300 MHz, CD₃OD) δ 7.76 (d, J=J=6.3 Hz, 2H), 7.46 (s, 1H), 7.14 (s, 1H), 7.04 (apparent t, J=1.8 Hz, 1H), 4.72 (s, 2H), 4.49 (s, 2H), 3.63 (septet, J=4.5 Hz, 1H), 1.28 (d, J=4.5 Hz, 6H); LRMS (ESI) [M+H]⁺=478.

### EXAMPLE 3

3-Amino-5-[1-[2-({4-[amino(imino)methyl]benzyl}amino)-2-oxoethyl]-3-bromo-5-(isopropylamino)-6-oxo-1,6-dihydropyrazin-2-yl]benzoic Acid

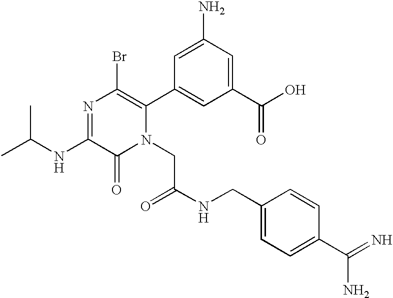

Ex-3) The crude product from Ex-4 (0.21 g, 0.37 mmol) was taken up in 2.5 mL of MeOH. Water (1.1 mL) was added, followed by LiOH (82 mg, 2 mmol) in 1.85 mL of water. The reaction was stirred for 3.5 h.

The volatiles were removed under reduced pressure. The crude residue was purified by reverse-phase HPLC with a gradient of 5/95%-95/5% acetonitrile/water (+0.1% TFA). The product-containing fractions were concentrated and dried under high vacuum to give 80 mg of an off-white solid: LRMS m/z 556, 558 (M⁺+H); HPLC purity (retention time): >99% (2.3 min); ¹H NMR (400 MHz, CD₃OD) δ 1.25 (d, 6H, J=6.8 Hz), 4.16 (m, 1H), 4.32-4.55 (m, 4H), 7.21 (m, 1H), 7.40 (d, 2H, J=8.4 Hz), 7.60 (m, 1H), 7.71 (d, 2H, J=8.4 Hz), 7.79 (m, 1H).

### EXAMPLE 4

Methyl 3-amino-5-[1-[2-({4-[amino(imino)methyl]benzyl}amino)-2-oxoethyl]-3-bromo-5-(isopropylamino)-6-oxo-1,6-dihydropyrazin-2-yl]benzoate

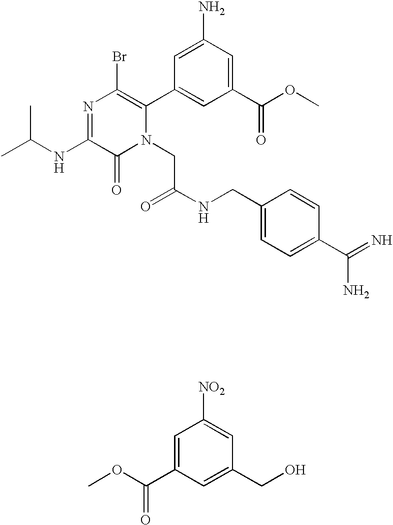

Methyl 3-(hydroxymethyl)-5-nitrobenzoate

Ex-4a) Anhydrous THF (600 mL) was cooled in a salt/ice bath under N₂. Mono-methyl-5-nitroisophthalate (175 g, 777 mmol) was added and the solution was cooled to −10° C. BH₃.THF (800 mL of a 1M solution) was added dropwise over 1 h 20 mins. The solution was warmed to room temperature, then heated to 35° C. The reaction slowly exothermed to 61° C. The reaction was stirred at 55° C. overnight.

The reaction solution was diluted with 200 mL of EtOAc and concentrated under reduced pressure. The yellow residue was diluted with 800 mL of EtOAc and washed with aqueous saturated NaHCO₃ 2×450 mL, brine 1×450 mL, dried over Na₂SO₄ and concentrated under reduced pressure to give 146.02 g (89%) of a pale yellow solid: LRMS m/z 212.0 (M⁺+H); HPLC purity (retention time): >80% (2.3 min); ¹H NMR (300 MHz, CDCl₃) δ 2.52 (s, 1H), 3.93 (s, 3H) 4.82 (s, 2H), 8.28 (s, 1H), 8.37 (s, 1H), 8.67 (s, 1H).

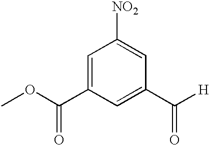

Methyl 3-formyl-5-nitrobenzoate

Ex-4b) CH₂Cl₂ was cooled to −78° C. under N₂ in a 5-L flask equipped with an overhead stirrer. Oxalyl chloride (69 mL, 791 mmol) was added in a steady stream to the CH₂Cl₂. DMSO (91 mL, 1282 mmol) in 400 mL of CH₂Cl₂ was added rapidly dropwise over 45 mins, keeping the reaction temperature below −70° C. The reaction was stirred at −78° C. for 10 mins. The product from Ex-4a (132.6 g, 628 mmol) in 850 mL of CH₂Cl₂ was added dropwise over 55 mins, keeping the reaction temperature below −70° C. The reaction was stirred at −78° C. for 30 mins. Triethylamine (350 mL, 2511 mmol) was added in a steady stream to the reaction, keeping the temperature below −60° C. The reaction was warmed to room temperature overnight.

The layers were allowed to separate. The CH₂Cl₂ layer was washed with 2 L of 1M KHSO₄, aqueous saturated NaHCO₃, brine, dried over Na₂SO₄, filtered, and concentrated under reduced pressure to give 126.4 g (96%) of a yellow solid: ¹H NMR (300 MHz, CDCl₃) δ 4.01 (s, 3H), 8.82 (m, 1H) 8.86 (m, 1H), 9.06 (m, 1H), 10.14 (s, 1H).

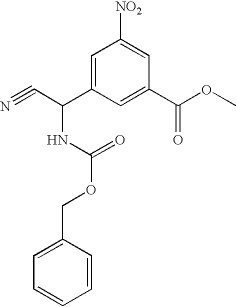

Methyl 3-[[[(benzyloxy)carbonyl](methyl)amino](cyano)methyl]-5-nitrobenzoate

Ex-4c) Glycine benzyl ester hydrochloride (38.14 g, 189 mmol) was dissolved in 250 mL of aqueous saturated Na₂CO₃ and 500 mL of brine. The aqueous phase was extracted with EtOAc 2×500 mL. The combined organics were dried over Na₂CO₃, concentrated under reduced pressure, dissolved in CH₂Cl₂ and concentrated again to give 29.9 g (96%) of a pale yellow oil which was used directly in the next step.

The glycine benzyl ester (29.9 g, 181 mmol) was dissolved in 80 mL of CH₂Cl₂. The product from Ex-4b (32.9 g, 157 mmol) was added, followed by 10 mL of CH₂Cl₂. TMSCN (25 g, 252 mmol) was added neat, dropwise, over 20 mins. The reaction exothermed from 26 to 38° C. The reaction was stirred at room temperature for 3 h.

The reaction solution was washed with brine 1×350 mL, dried over MgSO4, and the volatiles were removed under reduced pressure to give 83.78 g of a crude orangish-yellow oil, which was carried on without purification to the next step.

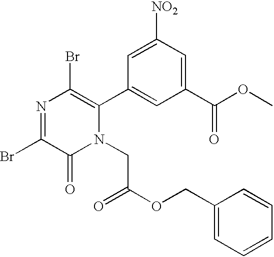

Methyl 3-{1-[2-(benzyloxy)-2-oxoethyl]-3,5-dibromo-6-oxo-1,6-dihydropyrazin-2-yl}-5-nitrobenzoate

Ex-4d) The crude product from Ex-4c (24.3 g, 63 mmol) was dissolved in 75 mL of CH₂Cl₂ and added dropwise to a solution of oxalyl bromide (59.7 g, 277 mmol) in 30 mL of CH₂Cl₂. The temperature during the addition was kept below 40° C. The reaction was stirred at 50-55° C. for 2 h.

The reaction solution was cooled, then washed with water 1×150 mL, brine 1×150 mL, dried over MgSO₄, filtered, and concentrated. The residue was dried under high vacuum and stored under N₂ to give 34.8 g of the crude, desired product: LRMS m/z 580,582,584 (M⁺+H); HPLC purity (retention time): <50% (4.3 min).

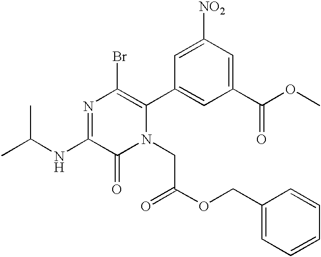

Methyl 3-[1-[2-(benzyloxy)-2-oxoethyl]-3-bromo-5-(isopropylamino)-6-oxo-1,6-dihydropyrazin-2-yl]-5-nitrobenzoate

Ex-4e) The crude product from Ex-4d (33 g crude, 57 mmol) was dissolved in 100 mL of EtOAc. Isopropylamine (20 mL 235 mmol) was added rapidly dropwise. The reaction exothermed from 33 to 55° C. The reaction was stirred at room temperature for 1 h 15 mins.

The reaction mixture was filtered through a pad of Celite, then washed with brine 1×125 mL, dried over MgSO₄, and concentrated under reduced pressure. The crude, reddish-brown residue was purified by normal phase HPLC using 30/70% EtOAc/hex, isocratic. The product-containing fractions were concentrated, then triturated with Et₂O to give 3.6 g of a yellow solid: LRMS m/z 559, 561 (M⁺+H); HPLC purity (retention time): >95% (4.5 min).

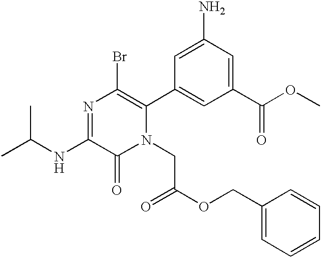

Methyl 3-amino-5-[1-[2-(benzyloxy)-2-oxoethyl]-3-bromo-5-(isopropylamino)-6-oxo-1,6-dihydropyrazin-2-yl]benzoate

Ex-4f) The product from Ex-4e (2.93 g, 5.24 mmol) was taken up in 40 mL of 50/50% EtOH/H₂O (w/w) and 10 mL of ACN and heated to 55° C. Another 40 mL of 50/50% EtOH/H₂O (w/w), 60 mL of ACN, and 40 mL of 100% EtOH were added. Iron powder (4.16 g, 74 mmol) and 12M HCl (1.44 mL, 17 mmol, in 50/50% EtOH/H₂O) were added portion-wise to the 55° C. reaction over two days.

The reaction mixture was cooled, filtered through a pad of Celite, and partially concentrated under reduced pressure. The solid that precipitated was filtered and dried under high vacuum overnight to give 2.50 g (90%) of desired product: LRMS m/z 529, 531 (M⁺+H); HPLC purity (retention time): >99% (3.8 min).

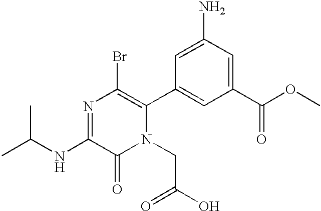

[6-[3-amino-5-(methoxycarbonyl)phenyl]-5-bromo-3-(isopropylamino)-2-oxopyrazin-1 (2H)-yl]acetic Acid

Ex-4 g) The product from Ex-4f (0.52 g, 0.98 mmol) was taken up in 5 mL of CH₂Cl₂. TFA (0.5 mL, 6.5 mmol) was added, followed by triflic acid (0.42 mL, 4.7 mmol) and anisole (0.15 mL, 1.4 mmol). The biphasic solution was stirred vigorously for 15 mins.

The solution was extracted with water 3×15 mL and aqueous saturated NaHCO₃ 2×10 mL. The combined aqueous extracts were acidified with 2 M HCl, then extracted with EtOAc 2×25 mL. The combined organic extracts were dried over MgSO₄, filtered, and concentrated under reduced pressure. The yellow residue was dissolved in Et₂O and concentrated. The resulting solid was dissolved in MeOH and filtered. The filtrate was concentrated and dried under high vacuum for 30 mins to give 0.41 g (96%) of a pale yellow solid: LRMS m/z 439, 441 (M⁺+H); HPLC purity (retention time): >99% (2.8 min).

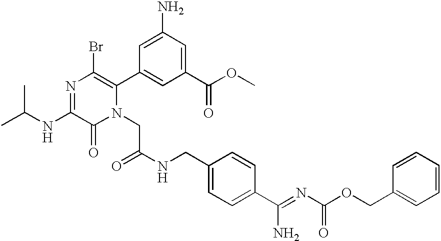

Methyl 3-amino-5-[1-(2-{[4-((Z)-amino{[(benzyloxy)carbonyl]imino}methyl)benzyl]amino}-2-oxoethyl)-3-bromo-5-(isopropylamino)-6-oxo-1,6-dihydropyrazin-2-yl]benzoate

Ex-4 h) LRMS m/z 704, 706 (M⁺+H); HPLC purity (retention time): >95% (3.2 min).

Ex-4) The crude product from Ex-4 h (0.64 g, 0.91 mmol) was dissolved in 10 mL of CH₂Cl₂. Triflic acid (0.60 mL, 6.8 mmol) and anisole (0.15 mL, 1.4 mmol) were added, followed by TFA (0.44 mL, 5.7 mmol). After stirring for 1 h 10 mins, another 0.6 mL of TFA (7.8 mmol) and 0.3 mL (3.4 mmol) of triflic acid were added. The reaction was stirred another 15 mins.

The reaction solution was extracted with water 5×30 mL. The combined aqueous extracts were neutralized with aqueous saturated NaHCO₃, then extracted with EtOAc 2×75 mL. The combined organics were washed with brine 1×50 mL, dried over MgSO₄, concentrated under reduced pressure and stored under N₂ to give 0.32 g of a pale yellow residue: LRMS m/z 570, 572 (M⁺+H); HPLC purity (retention time): >95% (2.6 min).

One third of the residue was purified by reverse-phase HPLC with a gradient of 5/95%-95/5% acetonitrile/water (+0.1% TFA) over 10 mins. Product eluted at 4.9 mins, giving 59 mg of a pale yellow solid:

LRMS m/z 570, 572 (M⁺+H); HPLC purity (retention time) >99% (2.6 min); ¹H NMR (400 MHz, CD₃OD) δ 1.25 (d, 6H, J=6.5 Hz), 3.86 (s, 3H), 4.16 (m, 1H), 4.33-4.52 (m, 4H), 7.19 (m, 1H), 7.40 (d, 2H, J=8.5 Hz), 7.56 (m, 1H), 7.70-7.72 (m, 3H)

### Amide Library

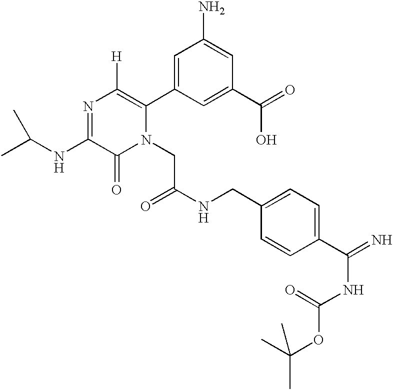

### Scaffold

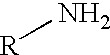

### Generic Amine

Scaffold (0.1 mmol) was reacted. Each well contained the scaffold (0.1 mmol), P-CD resin (0.2 gram, 1 meq/g), HOBT (13.5 mg), NMM (0.20 mL), Generic Amine (0.1 mmol), and DCM (6.0 mL). Each well was shaken for 6 hours. DCM (3.0 mL) was added along with PolyAmine Resin (200 mg, 230 meq/g) and Aldehyde Resin (75 mg). The wells were shaken for 0.5 hours. Each well was filtered and the solution was dried under Nitrogen stream. Each well was re-dissolved in DCM (3.0 mL) and TFA (0.5 mL). The wells were shaken (2 hours), and then dried under a Nitrogen stream. Each well was re-dissolved in methanol and purified as fractions on the Gilson as needed.

General: HPLC purities were determined with a Hewlett Packard HP1100 using an XDB-C183.5 ?M 2.1×30 mm column, eluting with a gradient system of 5/95% to 95/5% acetonitrile/H₂O (+0.1% TFA buffer) over 4.5 min at 1 mL/min, and detected by UV at 254 nm using a diode array detector.

### EXAMPLE 5

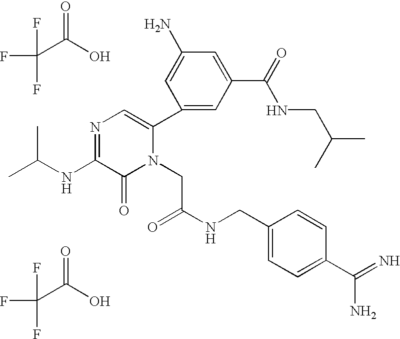

LC/MS Purity—>80%, Retention Time—2.136, M+H—533.3

### EXAMPLE 6

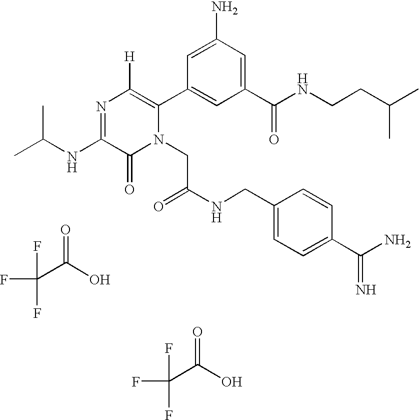

LC/MS Purity—>80%, Retention Time—2.371, M+H—547.5

### EXAMPLE 7

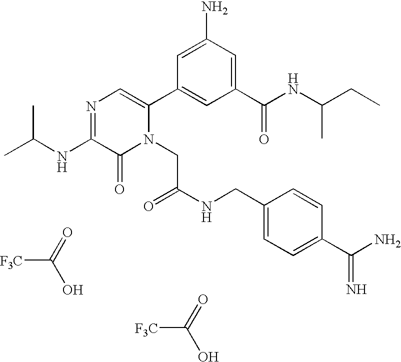

LC/MS Purity—>80%, Retention Time—2.093, M+H—533.4

### EXAMPLE 8

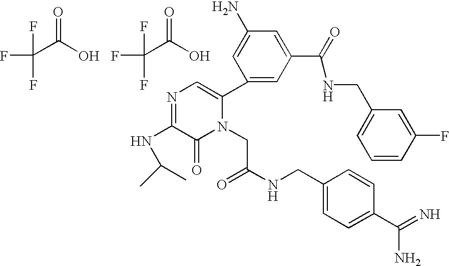

LC/MS Purity—58%, Retention Time—2.350, M+H—585.4

### EXAMPLE 9

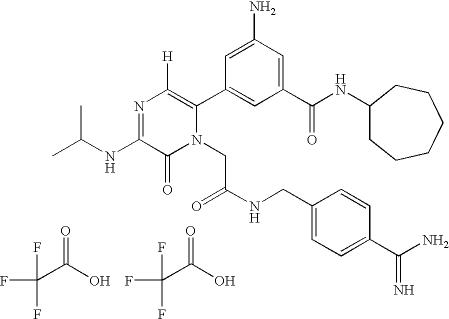

LC/MS Purity—>80%, Retention Time—2.594, M+H—573.0

### EXAMPLE 10

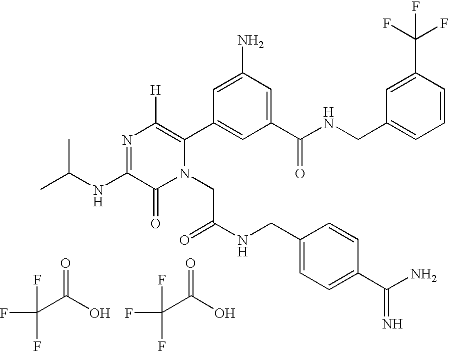

LC/MS Purity—>80%, Retention Time—2.599, M+H—635.1

### EXAMPLE 11

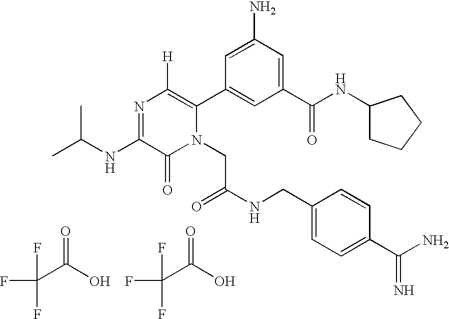

LC/MS Purity—>80%, Retention Time—2.190, M+H—545.4

### EXAMPLE 12

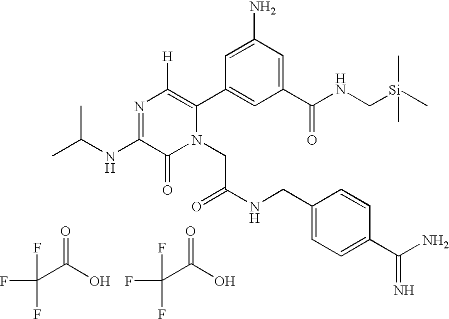

LC/MS Purity—>80%, Retention Time—2.479, M+H—563.2

### EXAMPLE 13

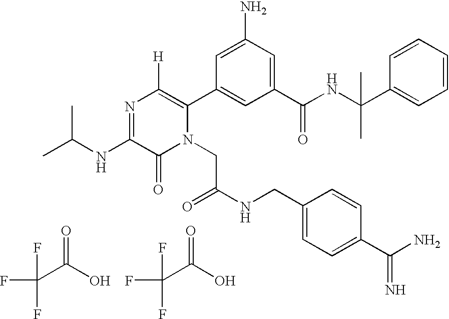

LC/MS Purity—>80%, Retention Time—2.582, M+H—595.4

### EXAMPLE 14

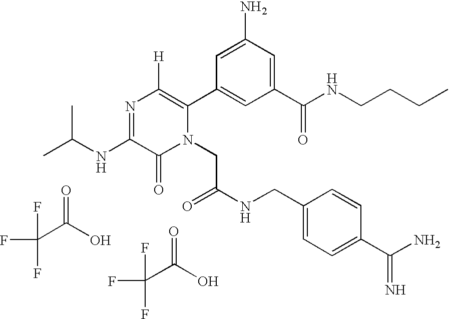

LC/MS Purity—>80%, Retention Time—2.199, M+H—533.5

### EXAMPLE 15

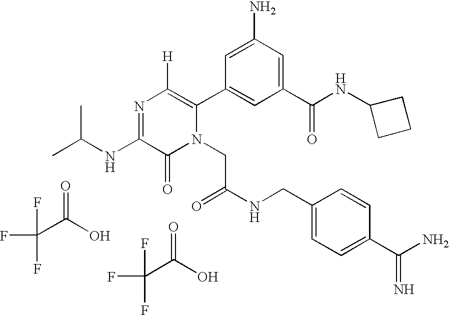

LC/MS Purity—90%, Retention Time—1.395, M+H—531.2

### EXAMPLES 16/17

3-amino-5-[1-[2-({4-[amino(imino)methyl]benzyl}amino)-2-oxoethyl]-5-(isopropylamino)-6-oxo-1,6-dihydropyrazin-2-yl]benzoic Acid Trifluoracetate

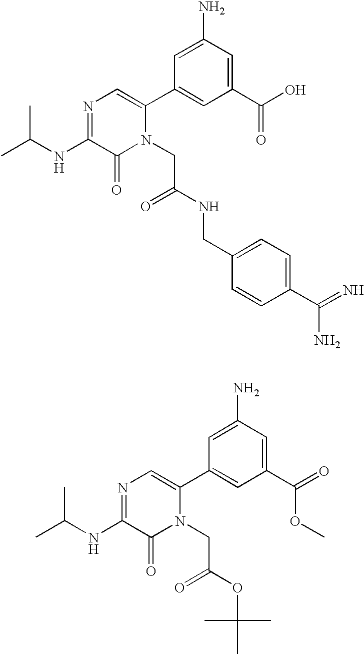

methyl 3-[1-(2-tert-butoxy-2-oxoethyl)-5-(isopropylamino)-6-oxo-1,6-dihydropyrazin-2-yl]-5-nitrobenzoate

Ex-17a) A solution of tert-butyl [6-bromo-3-(isopropylamino)-2-oxopyrazin-1 (2H)-yl]acetate (50.0 g, 144.4 mmol), 3-(methoxycarbonyl)-5-nitrophenylboronic acid (35.7 g, 158.9 mmol), and sodium carbonate (61.2 g, 0.6 mol) in 2 L THF and 200 mL DI H₂O stirred in a 3 neck flask equipped with a cold water condenser, heating mantle and stir bar stirred for 1 h while N₂₍₉₎ bubbled through the solution.

Tetrakis(triphenylphospine)palladium (0) (16.7 g, 14.4 mmol) under a blanket of N_{2(g)}, and the reaction was warmed to reflux (64° C.) for 16 h. The reaction mixture cooled to room temperature, filtered, reduced, diluted with 1 L ethyl acetate and washed with 2×500 mL DI H₂O. The organic portion was dried over Magnesium sulfate, filtered and concentrated to give 80 g crude brown oil. The crude product was chromatographed on silica, eluted with 20% ethyl acetate/80% hexanes. 26.0 g (40.3%) of yellow solid was recovered.

LRMS m/z 447.2 (M⁺+H).

¹H NMR (300 MHz, CDCl₃) δ 1.30 (d, 6H, J=6.3 Hz), 1.46 (s, 9H), 4.00 (s, 3H), 4.09-4.23 (m, 1H), 4.38 (s, 2H), 5.31 (s, 2H), 6.85 (s, 1H), 8.38 (s, 1H), 8.44 (s, 1H), 8.90 (s, 1H).

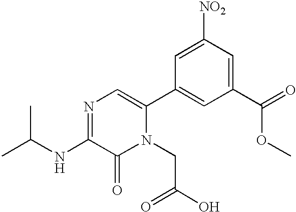

[3-(isopropylamino)-6-[3-(methoxycarbonyl)-5-nitrophenyl]-2-oxopyrazin-1 (2H)-yl]acetic Acid

Ex-17b) 3.2 g (7.2 mmol) of the product of 17a was hydrolyzed with stirring in 50 mL TFA and 50 mL CH₂Cl₂ for 2 h. The solvents were evaporated to give 2.3 g (81%) of brown foam.

¹H NMR (400 MHz, CD₃COCD₃) δ 1.46 (d, 6H, J=6.4 Hz), 3.96 (s, 3H), 4.22-4.38 (m, 1H), 4.59 (s, 2H), 7.01 (s, 1H), 8.42 (s, 1H), 8.59 (s, 1H), 8.82 (s, 1H)

LRMS m/z 391.4 (M⁺+H)

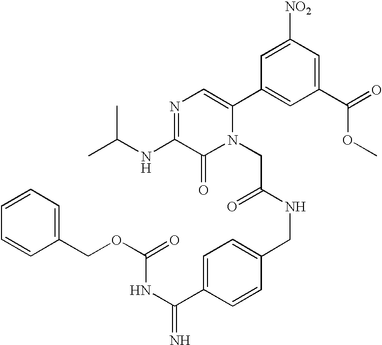

methyl 3-[1-[2-({4-[{[(benzyloxy)carbonyl]amino}(imino)methyl]benzyl}amino)-2-oxoethyl]-5-(isopropylamino)-6-oxo-1,6-dihydropyrazin-2-yl]-5-nitrobenzoate

Ex-17c) A solution of 17b (36.3 g, 59.0 mmol), benzyl [4-(aminomethyl)phenyl](imino)methylcarbamate dihydrochloride (23.1 g, 64.9 mmol), HOBt (4.0 g, 29.5 mmol), and NMM (38.9 mL, 353.9 mmol) with 500 mL CH₂Cl₂ and 200 mL DMF was shaken in a glass bottle for 30 min. Polystyrene resin bound carbodiimide (73.1 g, 88.4 mmol) was added and the reaction shook for 12 h. PS-diethylene triamine (21.0 g, 59.0 mmol) and Aldehyde wang (20.7 g, 59.0 mmol) scavenging resins were added and shook for 30 min. Reaction mixture was filtered, resins rinsed with copious amounts of CH₂Cl2 and DMF, and solvents evaporated in vacuò to afford a brown semi-solid. No yield determined, >95% pure by HPLC and ¹H NMR.

¹H NMR (400 MHz, CD₃OD) δ 1.28 (d, 6H, J=6.4 Hz), 3.96 (s, 3H), 4.10-4.19 (m, 1H), 4.43 (s, 2H), 4.55 (s, 2H), 6.82 (s, 1H), 7.33-7.79 (m, 9H), 8.42 (s, 1H), 8.54 (s, 1H), 8.80 (s, 1H)

LRMS m/z 656.2 (M⁺+H).

Ex-17) Crude product 17c (36.7 g, 56.0 mmol) was dissolved in 1 L methanol and 4 eq. of 2M LIOH in H₂O. In addition, Pd/C (10%, 3.6 g) was added, and the reaction hydrogenated with a H_{2(g)} balloon, room temperature, stirring overnight. The reaction filtered through Celite 545, neutralized to pH 7 with 6 M HCl, and reduced in vacuo. Crude material was purified by reverse phase HPLC with 5-20% acetonitrile/water with 0.1% TFA over 30 min. Product collected at 8-12 min, yield 19.8 g (48%).

¹H NMR (300 MHz, CD₃OD) δ 1.42 (d, 6H, J=6.6 Hz), 4.03-4.17 (m, 1H), 4.46 (s, 2H), 4.62 (s, 2H), 6.71 (s, 1H), 7.39-7.86 (m, 7H).

¹³C NMR (300 MHz, CD₃OD) δ 20.23, 42.60, 45.74, 108.72, 121.63, 125.58, 127.72, 127.21, 127.93, 128.04, 128.97, 132.03, 133.16, 145.10, 146.33, 152.58, 167.04, 167.11, 167.44.

LRMS m/z 478.2 (M⁺+H).

Two salts were prepared for this compound and data for each is reported in Table 3.

### EXAMPLES 18/19

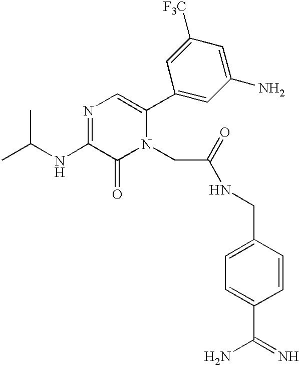

HPLC/LRMS: >97%, 502 (M+H)+; ¹⁹F NMR(DMF-d₇, 375 MHz) δ −63.28 ppm; ¹H NMR(DMF-d₇, 400 MHz) δ 9.99 (2H, s), 9.75 (2H, s), 9.44 (1H, br s), 9.05 (1H, m), 8.00 (2H, m), 7.42 (2H, m), 7.34 (1H, s), 7.16 (1H, s), 7.04 (1H, s), 6.71 (1H, s), 4.66 (3H, m), 4.43 (2H(, m), 1.38 (6H, d); HRMS(ES+) calcd. for C₂₄H₂₇N₇O₂ 502.2178, found 502.2214.

Two salts were prepared for this compound and data for each is reported in Table 3.

### EXAMPLE 20

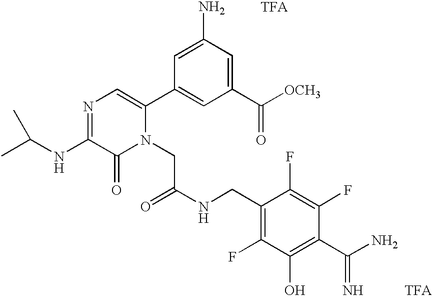

LCMS (RP, 5-90% acetonitrile in 0.1% TFA over 14 min): retention time: 3.63 min;(M+H)⁺=562.

¹H-NMR, 400 MHz, CD₃OD, δ7.50 (s, 1H), 7.35 (s, 1H), 6.98 (s, 1H), 6.60 (s, 1H), 4.46 (s, 2H), 4.43 (s, 2H), 4.03 (septet, J=6.4 Hz, 1H), 3.86 (s, 3H), 1.36 (d, J=6.4 Hz, 6H).

¹⁹F-NMR, 376 MHz, CD₃OD, δ −77.6 (TFA), −142.64 (d, J=13.5 Hz, 1H), −146.10 (dd, J=13.5 Hz, J=21.4 Hz, 1H), −152.81 (d, J=21.4 Hz, 1H).

### EXAMPLE 21

Methyl 3-amino-5-[1-[2-({4-[amino(imino)methyl]benzyl}amino)-2-oxoethyl]-5-(isopropylamino)-6-oxo-1,6-dihydropyrazin-2-yl]benzoate Trifluoroacetate

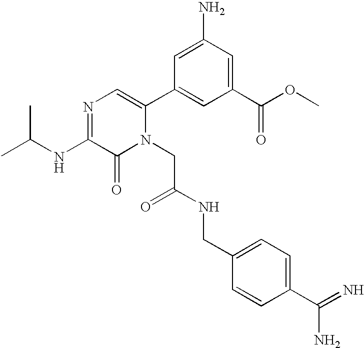

¹H NMR (300 MHz, CD₃OD) δ 1.39 (d, 6H, J=6.6 Hz), 3.87 (s, 3H), 4.03-4.17 (m, 1H), 4.49 (s, 2H), 4.62 (s, 2H), 6.65 (s, 1H), 6.91-7.79 (m, 7H).

LRMS m/z 492.2 (M⁺+H).

### EXAMPLE 22

Methyl {3-amino-5-[1-[2-({4-[amino(imino)methyl]benzyl}amino)-2-oxoethyl]-5-(isopropylamino)-6-oxo-1,6-dihydropyrazin-2-yl]-4-methoxyphenyl}acetate

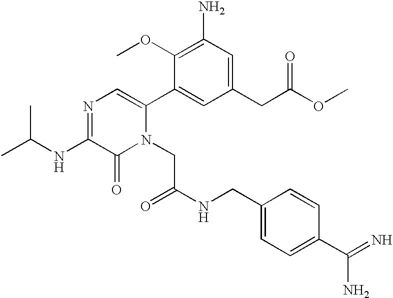

Methyl 4-Hydroxy-3-iodo-5-nitrophenylacetate

Ex-22a) 4-Hydroxy-3-iodo-5-nitrophenylacetic acid (2.48 g, 7.68 mmol) was taken up in 14 mL of MeOH. H₂SO₄ (18M, 1.4 mL, 25 mmol) in 4 mL of MeOH was added, along with 2.5 mL of THF. The mixture was heated to 60° C., and another 1 mL of THF was added to make the reaction homogeneous. The reaction was stirred for 50 mins.

The reaction was cooled to 30° C., then cooled in an ice bath. The bright yellow solid was filtered, washed with water until the pH was neutral, then dried under high vacuum at 40° C. to give 2.55 g (95%) of a bright yellow solid: LRMS m/z 337.9 (M⁺+H); HPLC purity (retention time): >98% (3.4 min); ¹H NMR (400 MHz, CDCl₃) □ 3.57 (s, 2H), 3.70 (s, 3H), 8.00 (d, 1H, J=1.9 Hz), 8.02 (d, 1H, J=1.6 Hz)

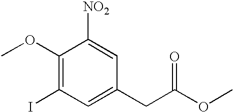

Methyl 3-iodo-4-methoxy-5-nitrophenylacetate

Ex-22b) The product from Ex-22a (2.50 g, 7.42 mmol) was dissolved in 33 mL of acetone. K₂CO₃ (5.13 g, 37.1 mmol) and CH₃I (4.62 mL, 74.2 mmol) were added and the reaction mixture was stirred at 50° C. for 3 h 20 mins.

The reaction was cooled, then diluted with 50 mL of water. The acetone was removed under reduced pressure. The remaining aqueous mixture was acidified with 2 M HCl. The mixture was extracted with EtOAc 2×75 mL. The combined organic extracts were washed with brine 1×75 mL, dried over MgSO₄, concentrated, and dried under high vacuum for 3 h to give 2.60 g (99%) of a yellow solid: ¹H NMR (400 MHz, CDCl₃) δ 3.59 (s, 2H), 3.71 (s, 3H), 3.94 (s, 3H), 7.71 (d, 1H, J=2.2 Hz), 7.92 (1H, d, 2.2 Hz).

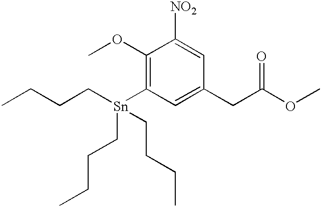

Methyl [4-methoxy-3-nitro-5-(tributylstannyl)phenyl]acetate

Ex-22c) The product from Ex-22b (3.02 g, 8.60 mmol) was dissolved in 15 mL of toluene. The solution was evacuated, then flushed with N₂. Repeated 4×. Bis(tributyl)tin (13 mL, 25.7 mmol) was added, followed by Pd(PPh₃)₄ (105 mg, 0.09 mmol). The homogeneous solution was evacuated and flushed with N₂. Repeated 4×. The solution was stirred at 90° C. under N₂ until the reaction was complete by TLC (2 days).

The reaction solution was cooled and diluted with EtOAc. The solution was washed with brine 1×50 mL, aqueous saturated KF 1×30 mL, brine 1×30 mL, dried over MgSO₄, and concentrated under reduced pressure. The crude was purified on silica, eluting with 10/90% EtOAc/hex, to give 2.64 g (60%) of a yellow oil: ¹H NMR (400 MHz, CDCl₃) δ 0.82-0.92 (m, 9H), 1.02-1.11 (m, 6H) 1.25-1.38 (m, ˜6H), 1.45-1.56 (m, 6H), 3.61 (s, 2H), 3.70 (s, 3H), 3.77 (s, 3H), 7.42 (d, 1H, J=2.3 Hz), 7.69 (d, 1H, J=2.1 Hz).

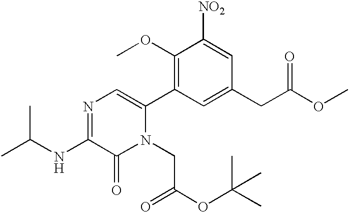

Methyl {3-[1-(2-tert-butoxy-2-oxoethyl)-5-(isopropylamino)-6-oxo-1,6-dihydropyrazin-2-yl]-4-methoxy-5-nitrophenyl}acetate

Ex-22d) The product from Ex-22c (0.60 g, 1.2 mmol) was dissolved in 5 mL of N₂-flushed DMF. tert-Butyl [6-bromo-3-(isopropylamino)-2-oxopyrazin-1 (2H)-yl]acetate (1.14 g, 3.3 mmol), PPh₃ (60 mg, 0.23 mmol), Cu(I)Br (82 mg, 0.57 mmol), 2,6-di-t-butyl-4-methylphenol (14 mg, 0.06 mmol), and Pd(PPh₃)₄ (117 mg, 0.10 mmol) were added, along with another 5 mL of N₂-flushed DMF. The reaction was stirred at 110° C. for 3.5 h.

The dark red solution was cooled to room temperature, then diluted with 100 mL of EtOAc and washed with aqueous KF 1×100 mL, aqueous saturated NaHCO₃ 1×75 mL, and brine 1×75 mL. The organic phase was dried over MgSO₄, filtered, and concentrated.

The residue was purified on silica, eluting with 30/70% EtOAc/hexanes, to give 0.24 g (41%) of a yellow film: LRMS m/z 491.2, 533.3 (dimer) (M⁺+H), (M⁺+H); HPLC purity (retention time): 80% (3.2 min), 20% (3.4 min, dimer).

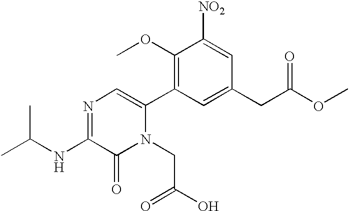

[3-(Isopropylamino)-6-[2-methoxy-5-(2-methoxy-2-oxoethyl)-3-nitrophenyl]-2-oxopyrazin-1 (2H)-yl]acetic Acid

Ex-22e) The product from Ex-22d (43 mg, 0.09 mmol) was dissolved in 0.5 mL of CH₂Cl₂. TFA (0.05 mL, 0.65 mmol), triflic acid (0.044 mL, 0.5 mmol) and anisole (0.016 mL, 0.15 mmol) were added and the reaction was stirred at room temperature for 15 mins.

The reaction solution was extracted with water 2×, then aqueous saturated NaHCO₃ 2×. The combined aqueous extracts were acidified with 2 M HCl and extracted with EtOAc 3×. The combined organic extracts were washed with brine, dried over MgSO₄ and concentrated to give 38 mg of a yellow film: LRMS m/z 435.1 (M⁺+H); HPLC purity (retention time): 90% (1.9 min).

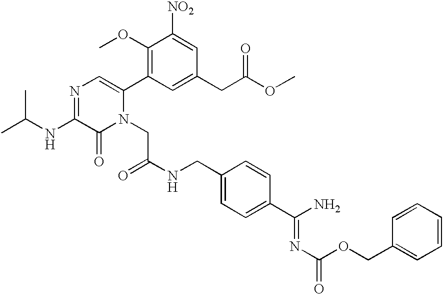

Methyl {3-[1-(2-{[4-((Z)-amino{[(benzyloxy)carbonyl]imino}methyl)benzyl]amino}-2-oxoethyl)-5-(isopropylamino)-6-oxo-1,6-dihydropyrazin-2-yl]-4-methoxy-5-nitrophenyl}acetate

Ex-22f) 38 mg (0.086 mmol) Ex-22e, 4.4 mg (0.03 mmol) HOBt, 28 mg (0.07 mmol) benzyl [4-(aminomethyl)phenyl](imino)methylcarbamate dihydrochloride, 57 □L (0.52 mmol) NMM, 127 mg (0.13 mmol) PS-carbodiimide, 1.5 mL CH₂Cl₂, and 0.3 mL DMF. Agitated for 1.5 h, then added 20 mg (0.06 mmol) PS-diethylenetriamine and 120 mg (0.3 mmol) Wang aldehyde scavenging resins for 2 h to give 0.11 g of crude, desired product: LRMS m/z 700.2 (M⁺+H); HPLC purity (retention time): >85% (2.7 min).

Ex-22) 0.11 g of the crude product from Ex-22f, 20 mg (0.009 mmol) of 10% Pd/C (50% water-wet) and 1 mL of MeOH. The reaction was stirred overnight.

The crude residue was purified by reverse-phase HPLC with a gradient of 15/85%-85/15% acetonitrile/water (+0.1% TFA) over 10 min. Product eluted at 2.8 min, giving 20 mg of an off-white solid: LRMS m/z 536.2 (M⁺+H); HPLC purity (retention time): 97% (1.8 min); ¹H NMR (400 MHz, CD₃OD) δ 1.41 (m, 6H), 3.52 (m, 2H), 3.66 (2 s, 6H), 4.04 (m, 1H), 4.31-4.43 (m, 3H), 4.90-4.94 (m, 1H), 6.61 (m, 1H), 6.73 (s, 1H), 7.01 (m, 1H), 7.40 (d, 2H, J=8.4 Hz), 7.74 (d, 2H, J=8.4 Hz)

### EXAMPLE 23

{3-Amino-5-[1-[2-({4-[amino(imino)methyl]benzyl}amino)-2-oxoethyl]-5-(isopropylamino)-6-oxo-1,6-dihydropyrazin-2-yl]-4-methoxyphenyl}acetic Acid

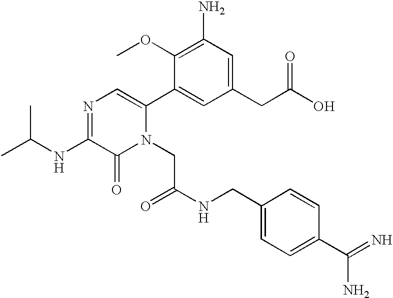

Ex-23) The crude product from Ex-22f (150 mg crude, 0.22 mmol crude) was dissolved in 1 mL of MeOH. LiOH (55 mg, 1.3 mmol) in 0.5 mL of water and a 0.5 mL MeOH rinse were added. Pd/C (10% Pd/C, 50% water-wet, 48 mg, 0.02 mmol) in N₂-flushed water was added. The mixture was evacuated, then flushed with N₂. Repeated several times with N₂, then H₂. Stirred under a H₂ balloon for 3 h. The mixture was filtered through a 0.2 μM frit, then concentrated.

The crude residue was purified by reverse-phase HPLC with a gradient of 15/85%-40/60% acetonitrile/water (+0.1% TFA) over 10 mins. Product eluted at 2.4 mins, giving 46 mg of an off-white solid: LRMS m/z 522.2 (M⁺+H); HPLC purity (retention time): >95% (1.6 min); ¹H NMR (400 MHz, CD₃OD) δ 1.38 (m, 6H), 3.45 (s, 2H), 3.64 (s, 3H), 4.02 (m, 1H), 4.29-4.41 (m, 2H), 4.91 (m, 2H), 6.59 (m, 1H), 6.72 (s, 1H), 7.00 (m, 1H), 7.38 (d, 2H, J=8.3 Hz), 7.72 (d, 2H, J=8.4 Hz).

### Examples 24/25

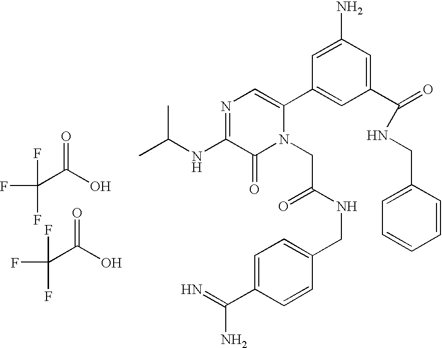

LC/MS Purity—99%, Retention Time—2.089, M+H—567.2

Two salts were prepared for this compound and data for each is reported in Table 3.

### EXAMPLE 26

3-amino-5-[1-[2-({4-[amino(imino)methyl]benzyl}amino)-2-oxoethyl]-5-(isopropylamino)-6-oxo-1,6-dihydropyrazin-2-yl]-N-(2-methoxyethyl)benzamide

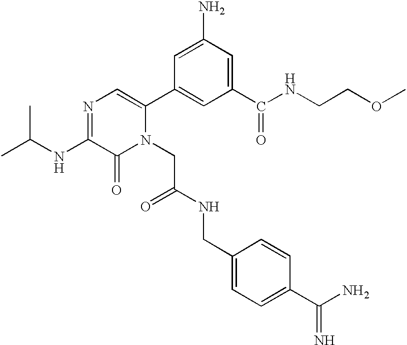

MS-ESI (M+H)=699.

### EXAMPLE 27

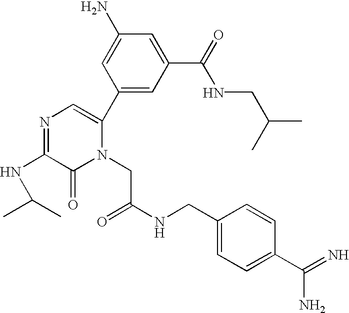

The compound of Example 27 was prepared in an analogous manner to that of Example 186.

### EXAMPLE 28

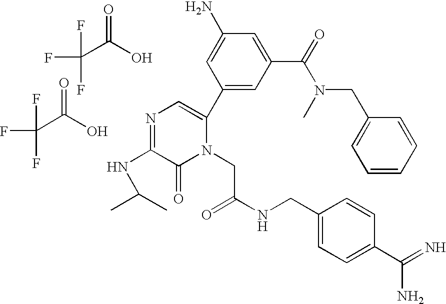

LC/MS Purity—>80%, Retention Time—2.354, M+H—581.3

### EXAMPLE 29

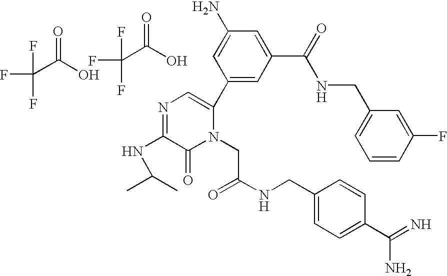

LC/MS Purity—>80%, Retention Time—2.370, M+H—585.4

### EXAMPLE 30

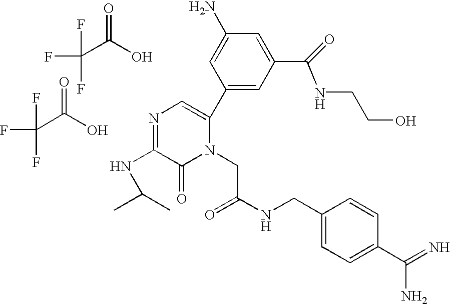

LC/MS Purity—>80%, Retention Time—1.492, M+H—522.6

### EXAMPLE 31

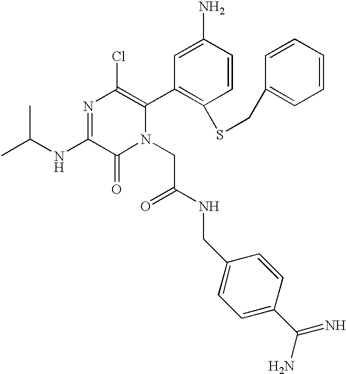

The compound of Example 31 was prepared in an analogous manner to that of Example 3.

### EXAMPLE 32

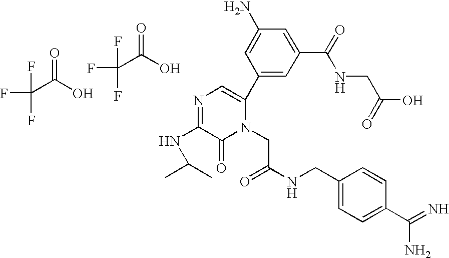

### EXAMPLE 33

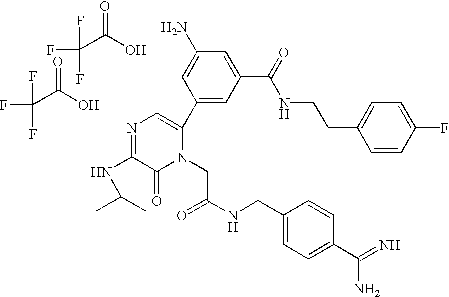

LC/MS Purity—>80%, Retention Time—2.484, M+H—600.9

### EXAMPLE 34

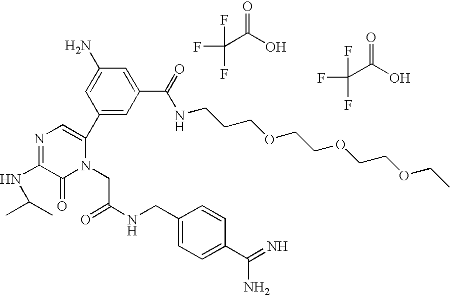

LC/MS Purity—>80%, Retention Time—2.213, M+H—652.0

### EXAMPLE 35

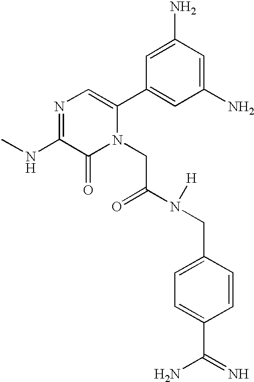

HPLC/LRMS: >96%, 421 (M+H)+; HRMS(ES+) calcd. for C₂₁H₂₅N₈O₂ 421.2100, found 421.2112.

### EXAMPLE 36

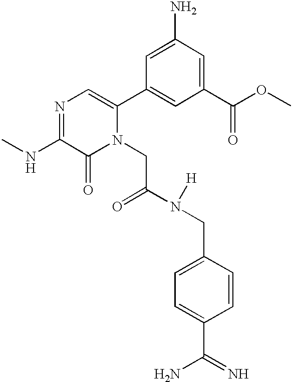

HPLC/LRMS: >98%, 464 (M+H)+; HRMS(ES+) calcd. for C₂₃H₂₆N₇O₄ 464.2046, found 464.2065.

### EXAMPLE 37

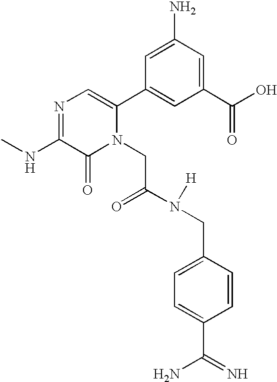

Ex-36 (500 mg) was stirred in MeOH/H₂O and LiOH—H₂O(Approxiately 5 eq., 167 mg). Purged mixture with nitrogen and added excess palladium on activated carbon, 10%wt. by dry basis. The reaction vessel was capped with a septum and a hydrogen balloon added. After 18 hrs reaction was filtered through Celite, the filtrae concentrated invacuo, and the residue chromatographed on Gilson HPLC-RP system with 0.1%TFA(H₂O/AN). HPLC/LRMS: >98%, 450 (M+H)+; HRMS(ES+) calcd. for C₂₂H₂₄₉N₇O₄ 450.1890, found 450.1930.

### EXAMPLE 38

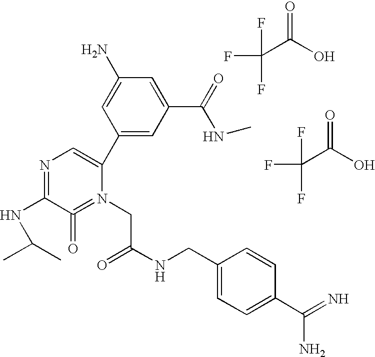

The compound of Example 38 was prepared in an analogous manner to that of Example 186.

### EXAMPLE 39

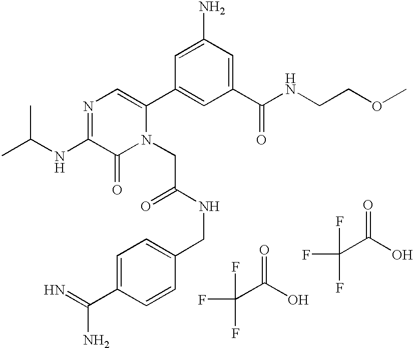

The compound of Example 39 is a salt of the compound of Example 26.

### EXAMPLE 40

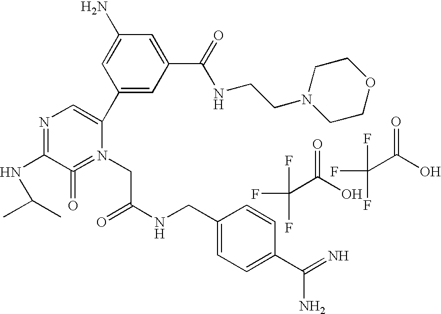

The compound of Example 40 was prepared in an analogous manner to that of Example 186.

### EXAMPLE 41

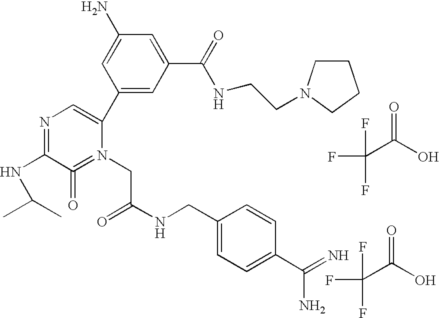

The compound of Example 41 was prepared in an analogous manner to that of Example 186.

### EXAMPLE 42

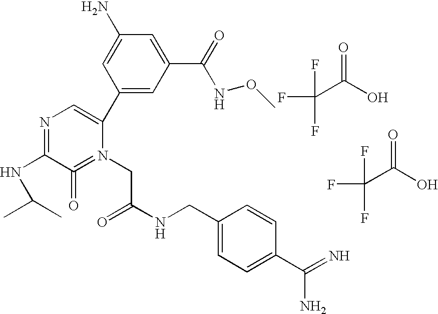

The compound of Example 42 was prepared in an analogous manner to that of Example 186.

### EXAMPLE 43

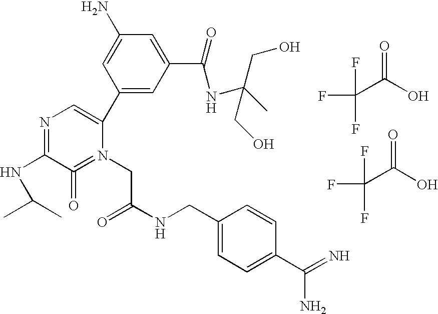

The compound of Example 43 was prepared in an analogous manner to that of Example 186.

### EXAMPLE 44

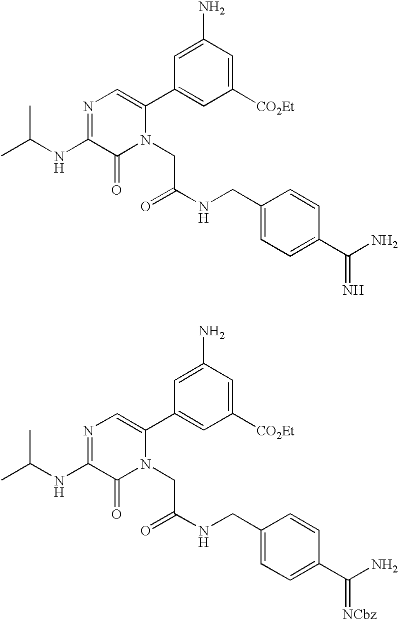

Ex-44a) m/z (M+H)⁺640

Ex44) m/z(M+H)⁺506

Analysis: C₂₆H₃₁N₇O₄+2.30 TFA+0.80H₂O calcd: C, 46.99; H, 4.50; N, 12.53; found: C, 46.97; H, 4.47; N, 12.55.

HRMS calcd: 506.2510; Found: 506.2479

¹H NMR(400 MHz, DMSO): 1.22 (6H, d), 1.28 (3H, t), 4.09 (1H, m), 4.25 (2H, q), 4.36 (2H, d), 4.39 (2H, s), 6.71 (1H, s), 6.78 (1H, t), 7.11 (1H, t), 7.28 (1H, t), 7.39 (2H, d), 7.50 (1H, br s), 7.73 (2H, d), 8.69 (1H, t), 9.14 (2H, br s), 9.27 (2H, br s).

### EXAMPLE 45

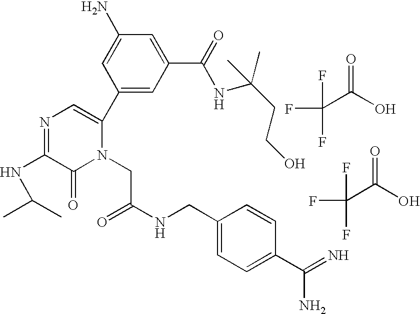

LC/MS Purity—99%, Retention Time—1.500, M+H—663.3

### EXAMPLE 46

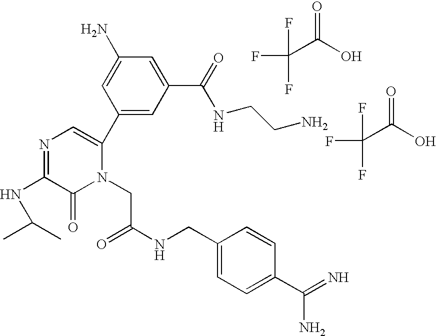

LC/MS Purity—99%, Retention Time—1.414, M+H—621.6

### EXAMPLE 47

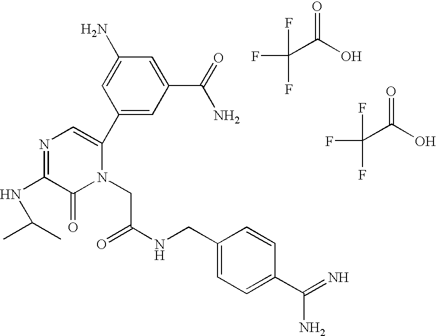

LC/MS Purity—99%, Retention Time—1.243, M+H—478.3

### EXAMPLE 48

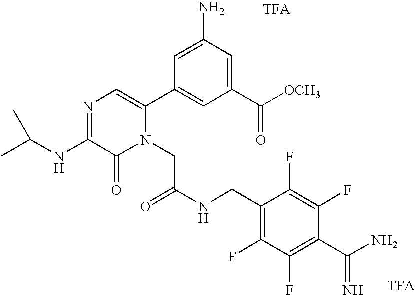

LCMS (RP, 5-90% acetonitrile in 0.1% TFA over 14 min): retention time: 3.80 min;(M+H)⁺=564, (M+Na)⁺=586.

### EXAMPLE 49

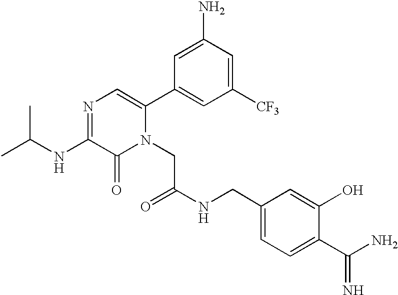

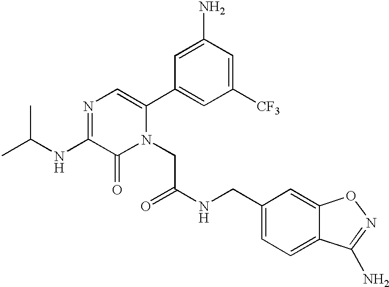

Ex-49a) m/z(M+H)⁺516

Ex-49) To the product from 49a (3.0 g, 5.35 mmol) in 60 ml of ethanol was added 1.2 g of 10% palladium on carbon and 1.5 ml of hydrogen chloride (conc.). The mixture was shaken on the Parr apparatus under 40 Psi of hydrogen for 2 hours. The mixture was filtered and concentrated in vacuo to give 3.0 g of a tan foam. A portion of this material (400 mg, 0.71 mmol) was then purified by reverse phase chromatography with 5-60% CH₃CN/H₂O to give 260 mg (46% yield) of a white solid. m/z(M+H)⁺518

Analysis: C₂₄H₂₆N₇O₃+2.35 TFA+0.55H₂O calcd: C, 43.34; H, 3.73; N, 12.33; found: C, 43.39; H, 3.80; N, 12.28.

HRMS calcd: 518.2122; Found: 518.2107

¹H NMR(400 MHz, DMSO): 1.22 (6H, d), 4.09 (1H, m), 4.23 (2H, d), 4.34 (2H, s), 6.72 (1H, s), 6.77-6.80 (3H, m), 6.9 (2H, s), 7.45 (1H, d), 7.48 (1H, br s), 8.69 (1H, t), 8.96 (4H, br s).

### EXAMPLE 50

N-{3-amino-5-[1-[2-({4-[amino(imino)methyl]benzyl}amino)-2-oxoethyl]-5-(isopropylamino)-6-oxo-1,6-dihydropyrazin-2-yl]phenyl}-2-methylpropanamide

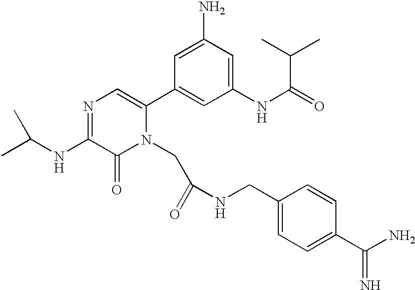

HRMS calcd for C₂₇H₃₄F₃N₈O₃ (M+H): 519.2827. Found: 519.2816.

Anal. Calcd for C₂₇H₃₄F₃N₈O₃+3.45TFA+2.65H₂O:

C: 42.42; H: 4.48; N: 11.67.

Found: C: 42.38; H: 4.37; N: 11.79.

¹H NMR (DMSO-d₆, 300 MHz) δ 1.10 (d, 6H), 1.24 (d, 6H) 2.59 (m, 1H), 4.11 (m, 1H), 4.39 (m, 2H), 4.46 (s, 2H), 6.35 (s, 1H), 6.68 (s, 1H), 6.87 (s, 1H), 7.16 (s, 1H), 7.43 (d, 2H), 7.76 (d, 2H), 8.70 (t, 1H), 9.13 (s, 2H), 9.28 (s, 2H), 9.77 (s, 1H)

### EXAMPLE 51

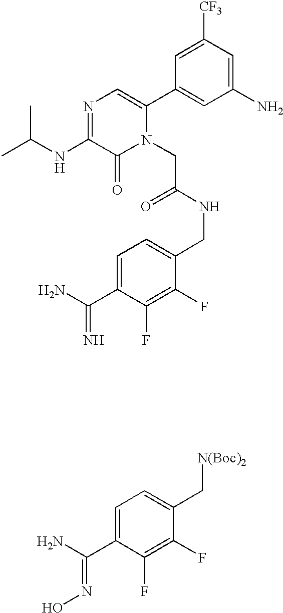

Ex-51a) A mixture of di-(tert-butyl) 4-cyano-2,3-difluorobenzylimidodicarbonate (0.5 g, 1.4 mmol), hydroxylamine hydrochloride (0.28 g, 4.1 mmol), and triethylamine (0.57 ml, 4.1 mmol) in ethanol was heated to reflux for 1 hour. The reaction was concentrated in vacuo and the residue mixed with ethyl acetate, washed with 1N potassium hydrogen sulfate, saturated sodium bicarbonate, brine, dried over magnesium sulfate, filtered, and evaporated in vacuo to give 0.5 g of Ex-51a (89% yield). LCMS (M+H) m/z 402. ¹HNMR (300 MHz, CDCl₃) δ 1.50 (s, 18H), 4.92 (s, 2H), 5.56 (br s, 2H), 7.05-7.14 (m, 1H), 7.42-7.53 (m, 1H). ¹⁹FNMR (282 MHz, CDCl₃) δ −142.53 to −142.32 (m, 1F), −141.3 to −141.0 (m, 1F).

LCMS (ES+) m/z M+H 402.

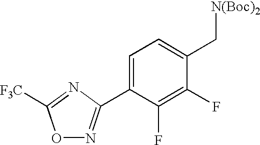

Ex-51b) To a stirred solution of 51a (0.45 g, 1.1 mmol) in pyridine (0.35 ml) and dichloromethane (0.5 ml) was added trifluoroacetic acid anhydride (0.35 ml, 2.5 mmol) while cooling in a water bath and stirring was continued at ambient temperature for 20 minutes. The reaction was concentrated in vacuo and the residue dissolved in ethyl acetate and washed with 1N sodium hydrogen sulfate, brine, dried over magnesium sulfate, filtered, and concentrated in vacuo to gave 0.43 g as an off-white solid. ¹HNMR (300 MHz, CDCl₃) δ 1.52 (s, 18H), 4.99 (s, 2H), 7.19-7.26 (m, 1H), 7.82-7.90 (m, 1H). ¹⁹FNMR (282 MHz, CDCl₃) δ −141.8 to −141.6 (m, 1F), −133.2 to −132.9 (m, 1F), −65.6 (s, 3F).

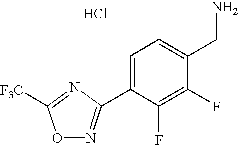

Ex-51c) LCMS (M+H) m/z 280. ¹HNMR (300 MHz, DMSO-d₆) δ 4.25 (s, 2H), 7.68-7.75 (m, 1H), 7.97-8.05 (m, 1H), 8.73 (br s, 3H). ¹⁹FNMR (282 MHz, DMSO-d₆) 6-139.5 to −139.4 (m, 1F), −134.7 to −134.6 (m, 1F), −65.0 (s, 3F).

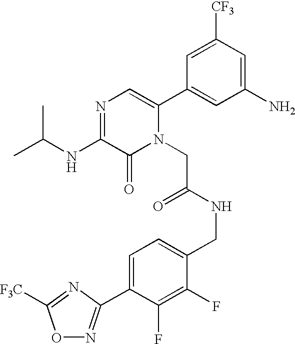

Ex-51d) ¹HNMR (300 MHz, DMSO-d₆) δ 1.23 (d, J=6.3 Hz, 6H), 4.05-4.20 (m, 1H), 4.41 (s, 2H), 4.46 (d, J=5.7 Hz, 2H), 5.81 (s, 2H), 6.72 (s, 1H), 6.80 (s, 1H), 6.89-6.95 (m, 2H), 7.33 (t, J=6.7 Hz, 1H), 7.82-7.90 (m, 1H), 8.78 (t, J=5.8 Hz, 1H). ¹⁹FNMR (282 MHz, DMSO-d₆) δ −142.7 to −142.5 (m, 1F), −135.4 to −135.2 (m, 1F), −65.0 (s, 3F), −62.0 (s, 3F). HRMS (ES) calcd for C₂₆H₂₂N₇O₃F₈ (M+H): 632.1651. Found: 632.1674. Anal. Calcd for C₂₆H₂₁N₇O₃F₈+0.15 CH₄OH: C, 49.36; H, 3.42; N, 15.40. Found: C, 49.47; H, 3.33; N, 15.27.

Ex-51) A suspension of 51d (67 mg, 0.10 mmol) and 10% palladium on carbon (100 mg) in ethanol (30 ml) was shaken under hydrogen at 40 psi for 1.25 hr. The reaction was filtered and concentrated under a nitrogen stream. Purification by reverse phase HPLC (10-60% acetonitrile/water) followed by concentration in vacuo. The residue was dissolved in methanol and concentrated in vacuo to give 46 mg (51% yield) of an off-white solid.

¹HNMR (300 MHz, DMSO-d₆) δ 1.25 (d, J=6.6 Hz, 6H), 4.07-4.20 (m, 1H), 4.40 (s, 2H), 4.44 (d, J=5.4 Hz, 2H), 6.73-6.85 (m, 3H), 6.94 (s, 1H), 7.22-7.42 (m, 2H), 7.44-7.52 (m, 1H), 8.85 (t, J=5.7 Hz, 1H), 9.41-9.61 (m, 3H). ¹⁹FNMR (282 MHz, DMSO-d₆) 6-142.9 to −142.7 (m, 1F), −139.3 to −139.1 (m, 1F), −62.0 (s, 3F). HRMS (ES) calcd for C₂₄H₂₅N₇O₂F₅ (M+H): 538.1984. Found: 538.1950. Anal. Calcd for C₂₄H₂₄N₇O₂F₅+2.5 TFA+0.5 CH₃OH: C, 41.31; H, 3.34; N, 11.43. Found: C, 41.35; H, 3.30; N, 11.36.

### EXAMPLE 52

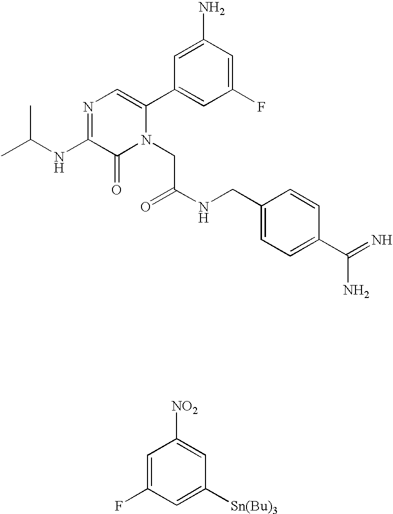

Tributyl(3-fluoro-5-nitrophenyl)stannane

Ex-52a) 1-Fluoro-3-iodo-5-nitrobenzene (1.02 g, 3.8 mmol) was dissolved in 5 mL of toluene. The system was evacuated, then flushed with N₂. Repeated 4×. Pd(PPh₃)₄ (47 mg, 0.041 mmol) and bis(tributyltin) (5.7 mL, 11.3 mmol) were added. The system was evacuated, then flushed with N₂. The reaction was stirred at 90° C. under N₂ until TLC showed disappearance of starting material (overnight).

The reaction solution was cooled to room temperature and diluted with 25 mL of EtOAc. The solution was washed with brine 1×, aqueous saturated KF 1×, and brine 1×. The organic phase was dried over MgSO₄ and concentrated under reduced pressure.

The residue was purified on silica, eluting with a 5/95%-10/90% EtOAc/hexanes gradient, to give 1.46 g (91%) of a yellow oil: ¹H NMR (400 MHz, CDCl₃) δ 0.82-0.91 (m, 9H), 1.09-1.15 (m, 6H), 1.23-1.38 (m, 6H), 1.42-1.55 (m, 6H), 7.46 (m, 1H), 7.79 (m, 1H), 8.06 (m, 1H); ¹⁹F NMR (375 Hz, CDCl₃) δ −111 (m).

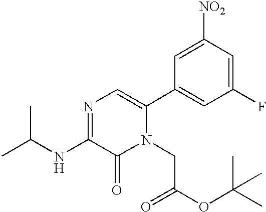

Tert-Butyl [6-(3-fluoro-5-nitrophenyl)-3-(isopropylamino)-2-oxopyrazin-1 (2H)-yl]acetate

Ex-52b) A flask containing 5 mL of DMF was evacuated and flushed with N₂. Repeated 4×. tert-Butyl [6-bromo-3-(isopropylamino)-2-oxopyrazin-1 (2H)-yl]acetate (1.12 g, 3.23 mmol), PPh₃ (54 mg, 0.21 mmol), and 2,6-di-t-butyl-4-methylphenol (13 mg, 0.06 mmol) were added in one portion. The system was evacuated and flushed with N₂. Repeated 3×. Cu(I) Br (61 mg, 0.43 mmol), Pd(PPh₃)₄ (134 mg, 0.12 mmol), the product from Ex-52a (0.48 g, 1.1 mmol), and 2.5 mL of DMF were added. The system was evacuated and flushed with N₂. Repeated 3×. The reaction was stirred at 110° C. under positive N₂ pressure until TLC showed disappearance of starting material (1 h 15 mins).

The dark red solution was cooled and diluted with 40 mL of EtOAc, then washed with aqueous KF 1×100 mL, aqueous saturated NaHCO₃ 1×75 mL, and brine 1×75 mL. The organic phase was dried over MgSO₄ and concentrated.

The dark orange oil was purified on silica, eluting with 20/80% EtOAc/hexanes, to give 0.2 g (44%) of an orange solid: LRMS m/z 407.1 (M⁺+H); HPLC purity (retention time): 90% (3.5 min).

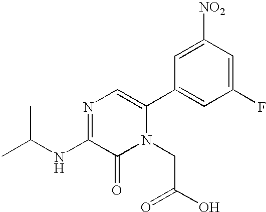

[6-(3-Fluoro-5-nitrophenyl)-3-(isopropylamino)-2-oxopyrazin-1 (2H)-yl]acetic Acid

Ex-52c) The product from Ex-52b (0.2 g, 0.5 mmol) was dissolved in 2 mL of CH₂Cl₂. Triflic acid (88 μL, 1 mmol) and TFA (60 μL, 0.78 mmol) were added. The reaction was stirred for 20 mins.

The solid that precipitated out was filtered and dried under house vacuum to give 168 mg (96%) of an off-white solid: LRMS m/z 351.1 (M⁺+H); HPLC purity (retention time): >99% (2.3 min).

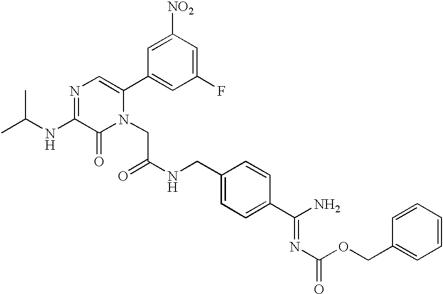

4-((Z)-Amino{[(benzyloxy)carbonyl]imino}methyl)benzyl [6-(3-fluoro-5-nitrophenyl)-3-(isopropylamino)-2-oxopyrazin-1 (2H)-yl]acetate

Ex-52d) 147 mg (0.42 mmol) of the product from Ex-52c; 8 mg (0.06 mmol) HOBt, 129 mg (0.36 mmol) benzyl [4-(aminomethyl)phenyl](imino)methylcarbamate dihydrochloride; 0.30 mL (2.7 mmol) NMM, 0.71 g (0.75 mmol) PS-carbodiimide, 6 mL CH₂Cl₂, and 2 mL DMF. Agitated for 2.5 h, then added 0.15 g (0.42 mmol) PS-diethylenetriamine and 0.17 g (0.48 mmol) Wang aldehyde scavenging resins for 1 h 10 mins to give 0.37 g of crude, desired product: LRMS m/z 616.2 (M⁺+H); HPLC purity (retention time): >90% (2.9 min).

Ex-52) 0.37 g of crude product from Ex-52d; 94 mg (0.04 mmol) of 10% Pd/C (50% water-wet) and 5 mL of MeOH. Stirred overnight.

The mixture was filtered through a 0.2 □M frit and concentrated. The crude residue was purified by reverse-phase HPLC with a gradient of 5/95%-60/40% acetonitrile/water (+0.1% TFA) over 10 mins. The product-containing fractions were concentrated and dried under high vacuum for 6 h to give 92 mg of a pale yellow solid: LRMS m/z 452.1 (M⁺+H); HPLC purity (retention time): >95% (1.8 min); HRMS (M+H): Calc'd for C₂₃H₂₆FN₇O₂: 452.2210; Found: 452.2225; ¹H NMR (400 MHz, CD₃OD) □ 1.39 (m, 6H), 4.02 (m, 1H), 4.48 (d, 2H, J=4.3 Hz), 4.60 (s, 2H), 6.37-6.39 (m, 1H), 6.51-6.56 (m, 2H), 6.61-6.62 (m, 1H), 7.46 (d, 2H, J=8.2 Hz), 7.77 (d, 2H, J=8.4 Hz).

### EXAMPLE 53

N-{4-[Amino(imino)methyl]benzyl}-2-[6-(3-amino-5-methoxyphenyl)-3-(isopropylamino)-2-oxopyrazin-1 (2H)-yl]acetamide

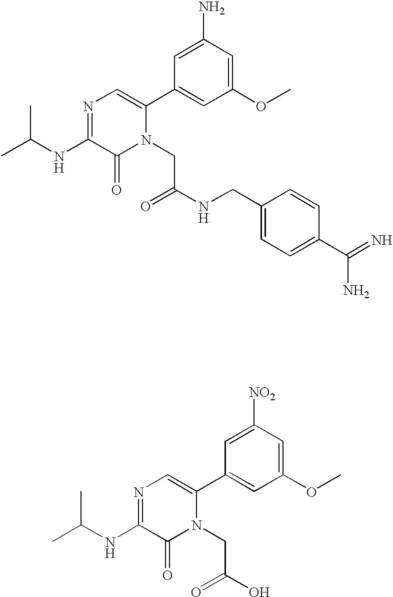

[3-(Isopropylamino)-6-(3-methoxy-5-nitrophenyl)-2-oxopyrazin-1 (2H)-yl]acetic Acid

Ex-53a) The product from Ex-52b (180 mg, 0.44 mmol), NaOCH₃ (250 mg, 4.6 mmol) and 3 mL of MeOH were stirred at 75° C. overnight.

The MeOH was removed under reduced pressure. The dark brown residue was dissolved in 20 mL of EtOAc. The product was extracted with aqueous saturated NaHCO₃ 4×10 mL. The combined aqueous extracts were acidified to pH˜2 with 2 M HCl, then extracted with EtOAc 4×50 mL. The combined organic extracts were washed with brine 1×, dried over MgSO₄, filtered, and concentrated to give 140 mg (88%) of a brown solid: LRMS m/z 363.1 (M⁺+H); HPLC purity (retention time): >80% (2.3 min).

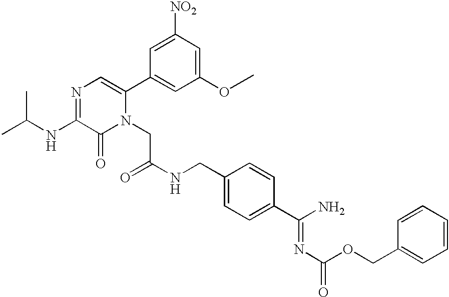

Benzyl (1Z)-amino{4-[({[3-(isopropylamino)-6-(3-methoxy-5-nitrophenyl)-2-oxopyrazin-1 (2H)-yl]acetyl}amino)methyl]phenyl}methylidenecarbamate

Ex-53b) 55 mg (0.15 mmol) of the product from Ex-53a; 3.4 mg (0.02 mmol) HOBt; 48 mg (0.13 mmol) benzyl [4-(aminomethyl)phenyl](imino)methylcarbamate dihydrochloride; 0.10 mL (0.91 mmol) NMM; 0.23 g (0.24 mmol) PS-carbodiimide, 3 mL CH₂Cl₂, and 0.7 mL DMF. Agitated for 1.5 h, then added 60 mg (0.17 mmol) PS-diethylenetriamine and 50 mg (0.14 mmol) Wang aldehyde scavenging resins for 1.5 h to give 0.11 g of crude, desired product: LRMS m/z 628.2 (M⁺+H); HPLC purity (retention time): >85% (2.75 min).

Ex-53) 0.11 g of crude product from Ex-53b; 40 mg (0.02 mmol) of 10% Pd/C (50% water-wet); 1.5 mL of MeOH, and enough THF to dissolve the pyrazinone (2.5 mL). Stirred for 3.5 h

The mixture was filtered through a 0.2 □M frit and concentrated. The crude residue was purified by reverse-phase HPLC with a gradient of 10/90%-50/50% acetonitrile/water (+0.1% TFA) to give 54 mg of a yellow, glassy solid: LRMS m/z 464.2 (M⁺+H); HPLC purity (retention time): 97% (1.6 min); HRMS (M+H): Calc'd for C₂₄H₂₉N₇O₃: 464.2405; Found: 464.2379; ¹H NMR (400 MHz, CD₃OD) δ 1.38 (d, 6H, 6.6 Hz), 3.74 (s, 3H), 4.04 (m, 1H), 4.48 (d, 2H, J=5.5 Hz), 4.59 (s, 2H), 6.55-6.56 (m, 2H), 6.63-6.65 (m, 2H), 7.47 (d, 2H, J=8.2 Hz), 7.76 (d, 2H, J=8.3 Hz), 8.82 (br t, 1H).

### EXAMPLES 54/55

4-[Amino(imino)methyl]benzyl [6-(3-amino-5-hydroxyphenyl)-3-(isopropylamino)-2-oxopyrazin-1 (2H)-yl]acetate

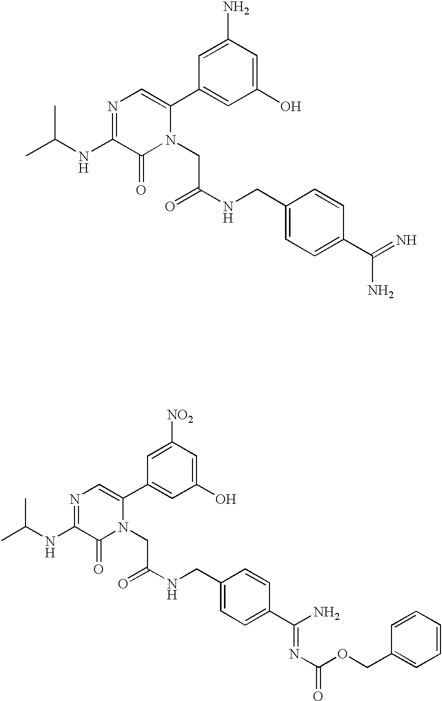

4-((Z)-amino{[(benzyloxy)carbonyl]imino}methyl)benzyl [6-(3-hydroxy-5-nitrophenyl)-3-(isopropylamino)-2-oxopyrazin-1 (2H)-yl]acetate

Ex-54a) LRMS m/z 614.2 (M⁺+H); HPLC purity (retention time): >90% (1.9 min).

Ex-54) 0.31 g of crude product from Ex-54a: 160 mg (0.08 mmol) of 10% Pd/C (50% water-wet) and 5 mL of MeOH. Stirred for 3.5 h.

The mixture was filtered through a 0.2 μM frit and concentrated. The crude oil was purified by reverse-phase HPLC with a gradient of 5/95%-30/70% acetonitrile/water (+0.1% TFA) to give 58 mg of a pale yellow solid: LRMS m/z 450.2 (M⁺+H); HPLC purity (retention time): >99% (1.0 min); HRMS (M+H): Calc'd for C₂₃H₂₇N₇O₃: 450.2248; Found: 450.2270; ¹H NMR (400 MHz, CD₃OD) δ 1.37 (d, 6H, 6.4 Hz), 4.04 (m, 1H), 4.49 (d, 2H, J=4.2 Hz), 4.60 (s, 2H), 6.46-6.64 (m, 4H), 7.47 (d, 2H, J=8.2 Hz), 7.76 (d, 2H, J=8.3 Hz), 8.79 (br t, 1H).

Two salts were prepared for this compound and data for each is reported in Table 3.

### Amide Library

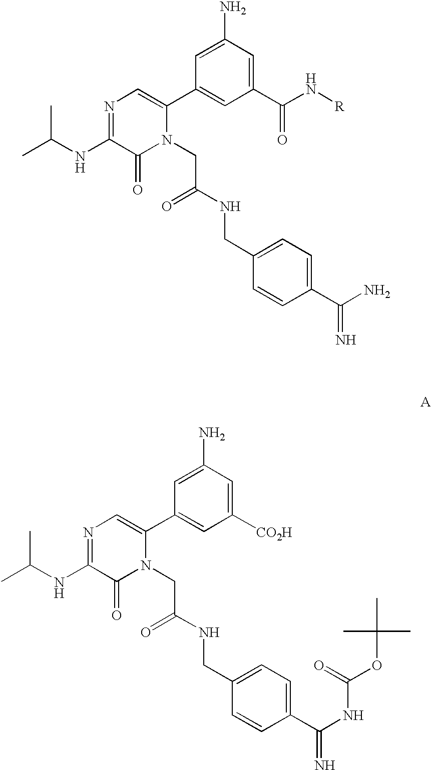

3-amino-5-[1-[2-({4-[[(tert-butoxycarbonyl)amino](imino)methyl]benzyl}amino)-2-oxoethyl]-5-(isopropylamino)-6-oxo-1,6-dihydropyrazin-2-yl]benzoic Acid

LRMS m/z 578.2 (M⁺+H).

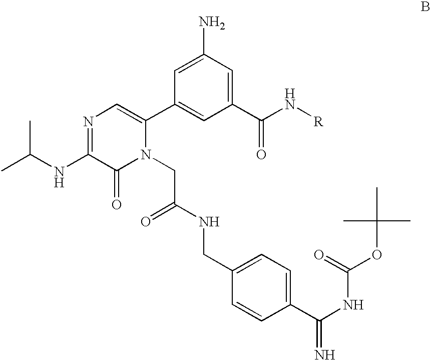

Amine 1 (+/−)-alpha-Methylbenzylamine

Amine 2 (S)-(−)-alpha-Methylbenzylamine

Amine 3 (R)-(+)-alpha-Methylbenzylamine

Amine 4 3.5-Bis(trifluormethyl)benzylamine

Amine 5 Isopropylamine

Amine 6 3-(Aminomethyl)pyridine

Amine 7 3-Phenyl-1-propylamine

Amine 8 Ethylamine

Intermediate A (154 mg, 0.3 mmol), amines 1 through 8 (0.3 mmol), HOBt (34 mg, 0.3 mmol), NMM (0.5 mL, 4. 5 mmol), PS-carbodiimide resin (480 mg, 0.5 mmol), PS-diethylene triamine (88 mg, 0.3 mmol), Aldehyde wang (88 mg, 0.3 mmol).

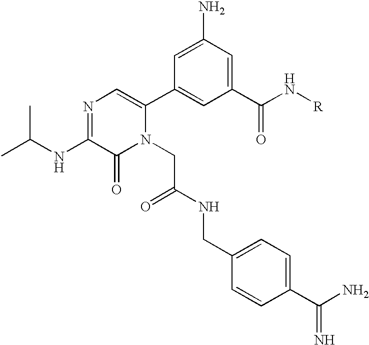

(0.3 mmol), TFA (1.5 mL), CH₂Cl₂ (5 mL). Reverse phase HPLC conditions 5-45% acetonitrile/water with 0.1% TFA over 10 min. to afford an average yield of 54 mg (27%).

### EXAMPLE 56

### NHR=(+/−)-alpha-Methylbenzylamine

LRMS m/z 581.3 (M⁺+H).

### EXAMPLE 57

### NHR=(S)-(−)-alpha-Methylbenzylamine

LRMS m/z 581.3 (M⁺+H).

### EXAMPLE 58

### NHR=(R)-(+)-alpha-Methylbenzylamine

LRMS m/z 581.3 (M⁺+H).

### EXAMPLE 59

### NHR=3,5-Bis(trifluormethyl)benzylamine

LRMS m/z 703.2 (M⁺+H).

### EXAMPLE 60

### NHR=Isopropylamine

LRMS m/z 619.2 (M⁺+H).

### EXAMPLE 61

### NHR=3-(Aminomethyl)pyridine

LRMS m/z 568.2 (M⁺+H).

### EXAMPLE 62

### NHR=3-Phenyl-1-propylamine

LRMS m/z 595.2 (M⁺+H).

### EXAMPLE 63

### NHR=Ethylamine

LRMS m/z 505.2 (M⁺+H).

### EXAMPLE 64

N-{4-[amino(imino)methyl]benzyl}-2-[6-(3-amino-5-isobutoxyphenyl)-3-(isopropylamino)-2-oxopyrazin-1 (2H)-yl]acetamide

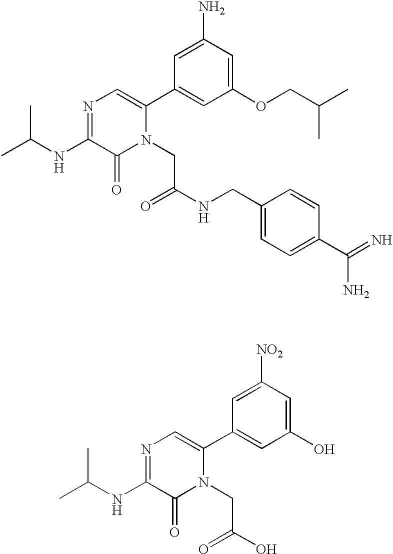

[6-(3-Hydroxy-5-nitrophenyl)-3-(isopropylamino)-2-oxopyrazin-1 (2H)-yl]acetic Acid

Ex-64a) BBr₃ (5 g, 20 mmol) in 18 mL of CH₂Cl₂ was cooled in an ice bath. The product from Ex-53a (1.7 g, 4.7 mmol) in 15 mL of CH₂Cl₂, followed by a 20 mL CH₂Cl₂ rinse, was added. The reaction was stirred at room temperature for 6 h. Additional BBr₃ (2 mL of a 1 M solution in CH₂Cl₂, 2 mmol) was added and the reaction was stirred overnight.

The reaction was quenched with MeOH and concentrated. The residue was taken up in EtOAc and aqueous saturated NaHCO₃. The layers were separated. The organic phase was extracted with aqueous saturated NaHCO₃ 2×100 mL, then 1.2 M aqueous NaOH 4×50 mL. The combined aqueous extracts were acidified to pH˜2 with 2 N HCl, then extracted with EtOAc 4×150 mL. The combined organics were washed with brine, dried over MgSO4, and concentrated to give 0.85 g of a brown solid (53%): LRMS m/z 349.0 (M⁺+H); HPLC purity (retention time): 75% (2.1 min). Additional product was recovered from the aqueous phase: The aqueous phase was concentrated to ˜100 mL. The solid that precipitated was removed by filtration. The filtrate was extracted with THF 5×100 mL. The combined organics were washed with brine, dried over MgSO₄, and concentrated to give 0.63 g (39%, 92% total yield) of a brown solid: LRMS m/z 349.1 (M⁺+H); HPLC purity (retention time): 100% (1.6 min).

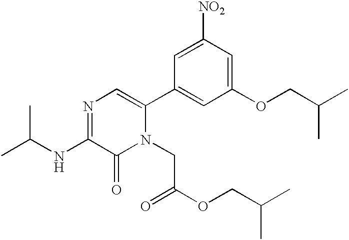

Isobutyl [6-(3-isobutoxy-5-nitrophenyl)-3-(isopropylamino)-2-oxopyrazin-1 (2H)-yl]acetate

Ex-64b) The product from Ex-64a (0.29 g, 0.84 mmol) was dissolved in 7.5 mL of DMSO. K₂CO₃ (0.71 g, 5.1 mmol) was added and the reaction was stirred at 90° C. for 2 h. The reaction was cooled to room temperature and 1-bromo-2-methylpropane (0.23 mL, 2.1 mmol) was added. The reaction was stirred at 90° C. for 1.5 h, then cooled to room temperature. The crude reaction mixture was used directly in the next step. (LRMS m/z 461.2 (M⁺+H); HPLC purity (retention time): 55% (3.5 min))

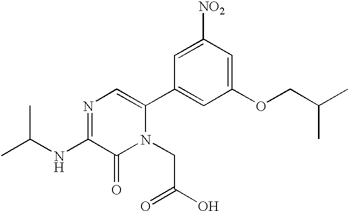

[6-(3-Isobutoxy-5-nitrophenyl)-3-(isopropylamino)-2-oxopyrazin-1 (2H)-yl]acetic Acid

Ex-64c) The crude reaction solution from Ex-64b (˜0.35 g, ˜0.8 mmol) mmol) was added to LiOH.H₂O (194 mg, 4.6 mmol) in 4 mL of H₂O. The reaction was stirred at room temperature for 45 mins. LC/MS analysis showed unreacted starting material. Another 70 mg (1.7 mmol) of LiOH.H₂O in 0.5 mL of H₂O was added.

The crude reaction was washed with EtOAc 3×60 mL. The aqueous phase was acidified with 2 N HCl and extracted with EtOAc 3×40 mL. The combined organics were washed with brine, dried over MgSO₄ and concentrated to give 20 mg of product.

LC/MS analysis of the EtOAc washes of the crude reaction showed starting material and desired product (˜1:1). Therefore, the EtOAc washes of the crude reaction were resubmitted to the reaction conditions: The residue was dissolved in 5 mL of THF. Aqueous LiOH.H₂O (240 mg, 5.7 mmol, in 5 mL of H₂O) was added and the reaction was stirred at room temperature. The reaction was stirred for 1 h. LC/MS analysis showed presence of starting material. Another 200 mg (4.8 mmol) of LiOH.H₂O in 2.5 mL of H₂O was added. The reaction was stirred for 1.5 h. LC/MS showed only aminor amount of starting material remaining.

The THF was removed under reduced pressure. The aqueous residue was extracted with EtOAc 3×. The combined organics were washed with brine, dried over MgSO₄, and concentrated to give 0.16 g (47%) of a brown film: LRMS m/z 405.1 (M⁺+H); HPLC purity (retention time): >90% (2.5 min).

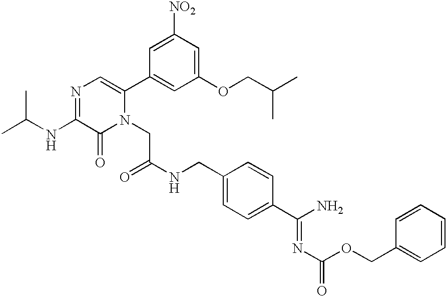

Benzyl (1Z)-amino{4-[({[6-(3-isobutoxy-5-nitrophenyl)-3-(isopropylamino)-2-oxopyrazin-1 (2H)-yl]acetyl}amino)methyl]phenyl}methylidenecarbamate

Ex-64d) 0.14 g (0.35 mmol) of the product from Ex-64c; 6.6 mg (0.05 mmol) HOBt; 107 mg (0.3 mmol) benzyl [4-(aminomethyl)phenyl](imino)methylcarbamate dihydrochloride; 0.23 mL (2.0 mmol) NMM; 0.52 g (0.55 mmol) PS-carbodiimide, 5 mL CH₂Cl₂, and 1.5 mL DMF. Agitated for 1.5 h, then added 0.61 g (1.7 mmol) PS-diethylenetriamine and 0.60 g (1.7 mmol) Wang aldehyde scavenging resins for 1 h 10 m to give 0.15 g of crude, desired product: LRMS m/z 670.2 (M⁺+H); HPLC purity (retention time): >80% (2.5 min).

Ex-64) 0.15 g of crude product from Ex-64d; 85 mg (0.04 mmol) of 10% Pd/C (50% water-wet); and 6 mL of MeOH. Stirred for 24 h.

The mixture was filtered and concentrated. The crude residue was purified by reverse-phase HPLC with a gradient of 10/90%-50/50% acetonitrile/water (+0.1% TFA) to give 53 mg of a pale yellow solid: LRMS m/z 506.2 (M⁺+H); HPLC purity (retention time): >95% (1.8 min); HRMS (M+H): Calc'd for C₂₇H₃₅N₇O₃: 506.2874; Found: 506.2918; ¹H NMR (400 MHz, CD₃OD) δ 1.01 (d, 6H, 6.7 Hz), 1.39 (d, 6H, 6.4 Hz), 2.05 (m, 1H), 3.76 (d, 2H, 6.4 Hz), 4.07 (m, 1H), 4.48 (s, 2H), 4.58 (s, 2H), 6.67 (s, 1H), 6.87-6.93 (m, 3H), 7.47 (d, 2H, J=8.2 Hz), 7.77 (d, 2H, J=8.3 Hz).

### EXAMPLE 65

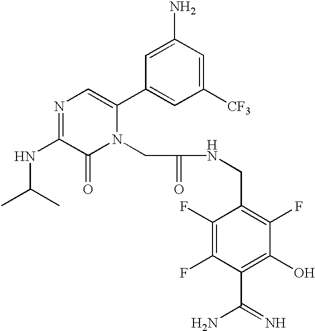

¹HNMR: 400 MHz, MeOD: (δ) 1.389, 1.373 (d, 6H); 4.018 (septet, J=6.4 Hz, 1H); 4.436 (s, 2H); 4.471 (s, 2H);6.623 (s, 1H); 6.85 to 6.84 (m, 1H); 6.866 (s, 1H); 7.05 to 7.03 (m, 1H).

¹⁹FNMR: 400 MHz, MeOD: (δ) −65.030; −77.953; −142.772, −142.808 (d); −146.090, −146.128, −146.149, −146.185 (d of d); −152.810, −152.869 (d).

Elemental analysis:

Found C: 37.07H: 3.29 N: 9.55

Calc. C: 36.90H: 3.41 N: 10.04

### EXAMPLE 66

3-Amino-5-[1-[2-({4-[amino(imino)methyl]benzyl}amino)-2-oxoethyl]-5-(isopropylamino)-6-oxo-1,6-dihydropyrazin-2-yl]benzenesulfonic Acid

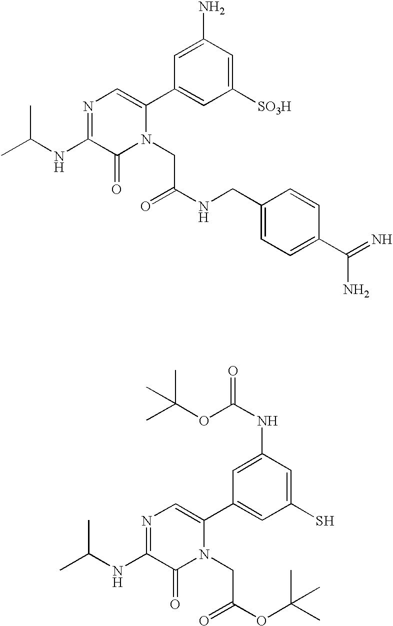

Tert-Butyl [6-{3-[tert-butcarbonyl)amino]-5-mercaptophenyl}-3-(isopropylamino)-2-oxopyrazin-1 (2H)-yl]acetate

Ex-66a) LRMS m/z 491.5 (M⁺+H); HPLC purity (retention time): >99% (2.7 min).

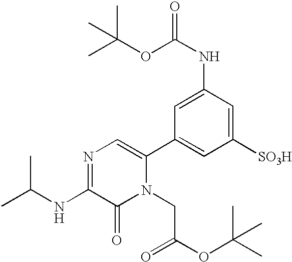

3-[(tert-Butoxycarbonyl)amino]-5-[1-(2-tert-butoxy-2-oxoethyl)-5-(isopropylamino)-6-oxo-1,6-dihydropyrazin-2-yl]benzenesulfonic Acid

Ex-66b) The product from Ex-66a (3.75 g, 7.6 mmol) was taken up in 30 mL of EtOH (not soluble). The mixture was cooled in an ice bath to 10° C. NaOH (1.46 g, 36 mmol) in 15 mL of H₂O was added. The reaction exothermed to 20° C. and became mostly homogeneous.

The reaction was cooled to 5° C. H₂O₂ (4.2 mL of a 30% aqueous solution, 37 mmol) was added dropwise. The reaction exothermed to 9° C. and a thick sludge formed. The reaction was warmed to room temperature, stirring resumed, and the remaining H₂O₂ was added. The reaction exothermed to 32° C. LC/MS analysis after 1 h 10 mins showed disappearance of starting material.

The reaction mixture was filtered through Celite. The filtrate was partially concentrated under reduced pressure. CH₂Cl₂ (100 mL) was added to the remaining basic, aqueous solution. It was swirled with a solution of 52 mL of brine, 3.1 mL of 12 M HCl (37.2 mmol), and 6 mL of H₂O (enough to dissolve the NaCl). The biphasic solution was transferred to a separatory funnel, diluted with 50 mL of CH₂Cl₂, shaken, and the layers were allowed to separate. The organic phase was concentrated. The resulting solid was triturated with hexanes, filtered, the filter cake was washed well with Et₂O, and the solid was dried under high vacuum for 40 mins to give 4.2 g (100% yield) of an orangish-yellow solid: LRMS m/z 539.5 (M⁺+H); HPLC purity (retention time): 85% (2.1 min).

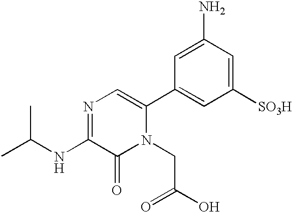

[6-(3-Amino-5-sulfophenyl)-3-(isopropylamino)-2-oxopyrazin-1 (2H)-yl]acetic Acid

Ex-66c) The product from Ex-66b (127 mg, 0.3 mmol) was taken up in 3 mL of CH₂Cl₂. TFA (1 mL, 13 mmol) was added, followed by triflic acid (55 μL, 0.6 mmol). The reaction was stirred at room temperature for 15 mins.

The volatiles were removed under reduced pressure. The residue was purified by reverse-phase HPLC with a gradient of 0/100%-10/90% acetonitrile/water (+0.1% TFA) to give 74 mg of desired product: LRMS m/z 383.0 (M⁺+H); HPLC purity (retention time): 95% (0.17 min).

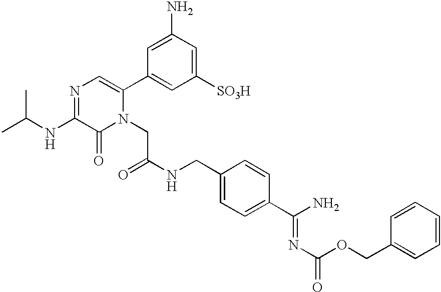

3-Amino-5-[1-(2-{[4-((Z)-amino{[(benzyloxy)carbonyl]imino}methyl)benzyl]amino}-2-oxoethyl)-5-(isopropylamino)-6-oxo-1,6-dihydropyrazin-2-yl]benzenesulfonic Acid

Ex-66d) 74 mg (0.15 mmol) of the product from Ex-66c; 2.9 mg (0.02 mmol) HOBt; 50.7 mg (0.14 mmol) benzyl [4-(aminomethyl)phenyl](imino)methylcarbamate dihydrochloride; 0.10 mL (0.91 mmol) NMM; 0.245 g (0.26 mmol) PS-carbodiimide, 3 mL CH₂Cl₂, and 1.5 mL DMF. Agitated for 45 mins, then added 0.27 g (0.77 mmol) Wang aldehyde scavenging resin for ˜2 h to give 0.14 g of crude, desired product: LRMS m/z 648.2 (M⁺+H); HPLC purity (retention time): 40% (1.6 min). (Impurity is the benzyl [4-(aminomethyl)phenyl](imino)methylcarbamate.)

Ex-66) The crude product from Ex-66d was taken up in CH₂Cl₂. Trific acid (40 □L, 0.45 mmol) was added and the reaction was stirred at room temperature overnight. LC/MS showed starting material still present. Another 150 □L (1.7 mmol) of triflic acid was added and the reaction was stirred for 3 h.

The reaction was diluted with MeOH and concentrated. The crude residue was purified by reverse-phase HPLC with a gradient of 5/95%-35/65% acetonitrile/water (+0.1% TFA) to give 1.7 mg of an off-white solid: LRMS m/z 514.1 (M⁺+H); HPLC purity (retention time): >95% (1.1 min).

### EXAMPLE 67

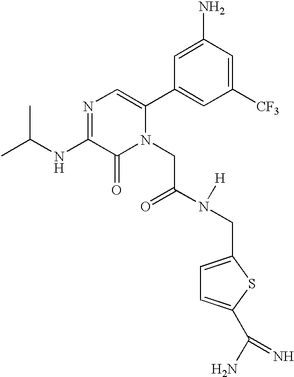

HPLC/LRMS: >97%, 508 (M+H)+; HRMS(ES+) calcd. for C₂₂H₂₅N₇O₂SF₃ 508.1737, found 508.1739.

### EXAMPLE 68

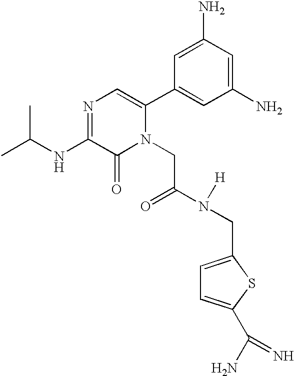

HPLC/LRMS: >95%, 455 (M+H)+; HRMS(ES+) calcd. for C₂₁H₂₇N₈O₂S 455.1972, found 455.1982.

### EXAMPLE 69

2-[6-(5-amino-1,1′-biphenyl-3-yl)-3-(isopropylamino)-2-oxopyrazin-1 (2H)-yl]-N-{4-[amino(imino)methyl]benzyl}acetamide

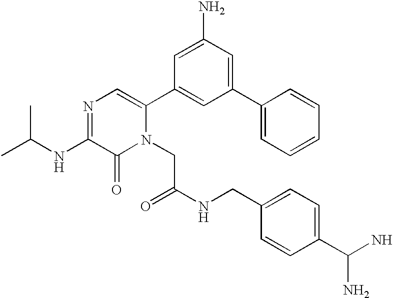

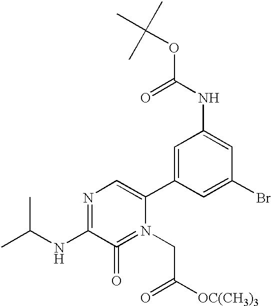

tert-butyl [6-{3-bromo-5-[(tert-butoxycarbonyl)amino]phenyl}-3-(isopropylamino)-2-oxopyrazin-1 (2H)-yl]acetate

Ex-69a) t-Butyl [6-bromo-3-(isopropylamino)-2-oxopyrazin-1 (2H)-yl]acetate (3.6 g, 10.5 mmol), (5.0 g, 12.5 mmol), sodium carbonate (4.4 g, 41.8 mmol), tetrakis(triphenylphospine)palladium (0) (1.2 g, 1.0 mmol),THF (600 mL), DI H₂O (20 mL). Yellow solid afforded 2.6 g (46%).

¹H NMR (300 MHz, CD₃Cl) δ 1.29 (d, 6H, J=6.6 Hz), 1.47 (s, 9H), 1.52 (s, 9H), 4.10-4.20 (m, 1H), 4.39 (s, 2H), 6.13 (b, NH), 6.79 (s, 1H), 7.12 (s, 1H), 7.20 (s, 1H), 7.79 (s, 1H).

LRMS m/z 538.2 (M⁺+H).

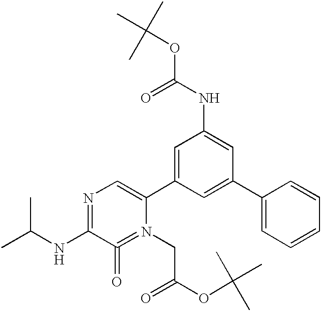

Tert-Butyl [6-{5-[(tert-butoxycarbonyl)amino]-1,1′-biphenyl-3-yl}-3-(isopropylamino)-2-oxopyrazin-1 (2H)-yl]acetate

Ex-69b) Ex-69a (500 mg, 0.9 mmol), phenyl boronic acid (227 mg, 1.9 mmol), sodium carbonate (308 mg, 2.9 mmol), tetrakis(triphenylphospine)palladium (0) (104 mg, 0.1 mmol),THF (15 mL), DI H2O (2 mL).

Yellow solid afforded 400 mg (80%).

¹H NMR (300 MHz, CD₃Cl) δ 1.30 (d, 6H, J=6.3 Hz), 1.39 (s, 9H), 1.55 (s, 9H), 4.08-4.24 (m, 1H), 4.46 (s, 2H), 6.89 (s, 1H), 7.27-7.77 (m, 8H).

LRMS m/z 535.2 (M⁺+H).

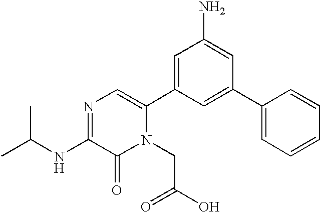

[6-(5-amino-1,1′-biphenyl-3-yl)-3-(isopropylamino)-2-oxopyrazin-1 (2H)-yl]acetic Acid

Ex-69c) Ex-69b (300 mg, 0.6 mmol), trifluoromethane sulfonic acid (0.23 mL, 0.3 mmol), CH₂Cl₂ (60 mL). Brown solid afforded 212 mg (98%).

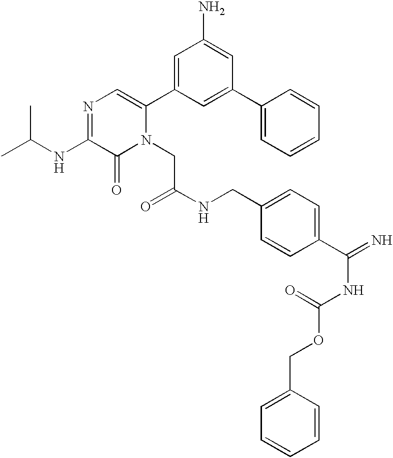

benzyl {4-[({[6-(5-amino-1,1′-biphenyl-3-yl)-3-(isopropylamino)-2-oxopyrazin-1 (2H)-yl]acetyl}amino)methyl]phenyl}(imino)methylcarbamate

Ex-69d) Ex-69c (212 mg, 0.6 mmol), benzyl [4-(aminomethyl)phenyl](imino)methylcarbamate dihydrochloride (249 mg, 0.7 mmol), HOBt (25 mg, 0.2 mmol), and NMM (0.4 mL, 3.4 mmol), PS-carbodiimide resin (1.0 g, 1.1 mmol), PS-diethylene triamine (0.4 g, 1.1 mmol), Aldehyde wang (0 g, 0 mmol), CH₂Cl₂ (5 mL) and DMF (3 mL).

Ex-69) Ex-69d (360 mg, 0.6 mmol), Pd/C (10%, 30 mg), MeOH (50 mL). White solid afforded 320 mg (78%). ¹H NMR (300 MHz, CD₃OD) δ 1.41 (d, 6H, J=6.6 Hz), 4.03-4.13 (m, 1H), 4.48 (s, 2H), 2.66 (s, 2H), 6.72 (s, 1 h), 6.89 (s, 1H), 7.15 (s, 1H), 7.27 (s, 1H), 7.38-7.66 (m, 9H)

HRMS m/z 510.3 (M⁺+H).

### EXAMPLE 70

N-{4-[amino(imino)methyl]benzyl}-2-[6-[3-amino-5-(isobutylthio)phenyl]-3-(isopropylamino)-2-oxopyrazin-1 (2H)-yl]acetamide

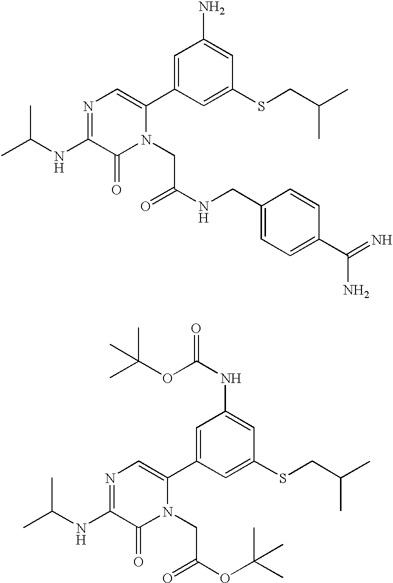

tert-Butyl [6-[3-[(tert-butoxycarbonyl)amino]-5-(isobutylthio)phenyl]-3-(isopropylamino)-2-oxopyrazin-1 (2H)-yl] acetate

Ex-70a) LRMS m/z 547.3 (M⁺+H); HPLC purity (retention time): 70% (3.5 min).

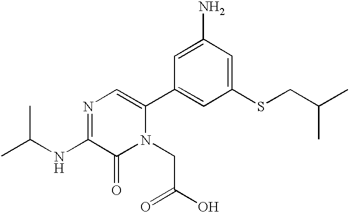

[6-[3-amino-5-(isobutylthio)phenyl]-3-(isopropylamino)-2-oxopyrazin-1 (2H)-yl]acetic Acid

Ex-70b) The product from Ex-70a (0.23 g, 0.4 mmol) was dissolved in 4 mL of CH₂Cl₂. Triflic acid (74 μL, 0.8 mmol) was added, followed by enough TFA to make the reaction homogeneous (0.6 mL, 7.8 mmol). LC/MS analysis after 15 mins showed some starting material with only the BOC protecting group removed. Another 74 μL (0.8 mmol) of triflic acid was added. The reaction was stirred for 1 h. LC/MS showed completion of the reaction.

The volatiles were removed under reduced pressure. The oil was dissolved in EtOAc. The organic phase was washed with aqueous saturated NaHCO₃ 1×, then 2.5 M aqueous NaOH 3×. Product is present in both the organic and aqueous phases.

The organic phase was concentrated to give 96 mg of a yellow solid: LRMS m/z 391.1 (M⁺+H); HPLC purity (retention time): 90% (1.9 min).

The aqueous phase was neutralized with 2 M HCl, then extracted with EtOAc 3×. The combined organics were dried over MgSO₄, filtered, and concentrated to give 0.28 g of an orange oil. The oil was purified by reverse-phase HPLC with a gradient of 15/85%-45/55% acetonitrile/water (+0.1% TFA) to give 60 mg of a yellow residue: LRMS m/z 391.1 (M⁺+H); HPLC purity (retention time): >99% (1.9 min).

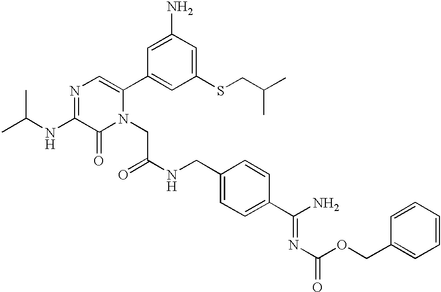

Benzyl (1Z)-amino{4-[({[6-[3-amino-5-(isobutylthio)phenyl]-3-(isopropylamino)-2-oxopyrazin-1 (2H)-yl]acetyl}amino)methyl]phenyl}methylidenecarbamate

Ex-70c) 70 mg (0.18 mmol) of the product from Ex-70b; 4 mg (0.03 mmol) HOBt; 60 mg (0.17 mmol) benzyl [4-(aminomethyl)phenyl](imino)methylcarbamate dihydrochloride; 0.12 mL (1.1 mmol) NMM; 0.275 g (0.29 mmol) PS-carbodiimide, 3 mL CH₂Cl₂, and 1.5 mL DMF. Agitated for 1 h 10 mins. Sequestering resins were not added. The mixture was filtered, rinsed, and concentrated to give 0.17 g of crude, desired product:

LRMS m/z 656 (M⁺+H); HPLC purity (retention time): >90% (2.1 min).

Ex-70) 0.17 g of crude product from Ex-70c; 43 mg (0.02 mmol) of 10% Pd/C (50% water-wet); and 1.5 mL of MeOH. Stirred for 30 h. LC/MS showed starting material still present.

The mixture was filtered and concentrated. The crude was taken up in CH₂Cl₂. Triflic acid (80 μL, 0.9 mmol) was added. The reaction was exothermic. The reaction was stirred for 1 h, then quenched with MeOH and concentrated. The crude residue was purified by reverse-phase HPLC with a gradient of 15/85%-45/55% acetonitrile/water (+0.1% TFA) to give 7 mg of a dark yellow solid: LRMS m/z 522.2 (M⁺+H); HPLC purity (retention time): >90% (1.7 min); HRMS (M+H): Calc'd for C₂₇H₃₅N₇O₂S: 522.2646; Found: 522.2645; ¹H NMR (400 MHz, CD₃OD) δ 1.02 (d, 6H, 6.6 Hz), 1.39 (d, 6H, 6.4 Hz), 1.85 (m, 1H), 2.81 (d, 2H, 6.8 Hz), 4.03 (m, 1H), 4.50 (s, 2H), 4.56 (s, 2H), 6.62 (s, 1H), 6.69 (m, 1H), 6.84 (m, 1H), 6.96 (m, 1H), 7.48 (d, 2H, J=8.2 Hz), 7.77 (d, 2H, J=8.2 Hz).

### EXAMPLE 71

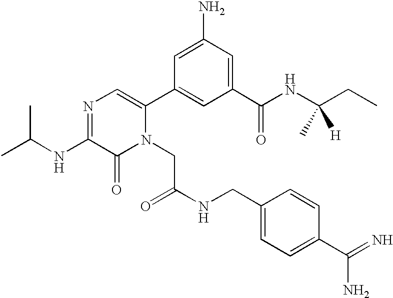

The compound of Example 71 was prepared in an analogous manner to that of Example 186.

### EXAMPLE 72

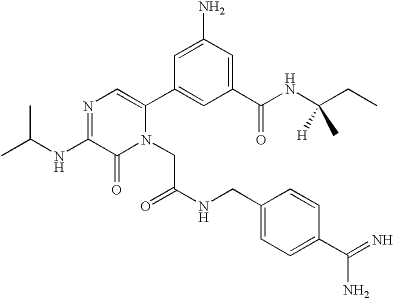

The compound of Example 72 was prepared in an analogous manner to that of Example 186.

### EXAMPLE 73

N-{4-[amino(imino)methyl]benzyl}-2-[6-[3-amino-5-(isobutylsulfinyl)phenyl]-3-(isopropylamino)-2-oxopyrazin-1 (2H)-yl]acetamide

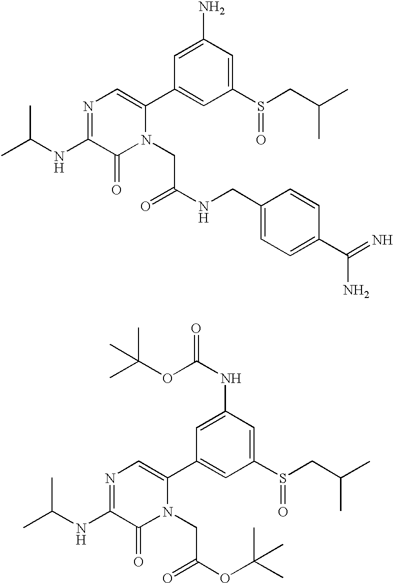

Tert-Butyl [6-[3-[(tert-butoxycarbonyl)amino]-5-(isobutylsulfinyl)phenyl]-3-(isopropylamino)-2-oxopyrazin-1 (2H)-yl]acetate

Ex-73a) The product from Ex-70a (780 mg, 1.4 mmol, ˜35% Br analog) was dissolved in 16 mL of MeOH and cooled in an ice bath. Oxone (0.44 g, 1.4 mmol KHSO₅) in 16 mL of H₂O was added dropwise over 2 mins. The reaction became cloudy. The reaction was stirred for 2 mins. Half of the reaction solution was removed and extracted with CH₂Cl₂. (The other half was oxidized to the sulfone. See Ex-74a.) The organic phase was washed with brine, dried over MgSO₄, filtered, and concentrated to give 224 mg of a white solid (with 10% sulfone impurity, 40% Br impurity, and 5% starting material): LRMS m/z 563.3 (M⁺+H); HPLC purity (retention time): 45% (2.7 min).

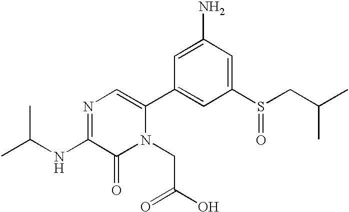

[6-[3-amino-5-(isobutylsulfinyl)phenyl]-3-(isopropylamino)-2-oxopyrazin-1 (2H)-yl]acetic Acid

Ex-73b) The crude product from Ex-73a (0.22 g crude, 0.4 mmol crude) was dissolved in 4 mL of CH₂Cl₂ and cooled in an ice bath. Triflic acid (180 μL, 2.0 mmol) was added, followed by enough TFA to make the reaction homogeneous (180 □L, 2.3 mmol). LC/MS analysis after 10 mins showed completion of the reaction.

The volatiles were removed under reduced pressure. The crude residue was purified by reverse-phase HPLC with a gradient of 15/85%-35/65% acetonitrile/water (+0.1% TFA) to give ˜42 mg of a yellow residue: LRMS m/z 407.1 (M⁺+H); HPLC purity (retention time): >90% (1.3 min).

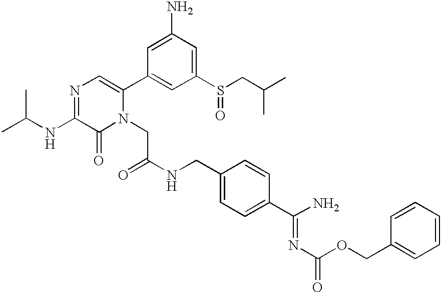

Benzyl (1Z)-amino{4-[({[6-[3-amino-5-(isobutylsulfinyl)phenyl]-3-(isopropylamino)-2-oxopyrazin-1 (2H)-yl]acetyl}amino)methyl]phenyl}methylidenecarbamate

Ex-73c) 81 mg (0.16 mmol) of the product from Ex-73b; 3 mg (0.02 mmol) HOBt; 54 mg (0.15 mmol) benzyl [4-(aminomethyl)phenyl](imino)methylcarbamate dihydrochloride; 0.106 mL (0.96 mmol) NMM; 0.305 g (0.30 mmol) PS-carbodiimide, 3 mL CH₂Cl₂, and 1.5 mL DMF. Agitated for 3 h. Sequestering resins were not added. The mixture was filtered, rinsed, and concentrated to give 0.20 g of crude, desired product: LRMS m/z 672.2 (M⁺+H); HPLC purity (retention time): 75% (2.0 min).

Ex-73) The crude product from Ex-73c was dissolved in 1.5 mL of CH₂Cl₂. Triflic acid (0.14 mL, 1.6 mmol, in two portions) and anisole (17 μL, 0.16 mmol) were added. No product was detected by LC/MS analysis after 1 h 10 mins.

The volatiles were removed under reduced pressure. 25 mg (0.012 mmol) of 10% Pd/C (50% water-wet) and 2 mL of MeOH. Stirred for 45 mins. LC/MS showed evidence of reduction of the sulfoxide to the sulfide.

The mixture was filtered and concentrated. The crude residue was purified by reverse-phase HPLC with a gradient of 20/80%-60/40% acetonitrile/water (+0.1% TFA) to give 20 mg of a yellow glassy solid: LRMS m/z 538.2 (M⁺+H); HPLC purity (retention time): >95% (1.3 min); HRMS (M+H): Calc'd for C₂₇H₃₅N₇O₃S: 538.2595; Found: 538.2615; ¹H NMR (400 MHz, CD₃OD) δ 1.06 (d, 3H, 6.8 Hz), 1.13 (d, 3H, 6.6 Hz), 1.40 (d, 6H, 6.4 Hz), 2.14 (m, 1H), 2.62-2.67 (m, 1H), 2.79-2.85 (m, 1H), 4.02 (m, 1H), 4.40-4.68 (m, 4H), 6.64 (s, 1H), 6.88 (m, 2H), 7.11 (m, 1H), 7.42 (d, 2H, J=8.3 Hz), 7.76 (d, 2H, J=8.3 Hz), 8.84 (br t, 1H).

### EXAMPLES 74/75

N-{4-[amino(imino)methyl]benzyl}-2-[6-[3-amino-5-(isobutylsulfonyl)phenyl]-3-(isopropylamino)-2-oxopyrazin-1 (2H)-yl]acetamide

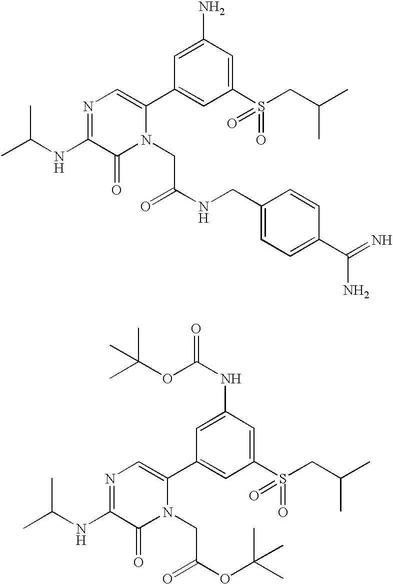

Tert-Butyl [6-[3-[(tert-butoxycarbonyl)amino]-5-(isobutylsulfonyl)phenyl]-3-(isopropylamino)-2-oxopyrazin-1 (2H)-yl]acetate

Ex-74a) The product from Ex-70a (780 mg, 1.4 mmol, ˜35% Br analog) was dissolved in 16 mL of MeOH and cooled in an ice bath. Oxone (0.44 g, 1.4 mmol KHSO5) in 16 mL of H₂O was added dropwise over 2 mins. The reaction became cloudy. The reaction was stirred for 2 mins. Half of the reaction solution was removed. (Used to make the sulfoxide. See Ex-73a.) The reaction was stirred for another 45 mins. LC/MS analysis showed starting material present. Another 42 mg (0.14 mmol KHSO₅) of Oxone was added and the reaction was stirred for 45 mins.

The volatiles were removed under reduced pressure. The aqueous residue was extracted with CH₂Cl₂ 3×15 mL. The combined organic extracts were washed with brine, dried over MgSO₄, filtered, and concentrated to give ˜465 mg of a white solid (with 15% sulfoxide, 40% bromo analog, and 10% starting material): LRMS m/z 579.2 (M⁺+H); HPLC purity (retention time): 35% (3.0 min).

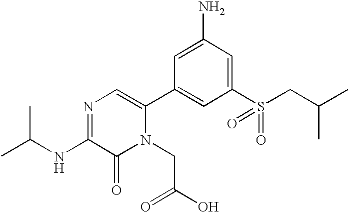

[6-[3-amino-5-(isobutylsulfonyl)phenyl]-3-(isopropylamino)-2-oxopyrazin-1 (2H)-yl]acetic Acid

Ex-74b) The crude product from Ex-74a (0.62 g crude, 1.1 mmol crude) was dissolved in 10 mL of CH₂Cl₂ and cooled in an ice bath Triflic acid (490 μL, 5.5 mmol) was added, followed by enough TFA to make the reaction homogeneous (980 μL, 13 mmol). LC/MS analysis after 10 mins showed completion of the reaction.

The volatiles were removed under reduced pressure. The crude residue was purified by reverse-phase HPLC with a gradient of 15/85%-35/65% acetonitrile/water (+0.1% TFA) to give 130 mg of a yellow solid: LRMS m/z 423.1 (M⁺+H); HPLC purity (retention time): >95% (1.5 min).

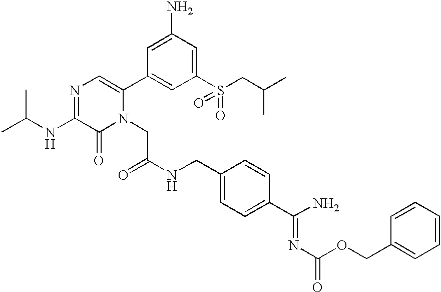

Benzyl (1Z)-amino{4-[({[6-[3-amino-5-(isobutylsulfonyl)phenyl]-3-(isopropylamino)-2-oxopyrazin-1 (2H)-yl]acetyl}amino)methyl]phenyl}methylidenecarbamate

Ex-74c) 121 mg (0.22 mmol) of the product from Ex-74b; 2.8 mg (0.02 mmol) HOBt; 78.8 mg (0.22 mmol) benzyl [4-(aminomethyl)phenyl](imino)methylcarbamate dihydrochloride; 0.15 mL (1.4 mmol) NMM; 0.43 g (0.45 mmol) PS-carbodiimide, 3 mL CH₂Cl₂, and 1.5 mL DMF. Agitated for 3 h. Sequestering resins were not added. The mixture was filtered, rinsed, and concentrated to give 0.30 g of crude, desired product: LRMS m/z 688.3 (M⁺+H); HPLC purity (retention time): 85% (2.0 min).

Ex-74) The crude product from Ex-74c was dissolved in 2.2 mL of CH₂Cl₂. Triflic acid (0.19 μL, 2.2 mmol, in two portions) and anisole (24 μL, 0.22 mmol) were added. No product was detected by LC/MS analysis after 1 h 10 mins.

The volatiles were removed under reduced pressure. 25 mg (0.012 mmol) of 10% Pd/C (50% water-wet) and 2 mL of MeOH. Stirred for 45 mins.

The mixture was filtered and concentrated. The crude residue was purified by reverse-phase HPLC with a gradient of 20/80%-50/50% acetonitrile/water (+0.1% TFA) to give 50 mg of a pale yellow solid: LRMS m/z 554.2 (M⁺+H); HPLC purity (retention time): >98% (1.4 min); HRMS (M+H): Calc'd for C₂₇H₃₅N₇O₄S: 554.2544; Found: 544.2576; ¹H NMR (400 MHz, CD₃OD) δ 1.04 (d, 6H, J=6.7 Hz), 1.40 (d, 6H, J=6.4 Hz), 2.15 (m, 1H), 3.07 (m, 2H), 4.04 (m, 1H), 4.48 (s, 2H), 4.57 (s, 2H), 6.67 (s, 1H), 6.95 (m, 1H), 7.13 (m, 1H), 7.27 (m, 1H), 7.45 (d, 2H, J=8.4 Hz), 7.76 (d, 2H, J=8.5 Hz), 8.78 (br t, ˜1H).

Two salts were prepared for this compound and the data for each is reported in Table 3.

### EXAMPLE 76

N-{4-[amino(imino)methyl]benzyl}-2-[6-[3-amino-5-(3-phenylpropyl)phenyl]-3-(isopropylamino)-2-oxopyrazin-1 (2H)-yl]acetamide

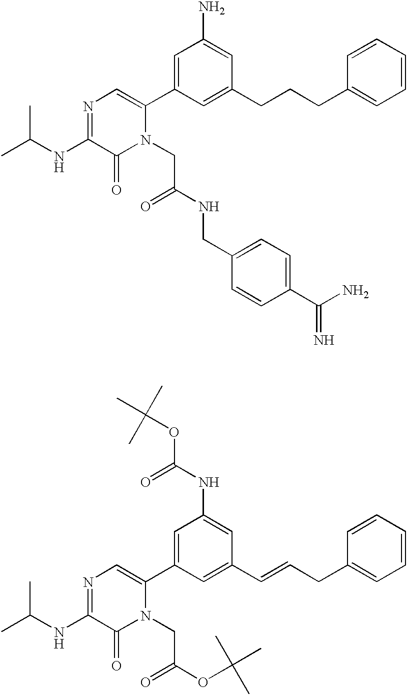

tert-butyl [6-{3-[(tert-butoxycarbonyl)amino]-5-[(1E)-3-phenylprop-1-enyl]phenyl}-3-(isopropylamino)-2-oxopyrazin-1 (2H)-yl]acetate

Ex-76a) ¹H NMR (300 MHz, CD₃Cl) 61.29 (d, 6H, J=6.6 Hz), 1.43 (s, 9H), 1.52 (s, 9H), 3.53 (d, 1H, J=5.7 Hz), 4.11=4.21 (m, 1H), 4.41 (d, 2H, J=9.0 Hz), 6.01 (d, 1H, J=7.8 Hz), 7.18-7.37 (m, 5H).

LRMS m/z 535.2 (M⁺+H).

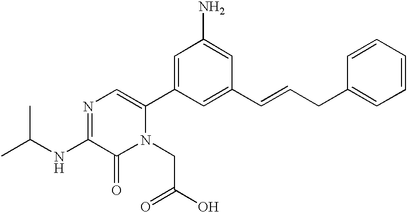

[6-{3-amino-5-[(1E)-3-phenylprop-1-enyl]phenyl}-3-(isopropylamino)-2-oxopyrazin-1 (2H)-yl]acetic Acid

Ex-76b) 76a (350 mg, 0.6 mmol), trifluoromethane sulfonic acid (0.24 mL, 2.7 mmol), CH2Cl2 (60 mL). Purified by reverse phase HPLC 25-70% acetonitrile/DI H2O 0.1% TFA over 10 min.

Product as tan solid (80 mg, 31%).

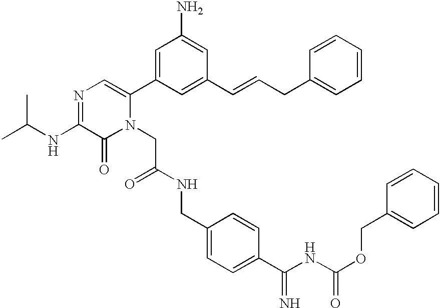

Benzyl {4-[({[6-{3-amino-5-[(1E)-3-phenylprop-1-enyl]phenyl}-3-(isopropylamino)-2-oxopyrazin-1 (2H)-yl]acetyl}amino)methyl]phenyl}(imino)methylcarbamate

EX-76c) 76b (80 mg, 0.2 mmol), benzyl [4-(aminomethyl)phenyl](imino)methylcarbamate dihydrochloride (85 mg, 0.2 mmol), HOBt (9 mg, 0.1 mmol), NMM (0.1 mL, 1.1 mmol), PS-carbodiimide resin (320 mg, 0.3 mmol), PS-diethylene triamine (135 mg, 0.4 mmol), Aldehyde wang (0 g, 0 mmol). Afforded brown semi-solid.

Ex-76) 76c (300 mg, 0.4 mmol), Pd/C (10%, 25 mg), methanol (50 mL). Reaction purified by reverse phase HPLC 15-50% acetonitrile/DI H2O 0.1% TFA to afford tan solid (64 mg, 29%). ¹H NMR (300 MHz, CD₃OD) δ 1.42 (d, 6H, J=6.3 Hz), 1.90-2.03 (m, 2H), 2.64-2.74 (m, 4H), 4.05-4.14 (m, 1H), 4.40 (s, 2H), 4.56 (s, 2H), 6.70 (s, 1H), 7.17-7.30 (m, 8H), 7.44-7.77 (abq, 4H, J=8.7 Hz).

HRMS m/z 552.3 (M⁺+H).

### EXAMPLE 77

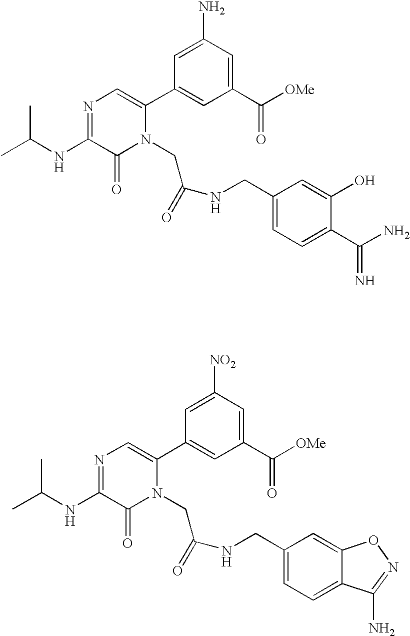

Ex-77a)

C₂₅H₂₅N₇O₇ (M.W. 535.51).

M+H=536

¹HNMR: 400 MHz, DMSO: (□) 1.186, 1.170 (d, 6H); 4.098 (septet, J=6.4 Hz, 1H); 4.324 (s, 2H); 4.449 (s, 2H); 6.320 (s, 1H); 6.790 (s, 1H); 7.018, 6.997 (d, 1H); 7.117 (s, 1H); 7.672, 7.652 (D, 1H); 8.302 (t, J=1.6 Hz, 1H); 8.507 (t, J=1.6 Hz, 1H); 8.584 to 8.507 (m, 1H).

Ex-77) 77a (535 mg; 0.001 mole) and Pd-black (50 mg) were dissolved/suspended in MeOH (10 ml). Ammonium formate (504 mg; 0.008 mole) was added and the mixture was stirred for 4 hr and the mixture filtered and concentrated. The residue was chromatographed using reverse phase, eluting with H₂O/MeCN. 180 mg of product. C₂₅H₂₉N₇O₅ (M.W. 507.54):*3.5 TFA * 2.0H₂O: F.W. 942.66. M+H=508

¹HNMR: 400 MHz, MeOD: (δ) 1.410, 1.389 (d, 6H); 3.869 (s, 3H); 4.060 (septet, J=6.4 Hz, 1H); 4.344 (s, 2H); 4.575 (s, 2H); 6.647 (s, 1H); 6.857, 6.851 & 6.829, 6.824 (d of d, 1H); 6.912, 6.908 (d, 1H); 7.095 (t, J=2.4 Hz, 1H); 7.470 (t, J=2.4 Hz, 1H); 7.64 to 7.571 (m, 2H).

Elemental analysis:

Found C: 40.46H: 3.76 N: 10.45

Calc. C: 40.77H: 3.90 N: 10.40

### EXAMPLE 78

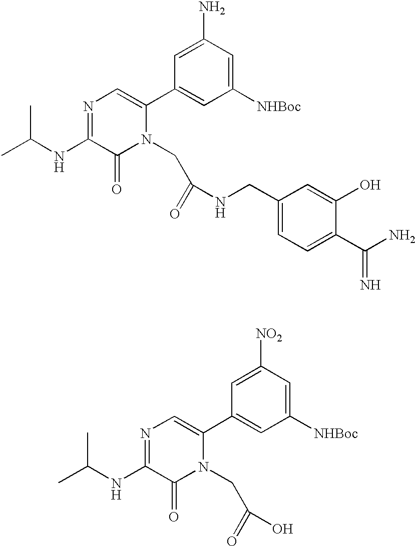

Ex-78a)

C₂₀H₂₅N₅O₈ (M.W. 447.44).

M+H=448

¹HNMR: 400 MHz, DMSO: (δ) 1.174, 1.158 (d of d, 6H); 1.461 (s, 9H); 4.079 (septet, J=6.4 Hz, 1H); 4.364 (s, 2H); 60725 (s, 1H); 7.050, 7.030 (d, 1H); 7.721 (s, 1H); 7.762 (t, J=2.4 Hz, 1H); 8.456 (s, 1H).

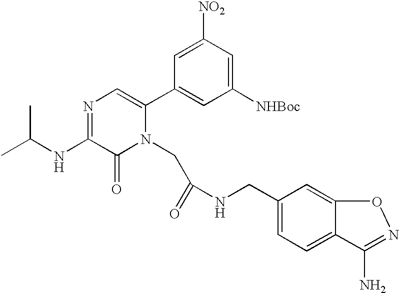

Ex-78b)

C₂₈H₃₂N₈O₅ (M.W. 592.6).

M+H=593

Ex-78) 78b (592 mg; 0.001 mole) and Pd-black (50 mg) were dissolved/suspended in MeOH (10 ml). Ammonium formate (504 mg; 0.008 mole) was added and the mixture was stirred for 4 hr and the mixture filtered and concentrated. The residue was chromatographed using reverse phase, eluting with H₂O/MeCN. 400 mg of product. C₂₈H₃₆N₈O₅ (M.W. 564.63):*3.5 TFA * 3.0H₂O: F.W. 1014.74. M+H=565

¹HNMR: 400 MHz, MeOD: (δ) 1.379, 1.363 (d, 6H); 1.491 (s, 9H); 4.047 (septet, J=6.4 Hz, 1H); 4.358 (s, 2H); 4.482 (s, 2H); 6.648 (s, 1H); 6.774 (t, J=1.6 Hz, 1H); 6.863, 6.860 & 6.842, 6.839 (d of d, 1H); 6.904 (s, 1H; 7.228 (s, 1H); 7.400 (s, 1H); 7.593, 7.573 (d of d, 1H).

Elemental analysis:

Found C: 41.23H: 4.12 N: 10.72

Calc. C: 41.30H: 4.51 N: 11.01

### EXAMPLE 79

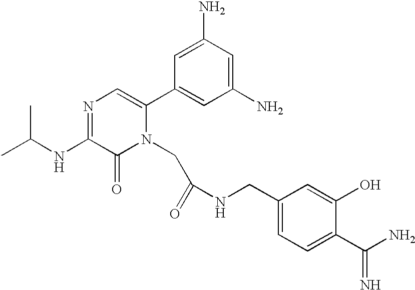

C₂₈H₃₆N₈O₅ (M.W. 464.52):*4.5 HCl * 2.75H₂O: F.W. 697.37.

M+H=465

¹HNMR: 400 MHz, MeOD: (6) 1.410, 1.394 (d, 6H); 4.082 (t, J=6.4 Hz, 1H); 4.356 (s, 2H); 4.603 (s, 2H); 6.701 (s, 1H); 6.928, 6.924 & 6.908, 6.904 (d of d, 1H); 6.961 (s, 1H); 7.173 to 7.163 (m, 1H);

Elemental analysis:

Found C: 41.01H: 5.45 N: 15.70

Calc. C: 40.65H: 5.84 N: 16.07

### EXAMPLE 80

N-{4-[amino(imino)methyl]benzyl}-2-[6-{3-amino-5-[(1E)-3-phenylprop-1-enyl]phenyl}-3-(isopropylamino)-2-oxopyrazin-1 (2H)-yl]acetamide

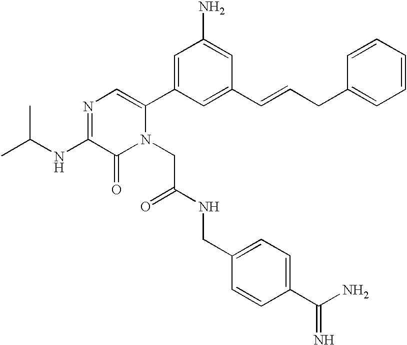

Ex-80) 76c (300 mg, 0.4 mmol) was dissolved in methanol (50 mL) and stirred with lithium hydroxide monohydrate (250 mg) at room temperature for 5 h. The solution was neutralized to pH 7 with 2 M HCl then purified by reverse phase HPLC 15-50% acetonitrile/DI H2O 0.1% TFA over 10 min. Afforded yellow solid product (39 mg, 18%).

### EXAMPLE 81

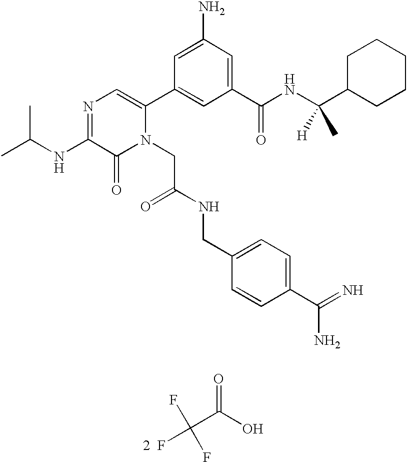

¹H NMR (300 MHz, CD₃OD) δ 0.92-1.07 (m, 2H), 1.17 (d, 3H, J=6.9 Hz), 1.42 (d, 6H, J=6.3 Hz), 1.69-1.83 (m, 5H), 3.83-3.94 (m, 1H), 3.99-4.13 (m, 1H), 4.48 (s, 2H), 4.66 (d, 2H, J=4.8 Hz), 6.67 (s, 1H), 6.95 (s, 1H), 7.14 (s, 1H), 7.31 (s, 1H), 7.38-7.78 (abq, 4H, J=8.4 Hz).

LRMS m/z 587.2 (M⁺+H).

### EXAMPLE 82

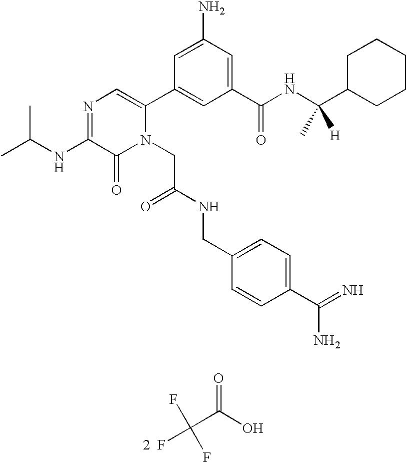

¹H NMR (300 MHz, CD₃OD) δ 0.97-1.08 (m, 3H), 1.17 (d, 3H, J=6.9 Hz), 1.17-1.22 (m, 2H), 1.42 (d, 6H, J=6.3 Hz), 1.69-1.83 (m, 5H), 3.84-3.96 (m, 1H), 4.02-4.14 (m, 1H), 4.47 (s, 2H), 4.66 (d, 2H, J=4.8 Hz), 6.68 (s, 1H), 6.98 (s, 1H), 7.17 (s, 1H), 7.34 (s, 1H), 7.38-7.78 (abq, 4H, J=8.1 Hz).

LRMS m/z 587.2 (M⁺+H).

### EXAMPLE 83

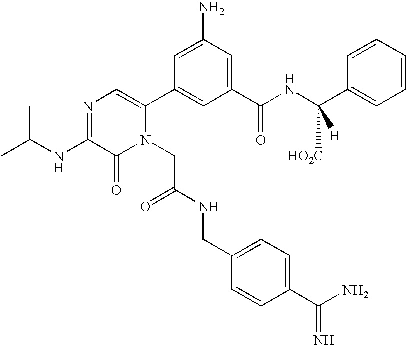

¹H NMR (300 MHz, CD₃OD) δ 0.96 (d, 3H, J=6.0 Hz), 0.99 (d, 3H, J=6.0 Hz), 1.42 (d, 6H, J=6.6 Hz), 1.72-1.81 (m, 2H), 4.02-4.13 (m, 1H), 4.47 (s, 2H), 4.61-4.67 (m, 1H), 4.68 (d, 2H, J=3.9 Hz), 6.69 (s, 1H), 7.22 (s, 1H), 7.37 (s, 1H), 7.39-7.78 (abq, 4H, J=8.4 Hz).

HRMS m/z 611.3 (M⁺+H).

### EXAMPLE 84

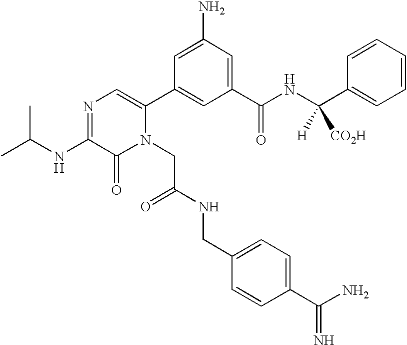

HRMS m/z 611.3 (M⁺+H).

### EXAMPLE 85

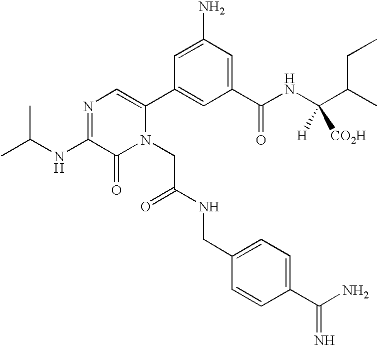

¹H NMR (300 MHz, CD₃OD) δ 0.96 (d, 3H, J=6.0 Hz), 0.99 (d, 3H, J=6.0 Hz), 1.42 (d, 6H, J=6.6 Hz), 1.72-1.81 (m, 2H), 4.02-4.13 (m, 1H), 4.47 (s, 2H), 4.61-4.67 (m, 1H), 4.68 (d, 2H, J=3.9 Hz), 6.69 (s, 1H), 7.22 (s, 1H), 7.37 (s, 1H), 7.39-7.78 (abq, 4H, J=8.4 Hz)

HRMS m/z 591.3 (M⁺+H).

### EXAMPLE 86

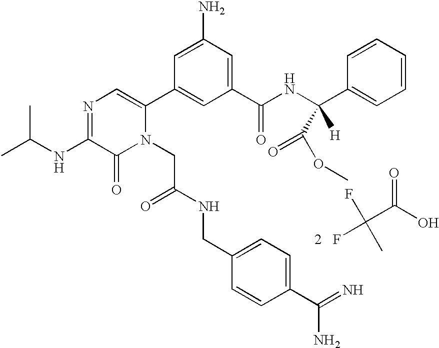

¹H NMR (300 MHz, CD₃OD) δ 1.41 (d, 6H, J=6.6 Hz), 3.75 (s, 3H), 4.00-4.11 (m, 1H), 4.44 (s, 2H), 4.66 (s, 2H), 5.68 (s, 1H), 6.67 (s, 1H), 6.92 (s, 2H), 7.15 (s, 2H), 7.31 (s, 2H), 7.36-7.47 (m, 5H), 7.45-7.75 (abq 4H, J=8.4 Hz).

LRMS m/z 625.2 (M⁺+H).

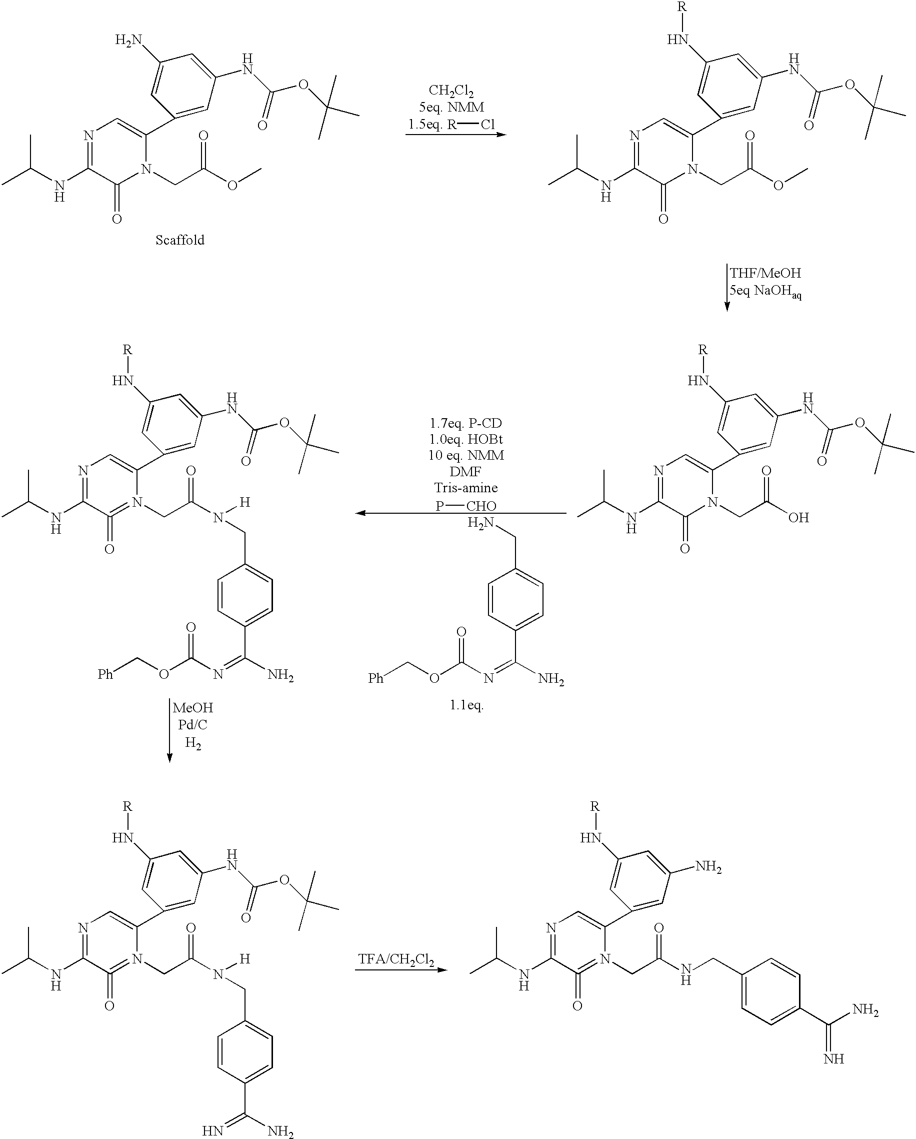

General Library Protocol

Shaken on an orbital shaker was 1 eq. of scaffold (0.20 g, 0.464 mmoles) with 1.5 eq. of 4-fluorobenzenesulfonyl chloride (0.135 g) and 5 eq. of N-Methyl Morpholine(0.25 mL) in dichloromethane (25 mL). After 6hours of shaking the reaction was concentrated under a nitrogen stream and dried under high vacuum. The residue was shaken with methanol (3 mL), tetrahydron furan (3 mL), and 2.5N sodium hydroxide (1 mL). Once hydrolysis was complete the reaction was blown dry under a nitrogen stream. The resulting basic residue was chromatographied on Gilson HPLC-RP to reduce salt load and dried under nitrogen stream. The resulting carboxylate residue was activated in N,N-dimethylformamide (25 mL) with N-methyl morpholine (10 eq., 1 mL), PS-Carbodiimide (1.7 eq) from Argonaut Technologies Inc., and 1-hydroxybenzotriazole (1.0 eq, 120 mg). After 15 minutes the benzamidine (1.1 eq., 360 mg) was added and shaken for 4 hours. Added excess polymer bound Tris-amine and aldehyde resins, and then shaken for an additional hour. The reaction was then filtered and the resins rinsed with dichloromethane. The filtrate was concentrated invacuo. The residue was taken up in methanol (50 mL) and purged with nitrogen. Excess palladium, 10 wt. % (dry basis), on activated carbon was added, tube capped with a septum, and a hydrogen balloon added. Once hydrogenolysis was complete the reaction was filtered through Celite, the filtrate concentrated invacuo, and dried under high vacuum. The residue was taken up in dichloromethane (25 mL) and trifluoroacetic acid (5 mL). Once the t-butyl ester was cleaved the reaction was concentrated invacuo. The residue was then chromatographed on Gilson HPLC-RP with 0.1%TFA(AN/H₂O) to yield the desired product as the TFA salts.

### EXAMPLE 87

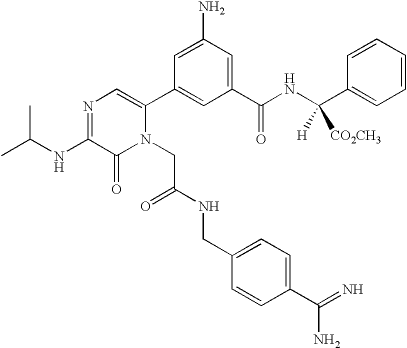

The compound of Example 87 was prepared in an analogous manner to that of Example 186.

### EXAMPLE 88

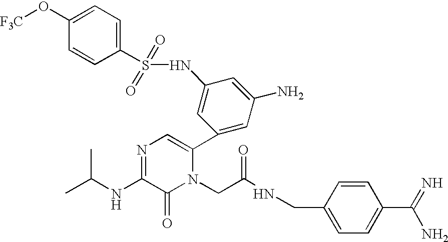

Using 4-(trifluoromethoxy)benzenesulfonyl chloride and following the general library protocol listed above the desired product was obtained in 11% yield as an off-white solid. HPLC/LRMS: >97%, 673 (M+H)+; HRMS(ES+) calcd. for C₃₀H₃₂N₈O₅SF₃ 673.2163, found 673.2194.

### EXAMPLE 89

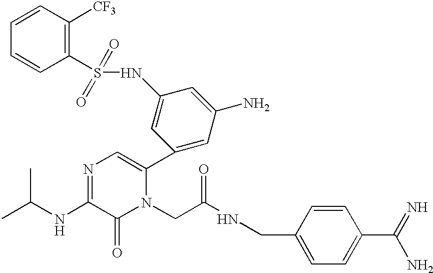

Using 2-(trifluoromethyl)benzenesulfonyl chloride and following the general library protocol listed above the desired product was obtained in 14% yield as an off-white solid. HPLC/LRMS: >95%, 657 (M+H)+; HRMS(ES+) calcd. for C₃₀H₃₂N₈O₄SF₃ 657.2214, found 657.2231.

### EXAMPLE 90

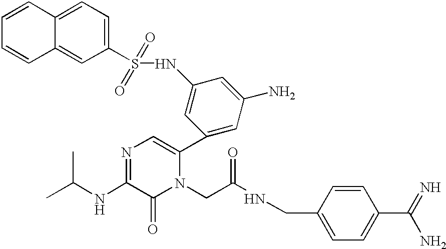

Using 2-naphthalenesulfonyl chloride and following the general library protocol listed above the desired product was obtained in 8.3% yield as an off-white solid. HPLC/LRMS: >98%, 639 (M+H)+; HRMS(ES+) calcd. for C₃₃H₃₅N₈O₄S 639.2497, found 639.2530.

### EXAMPLE 91

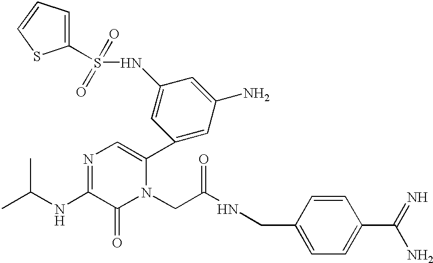

Using 2-thiophenesulfonyl chloride and following the general library protocol listed above the desired product was obtained in 2.6% yield as an off-white solid. HPLC/LRMS: >95%, 595 (M+H)+; HRMS(ES+) calcd. for C₂₇H₃₁N₈O₄S₂ 595.1904, found 595.1894.

### EXAMPLE 92

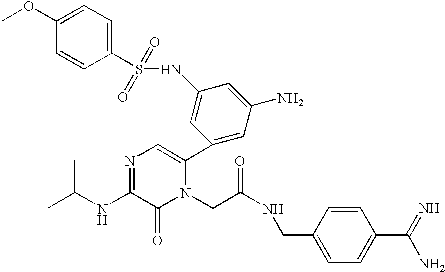

Using 4-methoxybenzenesulfonyl chloride and following the general library protocol listed above the desired product was obtained in 31% yield as an off-white solid. HPLC/LRMS: >98%, 619 (M+H)+; HRMS(ES+) calcd. for C₃₀H₃₅N₈O₈S 619.2446, found 619.2479.

### EXAMPLE 93

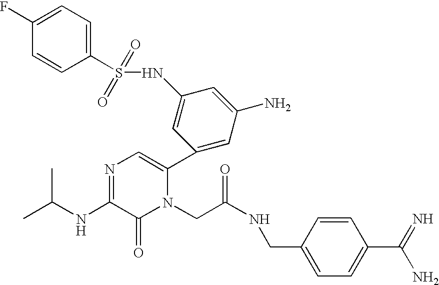

Using 4-fluorobenzenesulfonyl chloride and following the general library protocol listed above the desired product was obtained in 28% yield as an off-white solid. HPLC/LRMS: >97, 607 (M+H)+; ¹⁹F NMR(282 MHz, DMF-d₇) δ −107.57; ¹H NMR(300 MHz, DMF-d₇) δ 10.18 (3H, br m), 9.43 (2H, br s), 8.88 (1H, br m), 8.00-7.94 (6H, m), 7.54 (2H, m), 7.43 (2H, m), 6.76 (2H, m), 6.62 (1H, s), 6.49 (1H, s), 4.57-4.53 (4H, m), 4.22 (1H, m), 1.34 (6H, d);

HRMS(ES+) calcd. for C₂₉H₃₂N₈O₄SF 607.2246, 607.2282 found

### EXAMPLE 94

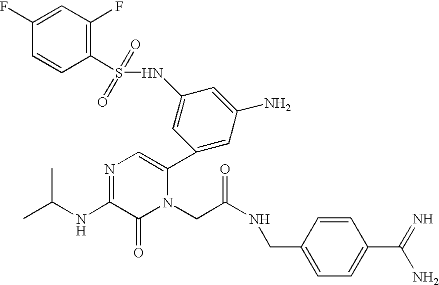

Using 2,4-difluorobenzenesulfonyl chloride and following the general library protocol listed above the desired product was obtained in 7.8% yield as an off-white solid. HPLC/LRMS: >95%, 625 (M+H)+; HRMS(ES+) calcd. for C₂₉H₃₁N₈O₄SF₂ 625.2152, found 625.2169.

### EXAMPLE 95

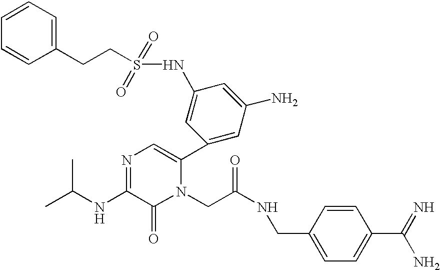

Using trans-δ-styrenesulfonyl chloride and following the general library protocol listed above the desired product was obtained in 11% yield as an off-white solid. HPLC/LRMS: >98%, 617 (M+H)+; HRMS(ES+) calcd. for C₃₁H₃₇N₈O₄S 617.2653, found 617.2675.

### EXAMPLE 96

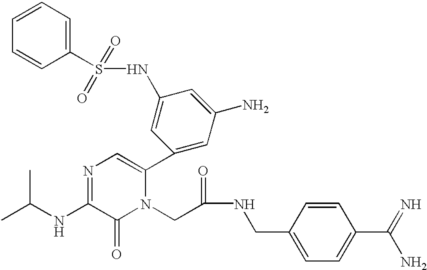

Using benzenesulfonyl chloride and following the general library protocol listed above the desired product was obtained in 28% yield as an off-white solid. HPLC/LRMS: >95%, 589 (M+H)+; HRMS(ES+) calcd. for C₂₉H₃₃N₈O₄S 589.2340, found 589.2325.

### EXAMPLE 97

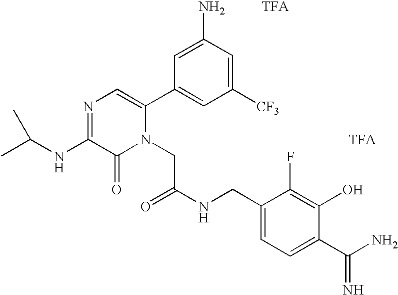

LCMS (RP, 15-90% acetonitrile in 0.1% ammonium acetate over 14 min): retention time: 5.90 min; (M+H)⁺=536, Negative Ion mode (M−H)⁻=534.

Reductive Amination Library

### EXAMPLE 98

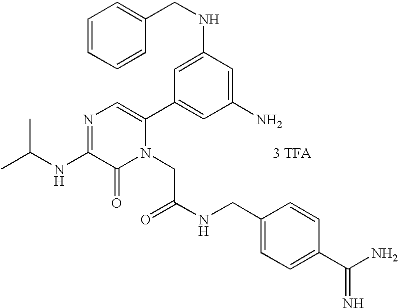

By following the method of Example 110 and substituting benzaldehyde for phenylacetaldehyde, Example 98 was prepared: ¹H NMR (400 MHz, DMF-d₇) δ 10.24 (s, 2H), 9.72 (br s, 5H), 9.41 (s, 2H), 8.80-8.79 (m, 1H), 7.92 (d, J=8.5 Hz, 2H), 7.50 (d, J=8.3 Hz, 2H), 7.40-7.38 (m, 2H), 7.34-7.30 (m, 2H), 7.26-7.23 (m, 1H), 6.78 (s, 1H), 6.65 (s, 1H), 6.50-6.47 (m, 2H), 4.62 (s, 2H), 4.50 (d, J=5.8 Hz, 2H), 4.32 (s, 2H), 4.22-4.20 (m, 1H), 1.31 (d, J=6.4 Hz, 6H); C NMR (100 MHz, DMF-d₇) δ 167.38, 167.31, 152.5, 150.3, 148.4, 146.4, 140.3, 134.2, 130.3, 128.87, 128.70, 128.07, 128.01, 127.67, 127.39, 109.2, 104.4, 49.01, 47.5, 44.1, 42.8, 21.8; HRMS (EI) calcd for C₃₀H₃₅N₈O₂ 539.2877, found 539.2870.

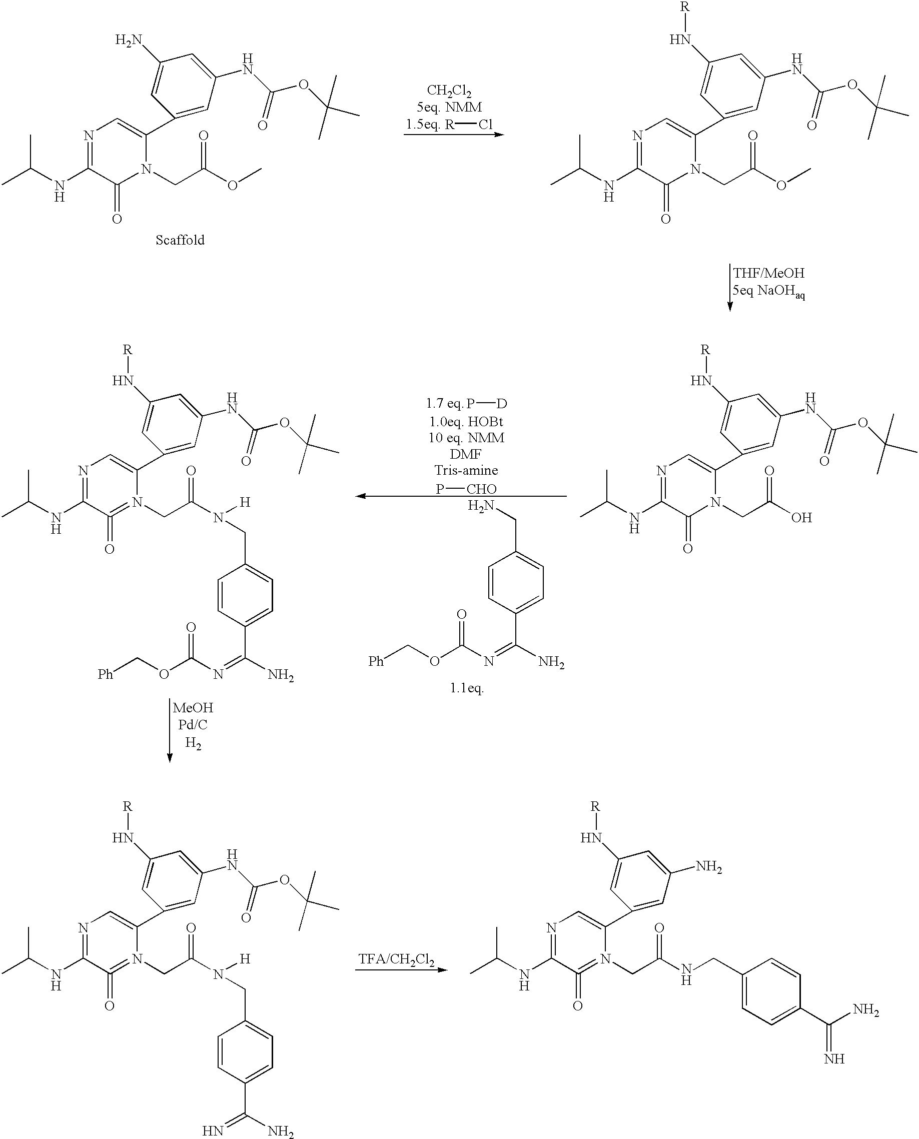

General Library Protocol

Shaken on an orbital shaker was 1 eq. of scaffold (0.20 g, 0.464 mmoles) with 1.5 eq. of 4-fluorobenzenesulfonyl chloride (0.135 g) and 5 eq. of N-Methyl Morpholine(0.25 mL) in dichloromethane (25 mL). After 6hours of shaking the reaction was concentrated under a nitrogen stream and dried under high vacuum. The residue was shaken with methanol (3 mL), tetrahydron furan (3 mL), and 2.5N sodium hydroxide (1 mL). Once hydrolysis was complete the reaction was blown dry under a nitrogen stream. The resulting basic residue was chromatographied on Gilson HPLC-RP to reduce salt load and dried under nitrogen stream. The resulting carboxylate residue was activated in N,N-dimethylformamide (25 mL) with N-methyl morpholine (10 eq., 1 mL), PS-Carbodiimide (1.7 eq) from Argonaut Technologies Inc., and 1-hydroxybenzotriazole (1.0 eq, 120 mg). After 15 minutes the benzamidine (1.1 eq., 360 mg) was added and shaken for 4 hours. Added excess polymer bound Tris-amine and aldehyde resins, and then shaken for an additional hour. The reaction was then filtered and the resins rinsed with dichloromethane. The filtrate was concentrated invacuo. The residue was taken up in methanol (50 mL) and purged with nitrogen. Excess palladium, 10 wt. % (dry basis), on activated carbon was added, tube capped with a septum, and a hydrogen balloon added. Once hydrogenolysis was complete the reaction was filtered through Celite, the filtrate concentrated invacuo, and dried under high vacuum. The residue was taken up in dichloromethane (25 mL) and trifluoroacetic acid (5 mL). Once the t-butyl ester was cleaved the reaction was concentrated invacuo. The residue was then chromatographed on Gilson HPLC-RP with 0.1%TFA(AN/H₂O) to yield the desired product as the TFA salts.

### EXAMPLE 99

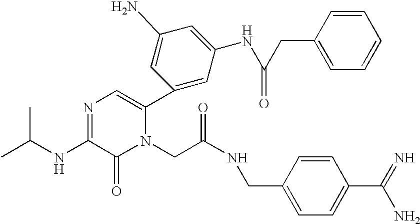

Using phenylacetyl chloride and following the reverse amide general library protocol the the desired product was obtain. HPLC/LRMS: >98%, 567 (M+H)+;

HRMS(ES+) calcd. for C₃₁H₃₅N₈O₃ 567.2827, found 567.2849.

### EXAMPLE 100

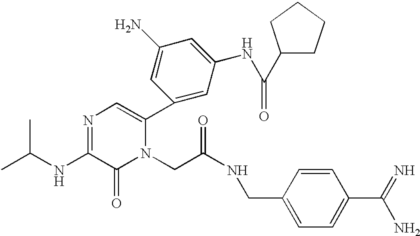

Using cyclopentanecarbonyl chloride and following the reverse amide general library protocol the the desired product was obtained. HPLC/LRMS: >98%, 545 (M+H)+; HRMS(ES+) calcd. for C₂₉H₃₇N₈O₃ 545.2983, found 545.2995.

### EXAMPLE 101

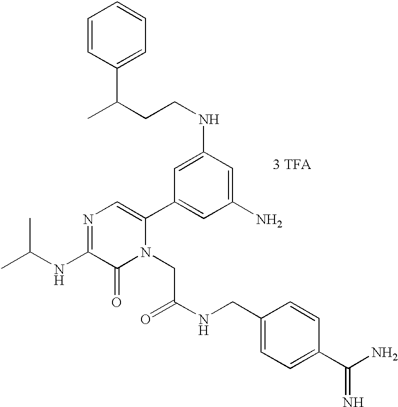

By following the method of Example 110 and substituting 3-phenylbutyraldehyde for phenylacetaldehyde, Example 101 was prepared: ¹H NMR (400 MHz, DMF-d₇) δ 10.24 (br s, 7H), 9.43 (br s, 2H), 8.80-8.78 (m, 1H), 8.10 (br s, 1H), 7.93 (d, J=8.3 Hz, 2H), 7.48 (d, J=8.2 Hz, 2H), 7.32-7.25 (m, 4H), 7.20-7.16 (m, 1H), 6.82 (s, 1H), 6.68 (s, 1H), 6.55 (s, 1H), 6.48 (s, 1H), 4.63 (s, 2H), 4.48 (d, J=5.9 Hz, 2H), 4.24-4.23 (m, 1H), 3.07-2.85 (m, 3H), 1.94-1.88 (m, 2H), 1.33 (d, J=6.5 Hz, 6H), 1.24 (d, J=6.9 Hz, 3H); ¹³C NMR (100 MHz, DMF-d₇) δ 167.36, 167.26, 152.6, 149.7, 148.3, 147.5, 146.3, 134.3, 130.1, 128.98, 128.71, 128.06, 127.65, 127.47, 126.6, 118.5, 115.71, 115.60, 110.0, 105.4, 49.0, 44.2, 42.95, 42.79, 37.82, 37.25, 22.4, 21.7; HRMS (EI) calcd for C₃₃H₄₁N₈O₂ 581.3347, found 581.3370.

### EXAMPLE 102

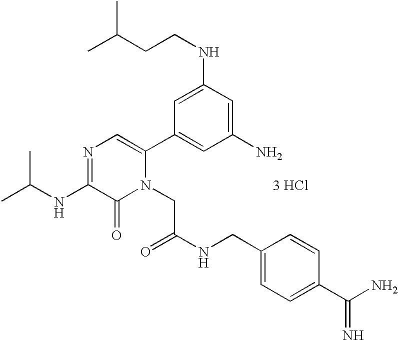

By following the method of Example 110 and substituting isovaleraldehyde for phenylacetaldehyde, Example 102 was prepared: ¹H NMR (400 MHz, DMF-d₇) δ 9.74 (br s, 7H), 9.09-9.06 (m, 1H), 8.03 (d, J=8.6 Hz, 2H), 7.35 (d, J=8.2 Hz, 2H), 7.27 (s, 1H), 6.94-6.92 (m, 2H), 6.68 (s, 1H), 4.76 (s, 2H), 4.64-4.59 (m, 1H), 4.44 (d, J=5.2 Hz, 2H), 3.14-3.11 (m, 2H), 1.71-1.56 (m, 3H), 1.40 (d, J=6.3 Hz, 6H), 0.85 (d, J=6.3 Hz, 6H); C NMR (100 MHz, DMF-d₇) δ 166.89, 166.54, 153.2, 146.21, 146.03, 133.1, 129.7, 128.9, 127.9, 126.8, 109.1, 46.6, 42.7, 36.7, 35.6, 26.2, 22.5, 21.4; HRMS (EI) calcd for C₂₈H₃₉N₈O₂ 519.3190, found 519.3191.

### EXAMPLE 103

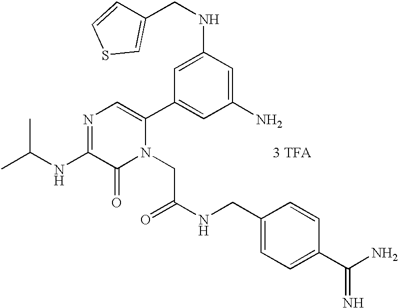

By following the method of Example 110 and substituting 3-thiophenecarboxaldhyde for phenylacetaldehyde, Example 103 was prepared: ¹H NMR (400 MHz, DMF-d₇) δ 10.63 (br s, 5H), 10.18 (s, 2H), 9.41 (s, 2H), 8.83-8.80 (m, 1H), 8.25 (br s, 1H), 7.92 (d, J=8.3 Hz, 2H), 7.51-7.49 (m, 3H), 7.41-7.40 (m, 1H), 7.14 (dd, J=1.2, 5.0 Hz, 1H), 6.82 (s, 1H), 6.77-6.76 (m, 1H), 6.60 (s, 1H), 6.56 (s, 1H), 4.63 (s, 2H), 4.51 (d, J=5.8 Hz, 2H), 4.33 (s, 2H), 4.24-4.23 (m, 1H), 1.33 (d, J=6.5 Hz, 6H); ¹³C NMR (100 MHz, DMF-d₇) δ 167.34, 167.25, 152.6, 150.2, 148.1, 146.3, 140.9, 134.1, 130.2, 128.71, 128.13, 128.08, 127.7, 126.6, 122.5, 110.14, 109.9, 49.1, 44.4, 43.1, 42.8, 21.7; HRMS (EI) calcd for C₂₈H₃₃N₈O₂S 545.2442, found 545.2444.

### EXAMPLE 104

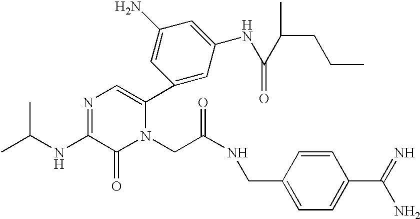

Using 2-methylvaleryl chloride and following the reverse amide general library protocol the the desired product was obtained. HPLC/LRMS: >98%, 547 (M+H)+;

HRMS(ES+) calcd. for C₂₉H₃₉N₈O₃ 547.3140, found 547.3124.

### EXAMPLE 105

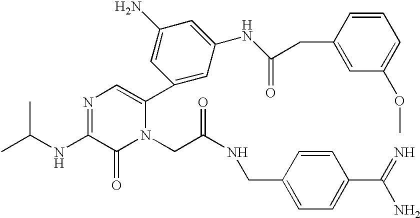

Using 3-methoxyphenylacetyl chloride and following the reverse amide general library protocol the the desired product was obtained. HPLC/LRMS: >98%, 597 (M+H)+; HRMS(ES+) calcd. for C₃₂H₃₇N₈O₄ 597.2932, found 597.2942.

### EXAMPLE 106

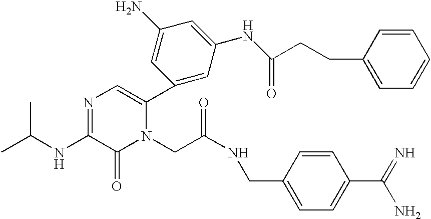

Using hydrocinnamoyl chloride and following the reverse amide general library protocol the the desired product was obtained. HPLC/LRMS: >98%, 581 (M+H)+;

HRMS(ES+) calcd. for C₃₂H₃₇N₈O₃ 581.2983, found 581.2990.

### EXAMPLE 107

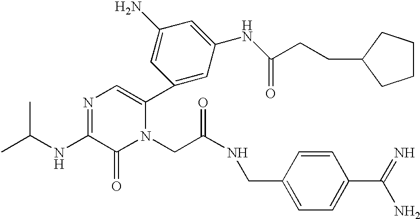

Using 3-cyclopentylpropionyl chloride and following the reverse amide general library protocol the desired product was obtained. HPLC/LRMS: >98%, 573 (M+H)+;

HRMS(ES+) calcd. for C₃₁H₄₁N₈O₃ 573.3296, found 573.3322.

### EXAMPLE 108

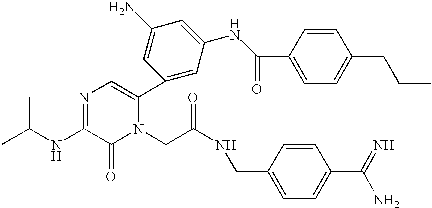

Using 4-propylbenzoyl chloride and following the reverse amide general library protocol the desired product was obtained. HPLC/LRMS: >98%, 595 (M+H)+; ¹H NMR (DMF-d₇, 300 MHz) δ 10.20 (3H, br m), 9.35 (2H, br s), 8.78 (1H, br m), 8.21 (1H, br m), 7.99 (2H, m), 7.91 (2H, m), 7.61 (1H, s), 7.49 (2H, m), 7.36 (3H, m), 6.89 (1H, s), 6.68 (1H, s), 4.73 (2H, m), 4.53 (2H, m), 4.28 (1H, m), 2.67 (2H, m), 1.67 (2H, m), 1.37 (6H, m), 0.94 (3H, m);

HRMS(ES+) calcd. for C₃₃H₃₉N₈O₃ 959.3140, found 595.3147.

### EXAMPLE 109

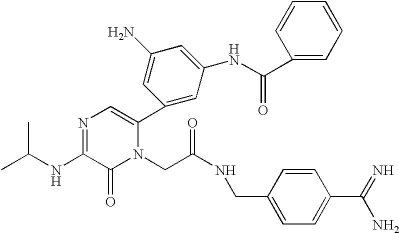

Using benzoyl chloride and following the general library protocol listed above the reverse amide general library protocol the desired product was obtained.

HPLC/LRMS: >97%, 553 (M+H)+; HRMS(ES+) calcd. for C₃₀H₃₃N₈O₃ 553.2676, found 553.2641.

### EXAMPLE 110

N-{4-[amino(imino)methyl]benzyl}-2-[6-{3-amino-5-[(2-phenylethyl)amino]phenyl}-3-(isopropylamino)-2-oxopyrazin-1 (2H)-yl]acetamide trifluoroacetate

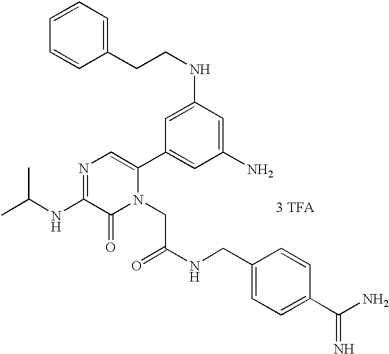

Ex-110a) 6-bromo-1-methoxycarbonylmethyl-3-(N-isopropylamino)pyrazinone was prepared: ¹H NMR (300 MHz, CDCl₃) δ 7.04 (s, 1H), 5.96 (d, J=7.5 Hz, 1H), 4.94 (s, 2H), 4.16-4.00 (m, 1H), 3.80 (s, 3H), 1.25 (d, J=6.5 Hz, 6H); C NMR (75 MHz, CDCl₃) δ 167.1, 152.3, 148.9, 124.4, 104.2, 52.8, 48.7, 42.6, 22.2; HRMS (EI) calcd for C₁₀H₁₅BrN₃O₃ 304.0297, found 304.0340.

Ex-110b) A mixture of 6-bromo-1-methoxycarbonylmethyl-3-(N-isopropylamino)pyrazinone (9.31 g, 30.61 mmol) and 3-[(tert-butoxylcarbonyl)amino]-5-nitrophenylboronic acid (10.41 g, 36.91 mmol) in 200 mL THF (0.15 M) was allowed to stir for 5 minutes at room temperature with argon flushing. The solution was then added 37 mL of 2.0 M solution of sodium carbonate (73.87 mmol) followed by tertakis(triphenylphosphine)palladium (0) (1.8111 g, 5 mol %). The resulting mixture was then heated to reflux and after approximately 4 hours, tertakis(triphenylphosphine)palladium (0) (1.8111 g, 5 mol %) was added in a second portion. The solution was allowed to reflux overnight (ca 18 hours). The reaction mixture was allowed to cool to room temperature and was diluted with ethylacetate (1 L). The organic solution was washed with saturated sodium bicarbonate (1×300 mL), brine (1×300 mL), dried (MgSO₄), filtered and concentrated. Purification of the crude product by MPLC (16.7% ethyl ether to 50% ethyl ether/25% ethyl acetate/hexanes) afforded pure methyl [6-{3-[(tert-butoxycarbonyl)amino]-5-nitrophenyl}-3-(isopropylamino)-2-oxopyrazin-1 (2H)-yl]acetate in 86% yield: ¹H NMR (400 MHz, CDCl₃) δ 8.32-8.31 (m, 1H), 7.84-7.83 (m, 1H), 7.78 (s, 1H), 7.19 (s, 1H), 6.85 (s, 1H), 6.13 (d, J=7.8 Hz, 1H), 4.51 (s, 2H), 4.23-4.13 (m, 1H), 3.78 (s, 3H), 1.53 (s, 9H), 1.29 (d, J=6.5 Hz, 6H); C NMR (100 MHz, CDCl₃) δ 167.8, 152.1, 151.7, 149.7, 148.8, 140.3, 134.4, 125.5, 124.6, 123.5, 118.1, 113.3, 81.9, 52.8, 47.2, 42.6, 28.1, 22.3; HRMS (EI) calcd for C₂₁H₂₈N₅O₇ 462.1989, found 462.1984.

Ex-110c) A solution of pure methyl [6-{3-[(tert-butoxycarbonyl)amino]-5-nitrophenyl}-3-(isopropylamino)-2-oxopyrazin-1 (2H)-yl]acetate (10.6022 g, 22.97 mmol) in 230.0 mL ethyl acetate and ethanol (1:1, 0.1 M) was added 0.7825 10% Pd-C (wet) in one portion. The resulting suspension was allowed to stir under an atmosphere of hydrogen gas (balloon) over night (ca. 18 hours). Filtration through a pad of Celite 545 followed by concentration afforded pure methyl [6-{3-amino-5-[(tert-butoxycarbonyl)amino]phenyl}-3-(isopropylamino)-2-oxopyrazin-1 (2H)-yl]acetate(EX-1ra3) in 96% yield as an off-white solid: ¹H NMR (400 MHz, CDCl₃) δ 6.95 (s, 1H), 6.80 (s, 1H), 6.77 (s, 1H), 6.56 (s, 1H), 6.28 (s, 1H), 5.99 (d, J=7.8 Hz, 1H), 4.52 (s, 2H), 4.19-4.10 (m, 1H), 3.85 (s, 3H), 3.85 (br s, 2H), 3.74 (s, 3H), 1.50 (s, 9H), 1.26 (d, J=6.4 Hz, 6H); C NMR (100 MHz, CDCl₃) δ 168.1, 152.5, 151.8, 149.2, 147.6, 139.8, 133.7, 127.8, 122.1, 110.6, 109.6, 105.1, 80.5, 52.5, 47.1, 42.3, 28.2, 22.4; HRMS (EI) calcd for C₂₁H₃₀N₅O₅ 432.2247, found 432.2237.

Ex-110d) A solution of methyl [6-{3-amino-5-[(tert-butoxycarbonyl)amino]phenyl}-3-(isopropylamino)-2-oxopyrazin-1 (2H)-yl]acetate (1.1721 g, 2.716 mmol) and phenylacetaldehyde (0.650 mL, 5.556 mmol) and 1.0 mL acetic acid in 30.0 mL THF (0.1 M) was added sodium triacetoxyborohydride (1.7433 g, 8.225 mmol) in one portion at room temperature. The resulting suspension was allowed to stir overnight. The reaction mixture was quenched with saturated sodium bicarbonate (50 mL). The aqueous solution was extracted with ethyl acetate (3×25 mL). The combined organic solutions were washed with with saturated sodium bicarbonate (1×25 mL), brine (2×25 mL), dried (MgSO₄), filtered and concentrated. Purification by MPLC (20% ethyl acetate to 40% ethyl acetate/hexanes) afforded pure methyl [6-{3-[(tert-butoxycarbonyl)amino]-5-[(2-phenylethyl)amino]phenyl}-3-(isopropylamino)-2-oxopyrazin-1 (2H)-yl]acetate in 92% yield: ¹H NMR (400 MHz, CDCl₃) δ 7.32-7.27 (m, 2H), 7.24-7.19 (m, 3H), 6.88 (br s, 1H), 6.82 (s, 1H), 6.67 (s, 1H), 6.51-6.50 (m, 1H), 6.21-6.20 (m, 1H), 5.99 (d, J=7.9 Hz, 1H), 4.51 (s, 2H), 4.19-4.10 (m, 1H), 3.88 (br s, 1H), 3.70 (s, 3H), 3.37-3.33 (m, 2H), 2.90-2.86 (m, 2H), 1.49 (s, 9H), 1.26 (d, J=6.4 Hz, 6H); C NMR (100 MHz, CDCl₃) δ 168.1, 152.5, 151.9, 149.2, 148.9, 139.8, 138.3, 133.7, 128.66, 128.56, 126.4, 108.65, 108.31, 103.1, 80.5, 52.4, 47.2, 44.7, 42.4, 35.3, 28.2, 22.4; HRMS (EI) calcd for C₂₉H₃₈N₅O₅ 536.2867, found 536.2859.

Ex-110-e) A solution of methyl [6-{3-[(tert-butoxycarbonyl)amino]-5-[(2-phenylethyl)amino]phenyl}-3-(isopropylamino)-2-oxopyrazin-1 (2H)-yl]acetate (1.1183 g, 2.087 mmol) in 25.0 mL THF/methanol (3:1, 0.1 M) was added 3.00 mL of 2.5 M sodium hydroxide (7.50 mmol) at room temperature. After stirring for approximately 3 hours, the solvent was removed under reduced pressure. The resulting residue was diluted with brine (25.0 mL) and cooled in an ice bath (ca. 0° C.). The solution was then acidified (pH approximately 4 as determined by indicating litmus paper). The aqueous solution was extracted with ethyl acetate (3×25 mL). The combined organic solutions were washed with brine (2×25 mL), dried (MgSO₄), filtered and concentrated to give pure [6-{3-[(tert-butoxycarbonyl)amino]-5-[(2-phenylethyl)amino]phenyl}-3-(isopropylamino)-2-oxopyrazin-1 (2H)-yl]acetic acid as a yellow solid in 90% yield: ¹H NMR (400 MHz, DMF-d₇) δ 9.25 (s, 1H), 7.34-7.28 (m, 4H), 7.24-7.19 (m, 1H), 7.12 (s, 1H), 7.02 (br s, 1H), 6.85 (s, 1H), 6.75 (s, 1H), 6.37-6.36 (m, 1H), 4.58 (s, 2H), 4.28-4.19 (m, 1H), 3.34-3.30 (m, 2H), 2.93-2.89 (m, 2H), 1.46 (s, 9H), 1.28 (d, J=6.5 Hz, 6H); ¹³C NMR (100 MHz, DMF-d₇) δ 169.5, 153.7, 152.2, 150.2, 149.4, 141.8, 140.7, 134.1, 130.1, 129.4, 128.92, 128.59, 126.6, 108.21, 107.95, 103.1, 79.5, 47.6, 45.8, 43.1, 28.3, 22.2; HRMS (EI) calcd for C₂₈H₃₆N₅O₅ 522.2711, found 522.2754.

Ex-110f) A solution of [6-{3-[(tert-butoxycarbonyl)amino]-5-[(2-phenylethyl)amino]phenyl}-3-(isopropylamino)-2-oxopyrazin-1 (2H)-yl]acetic acid (0.8875 g, 1.701 mmol) in 17.0 mL dry dichloromethane/DMF (3:1, 0.1 M) was added 1-hydroxybenzotriazole (0.3061 g, 2.265 mmol), N-methylmorpholine (2.00 mL, 18.19 mmol), and carbodiimde resin (2.46 g, 2.583 mmol). The resulting suspension was allowed to shake for 10 minutes and was then added 4-(N-benzyloxycarbonylamidino)benzylamine hydrogen chloride salt (0.6574 g, 2.0557 mmol) in one portion. The resulting suspension was allowed to shake for 3 hours. The reaction mixture was then added aldehyde resin (2.0 equivalents) and the reaction was shook an additional one hour. The reaction was filtered and rinsed with dichloromethane (3×10 mL) and DMF (1×10 mL). The solvent was removed under reduced pressure. Purification by MPLC (50% ethyl acetate to 80% ethyl acetate/hexanes) afforded pure benzyl {4-[({[6-{3-[(tert-butoxycarbonyl)amino]-5-[(2-phenylethyl)amino]phenyl}-3-(isopropylamino)-2-oxopyrazin-1 (2H)-yl]acetyl}amino)methyl]phenyl}(imino)methylcarbamate in 75% yield: ¹H NMR (400 MHz, DMF-d₇) δ 9.43 (s, 1H), 8.78-8.75 (m, 1H), 8.26-8.15 (m, 3H), 7.71-7.46 (m, 10H), 7.42-7.38 (m, 1H), 7.32 (s, 1H), 7.06 (s, 1H), 6.94 (s, 1H), 6.78 (d, J=8.1 Hz, 1H), 6.63 (s, 1H), 6.07 (br s, 0.5H), 5.39 (s, 2H), 4.80 (s, 2H), 4.62 (d, J=5.6 Hz, 2H), 4.41-4.29 (m, 1H), 3.52-3.47 (m, 2H), 3.13-3.08 (m, 2H), 1.66 (S, 9H), 1.45 (d, J=6.4 Hz, 6H); C NMR (100 MHz, DMF-d₇) δ 167.95, 167.61, 164.9, 162.9, 153.7, 152.3, 150.16, 150.05, 144.4, 141.6, 140.7, 138.2, 134.6, 133.7, 130.4, 129.47, 129.00, 128.93, 128.59, 128.47, 128.39, 127.74, 127.63, 126.7, 124.9, 121.6, 119.7, 110.2, 108.38, 108.05, 107.1, 103.0, 79.5, 66.9, 48.9, 45.9, 42.97, 42.61, 35.8, 28.4, 22.4; HRMS (EI) calcd for C₄₄H₅₁N₈O₆ 787.3926, found 787.3921. Ex-110) A solution of benzyl {4-[({[6-{3-[(tert-butoxycarbonyl)amino]-5-[(2-phenylethyl)amino]phenyl}-3-(isopropylamino)-2-oxopyrazin-1 (2H)-yl]acetyl}amino)methyl]phenyl)(imino)methylcarbamate (0.6532 g, 0.8300 mmol) in 8.0 mL methanol (0.1 M) was added 10% Pd-C (wet) (0.1121 g) in one portion. The resulting suspension was purged with hydrogen gas, and then the reaction mixture was allowed to stir under an atmosphere of hydrogen (balloon). After approximately 3 hours, the reation was filtered through a pad of Celite 545 and the solvent was removed under reduced pressure. The residue was added dry chloroform (4.0 mL, 0.2 M) followed bytrifluoroacetic acid (1.60 mL, 20.77 mmol). After approximately 2 hours, the solvent was removed under reduced pressure. Purification by reverse phase HPLC (5% acetonitrile to 50% acetonitrile/water/0.1%trifluoroacetic acid) afforded pure N-{4-[amino(imino)methyl]benzyl}-2-[6-{3-amino-5-[(2-phenylethyl)amino]phenyl}-3-(isopropylamino)-2-oxopyrazin-1 (2H)-yl]acetamide trifluoroacetate in 40% yield: ¹H NMR (400 MHz, DMF-d₇) δ 10.25 (br s, 7H), 9.42 (s, 2H), 8.81-8.78 (m, 1H), 7.92 (d, J=8.3 Hz, 3H), 7.47 (d, J=8.3 Hz, 3H), 7.31-7.28 (m, 4H), 7.24-7.22 (m, 1H), 6.83 (s, 1H), 6.68 (s, 1H), 6.52-6.50 (m, 2H), 4.65 (s, 2H), 4.47 (d, J=5.9 Hz, 2H), 4.25-4.20 (m, 1H), 3.35-3.11 (m, 2H), 2.92-2.89 (m, 2H), 1.32 (d, J=6.5 Hz, 6H); C NMR (100 MHz, DMF-d₇) δ 167.41, 167.40, 152.6, 150.1, 148.5, 146.3, 140.4, 134.4, 130.3, 129.4, 128.92, 128.73, 128.10, 127.7, 118.5, 115.6, 126.7, 109.39, 109.17, 104.7, 49.1, 46.0, 44.1, 52.8, 35.4, 21.8; HRMS (EI) calcd for C₃₁H₃₇N₈O₂ 553.3034, found 553.3064.

### EXAMPLE 111

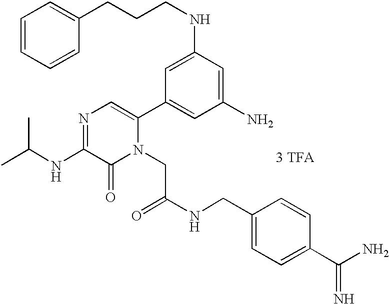

By following the method of Example 110 and substituting hydrocinnamaldehyde for phenylacetaldehyde, Example 111 was prepared: ¹H NMR (400 MHz, DMF-d₇) δ 10.72 (br s, 5H), 10.25 (br s, 2H), 9.43 (s, 2H), 8.82-8.80 (m, 1H), 8.12 (br s, 1H), 7.93 (d, J=8.3 Hz, 2H), 7.48 (d, J=8.2 Hz, 2H), 7.30-7.16 (m, 5H), 6.85 (s, 1H), 6.74 (s, 1H), 6.57-6.53 (m, 2H), 4.65 (s, 2H), 4.49 (d, J=5.8 Hz, 2H), 4.25-4.23 (m, 1H), 3.15-3.12 (m, 2H), 2.73-2.70 (m, 2H), 1.97-1.89 (m, 2H), 1.33 (d, J=6.4 Hz, 6H); ¹³C NMR (100 MHz, DMF-d₇) δ 167.34, 167.27, 152.6, 149.8, 148.3, 146.3, 142.5, 134.3, 130.1, 128.89, 128.86, 128.68, 128.05, 127.6, 126.3, 115.7, 110.0, 105.5, 49.0, 44.23, 44.08, 42.8, 33.4, 30.8, 21.7; HRMS (EI) calcd for C₃₂H₃₉N₈O₂ 567.3190, found 567.3212.

### EXAMPLE 112

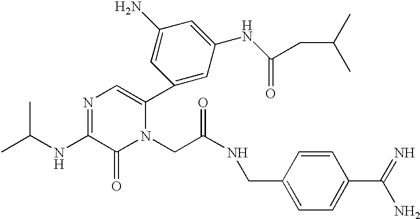

Using isovaleryl chloride and following the reverse amide general library protocol the desired product was obtained. HPLC/LRMS: >98%, 533 (M+H)+; HRMS(ES+) calcd. for C₂₈H₃₇N₈O₃ 533.2960, found 533.2989.

### EXAMPLE 113

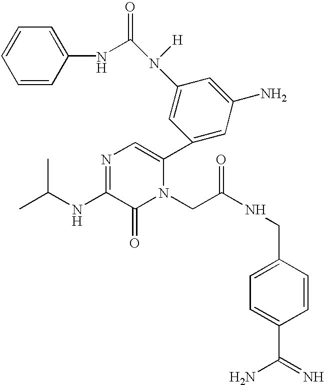

Using phenyl isocyanate and following the reverse amide library protocol the desired product was obtained. HPLC/LRMS: >98%, 568 (M+H)+; ¹H NMR (DMF-d₇, 300 MHz) □ 10.02 (1H, br s), 9.65 (1H, br s), 9.60 (1H, br s), 9.42 (1H, br s), 8.77 (1H, br m), 7.94 (2H, m), 7.64 (2H, m), 7.48 (5H, m), 7.30 (2H, m), 7.17 (1H, m) 6.99 (1H, m), 6.90 (1H, m), 6.64 (1H, s), 4.70 (2H, s), 4.55 (2H, m), 4.28 (1H, m), 1.38 (6H, d); HRMS(ES+) calcd. for C₃₀H₃₄N₉O₃ 568.2784, found 568.2784.

### EXAMPLE 114

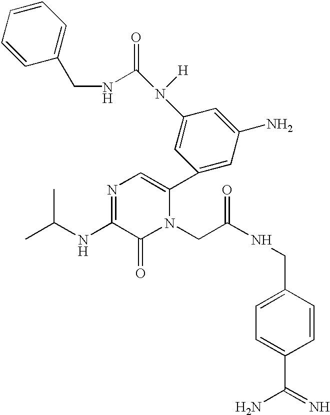

Using benzoyl isocyanate and following the reverse amide library protocol the desired product was obtained. HPLC/LRMS: >98%, 582 (M+H)+; ¹H NMR (DMF-d₇, 300 MHz) δ 10.14 (2H, br s), 9.43 (2H, br s), 9.15 (1H, br m), 8.76 (1H, br m), 8.21 (1H, br m), 7.94 (2H, m), 7.46 (3H, m), 7.36 (4H, m), 7.26 (1H, m), 7.18 (1H, m), 7.03 (1H, m), 6.84 (1H, m), 6.60 (1H, m), 4.69 (2H, s), 4.51 (2H, m), 4.43 (2H, m), 4.26 (1H, br m), 1.36 (6H, d); HRMS(ES+) calcd. for C₃₁H₃₆N₉O₃ 582.2941, found 582.2975.

### EXAMPLE 115

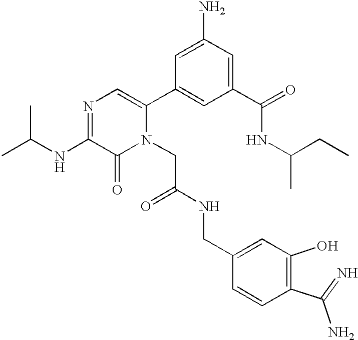

The compound of Example 115 was prepared in an analogous manner to that of Example 186.

### EXAMPLE 116

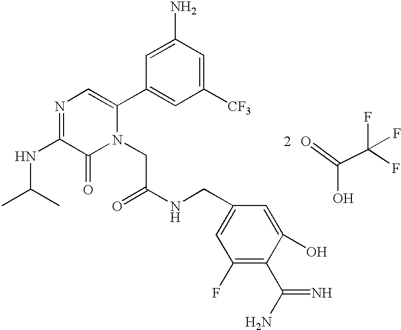

EX-116) To a solution of N-[(3-amino-4-fluoro-1,2-benzisoxazol-6-yl)methyl]-2-[6-[3-amino-5-(trifluoromethyl)phenyl]-3-(isopropylamino)-2-oxopyrazin-1 (2H)-yl]acetamide (230 mg, 0.43 mmol) in 95% ethanol (60 ml) and conc. HCl (6 ml) was added 10% Pd/C catalyst (150 mg) under argon flow. This mixture was stirred under 55 psi of hydrogen at room temperature for 4 hours. The catalyst was removed by filtration through Celite. The filtrate was concentrated and the residue was purified by RP HPLC (15-85 gradient, acetonitrile in 0.1% TFA) to give 170 mg of Ex-116 as an amorphous powder.

HRMS (M+H)+536.2015 found for C₂₄H₂₅F₄N₇O₃; 536.2028 calc'd.

¹H-NMR, 400 MHz, DMSO-d₆ δ 9.30 (s, 2H), 9.08 (s, 2H), 8.70 (bt, 1H), 6.90 (s, 1H), 6.75 (s,1H), 6.70 (bs, 2H), 4.36 (s, 2H), 4.25 (d, J=6.0 Hz, 2H0, 4.07 (m, 1H), 1.20 (d, J=7.3 Hz, 6H).

### EXAMPLE 117

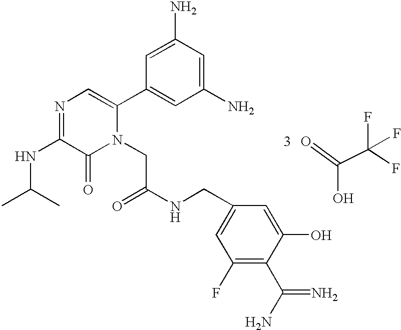

EX-117) The carboxylic acid, [6-(3-[(tert-butoxycarbonyl)amino]-5-nitrophenyl}-3-(isopropylamino)-2-oxopyrazin-1 (2H)-yl]acetic acid (852 mg, 1.91 mmol), 6-(aminomethyl-4-fluoro-1,2-benzisoxazol-3-amine (320 mg, 2.1 mmol) and HOBt-H₂O (2.7 g, 20 mmol) were placed in a flask. DMF (60 ml) and CH₂Cl₂ (40 ml) were added. To this stirred solution was added polymeric DCC resin (8.28 g, loading 1.38 mmol/g, 11.5 mmol) and triethylamine (1.39 ml) and the resulting mixture stirred over night. The resin was removed by filtration and the filtrate concentrated. The crude residue was purified by flash chromatography (Merck 230-400 mesh SiO₂, Hexane:Ethyl acetate; 2:1) to give 750 mg of the product as a white solid. This material (700 mg, 1.15 mmol) was dissolved in methanol (15 ml) and sparged with nitrogen. To this solution was added 10% Pd/C (200 mg) followed by 4N HCl in dioxane (15 ml). This mixture was stirred under 60 psi of hydrogen at room temperature for 4 hours. The catalyst was removed by filtration through Celite. The filtrate was concentrated and the residue was purified by prep HPLC (10-90% gradient, acetonitrile in 0.1% TFA) to give 115 mg of Ex-117 as amorphous solid.

HRMS (M+H)+483.2264 found for C₂₃H₂₇FN₈O₃; 483.2263 calc'd.

¹H-NMR: 400 MHz, DMSO-d₆ δ 9.28 (s, 2H), 9.15 (s, 2H), 8.84 (bt, 1H), 6.74 (s, 1H), 6.61 (s, 1H), 6.56 (s, 2H), 6.49 (s, 2H), 4.32 (s, 2H), 4.15 (d, J=5.77 Hz, 2H), 1.21 (d, J=6.31 Hz, 6H).

### EXAMPLE 118

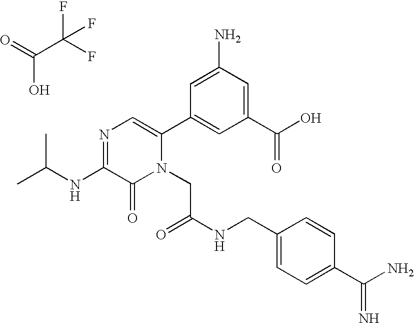

The compound of Example 118 was prepared using the procedures outlined in Examples 16/17 and is merely a different salt thereof.

### EXAMPLE 119

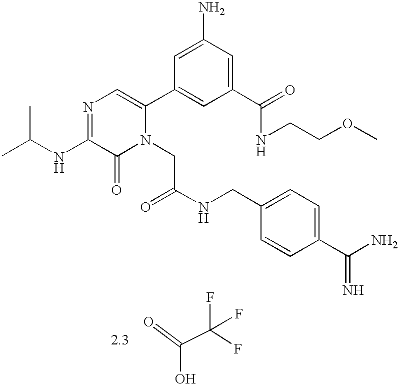

The compound of Example 119 was prepared using the procedures outlined in Example 26 and is merely a different salt thereof.

### EXAMPLE 120

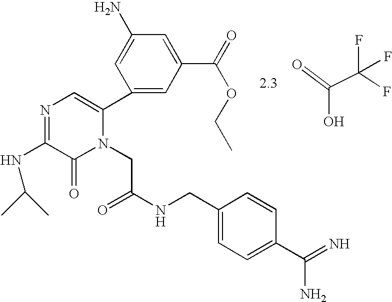

The compound of Example 120 was prepared using the procedures outlined in Example 44 and is merely a different salt thereof.

### EXAMPLE 121

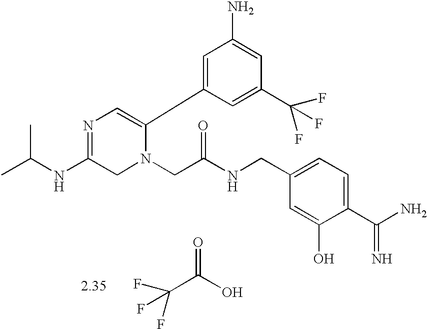

The compound of Example 121 was prepared using the procedures outlined in Example 49 and is merely a different salt thereof.

### EXAMPLE 122

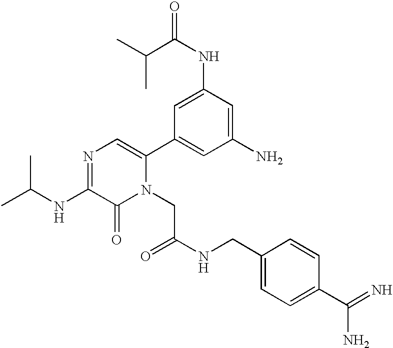

Using isobutyryl chloride and following the reverse amide general library protocol the desired product was obtained. HPLC/LRMS: >98%, 519 (M+H)+; HRMS (EI) calcd for C₂₇H₃₅N₈O₃ 519.2827, found 519.2806.

### EXAMPLE 123

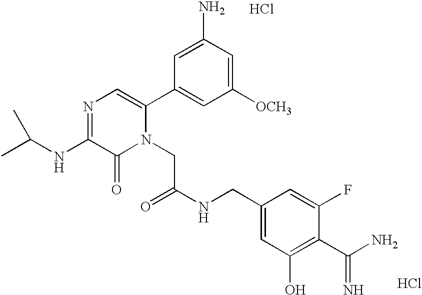

Ex-122) Prepared as previously described for Ex-117. LCMS (RP, 5-90% acetonitrile in 0.1% TFA over 14 min): retention time: 3.41 min;(M+H)⁺=498.

### EXAMPLE 124

Isopropyl 3-amino-5-[1-[2-({4-[amino(imino)methyl]benzyl}amino)-2-oxoethyl]-5-(isopropylamino)-6-oxo-1,6-dihydropyrazin-2-yl]benzoate

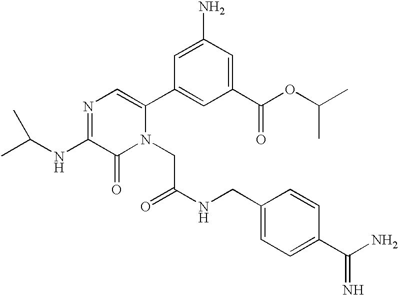

HRMS calcd for C₂₇H₃₃N₇O₄ (M+H): 520.2667. Found: 520.2667.

Anal. Calcd for C₂₇H₃₃N₇O₄+0.7H20+2.4TFA:

C: 47.39; H: 4.60; N: 12.16.

Found: C: 47.40; H: 4.59; N: 12.14.

¹H NMR (DMSO-d₆, 300 MHz) δ 1.25 (d, 6H), 1.30 (d, 6H), 4.12 (m, 3H), 5.11 (m, 2H), 6.73 (s, 1H), 6.80 (s, 1H), 7.12 (s, 1H), 7.28 (s, 1H), 7.43 (d, 2H), 7.76 (d, 2H), 8.71 (t, 1H), 9.05 (s, 2H), 9.28 (s, 2H).

### EXAMPLE 125

3-amino-5-[1-[2-({4-[amino(imino)methyl]-3-hydroxybenzyl}amino)-2-oxoethyl]-5-(isopropylamino)-6-oxo-1,6-dihydropyrazin-2-yl]-N-[(1S)-1-methylpropyl]benzamide

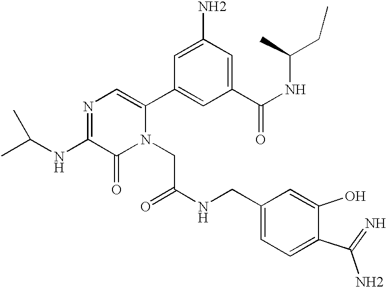

HRMS calcd for C₂₈H₃₆N₈O₄ (M+H): 549.2932. Found: 549.2921.

Anal. Calcd for C₂₈H₃₆N₈O₄+2.25TFA+0.75H₂O:

C: 47.67; H: 4.89; N: 13.68.

Found: C: 47.70; H: 4.87; N: 13.66.

¹H NMR (DMSO-d₆, 300 MHz) δ 0.85 (t, 3H), 1.11 (d, 3H), 1.22 (d, 6H), 1.48 (m, 2H), 3.87 (m, 2H), 4.10 (m, 3H), 4.22 (m, 3H), 4.38 (s, 2H), 6.69 (m, 2H), 6.76 (d, 1H), 6.89 (s, 1H), 6.99 (s, 1H), 7.10 (s, 1H), 7.44 (d, 1H), 8.02 (d, 1H), 8.62 (t, 1H), 8.79 (bs, 2H), 8.95 (bs, 2H).

### EXAMPLE 126

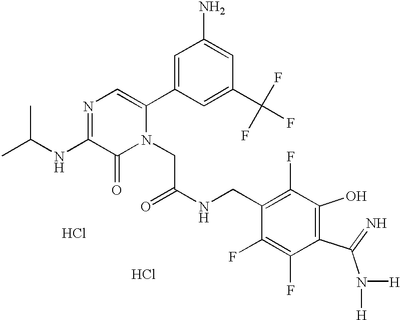

The compound of Example 126 was prepared in an analogous manner to that of Example 186.

### EXAMPLE 127

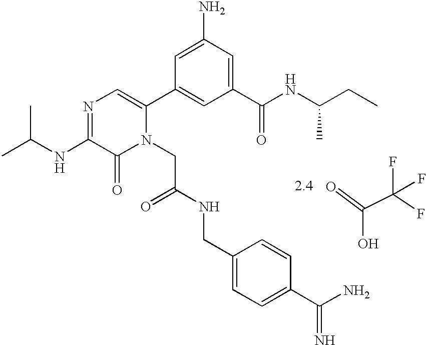

The compound of Example 127 was prepared in an analogous manner to that of Example 186.

### EXAMPLES 128/129

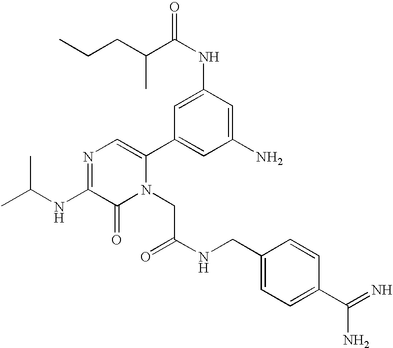

Using 2-methylvaleryl chloride and following the reverse amide general library protocol the desired product was obtained. HPLC/LRMS: >98%, 547 (M+H)+; HRMS (EI) calcd for C₂₉H₃₉N₈O₃ 547.3140, found 547.3109

### EXAMPLE 130

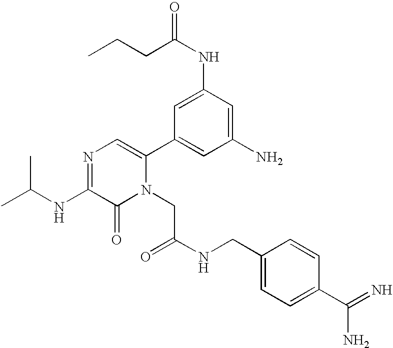

Using butyryl chloride and following the reverse amide general library protocol the desired product was obtained. HPLC/LRMS: >98%, 519 (M+H)+; HRMS (EI) calcd for C₂₇H₃₅N₈O₃ 519.2827, found 519.2802.

### EXAMPLE 131

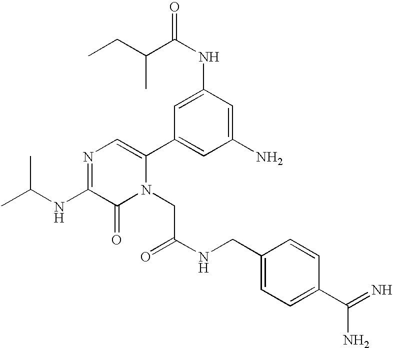

Using 2-methylbutyryl chloride and following the reverse amide general library protocol the desired product was obtained. HPLC/LRMS: >98%, 533 (M+H)+; HRMS (EI) calcd for C₂₈H₃₇N₈O₃ 533.2983, found 533.2965.

### EXAMPLE 132

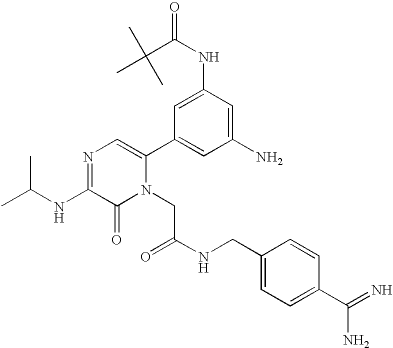

Using trimethyl acetyl chloride and following the reverse amide general library protocol the desired product was obtained. HPLC/LRMS: >98%, 533 (M+H)+; HRMS (EI) calcd for C₂₈H₃₇N₈O₃ 533.2983, found 533.2968.

### EXAMPLE 133

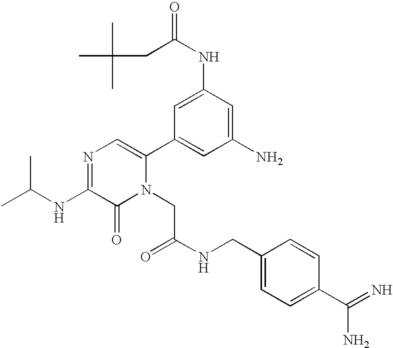

Using tert-butyl acetyl chloride chloride and following the reverse amide general library protocol the desired product was obtained. HPLC/LRMS: >98%, 547 (M+H)+; HRMS (EI) calcd for C₂₉H₃₉N₈O₃ 547.3140, found 547.3098.

### EXAMPLE 134

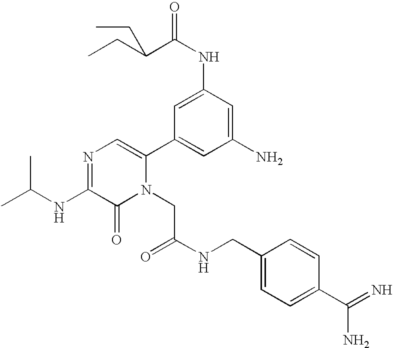

Using 2-ethylbutyryl chloride and following the reverse amide general library protocol the desired product was obtained. HPLC/LRMS: >98%, 547 (M+H)+; HRMS (EI) calcd for C₂₉H₃₉N₈O₃ 547.3140, found 547.3147

### EXAMPLE 135

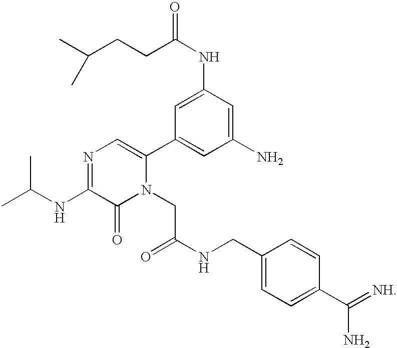

Using 4-methyl pentanoyl chloride and following the reverse amide general library protocol the desired product was obtained. HPLC/LRMS: >98%, 547 (M+H)+; HRMS (EI) calcd for C₂₉H₃₉N₈O₃ 547.3140, found 547.3117

### EXAMPLE 136

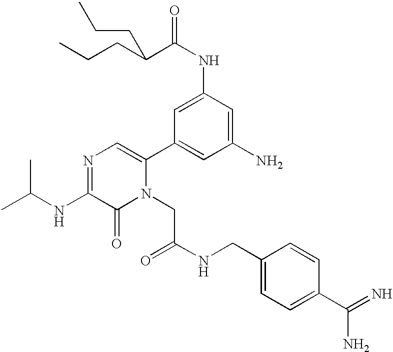

Using 2,2-di-n-propylacetyl chloride and following the reverse amide general library protocol the desired product was obtained. HPLC/LRMS: >98%, 575 (M+H)+; HRMS (EI) calcd for C₃₁H₄₃N₈O₃ 575.3453, found 575.3494.

### EXAMPLE 137

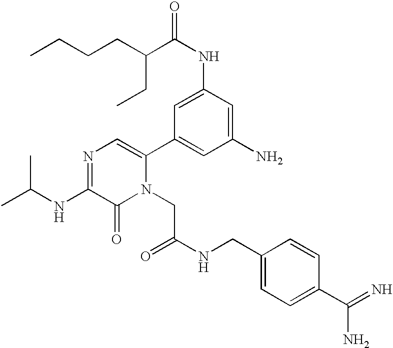

Using 2-ethylhexanoyl chloride and following the reverse amide general library protocol the desired product was obtained. HPLC/LRMS: >98%, 575 (M+H)+; HRMS (EI) calcd for C₃₁H₄₃N₈O₃ 575.3453, found 575.3446.

### EXAMPLE 138

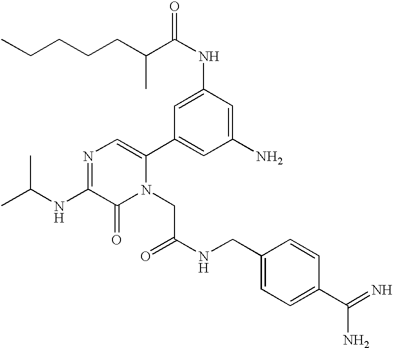

Using 2-methylheptanoyl chloride and following the reverse amide general library protocol the desired product was obtained. HPLC/LRMS: >98%, 575 (M+H)+; HRMS (EI) calcd for C₃₁H₄₃N₈O₃ 575.3453, found 575.3453.

### EXAMPLE 139

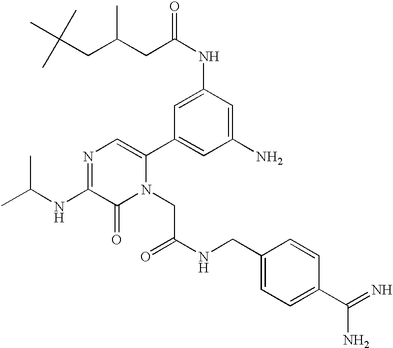

Using 3,5,5-trimethylhexanoyl chloride and following the reverse amide general library protocol the desired product was obtained. HPLC/LRMS: >98%, 589 (M+H)+; HRMS (EI) calcd for C₃₂H₄₅N₈O₃ 589.3609, found 589.3606.

### EXAMPLE 140

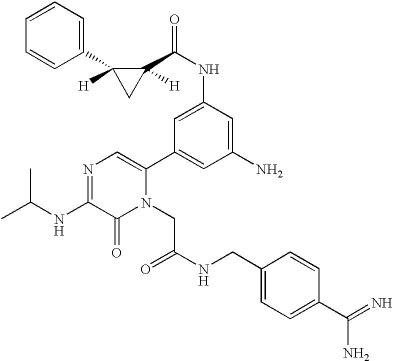

Using trans-2-phenyl-cyclopropane carbonyl chloride and following the reverse amide general library protocol the desired product was obtained. HPLC/LRMS: >98%, 593 (M+H)+; HRMS (EI) calcd for C₃₃H₃₇N₈O₃ 593.2983, found 593.3020.

### EXAMPLE 141

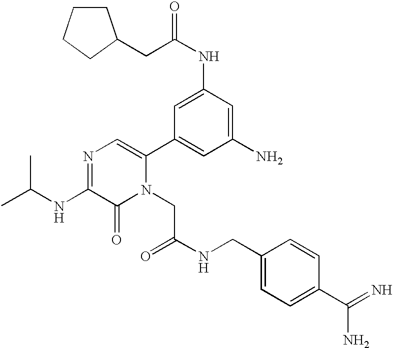

Using cyclopentylacetyl chloride and following the reverse amide general library protocol the desired product was obtained. HPLC/LRMS: >98%, 559 (M+H)+;HRMS (EI) calcd for C₃₀H₃₉N₈O₃ 559.3140, found 559.3123.

### EXAMPLE 142

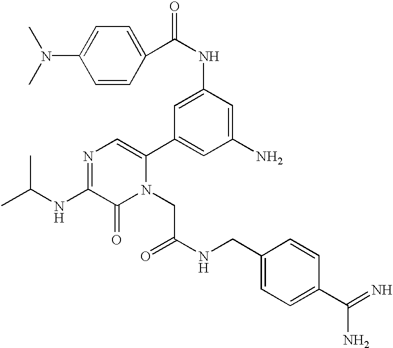

Using 4-dimethylaminobenzoyl chloride and following the reverse amide general library protocol the desired product was obtained. HPLC/LRMS: >98%, 559 (M+H)+;HRMS (EI) calcd for C₃₂H₃₈N₉O₃ 559.3140, found 559.3123.

### EXAMPLE 143

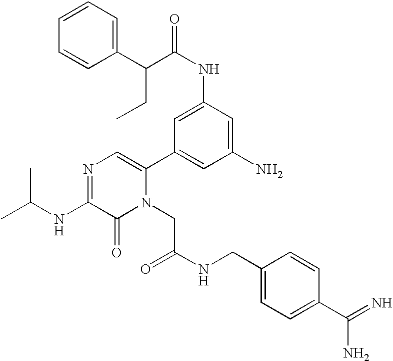

Using 2-phenylbutyryl chloride and following the reverse amide general library protocol the desired product was obtained. HPLC/LRMS: >98%, 595 (M+H)+;HRMS (EI) calcd for C₃₃H₃₉N₈O₃ 595.3140, found 595.3156.

### EXAMPLE 144

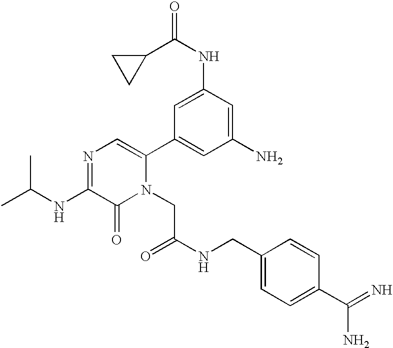

Using cyclopropanecarbonyl chloride and following the reverse amide general library protocol the desired product was obtained. HPLC/LRMS: >98%, 517 (M+H)+;HRMS (EI) calcd for C₂₇H₃₃N₈O 517.2670, found 517.2690.

### EXAMPLE 145

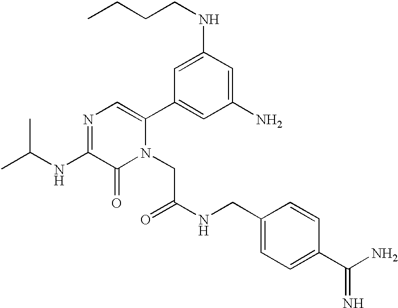

By following the method of Example 110 and substituting butyraldehyde for phenylacetaldehyde, Example 145 was prepared: HPLC/LRMS: >98%, 505 (M+H)+; HRMS(ES+) calcd. for C₂₇H₃₇N₈O₂ 505.3034, found 505.3045.

### EXAMPLE 146

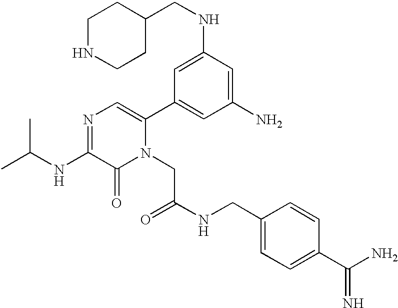

By following the method of Example 110 and substituting N-Boc-4-piperidinylcarboxaldehyde for phenylacetaldehyde, Example 146 was prepared: HPLC/LRMS: >97%, 546 (M+H)+; HRMS(ES+) calcd. for C₂₉H₄₀N₉O₂ 546.3299, found 546.3268.

### EXAMPLE 147

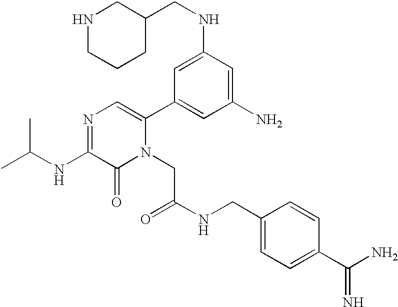

By following the method of Example 110 and substituting N-Boc-4-piperidinylcarboxaldehyde for phenylacetaldehyde, Example 147 was prepared: HPLC/LRMS: >98%, 546 (M+H)+; HRMS(ES+) calcd. for C₂₉H₄₀N₉O₂ 546.3299, found 546.3255.

### EXAMPLE 148

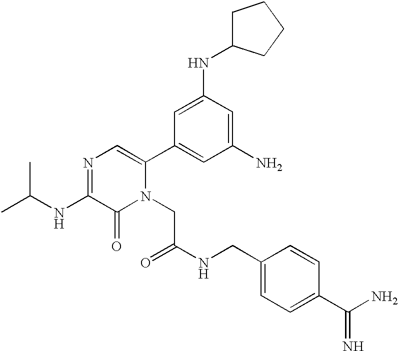

By following the method of Example 110 and substituting cyclopentanone for phenylacetaldehyde, Example 148 was prepared: HPLC/LRMS: >98%, 517 (M+H)+; HRMS(ES+) calcd. for C₂₈H₃₇N₈O₂ 517.3034, found 517.3038.

### EXAMPLE 149

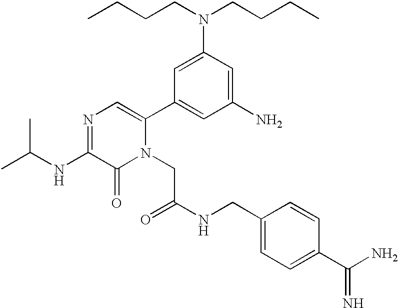

By following the method of Example 110 and substituting butyraldehyde (2.0 equivalents) for phenylacetaldehyde, Example 149 was prepared: HPLC/LRMS: >97%, 561 (M+H)+; HRMS(ES+) calcd. for C₃₁H₄₅N₈O₂ 561.3660, found 561.3653.

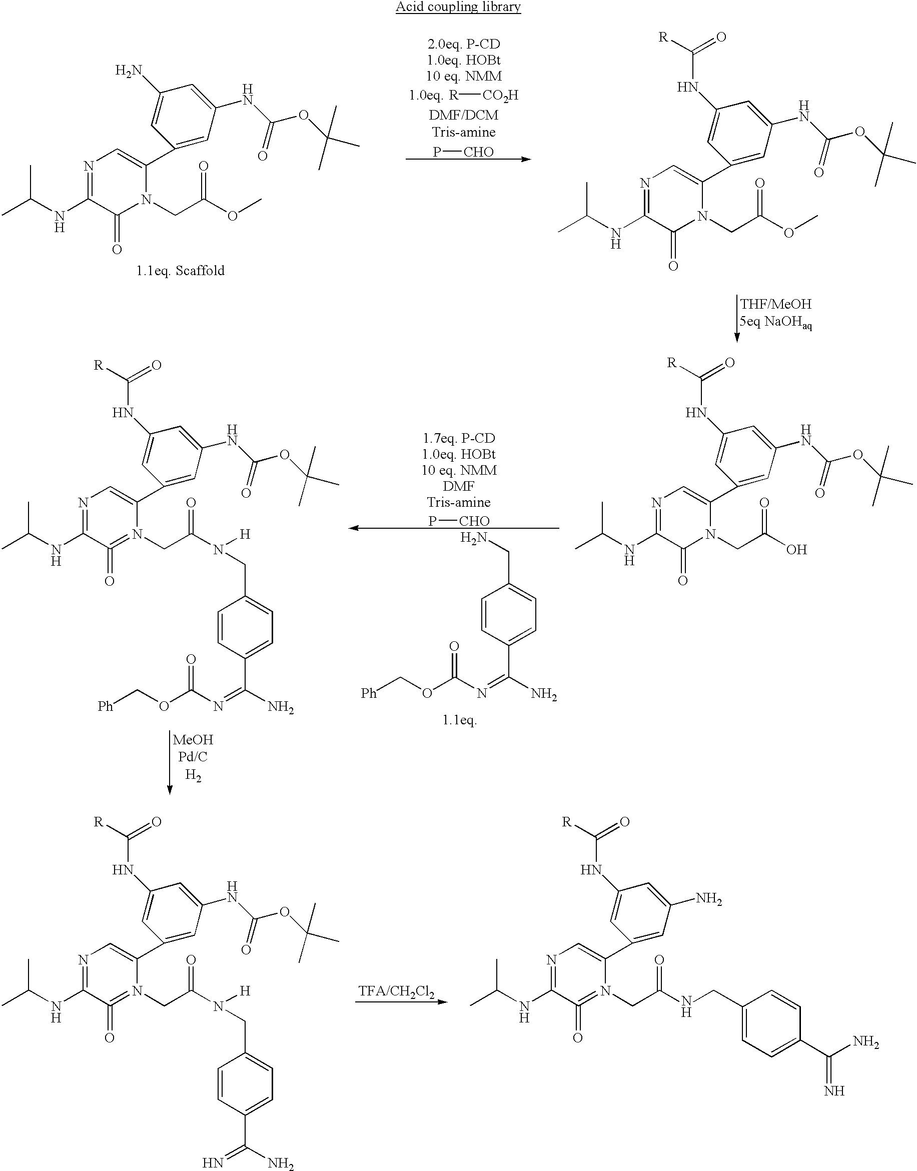

General Library Protocol Modification:

Follows the same General Library Protocol outlined previously except the first step is a peptide coupling with a commercially available carboxylic acid.

### EXAMPLE 150

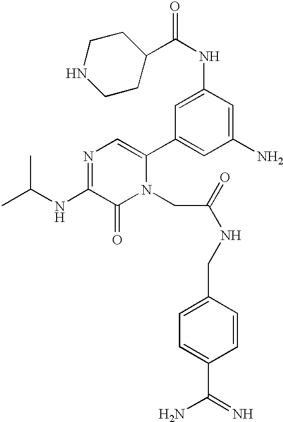

Using 1-(tert-butoxycarbonyl)-4-piperidinecarboxylic acid and following the acid coupling library protocol the desired product was obtained. HPLC/LRMS: >97%, 561 (M+H)+; HRMS(ES+) calcd. for C₂₉H₃₈N₉O₃ 560.3092, found 560.3114.

### EXAMPLE 151

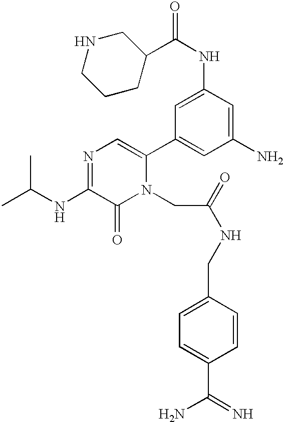

Using 1-(tert-butoxycarbonyl)-3-piperidinecarboxylic acid and following the acid coupling library protocol the desired product was obtained. HPLC/LRMS: >98%, 560 (M+H)+; HRMS(ES+) calcd. for C₂₉H₃₈N₉O₃ 560.3092, found 560.3111.

### EXAMPLE 152

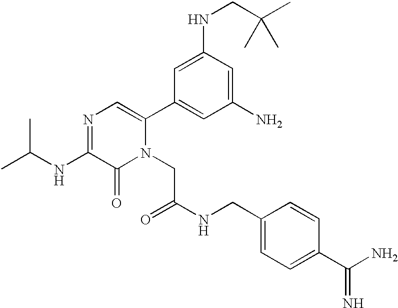

By following the method of Example 110 and substituting trimethylacetaldehyde for phenylacetaldehyde, Example 152 was prepared: HPLC/LRMS: >97%, 519 (M+H)+; HRMS(ES+) calcd. for C₂₈H₃₉N₈O₂ 519.3190, found 519.3206.

### EXAMPLE 153

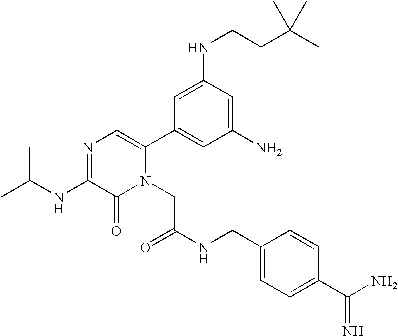

By following the method of Example 110 and substituting 3,3-dimethylbutyraldehyde for phenylacetaldehyde, Example 153 was prepared: HPLC/LRMS: >75%, 533 (M+H)+; HRMS(ES+) calcd. for C₂₉H₄₁N₈O₂ 533.3347, found 533.3368.

### EXAMPLE 154

N-{4-[amino(imino)methyl]-3-fluorobenzyl}-2-[6-[3-amino-5-(trifluoromethyl)phenyl]-3-(isopropylamino)-2-oxopyrazin-1 (2H)-yl]acetamide

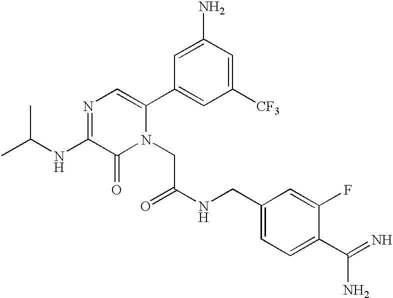

### EXAMPLE 155

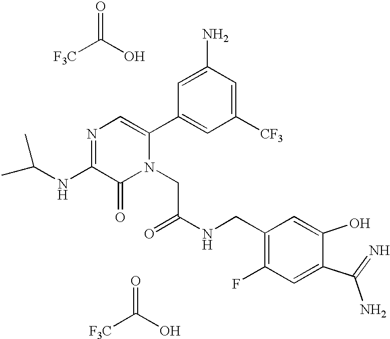

LCMS (RP, 15-90% gradient acetonitrile in 0.1% ammonium acetate over 6 min): retention time=6.05; (M+H)⁺=536; negative ion mode (M−H)⁻⁼534.

### EXAMPLE 156

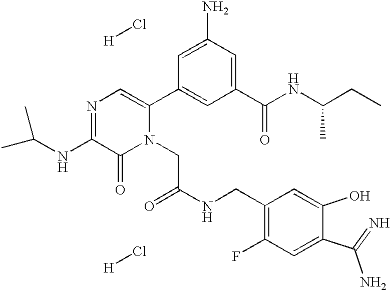

LCMS (RP, 15-50% acetonitrile in 0.1% TFA over 14 min) retention time=4.05; (M+H)⁺=567

### EXAMPLE 157

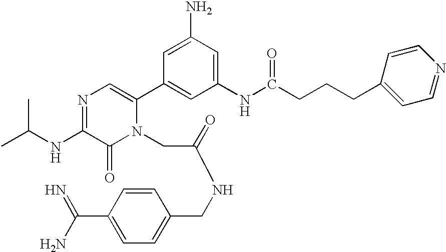

Using 4-pyridinebutanoic acid hydrochloride and following the acid coupling library protocol the desired product was obtained. HPLC/LRMS: >95%, 596 (M+H)+; HRMS(ES+) calcd. for C₃₂H₃₈N₉O₃ 596.3092, found 596.3075.

### EXAMPLE 158

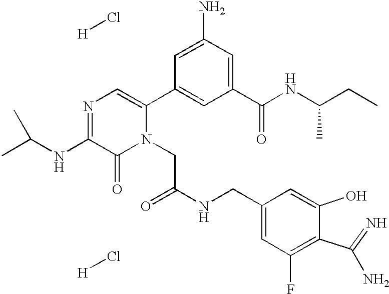

LCMS (RP, 15-90% acetonitrile gradient in ammonium acetate over 14 min): retention time=5.35 min; (M+H)⁺=567; negative ion mode (M−H)⁻=565

### EXAMPLES 159/160

3-amino-5-[1-[2-({4-[amino(imino)methyl]benzyl}amino)-2-oxoethyl]-5-(isopropylamino)-6-oxo-1,6-dihydropyrazin-2-yl]-N-(4-fluorobenzyl)benzamide

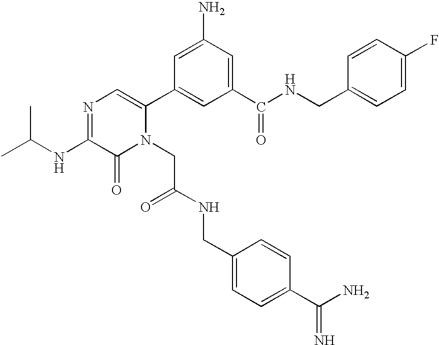

¹H NMR (400 MHz, CD₃OD): δ 7.78 (d, 2H), 7.33-7.30 (m, 4H), 7.23 (t, 1H), 7.04 (t, 1H), 6.99 (t, 2H), 6.84 (t, 1H), 6.65 (s, 1H), 4.62 (s, 2H), 4.48 (d, 2H), 4.40 (d, 2H), 4.05-4.02 (m, 1H), 1.34 (d, 6H); Analysis: C₃₁H₃₃FN₈O₃+2.45 TFA+1.15H₂O calcd: C, 48.73; H, 4.3; N, 12.66; found: C, 48.73; H, 4.28; N, 12.64.

Two salts were prepared for this compound and data for each is reported in Table 3.

### EXAMPLE 161

3-amino-5-[1-[2-({4-[amino(imino)methyl]-3-hydroxybenzyl}amino)-2-oxoethyl]-5-(isopropylamino)-6-oxo-1,6-dihydropyrazin-2-yl]-N-benzylbenzamide

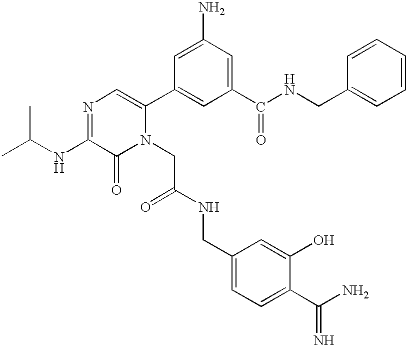

¹H NMR (400 MHz, CD₃OD): δ 7.53 (d, 2H), 7.31-7.21 (m, 5H), 7.16 (t, 1H), 6.92 (t, 1H), 6.87 (s, 1H), 6.76-6.73 (m, 1H), 6.64 (s, 1H), 4.59 (s, 2H), 4.53 (s, 2H), 4.29 (s, 2H), 4.05-4.02 (m, 1H), 1.37 (d, 6H); MS-ESI (M+H)=583; Analysis: C₃₁H₃₄N₈O₄+3.0 TFA+0.6H₂O calcd: C, 47.5; H, 4.11; N, 11.97; found: C, 47.52; H, 4.12; N, 11.84.

### EXAMPLE 162

3-amino-5-[1-[2-({4-[amino(imino)methyl]-3-hydroxybenzyl}amino)-2-oxoethyl]-5-(isopropylamino)-6-oxo-1,6-dihydropyrazin-2-yl]-N-(4-fluorobenzyl) benzamide

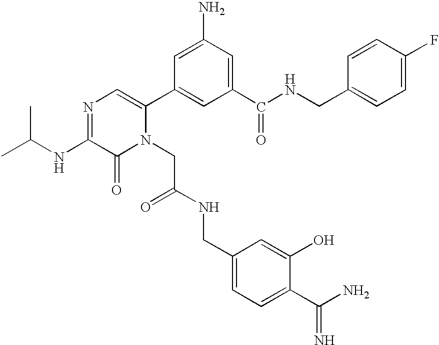

¹H NMR (400 MHz, CD₃OD): δ 7.53 (d, 1H), 7.34-7.30 (m, 2H), 7.26 (t, 1H), 7.11 (t, 1H), 7.0 (t, 2H), 6.88-6.86 (m, 2H), 6.76-6.73 (m, 1H), 6.64 (s, 1H), 4.59 (s, 2H), 4.49 (s, 2H), 4.30 (s, 2H), 4.06-4.02 (m, 1H), 1.36 (d, 6H); ¹⁹F NMR (371 MHz, CD₃OD): δ −77.05 (s, 8.7), −188.16 (sep, 1 F); MS-ESI (M+H)=601; Analysis: C₃₁H₃₃FN₈O₄+2.25 TFA+0.65H₂O calcd: C, 49.07; H, 4.23; N, 12.89; found: C, 49.1; H, 4.29; N, 12.8.

### EXAMPLE 163

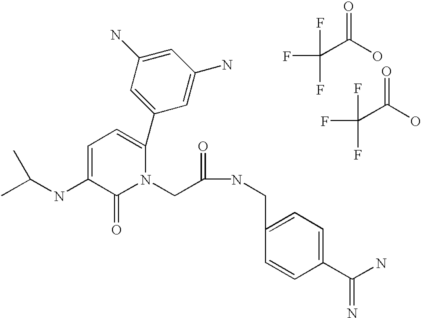

¹H NMR ppm (deuteromethanol): 1.26 (d, 6H), 3.63 (septet, 1H), 4.44 (d, 2H), 4.65 (d, 2H), 6.27 (dd, 1H), 6.78 (dd, 1H), 7.62 (m, 9H);

HPLC purity (retention time): >99% (2.39 min);

HRMS calcd for C₂₄H₂₉N₇O₂ (M⁺+H) 448.2456, found 448.2447.

### EXAMPLE 164

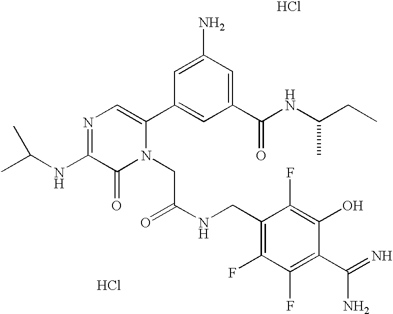

HRMS: (M+H)⁺ 603.2616 found for C₂₈H₃₃F₃N₈O₄; 603.2650 calc'd.

¹H-NMR: 300 MHz, DMSO-d₆ δ 9.63 (bs, 2H), 9.58 (bs, 2H), 9.30 (bm, 1H), 8.88 (t, J=5.6 Hz, 1H), 8.42 (d, J=8.4 Hz, 1H), 7.66 (s, 1H), 7.57 (s, 1H), 7.23 (s, 1H), 6.71 (s, 1H), 4.38 (bs, 2H), 4.27-4.34 (m, 3H), 3.88 (septet, J=6.9 Hz, 1H, 1.49 (m, 2H), 1.26 (d, J=6.9 Hz, 6H), 1.12 (d, J=6.9 Hz, 3H), 0.85 (t, J=6.9 Hz, 3H).

¹⁹F NMR: 282 MHz, DMSO-d₆ δ −138.45 (d, J=13.0 Hz, 1F), −144.72 (dd, J=13.0 Hz, J=24.0 Hz, 1F), −151.24 (d, J=24.0 Hz, 1F).

### EXAMPLE 166

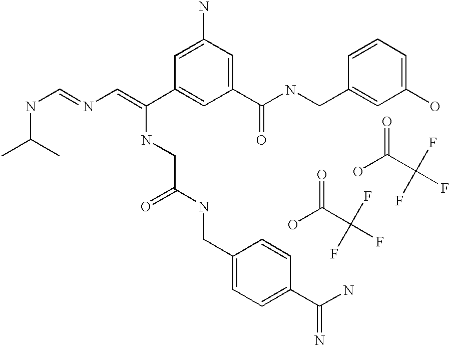

Prepared analogously to Example 110.

### EXAMPLE 167

3-amino-5-[1-[2-({4-[amino(imino)methyl]-2-hydroxybenzyl}amino)-2-oxoethyl]-5-(isopropylamino)-6-oxo-1,6-dihydropyrazin-2-yl]-N-[(1S)-1-methylpropyl]benzamide

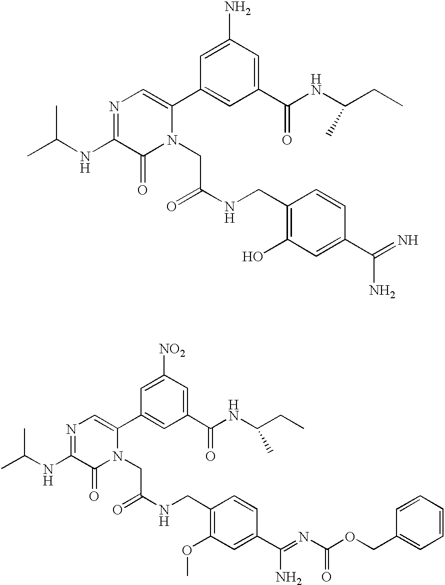

Benzyl (1Z)-amino{4-[({[6-[3-amino-5-({[(1S)-1-methylpropyl]amino}carbonyl)phenyl]-3-(isopropylamino)-2-oxopyrazin-1 (2H)-yl]acetyl}amino)methyl]-3-methoxyphenyl}methylidenecarbamate

Ex-167a) LRMS m/z 697.3 (M⁺+H); HPLC purity (retention time): 90% (1.9 min).

Ex-167) The crude product from Ex-167a (0.94 g crude, 1.3 mmol) was dissolved in 4 mL of CH₂Cl₂. BBr₃ (14 mL total of a 2M solution in CH₂Cl₂, 28 mmol) was added portionwise until the reaction was complete (3 portions over 8 h). The solid was filtered, then washed with CH₂Cl₂ and Et₂O.

The solid was purified by reverse phase HPLC with a gradient of 15/85-45/55% acetonitrile/water (+0.1% TFA) over 12 mins. to give 0.1 g of an off-white solid: LRMS m/z 549.2 (M⁺+H); HPLC purity (retention time): 98% (1.3 min); HRMS (M+H): Calc'd for C₂₈H₃₆N₈O₄: 549.2932; Found: 549.2898. ¹H NMR (400 MHz, CD₃OD) δ 0.92 (t, 3H, J=7.3 Hz), 1.19 (d, 3H, J=6.5 Hz), 1.40 (d, 6H, J=6.2 Hz), 1.56 (m, 2H), 3.97 (m, 1H), 4.05 (m, 1H), 4.38 (s, 2H), 4.64 (s, 2H), 6.65 (s, 1H), 6.96 (s, 1H), 7.16 (m, 4H), 7.30 (s, 1H), 8.62 (br t, 1H).

### EXAMPLE 168

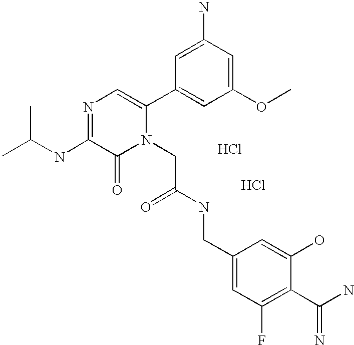

Prepared analogously to Examples 53 and 117.

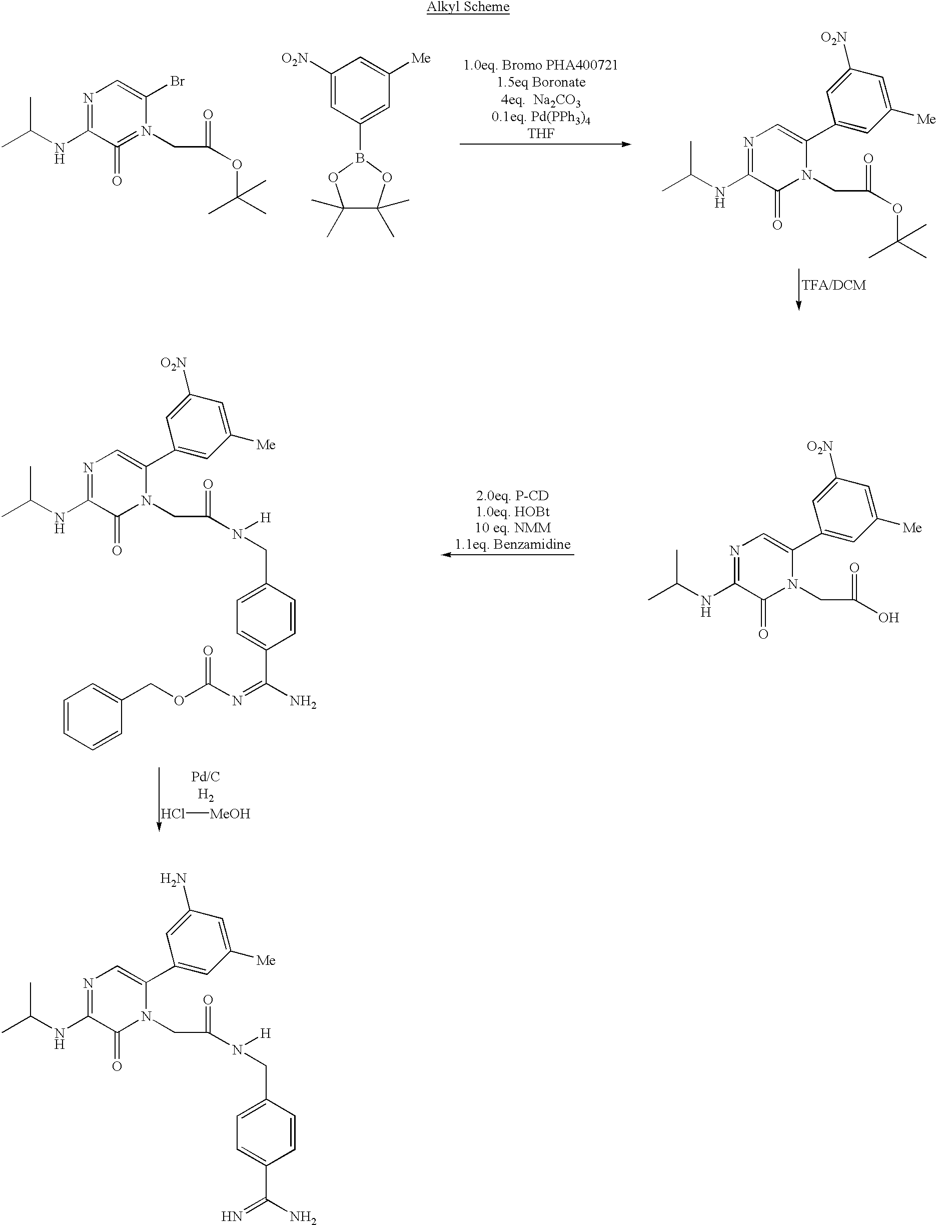

### EXAMPLE 170

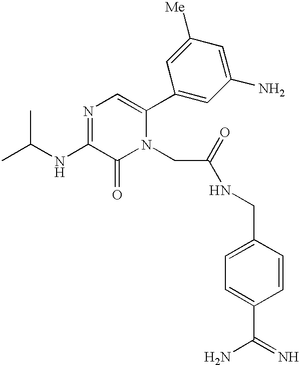

HPLC/LRMS: >97%, 448 (M+H)+; ¹H NMR(DMF-d₇, 400 MHz) δ 9.91 (2H, s), 9.74 (2H, s), 9.25 (1H, br s), 9.02 (1H, m), 8.01 (2H, m), 7.44 (1H, s), 7.37 (3H, m), 7.14 (1H, s), 6.68 (1H, s), 4.80 (2H, m), 4.61 (1H, m), 4.37 (2H, m), 2.27 (3H, s), 1.37 (6H, d); HRMS (ES+) calcd. for C₂₄H₃₀N₇O₂ 448.2456, found 448.2434.

### EXAMPLE 171

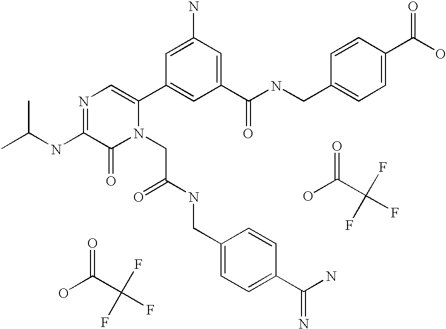

Prepared analogously to Example 186.

### EXAMPLE 172

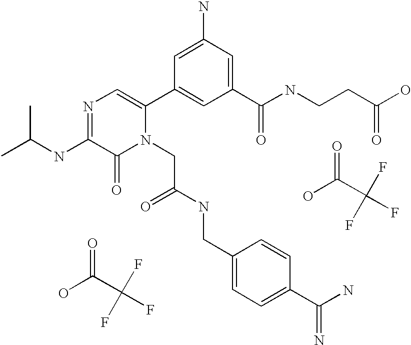

Prepared analogously to Example 186.

### EXAMPLE 173

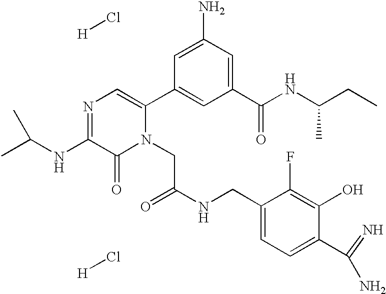

LCMS (RP, 15-90% acetonitrile in 0.1% ammonium acetate over 6 min): retention time 3.04 min; (M+H)⁺=567, Negative Ion mode (M−H)⁻=565.

Modified Amide Library

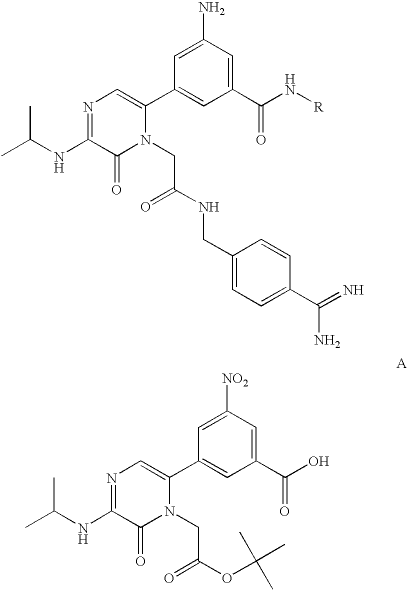

3-[1-(2-tert-butoxy-2-oxoethyl)-5-(isopropylamino)-6-oxo-1,6-dihydropyrazin-2-yl]-5-nitrobenzoic Acid

¹H NMR (300 MHz, CD₃OD) δ 1.37 (d, 6H, J=6.3 Hz), 1.45 (s, 9H), 4.21-4.36 (m, 1H), 4.41 (s, 2H), 7.00 (s, 1H), 8.45 (s, 1H), 8.49 (s, 1H), 8.97 (s, 1H).

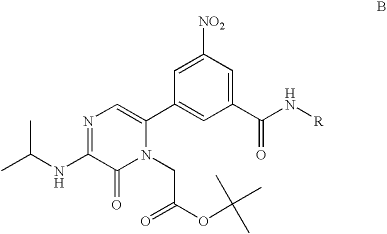

Amine 1 4-methoxy benzylamine

Amine 2 3-methoxy benzylamine

Amine 3 Beta analine methylester hydrochloride

Amine 4 Methyl-4-(aminomethyl)-benzoate hydrochloride

Intermediate A (3.9 g, 9.0 mmol), primary amines 1-8 (see above list, 9.9 mmol), HOBt (0.6 g, 4.5 mmol), NMM (5.9 mL, 54.0 mmol), PS-carbodiimide (11.1 g, 13.5 mmol), PS-diethylene triamine (9.6 g, 27.0 mmol), Aldehyde wang (9.5 g, 27.0 mmol), CH₂Cl₂ (90 mL0, DMF (50 mL).

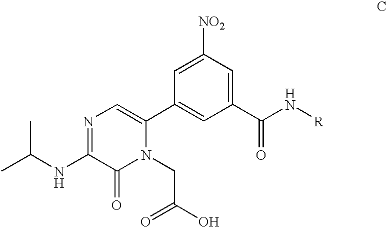

Intermediate B (9.0 mmol), TFA (30 mL), CH₂Cl₂ (100 ml).

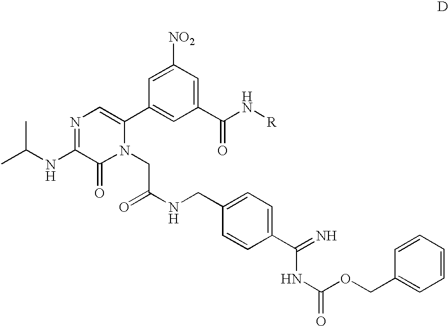

Intermediate C (4.5 mmol), benzyl [4-(aminomethyl)phenyl](imino)methylcarbamate dihydrochloride (1.7 g, 4.9 mmol), HOBt (0.3 g, 2.5 mmol), NMM (6.5 mL, 59.3 mmol), PS-carbodiimide resin (6.1 g, 7.4 mmol), PS-diethylene triamine (5.3 g, 14.8 mmol), Aldehyde wang (5.2, 14.8 mmol).

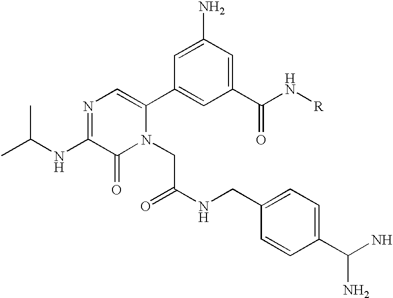

Intermediate D (4.5 mmol), Pd/C (10%, 0.8 g), methanol (100 mL).

### EXAMPLE 165

### NHR=Methyl-4-(aminomethyl)-benzoate Hydrochloride

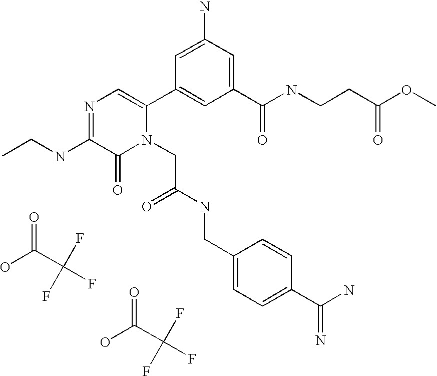

¹H NMR (300 MHz, CD₃OD) δ 1.42 (d, 6H, J=6.6 Hz), 2.62-2.70 (m, 2H), 3.60-3.65 (m, 2H), 3.68 (s, 3H), 4.07-4.14 (m, 1H), 4.46 (s, 2H), 4.67 (s, 2H), 6.71 (s, 1H), 7.20 (s, 1H), 7.52 (s, 1H), 7.41 (s, 1H), 7.38-7.81 (abq, 4H, J=8.1 Hz).

LRMS m/z 563.2 (M⁺+H).

### EXAMPLE 169

### NHR═(S)-(+)-sec-butylamine

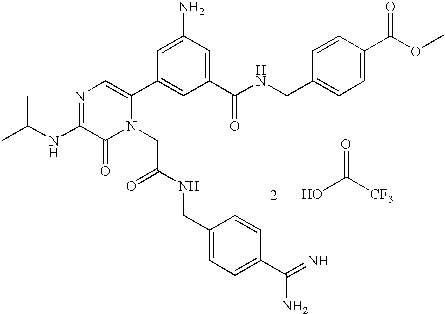

¹H NMR (300 MHz, CD₃OD) δ 1.44 (d, 6H, J=6.6 Hz), 3.92 (s, 3H), 4.05-4.14 (m, 1H), 4.45 (s, 2H), 4.63 (s, 2H), 4.72 (s, 2H), 6.70 (s, 1H), 7.04 (s, 1H), 7.26 (s, 1H), 7.31-7.46 (m, 5H), 7.71-7.98 (abq, 4H, J=8.4 Hz).

LRMS m/z (M⁺+H).

### EXAMPLE 174

### NHR=4-methoxy benzylamine

¹H NMR (400 MHz, CD₃OD) δ 1.38 (d, 6H, J=6.6 Hz), 3.73 (s, 3H), 4.01-4.05 (m, 1H), 4.38 (s, 2H), 4.44 (s, 2H), 4.63 (s, 2H), 6.63 (s, 1H), 6.82-7.28 (m, 7H), 7.31-7.69 (abq, 4H, J=8.4 Hz).

LRMS m/z 597.3 (M⁺+H).

### EXAMPLE 175

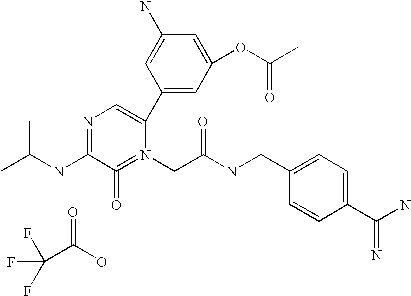

Prepared analogously to Example 55.

### EXAMPLE 176

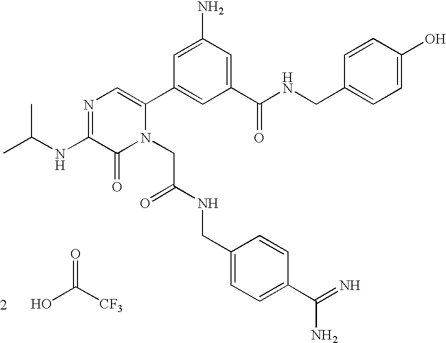

Prepared analogously to Example 186.

### EXAMPLE 177

### NHR=3-methoxy Benzylamine

¹H NMR (400 MHz, CD₃OD) δ 1.26 (d, 6H, J=6.4 Hz), 3.75 (s, 3H), 4.11-4.22 (m, 1H), 4.45 (d, 2H, J=6.0 Hz), 4.52 (d, 2H, J=6.0), 4.63 (s, 2H), 6.76 (s, 1H), 6.80-7.35 (m, 7H), 7.41-7.88 (abq, 4H, J=8.2 Hz).

LRMS m/z 597.3 (M⁺+H).

### EXAMPLE 178

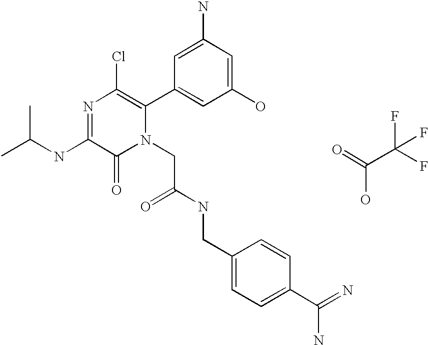

Prepared analogously to Example 4.

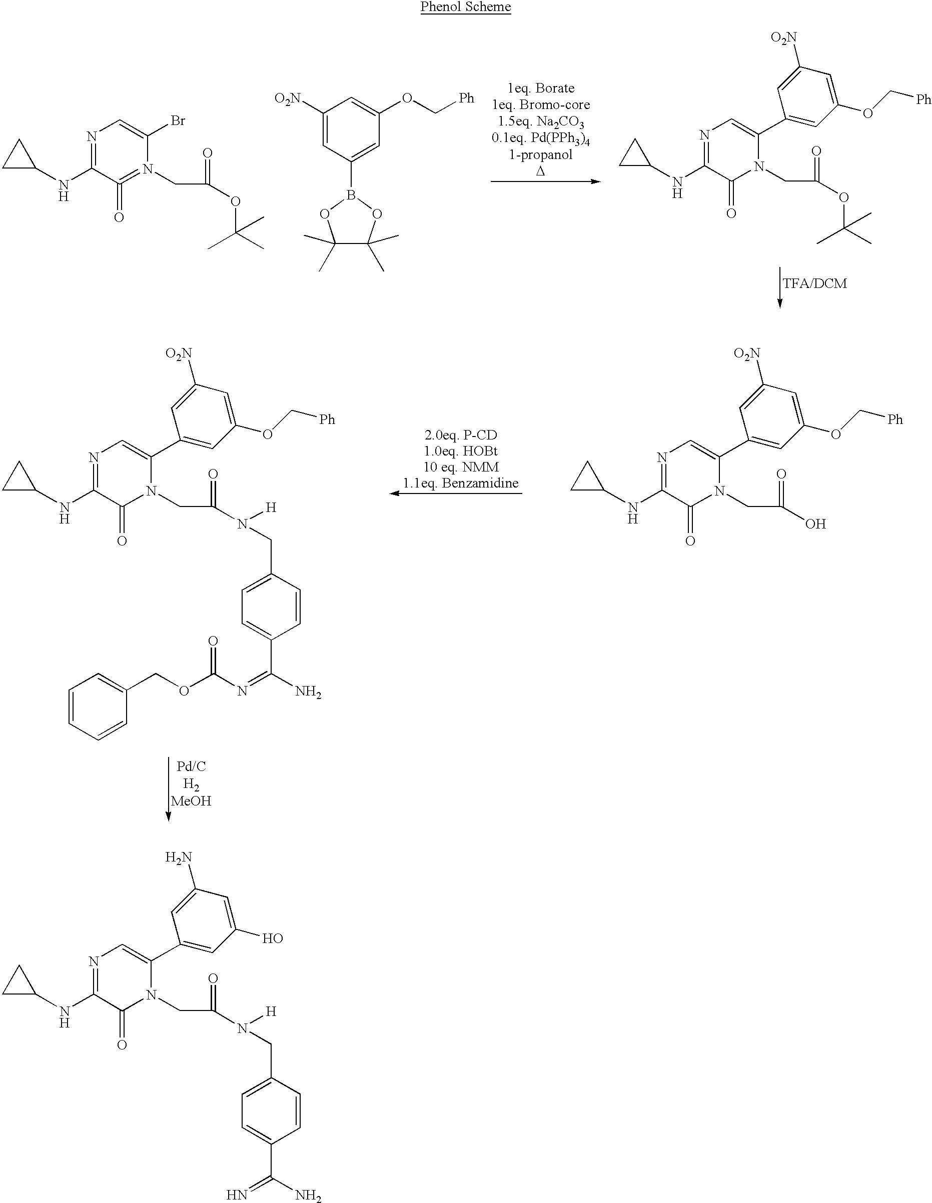

### EXAMPLE 179

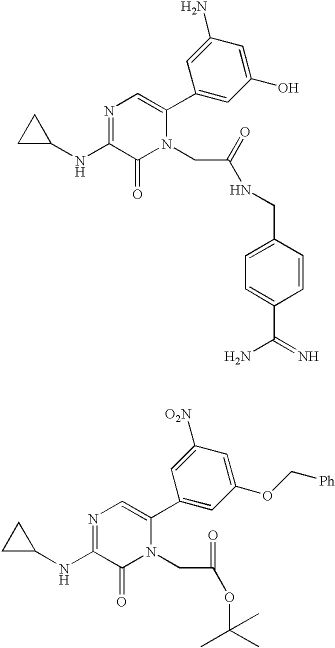

Ex-179a) HPLC/LRMS: >97%, 493 (M+H)+; ¹H NMR(CDCl₃, 400 MHz) δ 7.85-7.81 (2H, m), 7.39-7.28 (6H, m), 6.87 (1H, s), 6.34 (1H, m), 5.12 (2H, s), 4.34 (2H, s), 2.80 (1H, m), 1.42 (9H, s), 0.86 (2H, m), 0.61 (2H, m); ¹³C NMR (CDCl₃, 100 MHz) δ 166.56, 159.59, 151.96, 151.84, 135.40, 135.11, 129.05, 128.83, 1 27.80, 126.80, 123.54, 122.84, 117.06, 110.09, 83.46, 71.12, 48.1 4,28.13, 23.75, 7.17; HRMS(ES+) calcd. for C₂₆H₂₉N₄O₆ 493.2082, found 493.2052.

Ex-179) 179a (1.07grams) was stirred in DCM (50 mL) and TFA (10 mL) for 18 hrs. The reaction was concentrated invacuo and taken up repeatedly with heptane to reduce TFA load. Carboxylate residue was taken up in DMF and activated with P-CD(2.0 eq), HOBt(1.0 eq), and 10 eqNMM) on orbital shaker. The benzamidine (SC81368, 1.1 eq) was added and shaking continued for 4 hrs. The mixture was filtered and the resin cake rinsed with DCM. The filtrate was concentrated in-vacuo and the crude partitioned between chloroform and saturated sodium bicarbonate solution. The aqueous was extract twice more with equal amounts of chloroform. The combined organic layers were concentrated to dryness under reduced vacuum. After drying on high vacuum pump, the residue was taken up in methanol, and minimal 3N HCl-MeOH. Hydrogenolysis and reduction was completed on Parr Hydrogenator at 50 psi. The concentrated crude was triturated with diethyl ether and filtered. The cake was dried in vacuum desicator. The desired product was collected by further purification on Gilson HPLC-RP with 0.1%TFA(AN/H₂O) to yield an off-white solid (516 mg). HPLC/LRMS: >99%, 448 (M+H)+; ¹H NMR (DMF-d₇, 400 MHz) δ 10.21 (2H, br s), 9.41 (2H, br s), 8.76 (1H, m), 7.91 (2H, m), 7.47 (2H, m), 6.87 (1H, s), 6.52 (1H, s), 6.36 (1H, s), 6.30 (1H, s), 4.62 (2H, s), 4.49 (2H, d), 2.94 (1H, m), 0.94 (2H, m), 0.87 (2H, m); HRMS(ES+) calcd. for C₂₃H₂₆N₇O₃ 448.2092, found 448.2055.

### EXAMPLE 180

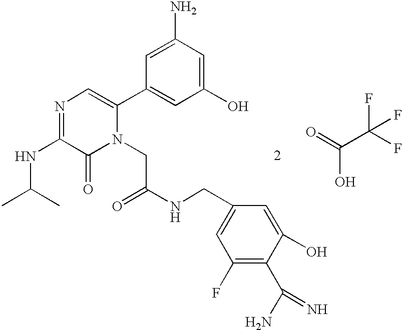

HRMS (M+H)⁺ 484.2070 found for C₂₃H₂₆FN₇O₄; 484.2103 calc'd.

### EXAMPLE 181

2-[6-(3-amino-5-hydroxyphenyl)-3-(isopropylamino)-2-oxopyrazin-1 (2H)-yl]-N-{4-[amino(imino)methyl]-3-hydroxybenzyl}acetamide

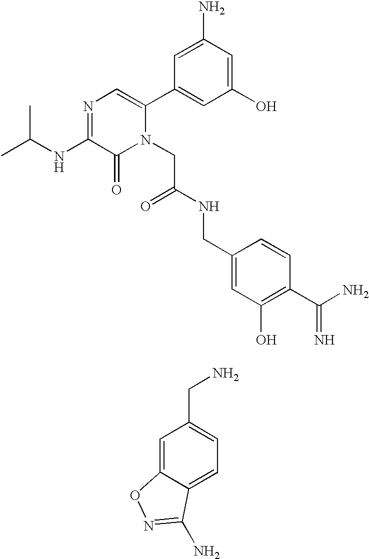

6-(aminomethyl)-1,2-benzisoxazol-3-amine

Ex-181a) To a 250 mL RBF was added di(tert-butyl) (3-amino-1,2-benzisoxazol-6-yl)methylimidodicarbonate (2.5 g, 6.8 mmol) in 4 N HCl in dioxane. The reaction stirred for 1 hour then checked by M.S. and L.C. The starting material was observed to be gone and a new product that corresponded to the mass of the product was observed. The excess HCl and dioxane was removed in vacuo toafford Ex-4a as a white solid that was used without further purification.

M.S. 163.4 (MH+164.5)

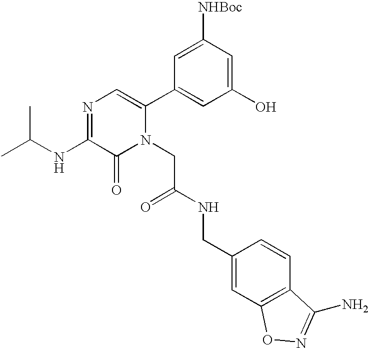

tert-butyl 3-[1-(2-{[(3-amino-1,2-benzisoxazol-6-yl)methyl]amino}-2-oxoethyl)-5-(isopropylamino)-6-oxo-1,6-dihydropyrazin-2-yl]-5-hydroxyphenylcarbamate

Ex-181b) To the 250 mL RBF with Ex-4a(2.0 g, 6.8 mmol) was added the DMF(40 mL) and potassium t-butoxide(6.5 g, 6.8 mmol). The reaction stirred for 20 minutes. To the reaction was added the acid(1.5 g, 3.6 mmol) and TBTU(1.3 g, 4.0 mmol). The reaction stirred at room temperature overnight. The reaction was poured into water and the ppt. ws filtered and washed with additional water. The solid was dissolved in methylene chloride and dried over MgSO4 then concentrated in vacuo to afford Ex-181b (0.85 g) in 19% yield.

M.S. 663.72 (MH+664.1)

NMR (400 MHZ, CDCl₃): ¹H 1.23 ppm (6H, d), 1.49 ppm (9H, s), 1.99 ppm(1H, d), 4.1 ppm (2H, q), 4.46 ppm (2H, s), 4.58 ppm (2H, s), 6.48 ppm (1H, t),6.70 ppm (1H, s), 6.75 ppm (1H, s), 6.98 ppm (1H, t),7.10 ppm (1H, d), 7.36 ppm (1H, t), 7.41 ppm (1H, d), 7.84 ppm (1H, d).

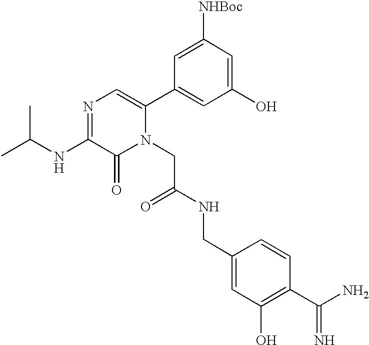

tert-butyl 3-[1-[2-({4-[amino(imino)methyl]-3-hydroxybenzyl}amino)-2-oxoethyl]-5-(isopropylamino)-6-oxo-1,6-dihydropyrazin-2-yl]-5-hydroxyphenylcarbamate

Ex-181c) To a 100 mL flask was added palladium black in MeOH(25 mL) under a blanket of nitrogen. To the slurry was added Ex-4b (1.43 g, 2.54 mmol) and ammonium fomate(2.0 g, 31.7 mmol). The reaction stirred at room temperature for 2 hours. The palladium was filtered off through ceilite and then the reaction was concentrated in vacuo. The resulting solid was tritrated with toluene then dried under a high vacuum. The resulting tan solid(1.40 g) was used without further purification.

M.S. 565.62 (MH+566.3)

Ex-181) To a 50 mL RBF was added Ex-4c (0.23 g, 0.41 mmol) in 20% TFA/methylene chloride(10 mL). The reaction stirred for 1 hour. The reaction was then concentrated in vacuo. The resulting yellow-tan solid was tritrated with diethyl ether then filtered and dried on a high vacuum to afford the title compound (0.18 g) in 95% yield.

Isolated as 2.6TFA and1 H₂O

Found C: 54.08H: 5.55 N: 16.35

Calc. C: 42.30H: 4.16 N: 10.18

### EXAMPLE 182

3-amino-5-[1-[2-({4-[amino(imino)methyl]-2-methoxybenzyl}amino)-2-oxoethyl]-5-(isopropylamino)-6-oxo-1,6-dihydropyrazin-2-yl]-N-[(1R)-1-methylpropyl]benzamide

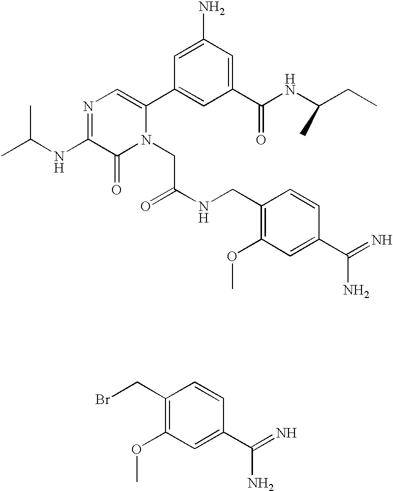

4-(bromomethyl)-3-methoxybenzenecarboximidamide

Ex-182a) NH₄Br (6.9 g, 70 mmol) was taken up in 70 mL of chlorobenzene. The mixture was cooled in an ice bath and flushed well with N₂. AlMe₃ (30 mL of a 2 M solution in heptane, 60 mmol) was added dropwise. The heterogeneous reaction was warmed to room temperature, then slowly heated to 55° C. Most of the NH₄Br dissolved. The methyl 4-bromomethyl-3-methoxybenzoate was added (3.14 g, 12 mmol), followed by 15 mL of chlorobenzene. The reaction was heated to 80° C. under positive N₂ pressure overnight.

The reaction was cooled in an ice bath and slowly quenched with ˜20 mL of MeOH. The mixture was stirred at room temperature ˜2 h, then filtered through Celite. The filtrate was concentrated to remove the MeOH. The solid that crashed out of the remaining chlorobenzene was filtered, washed well with Et₂O, and dried under high vacuum for 1 h to give 3.2 g of a pale yellow solid:

LRMS m/z 243, 245 (M⁺+H); HPLC purity (retention time): >90% (1.1 min).

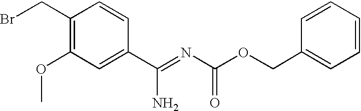

Benzyl (1Z)-amino[4-(bromomethyl)-3-methoxyphenyl]methylidenecarbamate

Ex-182b) The product from Ex-182a (2.9 g, 12 mmol) was taken up in 50 mL of THF. Et₃N (5 mL, 36 mmol) was added. Water (1.5 mL) was added until the solid partially dissolved. The mixture was cooled in an ice bath. Benzyl chloroformate (2.6 mL, 18.2 mmol) in 4 mL of THF (plus a 2 mL rinse) was added dropwise, keeping the temperature below 6° C. The ice bath was removed. LC/MS analysis after 30 mins showed complete reaction.

The reaction mixture was filtered through Celite. The filtrate was concentrated to a yellow oil. The oil was taken up in 50 mL of EtOAc, washed with water 1×25 mL, brine 1×25 mL, dried over MgSO₄, and concentrated. The oil solidified after standing overnight under N₂. The solid was dried under high vacuum for 3 hours to give 2.7 g (60%) of a pale yellow solid: LRMS m/z 377, 379 (M⁺+H); HPLC purity (retention time): 80% (1.9 min).

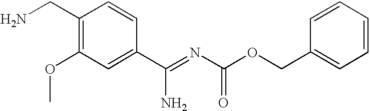

Benzyl (1Z)-amino[4-(aminomethyl)-3-methoxyphenyl]methylidenecarbamate

Ex-182c) The crude product from Ex-182b (2.6 g crude, 6.9 mmol crude) was added to ˜30 mL of NH₃ condensed in a pressure flask at ˜78° C. The pressure flask was tightly capped and warmed to room temperature. The heterogeneous reaction became homogeneous. LC/MS analysis after 1.5 hours showed complete reaction.

The mixture was filtered through Celite and concentrated to give 2.1 g (95% crude yield) of a yellow oil: LRMS m/z 314.1, 610.2 (dimer) (M⁺+H); HPLC purity (retention time): 70% (1.2 min), 10% (1.9 min, dimer).

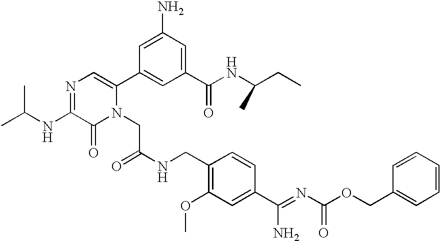

Benzyl (1Z)-amino{4-[({[3-(isopropylamino)-6-[3-({[(1R)-1-methylpropyl]amino}carbonyl)-5-nitrophenyl]-2-oxopyrazin-1 (2H)-yl]acetyl}amino)methyl]-3-methoxyphenyl}methylidenecarbamate

Ex-1d) LRMS m/z 727.3 (M⁺+H); HPLC purity (retention time): 60% (2.3 min).

Ex-182) The crude product from Ex-182d (0.25 g crude, 0.34 mmol) was dissolved in 2 mL of MeOH. Pd/C (10% Pd/C, 50% water-wet) (48 mg, 0.02 mmol) in 2 mL of N₂-flushed MeOH was added. The reaction was evacuated and flushed with N₂ several times. Repeated with H₂, then stirred under a H₂ balloon for 2 h. The mixture was filtered through Celite and the filtrate was concentrated.

The dark yellow oil was purified by reverse phase HPLC with a gradient of 15/85%-50/50% acetonitrile/water (+0.1% TFA) over 12 mins. Product eluted at 4 mins, 23% acetonitrile, giving 46 mg (24%) of a white solid: LRMS m/z 563.3 (M⁺+H); HPLC purity (retention time): 98% (1.4 min); HRMS (M+H): Calc'd for C₂₉H₃₈N₈O₄: 563.3089; Found: 563.3084; ¹H NMR (300 MHz, CD₃OD) δ 0.91 (t, 3H, J=7.35 Hz), 1.17 (d, 3H, J=6.6 Hz), 1.39 (d, 6H, J=6.45 Hz), 1.55 (m, 2H), 3.92 (s, 3H), 3.94-4.09 (m, 2H), 4.38 (s, 2H), 4.64 (s, 2H), 6.64 (s, 1H), 6.89 (s, 1H), 7.08 (s, 1H), 7.18-7.35 (m, 5H), 8.60 (br t, ˜1H).

### EXAMPLES 183/184

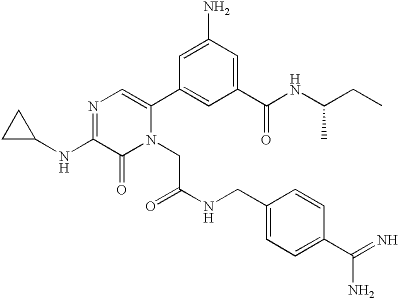

HPLC/LRMS: >95%, 531 (M+H)+; HRMS(ES+) calcd. for C₂₈H₃₅N₈O₃ 531.2827, found 531.2796.

Two salts were prepared for this compound and the data for each is recorded in Table 3.

### EXAMPLE 185

3-[1-[2-({4-[amino(imino)methyl]-2,3,6-trifluoro-5-hydroxybenzyl}amino)-2-oxoethyl]-5-(isopropylamino)-6-oxo-1,6-dihydropyrazin-2-yl]-N-[(1R)-1-methylpropyl]-5-aminobenzamide Dihydrochloride

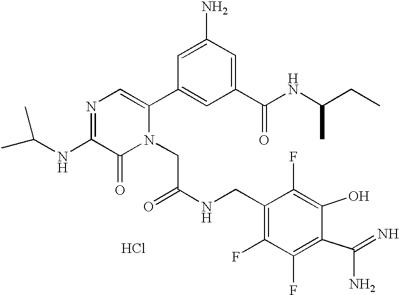

HRMS: (M+H)+603.2684 found for C₂₈H₃₃F₃N₈O₄; 603.2650 calc'd.

¹H-NMR: 400 MHz, DMSO-d₆ δ 9.57 (bs, 2H), 9.52 (bs, 2H), 9.19 (bm, 1H), 8.84 (t, J=5.5 Hz, 1H), 8.36 (d, J=8.2 Hz, 1H), 7.57 (s, 1H), 7.48 (s, 1H), 7.19 (s, 1H), 6.67 (s, 1H), 4.38 (bs, 2H), 4.24-4.34 (m, 3H), 3.84 (septet, J=6.8 Hz, 1H), 1.46 (m, 2H), 1.22 (d, J=6.8 Hz, 6H), 1.08 (d, J=6.8 Hz, 3H), 0.812 (t, J=6.8 Hz, 3H).

### EXAMPLE 186

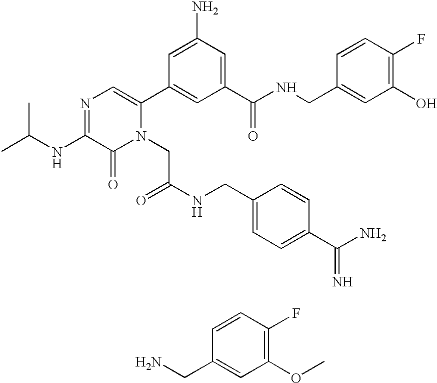

4-fluoro-3-methoxybenzylamine

Ex-186a) To 4-fluoro-3-methoxybenzonitrile (2.55 g, 16.9 mmol) in 75 ml of ethanol was added 0.85 g of 10% palladium on carbon and 7.5 ml of hydrogen chloride (conc.). The mixture was shaken on the Parr apparatus under 20 Psi of hydrogen for 5.5 hours. The mixture was filtered and concentrated in vacuo to give 3.19 g (99% yield) of a light pink solid. m/z(M+H)+156

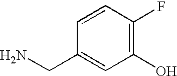

5-(aminomethyl)-2-fluorophenol

Ex-186b) The product from 186a (3.07 g, 16.1 mmol) in 9 ml of hydrogen chloride (conc.) was heated at 125° C. for 8 hours in a sealed tube. The solution was treated with 75 ml of ethanol and concentrated in vacuo to give 2.88 g (quantitative yield) of a tan solid. m/z(M+H)+142

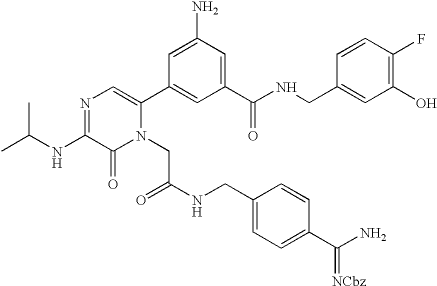

Ex-186c) m/z(M+H)+765

Ex-186) To the product from 186c (0.87 g, 1.14 mmol) and 0.28 g of 10% palladium on carbon under nitrogen was added 10 ml of methanol (anhyd.) and then ammonium formate (0.323 g, 5.12 mmol). The mixture was heated at reflux for 2 hours. The mixture was filtered and concentrated in vacuo and purified by reverse phase chromatography with 10-35% CH₃CN/H₂O to give 0.46 g (46% yield) of an off-white solid. m/z(M+H)⁺601

Analysis: C₃₁H₃₃N₈O₄+2.45 TFA+1.55H₂O calcd: C, 47.49; H, 4.28; N, 12.34; found: C, 47.45; H, 4.22; N, 12.42. HRMS calcd: 601.2682; Found: 601.2670

¹H NMR(400 MHz, DMSO): 1.22 (6H, d), 4.09 (1H, m), 4.33 (4H, m), 4.43 (2H, s), 6.71 (3H, m), 6.89 (1H, m), 7.03 (2H, m), 7.16 (1H, s), 7.37 (2H, d), 7.38 (1H, br s), 7.73 (2H, d), 8.68 (1H, t), 8.84 (1H, t), 9.08 (2H, s), 9.25 (2H, s).

### EXAMPLE 187

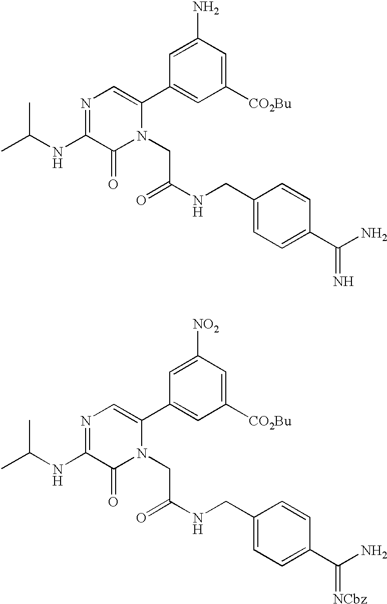

Ex-187a) m/z(M+H)⁺698

Ex-187) To the product from Ex-187a (1.11 g, 1.60 mmol) and 0.40 g of 10% palladium on carbon under nitrogen was added 14 ml of methanol (anhyd.) and then ammonium formate (0.462 g, 7.34 mmol). The mixture was heated at reflux for 2 hours. The mixture was filtered and concentrated in vacuo to give 0.80 g of a yellow solid (94% yield). A 100 mg portion of this solid was purified by reverse phase chromatography with 10-45% CH₃CN/H₂O to give 60 mg of a white solid. m/z(M+H)+534

Analysis: C₂₈H₃₅N₇O₄+1.90 TFA+1.15H₂O calcd: C, 49.54; H, 5.15; N, 12.72; found: C, 49.53; H, 5.15; N, 12.69.

HRMS calcd: 534.2801; Found: 534.2823

¹H NMR(400 MHz, DMSO): 0.91 (3H, t), 1.22 (6H, d), 1.40 (2H, sextet), 1.64 (2H, quintet), 4.09 (1H, m), 4.21 (2H, m), 4.38 (4H, br s), 6.69 (1H, s), 6.78 (1H, s), 7.11 (1H, s), 7.27 (1H, s), 7.30 (1H, br s), 7.39 (2H, d), 7.73 (2H, d), 8.68 (1H, br s) 9.12 (2H, br s), 9.27 (2H, br s)

### EXAMPLE 188

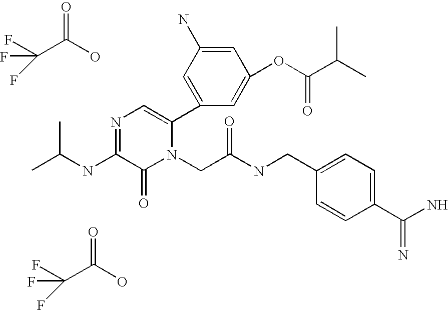

Prepared analogously to Example 55.

### EXAMPLE 189

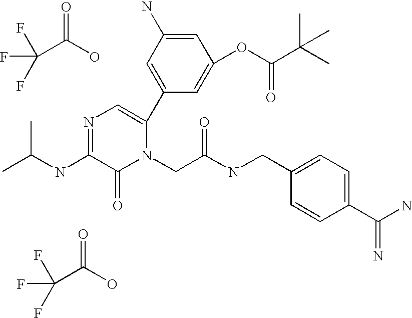

Prepared analogously to Example 55.

### EXAMPLE 190

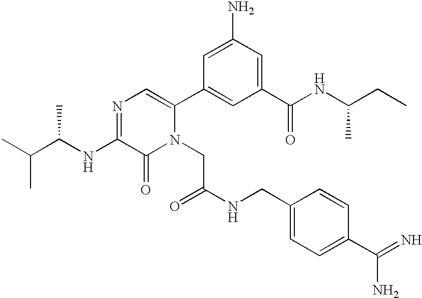

HPLC/LRMS: >97%, 561 (M+H)+; HRMS(ES+) calcd. for C₃₀H₄₁N₈O₃ 561.3296, found 561.3288.

### EXAMPLE 191

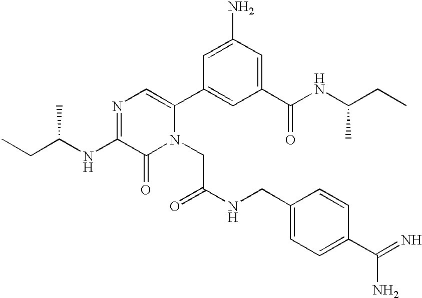

HPLC/LRMS: >98%, 547 (M+H)+; HRMS(ES+) calcd. for C₂₉H₃₉N₈O₃ 547.3140, found 547.3158.

### EXAMPLE 192

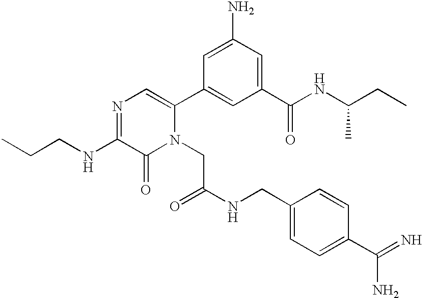

HPLC/LRMS: >98%, 533 (M+H)+; HRMS(ES+) calcd. for C₂₈H₃₇N₈O₃ 533.2983, found 533.2961.

### EXAMPLE 193

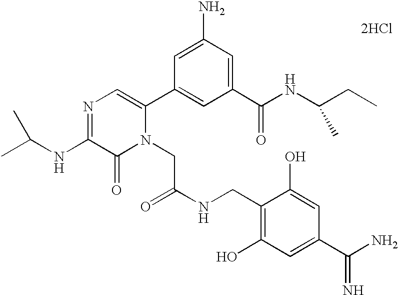

The product from Ex-196 (0.090 g, 0.00013 mol) was suspended in anhydrous CH₂Cl₂ (5 mL) and cooled to −80° C. BBr₃ (1M soln in CH₂Cl₂, 0.25 mL) was added and the solution was heated to reflux for 2 hours. After 2 hrs additional BBr₃ (0.25 mL) was added. After refluxing for a total of 4.5 hrs the solution was cooled to 0° C. and 5 mL of methanol was added. The solution was concentrated and the residue was coevaporated with methanol (4×5 mL). The crude product was purified by prep HPLC (RP, 15% isocratic, acetonitrile in 0.1% TFA). The purified TFA salt was coevaporated with 1 N HCl and lyophilized from water to yield Ex-193 (0.0217 g, 26.2%) as a yellow solid.

HRMS: (M+H)+565.2881 found for C₂₈H₃₆N₈O₅; 565.2809 calc'd. ¹H-NMR, 400 mHz, CD₃OD δ 7.61 (d, 2H, J=8.0 Hz), 7.34 (s 1H), 6.69 (s, 3H), 4.54 (s, 2H), 4.35 (s, 2H), 4.07-3.97 (m, 2H), 1.67-1.55 (m, 2H), 1.40 (d, 6H, J=6.4 Hz), 1.24 (d, 3H, J=6.6 Hz), 0.96 (t, 3H, J=7.4 Hz).

### EXAMPLE 194

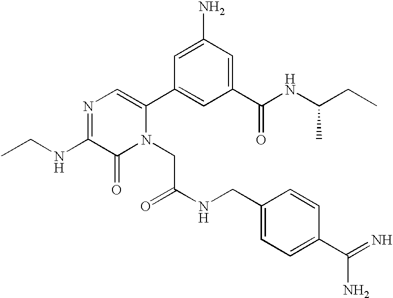

HPLC/LRMS: >97%, 519 (M+H)+; HRMS(ES+) calcd. for C₂₇H₃₅N₈O₃ 519.2827, found 519.2815.

### EXAMPLE 195

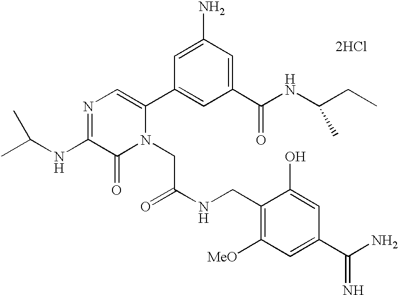

Ex-195 was prepared analogously to Ex-193.

HRMS: (M+H)+579.3038 found for C₂₉H₃₈N₈O₅; 579.2965 calc'd. ¹H-NMR, 400 mHz, CD₃OD δ 7.56 (s, 1H), 7.52 (s, 1H), 7.27 (s, 1H), 6.85 (s, 2H), 6.67 (s, 1H), 4.53 (s, 2H), 4.37 (s, 1H), 4.05-4.00 (m, 2H), 3.88 (s, 3H), 1.63-1.59 (m, 2H), 1.40 (d, 6H, J=5.6 Hz), 1.23 (d, 3H, J=6.7 Hz), 0.96 (t, 3H, J=7.4 Hz).

### EXAMPLE 196

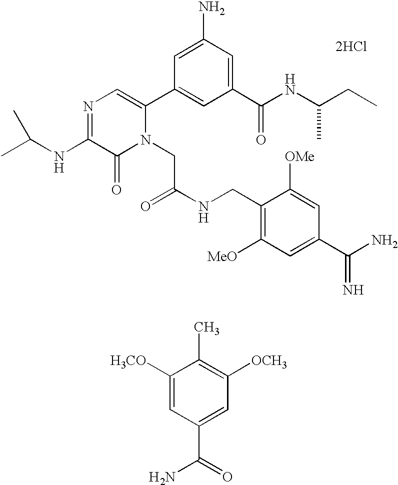

3,5-dimethoxy-4-methylbenzamide

Ex-196a) 3,5-dimethoxy-4-methylbenzoic acid (50 g, 0.255 mol) was dissolved in DMF (500 mL) to this solution was added DIEA (100 mL, 0.573 mol) and HBTU (100 g, 0.263 mol). The reaction was stirred at room temperature overnight. Additional HBTU (19 g, 0.05 mol) was added and the reaction stirred for an additional 30 min. The solution was cooled to 0□C. and NH₄OH (20 mL) was added dropwise over 6 min. The solvent was removed and the residue treated with CH₂Cl₂ (500 mL) and water (300 mL). The precipitate that formed was collected by filtration. The CH₂Cl₂ layer from the filtrate was concentrated and extracted with water. The precipitate and solid from the CH₂Cl₂ layer were combined to give Ex-196a (37.4 g, 75.2%) as a white solid.

¹H-NMR, 300 mHz, DMSO-d₆ □ 7.94 (s, 1H), 7.28 (s, 1H), 7.12 (s, 2H), 3.78 (s, 6H), 1.99 (s, 3H).

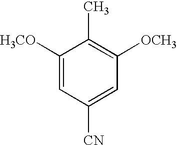

3,5-dimethoxy-4-methylbenzonitrile

Ex-196b) 3,5-dimethoxy-4-methylbenzamide, Ex-196a (30 g, 0.154 mol) was suspended in 550 mL of toluene. To this solution was added thionyl chloride (14.8 mL, 0.20 mol) and a catalytic amount of DMF (2 mL). The resulting solution was heated to 75° C. for 4 hours. After cooling to room temperature the solution was concentrated in vacuo. The residue was recrystallized from acetonitrile to give 21.6 g (79.4%) of Ex-196b as off-white crystals.

¹H-NMR, 300 mHz, CDCl₃ δ 6.72 (s, 2H), 3.78 (s, 6H), 2.05 (s, 3H)

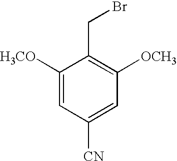

4-(bromomethyl)-3,5-dimethoxybenzonitrile

Ex-196c) 3,5-dimethoxy-4-methylbenzonitrile, Ex-196b (41.9 g, 0.24 mol) was dissolved in CCl₄ (1000 mL) followed by addition of N-bromosuccinimide (50.5 g, 0.28 mol) and benzoyl peroxide (9.2 g, 0.038 mol). The reaction mixture was refluxed for two hours. The reaction was cooled, filtered and concentrated. The residue was coevaporated two times with methanol. The crude product was recrystallized from acetonitrile/ether to give 41.9 g (69.4%) of an off-white solid.

¹H-NMR, 300 mHz, CDCl₃ δ 6.77 (s, 2H)₁ 4.57 (s, 2H), 3.89 (s, 6H).

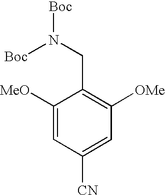

di(tert-butyl) 4-cyano-2,6-dimethoxybenzylimidodicarbonate

Ex-196d) Sodium hydride (3.84 g, 0.15 mol) was dissolved in anhydrous DMF (1000 mL). To this solution was added di-tert-butyl iminodicarboxylate (34.7 g, 0.16 mol). After stirring at room temperature for 45 min 4-(bromomethyl)-3,5-dimethoxybenzonitrile, Ex-196c (40 g, 0.16 mol) was added as a solution in DMF (100 mL). The solution was stirred for 2.5 days. The reaction mixture was cooled with an ice bath and diluted with water (500 mL). The entire mixture was extracted with ethyl acetate (3×650 mL). The ethyl acetate layers were combined, dried over MgSO₄, filtered and concentrated. The dark yellow oil obtained was recrystallized from hexane/ether to give 42.9 g (68.4%) of an off-white solid.

HRMS: (M+Na)+415.1840 found for C₂₀H₂₈N₂NaO₆; 415.1845 calc'd.

¹H-NMR, 300 mHz, CDCl₃ δ 6.75 (s, 2H), 4.86 (s, 2H), 3.77 (s, 6H), 1.39 (s, 18H)

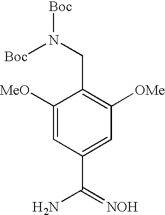

di(tert-butyl) 4[amino(hydroxyimino)methyl]-2,6-dimethoxybenzylimidodicarbonate

Ex-196e) Hydroxylamine hydrochloride (10.6 g, 0.15 mol) was dissolved in methanol (400 mL) followed by addition of diisopropylethylamine (26.7 mL 0.15 mol). To this solution was added di(tert-butyl) 4-cyano-2,6-dimethoxybenzylimidodicarbonate, Ex-196d (15 g, 0.038 mol). The solution was stirred at room temperature overnight. The reaction had not proceeded to completeness, therefore, additional hydroxylamine hydrochloride (0.019 mol) and diisopropylethylamine (0.076 mol) were added. The solution was stirred for an additional hour. The solvent was evaporated and the residue was partitioned between ethyl acetate (200 mL) and water (50 mL). The ethyl acetate was washed with saturated aqueous sodium chloride (60 mL), dried over MgSO₄, filtered and concentrated to give 15.5 g of Ex-196e (94.7%) of an off-white solid.

¹H-NMR, 300 mHz, CDCl₃ δ 6.77 (s, 2H), 4.88 (s, 2H), 3.79 (s, 6H), 1.40 (s, 18H).

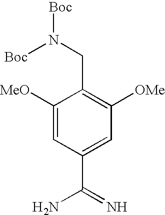

di(tert-butyl) 4-[amino(imino)methyl]-2,6-dimethoxybenzylimidodicarbonate

Ex-196f) Di(tert-butyl) 4[amino(hydroxyimino)methyl]-2,6-dimethoxybenzylimidodicarbonate, Ex-196e (14.5 g, 0.034 mol) was dissolved in acetic acid (200 mL) followed by addition of acetic anhydride (4.5 g, 0.044 mol) and 10% Pd/C (1.5 g). The mixture was placed at 50 psi of H₂ pressure overnight. The reaction mixture was filtered through celite and concentrated to give 12.9 g (92.7%) of Ex-196f as an off-white powder.

HRMS: (M+H)+410.2286 found for C₂₀H₃₁N₃O₆; 410.2213 calc'd.

¹H-NMR, 300 mHz, DMSO-d₆ δ 7.04 (s, 2H), 4.71 (s, 2H), 3.79 (s, 6H), 1.35 (s, 18H).

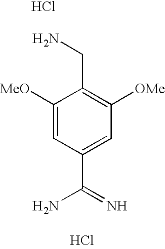

4-aminomethyl-3,5-dimethoxybenzamidine Hydrochloride

Ex-196 g) Di(tert-butyl) 4-[amino(imino)methyl]-2,6-dimethoxybenzylimidodicarbonate, Ex-196f (5.0 g, 0.012 mol) was dissolved in 4 N HCl/dioxane (20 mL). The solution was stirred for 2 hours at room temperature. The reaction mixture was filtered and the solid was washed with ether and dried to give 3.0 g (88.3%) of Ex-196 g as an off-white powder.

HRMS: (M+H)+210.1216 found for C₁₀H₁₅N₃O₂; 210.1164 calc'd.

¹H-NMR, 400 mHz, DMSO-d₆ δ 9.70 (s, 1H), 9.36 (s, 1H), 8.23 (NH2), 7.25 (s, 2H), 3.90 (s, 2H), 3.35 (s, 6H).

Ex-196) The carboxylic acid, [3-(isopropylamino)-6-[3-({[(1S)-1-methylpropyl]amino}carbonyl)-5-nitrophenyl]-2-oxopyrazin-1 (2H)-yl]acetic acid (0.500 g, 0.0012 mol) was dissolved in anhydrous DMF (15 mL). To this solution was added diisopropylethylamine (0.24 mL, 0.0014 mol) followed by HBTU (0.527 g, 0.0014 mol). The resulting solution was stirred for one hour followed by addition of the benzyl amine, 4-aminomethyl-3,5-dimethoxybenzenecarboximidamide dihydrochloride, Ex-196 g (0.397 g, 0.0014 mol). The solution was stirred for 4 hrs at room temperature. The solvent was evaporated and the crude product was dissolved in methanol (40 mL). Nitrogen was bubbled through the solution. A solution of 4 N HCl in dioxane (2 mL) was added followed by 10% Pd/C (0.30 g). The solution was placed at 40 psi of hydrogen pressure for 5 hours. The reaction mixture was filtered through celite and the celite was washed with methanol. The filtrate was concentrated and the crude residue was purified by prep HPLC (RP, 5-50% gradient, acetonitrile in 0.1% TFA). The purified TFA salt was coevaporated with 1 N HCl and lyophilized from water to give 0.45 g (56.3%) of Ex-196 as a yellow solid.

HRMS: (M+H)+593.3194 found for C₃₀H₄₀N₈05; 593.3122 calc'd.

¹H-NMR, 400 mHz, CD₃OD δ 9.26 (br s, NH), 8.84 (br s, NH), 7.98 (br s, 2H), 7.69 (s, 1H), 7.03 (s, 2H), 6.74 (s, 1H), 4.52 (s 2H), 4.38 (s, 2H), 4.12-4.06 (m, 1H), 4.06-3.99 (m, 1H), 3.89 (s, 6H), 1.69-1.57 (m, 2H), 1.41 (d, 6H, J=6.4 Hz), 1.25 (d, 3H, J=6.7 Hz), 0.96 (t, 3H, J=7.4 Hz).

### EXAMPLE 197

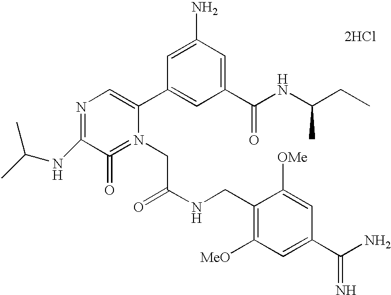

Ex-197 was prepared analogously to Ex-196, except the R-isomer of the carboxylic acid, [3-(isopropylamino)-6-[3-({[(1R)-1-methylpropyl]amino}carbonyl)-5-nitrophenyl]-2-oxopyrazin-1 (2H)-yl] acetic acid, was used. The purified TFA salt was coevaporated with 1N HCl to give Ex-197 (0.412 g, 53.6%) as a pale yellow solid.

HRMS: (M+H)+593.3181 found for C₃₀H₄₀N₈O₅; 593.3122 calc'd.

¹H-NMR, 400 mHz, CD₃OD δ 9.25 (br s), 8.82 (br s, 1H), 7.95 (br s, 2H), 7.66 (br s, 1H), 7.04 (s, 2H), 6.73 (s, 1H), 4.52 (s, 2H), 4.30 (s, 2H), 4.10-4.00 (m, 1H), 3.89 (s, 6H), 1.64-1.62 (m, 2H), 1.41 (d, 6H, J=6.4 Hz), 1.25 (d, 3H, J=6.6 Hz), 0.97 (t, 3H, J=7.4 Hz).

### EXAMPLE 198

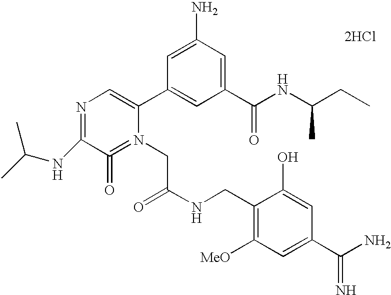

Ex-198 was prepared analogously to Ex-195, except that the product from Ex-195 was used as the starting material. The purified TFA salt was coevaporated with 1N HCl and lyophilized from water to give Ex-198 (0.028 g, 27.5%) as a pale yellow solid.

HRMS: (M+H)⁺579.3038 found for C₂₉H₃₈N₈O₅; 579.2965 calc'd.

¹H-NMR, 400 mHz, CD₃OD δ 7.75 (s, 2H), 7.48 (s, 1H), 6.86 (s, 2H), 6.71 (s, 1H), 4.54 (s, 2H), 4.36 (s, 2H), 4.08-4.01 (m, 1H), 3.88 (s, 3H), 1.65-1.60 (m, 1H), 1.41 (d, 6H, J=6.4 Hz), 1.24 (d, 3H, J=6.7 Hz), 0.96 (t, 3H, J=7.4 Hz).

### EXAMPLE 199

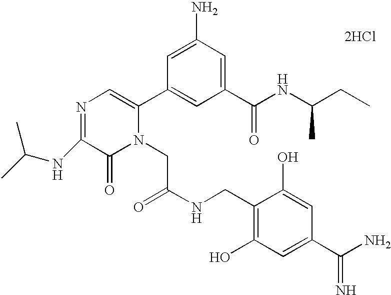

Ex-199 was prepared analogously to Ex-193 except the product from Ex-193 was used as the starting material. The purified TFA salt was co-evaporated with 1N HCl and lyophilized from water to give Ex-199 (0.144 g, 95.7%) as a pale yellow solid.

HRMS: (M+H)+565.2853 found for C₂₈H₃₆N₈O₅; 565.2809 calc'd.

¹H-NMR, 400 mHz, CD₃OD δ 7.76 (br s, 2H), 7.53 (s, 1H), 6.71 (s, 1H), 6.68 (d, 1H, J=7.6 Hz), 4.55 (s, 2H), 4.34 (s, 2H), 4.08-4.01 (m, 1H), 1.65-1.60 (m, 2H), 1.41 (d, 6H, J=6.4 Hz), 1.24 (d, 3H, J=6.8 Hz), 0.97 (t, 3H, J=7.4 Hz).

### EXAMPLE 200

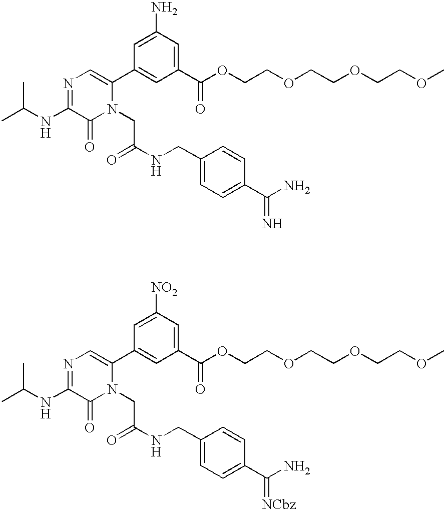

Ex-200a) m/z(M+H)+788

Ex-200) To the product from Ex-200a (1.38 g, 1.75 mmol) and 0.43 g of 10% palladium on carbon under nitrogen was added 15 ml of methanol (anhyd.) and then ammonium formate (0.497 g, 7.89 mmol). The mixture was heated at reflux for 2 hours. The mixture was filtered and concentrated in vacuo to give 1.10 g of a yellow foam (quantitative yield). A 120 mg portion of this solid was purified by reverse phase chromatography with 5-35% CH₃CN/H₂O to give 60 mg of a white solid. m/z(M+H)+624 Analysis: C₃₁H₄₁N₇O₇+2.25 TFA+1.15H₂O calcd: C, 47.33; H, 5.10; N, 10.88; found: C, 47.31; H, 5.03; N, 10.94.

HRMS calcd: 624.3140; Found: 624.3143

¹H NMR(400 MHz, DMSO): 1.22 (6H, d), 3.20 (3H, s), 3.38 (2H, m), 3.47-3.57 (6H, m), 3.70 (2H, m), 4.10 (1H, m), 4.30-4.39 (6H, m), 6.69 (1H, s), 6.78 (1H, t), 7.11 (1H, t), 7.27 (1H, t), 7.40 (2H, d), 7.73 (2H, d), 8.67 (1H, t), 9.04 (2H, s), 9.26 (2H, s).

### EXAMPLE 201

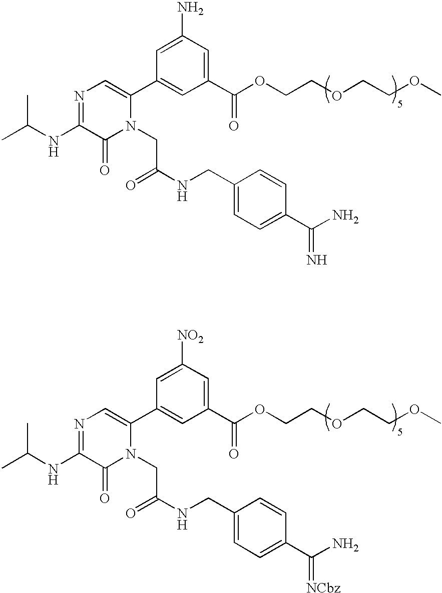

Ex-201a) m/z (M+H)+920

Ex-201) To the product from Ex-201a (1.59 g, 1.73 mmol) and ammonium formate (0.490 g, 7.78 mmol) in 15 ml of methanol (anhyd.) under nitrogen was added 0.43 g of 10% palladium on carbon. The mixture was heated at reflux for 2 hours. The mixture was filtered and concentrated in vacuo. The residue was purified by reverse phase chromatography with 5-35% CH₃CN/H₂O to give 270 mg (15% yield) of a foam. m/z(M+H)⁺756

Analysis: C₃₇H₅₃N₇O₁₀+2.60 TFA+1.25H₂O calcd: C, 47.16; H, 5.45; N, 9.12; found: C, 47.18; H, 5.53; N, 9.01.

HRMS calcd: 756.3927; Found: 756.3935

¹H NMR(400 MHz, DMSO): 1.22 (6H, d), 3.22 (3H, s), 3.39-3.58 (20H, m), 3.71 (2H, m), 4.09 (1H, m), 4.32-4.39 (6H, m), 6.70 (1H, s), 6.79 (1H, t), 7.11 (1H, t), 7.28 (1H, t), 7.40 (2H, d), 7.73 (2H, d), 8.68 (1H, t), 9.09 (2H, s), 9.26 (2H, s).

### EXAMPLE 202

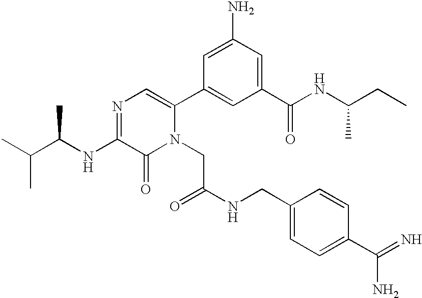

HPLC/LRMS: >98%, 561 (M+H)+; HRMS(ES+) calcd. for C₃₀H₄₁N₈O₃ 561.3296, found 561.3285.

### EXAMPLE 203

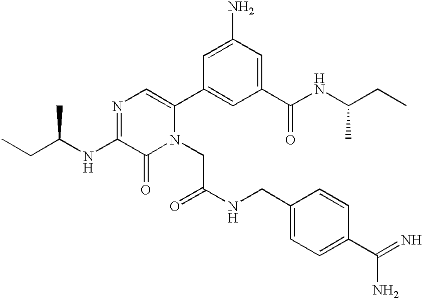

HPLC/LRMS: >98%, 547 (M+H)+; HRMS(ES+) calcd. for C₂₉H₃₉N₈O₃ 547.3140, found 547.3131.

### EXAMPLE 204

3-amino-5-[1-[2-({4-[amino(imino)methyl]benzyl}amino)-2-oxoethyl]-5-(cyclopropylamino)-6-oxo-1,6-dihydropyrazin-2-yl]benzoic Acid

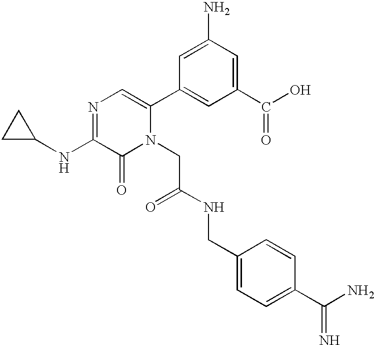

¹H NMR (400 MHz, CD₃OD): δ 7.73 (d, 2H), 7.47 (t, 1H), 7.39 (d, 2H), 7.31 (t, 1H), 6.91 (t, 1H), 6.68 (s, 1H), 4.59 (s, 2H), 4.44 (d, 2H), 2.80-2.76 (m, 1H), 1.09-1.04 (m, 2H), 0.88-0.84 (m, 2H); MS-ESI (M+H)=476; Analysis: C₂₄H₂₅N₇O₄+2.45 TFA+1.05H₂O calcd: C, 44.85; H, 3.84; N, 12.67; found: C, 44.85; H, 3.84; N, 12.65.

### EXAMPLE 205

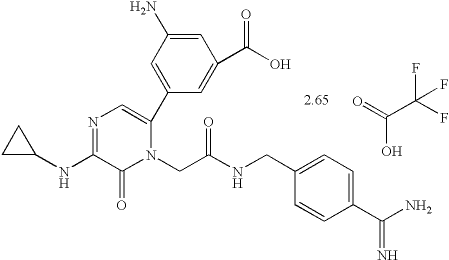

Salt of Example 204.

### EXAMPLE 206

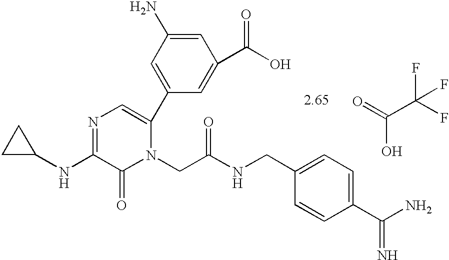

Salt of Example 204.

### EXAMPLE 207

Ethyl 3-amino-5-[1-[2-({4-[amino(imino)methyl]benzyl}amino)-2-oxoethyl]-5-(cyclopropylamino)-6-oxo-1,6-dihydropyrazin-2-yl]benzoate

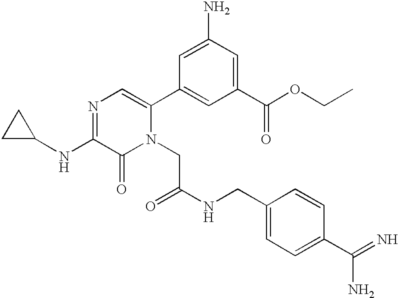

HRMS calcd for C₂₆H₂₉N₇O₄ (M+H): 504.2354. Found: 504.2318.

Anal. Calcd for C₂₆H₂₉N₇O₄+2.4TFA:

C: 46.77; H: 4.13; N: 12.81.

Found: C: 46.92; H: 4.32; N: 12.56.

¹H NMR (DMSO-d₆, 300 MHz) δ 0.66 (m, 2H), 0.75 (m, 2H), 1.27 (t, 3H), 2.75 (m, 1H), 4.25 (q, 2H), 4.36 (m, 4H), 6.74 (s, 1H), 6.77 (s, 1H), 7.11 (s, 1H), 7.27 (s, 1H), 7.38 (d, 2H), 7.71 (d, 2H), 8.67 (t, 1H), 9.02 (s, 2H), 9.25 (s, 2H).

### EXAMPLE 208

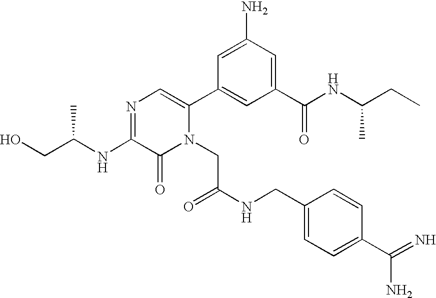

HPLC/LRMS: >97%, 549 (M+H)+; HRMS(ES+) calcd. for C₂₈H₃₇N₈O₄ 549.2932, found 549.2912.

### EXAMPLE 209

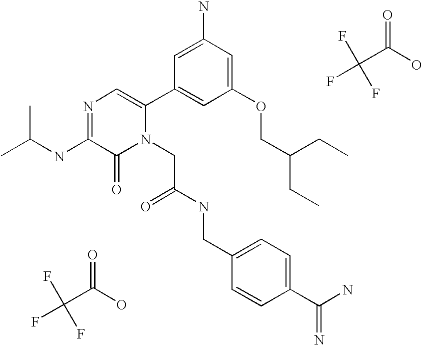

Prepared analogously to Example 64.

### EXAMPLE 210

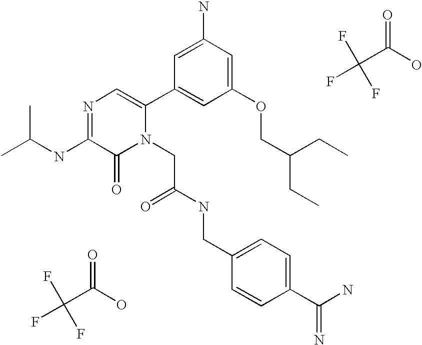

Prepared analogously to Example 64.

### EXAMPLE 211

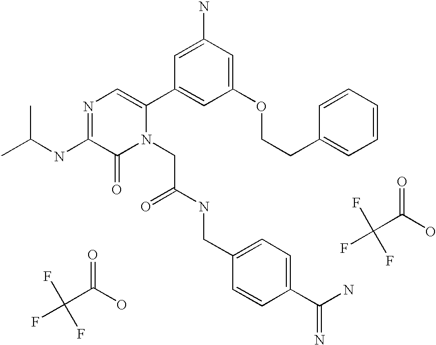

Prepared analogously to Example 64.

### EXAMPLE 212

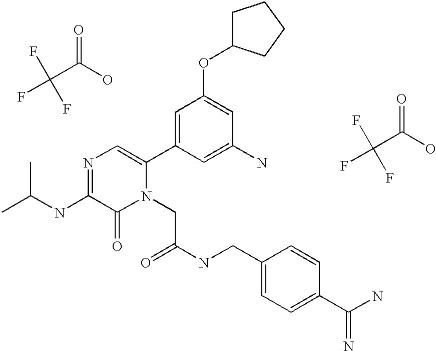

Prepared analogously to Example 64.

### EXAMPLE 213

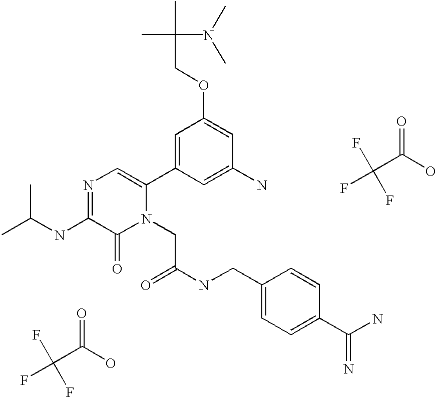

Prepared analogously to Example 64.

### EXAMPLE 214

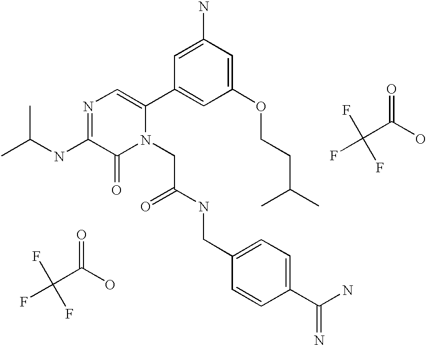

Prepared analogously to Example 64.

### EXAMPLE 215

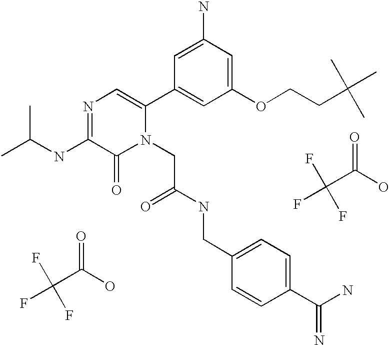

Prepared analogously to Example 64.

### EXAMPLE 216

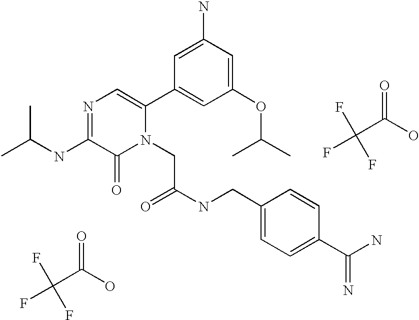

Prepared analogously to Example 64.

### EXAMPLE 217

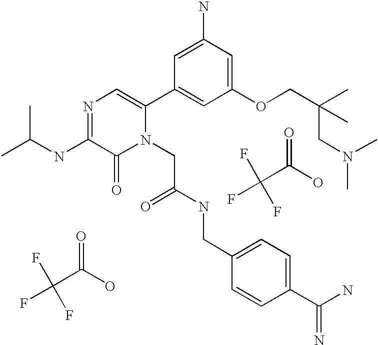

Prepared analogously to Example 64.

### EXAMPLE 218

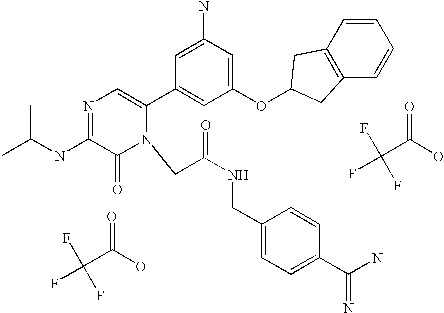

Prepared analogously to Example 64.

### EXAMPLE 219

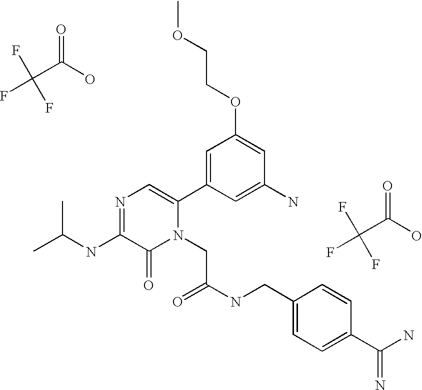

Prepared analogously to Example 64.

### EXAMPLE 220

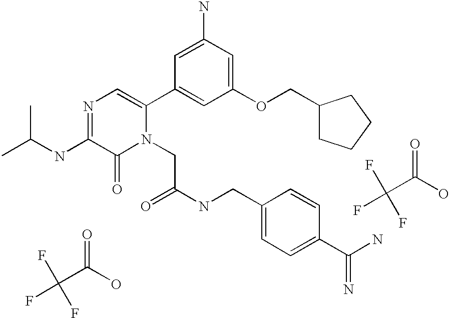

Prepared analogously to Example 64.

### EXAMPLE 221

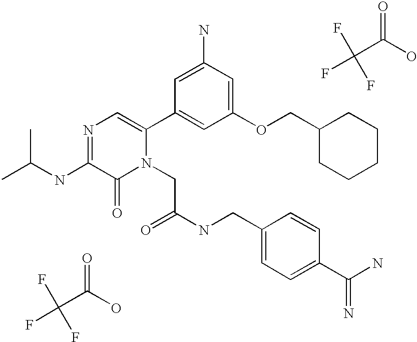

Prepared analogously to Example 64.

### EXAMPLE 222

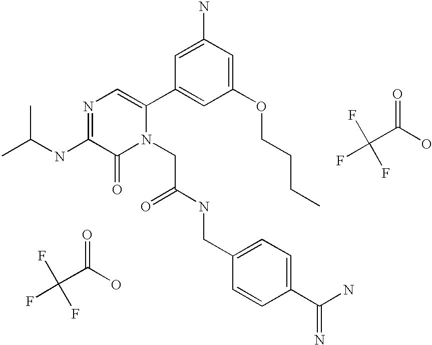

Prepared analogously to Example 64.

### EXAMPLE 223

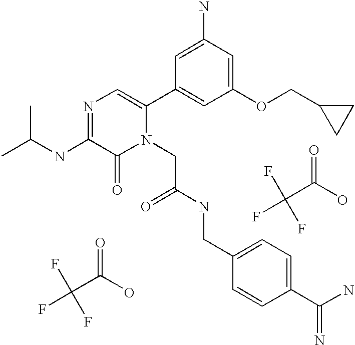

Prepared analogously to Example 64.

### EXAMPLE 224

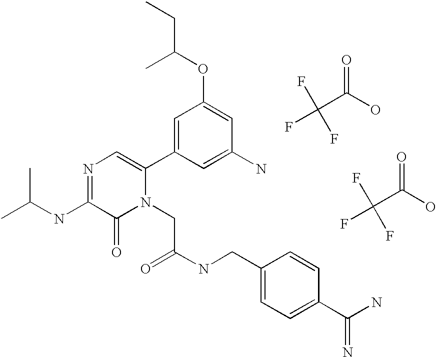

Prepared analogously to Example 64.

### EXAMPLE 225

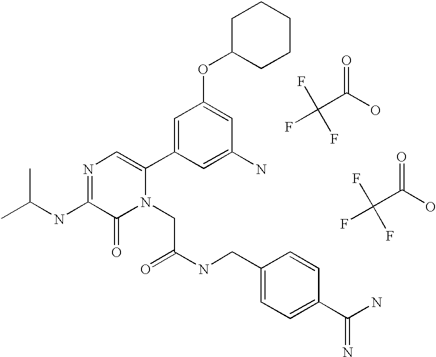

Prepared analogously to Example 64.

### EXAMPLE 226

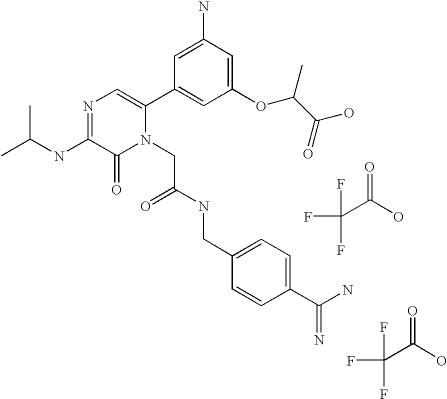

Prepared analogously to Example 64.

### EXAMPLE 227

N-{4-[amino(imino)methyl]benzyl}-2-[6-[3-amino-5-(1H-tetraazol-5-yl)phenyl]-3-(isopropylamino)-2-oxopyrazin-1 (2H)-yl]acetamide

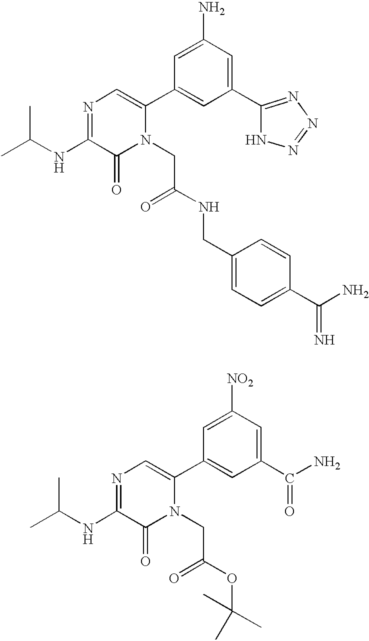

Tert-Butyl [6-[3-(aminocarbonyl)-5-nitrophenyl]-3-(isopropylamino)-2-oxopyrazin-1 (2H)-yl]acetate

Ex-227a) ¹H NMR (300 MHz, CDCl₃): δ 8.75 (t, 1H), 8.40 (t, 1H), 8.23 (t, 1H), 8.02 (s, 1H), 6.85 (bs, 2H), 6.15 (d, 1H), 4.36 (s, 2H), 4.18-4.11 (m, 1H), 1.45 (s, 9H), 1.29 (d, 6H); MS-ESI (M+H)=432.

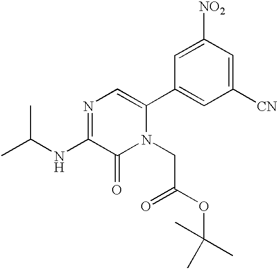

Tert-Butyl [6-(3-cyano-5-nitrophenyl)-3-(isopropylamino)-2-oxopyrazin-1 (2H)-yl]acetate

Ex-227b) To a solution of 227a (1.87 g, 4.3 mmol) in dichloromethane (20 mL) at 0° C. was added TEA (0.6 mL, 9.5 mmol) and TFAA (0.7 mL, 4.8 mmol). After stirring at room temperature for 1 hr the reaction mixture was poured into water and dichloromethane. The layers were separated and organic layer washed with sodium carbonate and brine. The solvent was removed in vacuo to give an oil. The oil was taken up in MeOH:H₂O (20 mL:2 mL). To the solution was added Na₂CO₃ (0.79 g, 7.5 mmol) at 0° C. The reaction was stirred at room temperature for 5 hrs and then acidified with 1N HCl and extracted with ethyl acetate. The organic extracts were combined and washed with brine and dried (Na₂SO₄). The solvent was removed in vacuo to give an orange oil (1.39 g, 98%). MS-ESI (M+H)=414.

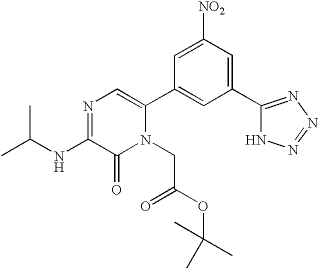

Tert-Butyl [3-(isopropylamino)-6-[3-nitro-5-(1H-tetraazol-5-yl)phenyl]-2-oxopyrazin-1 (2H)-yl]acetate

Ex-227c) To a solution of Ex-227b (1.39 g, 3.4 mmol) in dioxane (15 mL) at room temperature was added Me₃SnN₃ (2.0 g, 10.2 mmol). The reaction mixture was heated to reflux for 4 hrs and then cooled to room temperature. The mixture was diluted with water and ethyl acetate. The layers were separated and the organic layer washed with 10% KF and brine. The extract was dried (Na₂SO₄) and the solvent removed in vacuo to give a brown oil, which after RP-HPLC (CH₃CN:H₂O) gave the desired product (380 mg). ¹H NMR (400 MHz, CD₃OD): δ 9.01 (t, 1H), 8.52 (t, 1H), 8.44 (t, 1H), 6.88 (s, 1H), 4.49 (s, 2H), 4.12-4.07 (m, 1H), 1.37 (s, 9H); MS-ESI (M+H)=457.

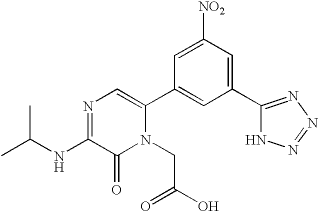

[3-(isopropylamino)-6-[3-nitro-5-(1H-tetraazol-5-yl)phenyl]-2-oxopyrazin-1 (2H)-yl]acetic Acid

Ex-227d) To a round bottom flask containing Ex-227c (0.38 g, 0.83 mmol) 25 mL of 4N HCl in dioxane was added. The reaction mixture was heated to 50° C. for 4 hrs and then cooled to room temperature. The reaction mixture was placed in the refrigerator (˜5° C.) to effect precipitation of the product. The mixture was filtered and dried on high vacuum to give the desired product as a yellow solid (0.31 g, 86%). ¹H NMR (400 MHz, CD₃OD): δ 8.86 (t, 1H), 8.44 (t, 1H), 8.36 (t, 1H), 8.10 (bs, 1H), 6.91 (s, 1H), 5.61-4.81 (bs, 3H), 4.45 (s, 2H), 4.15-4.10 (m, 1H), 1.21 (d, 6H); MS-ESI (M+H)=401.

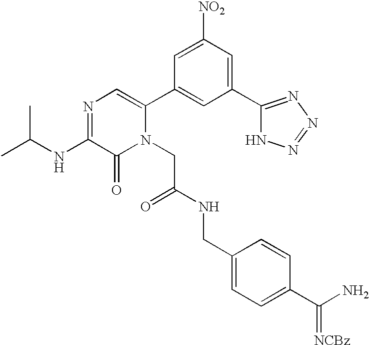

N-[4-(1-aminovinyl)benzyl]-2-[3-(isopropylamino)-6-[3-nitro-5-(1H-tetraazol-5-yl)phenyl]-2-oxopyrazin-1 (2H)-yl]acetamide

Ex-227e) To a solution of Ex-227d (0.31 g, 0.77 mmol) in DMF (10 mL) at 0° C. was added DIEA (0.59 mL, 3.38 mmol) and TBTU (0.37 g, 1.15 mmol). After 15 min, SC-81368 (0.37 g, 1.15 mmol) was added and the reaction stirred overnight at room temperature. The reaction was poured into water and ethyl acetate. The layers were separated and the organic layer washed with brine and dried (Na₂SO₄). The solvent was removed in vacuo to give a brown oil (0.51 g, 100%). MS-ESI (M+H)=666.

Ex-227) To a solution of Ex-227e (0.51 g, 0.76 mmol) in methanol (15 mL) was added 10% Pd/C (0.15 g) and NH₄CO₂H (0.14 g, 2.3 mmol). The reaction mixture was heated to reflux for 2 hrs and then cooled to room temperature. The mixture was filtered through Celite and rinsed with methanol. The solvent was removed in vacuo to give a yellow oil, which was purified by RP-HPLC (CH₃CN:H₂O) to give the desired product (139 mg). ¹H NMR (400 MHz, CD₃OD): δ 7.64 (d, 2H), 7.43 (s, 1H), 7.35 (d, 2H) 7.24 (s, 1H), 6.87 (1H), 6.68 (s, 1H), 4.46 (s, 2H), 4.43 (d, 2H), 4.07-4.03 (m, 1H), 1.37 (d, 6H); MS-ESI (M+H)=502; Analysis: C₂₄H₂₇N₁₁O₂+2.5 TFA+1.2H₂O calcd: C, 43.09; H, 3.97; N, 19.06; 0, 16.23; found: C, 43.17; H, 4.2; N, 18.96; 0, 16.26.

### EXAMPLE 228

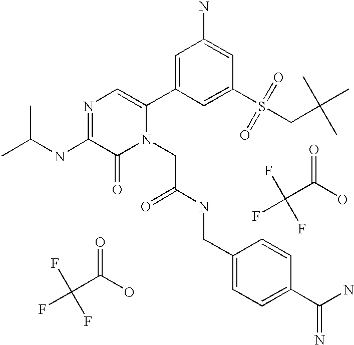

Prepared analogously to Example 74.

### EXAMPLE 229

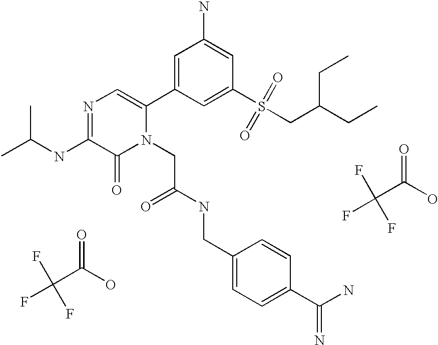

Prepared analogously to Example 74.

### EXAMPLE 230

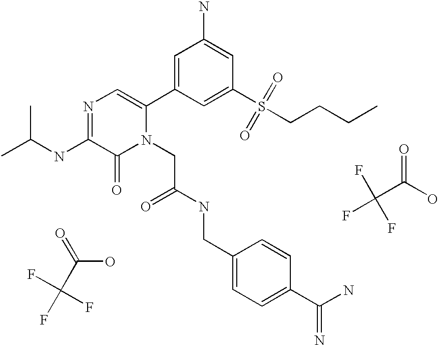

Prepared analogously to Example 74.

### EXAMPLE 231

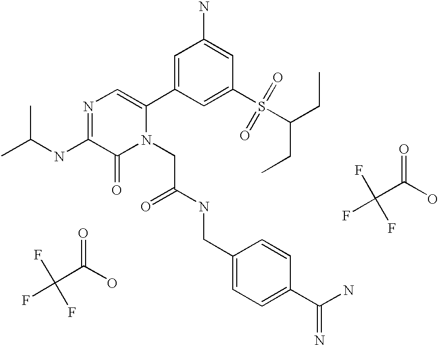

Prepared analogously to Example 74.

### EXAMPLE 232

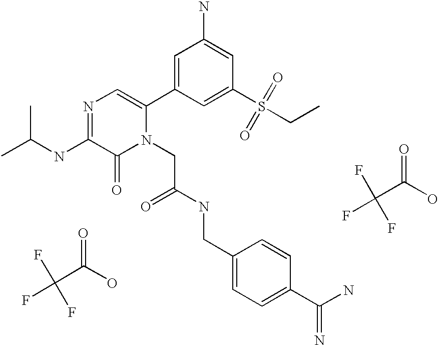

Prepared analogously to Example 74.

### EXAMPLE 233

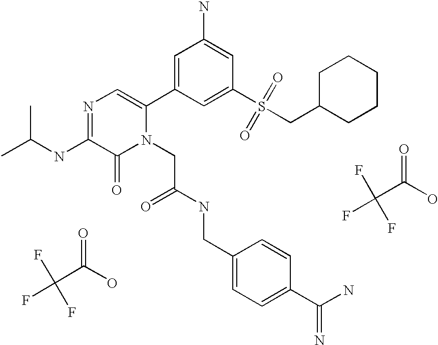

Prepared analogously to Example 74.

### EXAMPLE 234

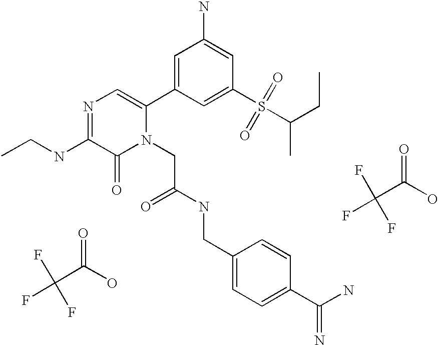

Prepared analogously to Example 74.

### EXAMPLE 235

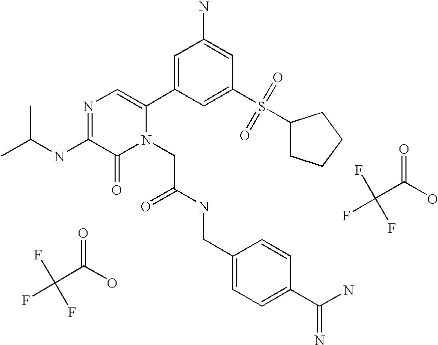

Prepared analogously to Example 74.

### EXAMPLE 236

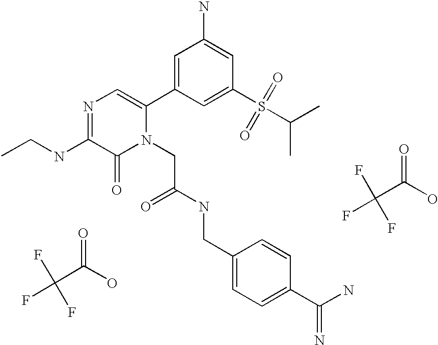

Prepared analogously to Example 74.

### EXAMPLE 237

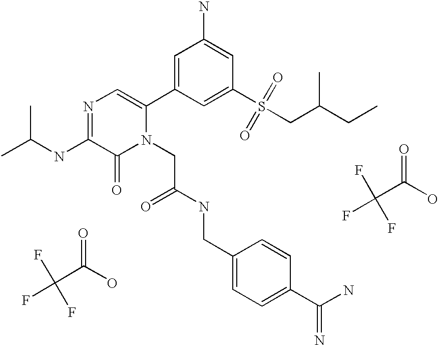

Prepared analogously to Example 74.

### EXAMPLE 238

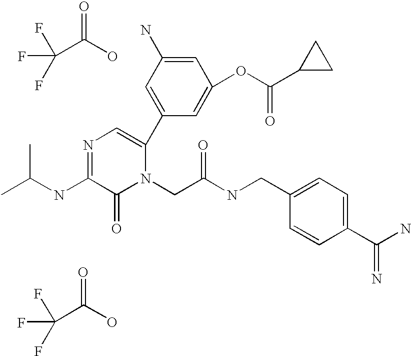

Prepared analogously to Example 55

### EXAMPLE 239

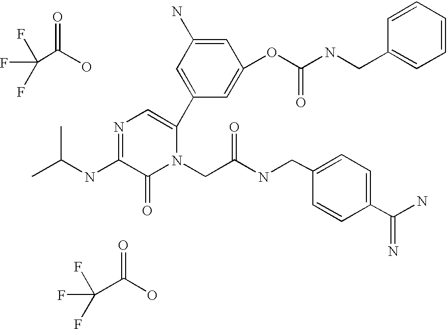

Prepared analogously to Example 55

### EXAMPLE 240

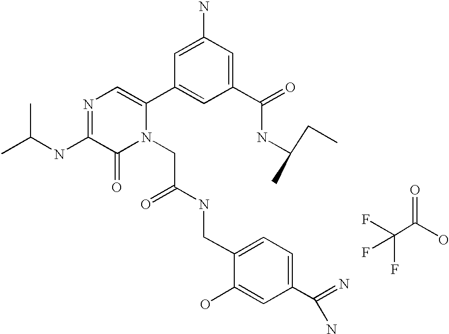

Prepared analogously to Example 182

### EXAMPLE 241

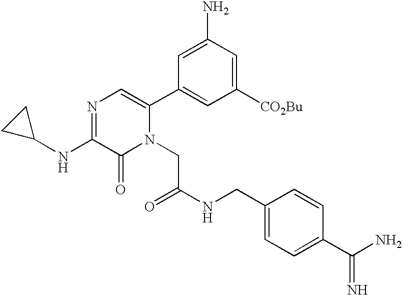

m/z(M+H)+532

Analysis: C₂₈H₃₃N₇O₄+2.35 TFA+0.90H₂O calcd: C, 48.15; H, 4.59; N, 12.02; found: C, 48.18; H, 4.63; N, 11.97. HRMS calcd: 532.2667; Found: 532.2683

¹H NMR(400 MHz, DMSO): 0.64 (2H, m), 0.73 (2H, m), 0.91 (3H, t), 1.40 (2H, sextet), 1.64 (2H, quintet), 2.75 (1H, m), 4.21 (2H, t), 4.36 (2H, d), 4.38 (2H, s), 6.73 (1H, s), 6.77 (1H, t), 7.11 (1H, s), 7.27 (1H, t), 7.38 (2H, d), 7.61 (1H, d), 7.72 (2H, d), 8.67 (1H, t), 9.08 (2H, s), 9.26 (2H, s).

### EXAMPLE 242

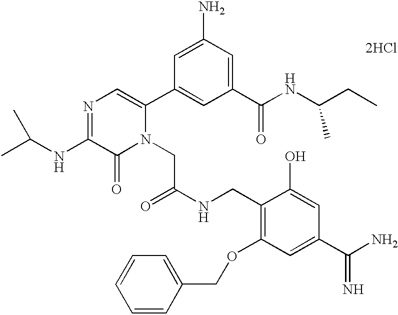

Ex-242a) The product from Ex-193 (0.300 g, 0.00047 mol) was dissolved in acetonitrile (5 mL). To this solution was added (Boc)₂O (0.206 g, 0.00094 mol), triethylamine (0.132 mL, 0.00094 mol), and a catalytic amount of DMAP (0.1%). The solution was stirred at room temperature overnight. An additional amount of (Boc)₂O (0.00037 mol) and triethylamine (0.040 mL, 0.00028 mol) was added and the reaction was stirred for an additional 1.5 hrs. The solvent was evaporated and the crude product purified flash chromatography (Merck 230-400 mesh, SiO₂, 4% methanol/CH₂Cl₂). Ex-242a was obtained (0.240 g, 57.4%) as pale yellow crystals.

HRMS: (M+H)⁺865.4454 found for C₄₃H₆₀N₈O₁₁; 865.4382 calc'd.

Ex-242) The product from Ex-242a (0.036 g, 0.000042 mol) was dissolved in anhydrous DMF (0.30 mL). To this solution was added a slurry of NaH in anhydrous DMF (0.20 mL) (0.0018 g, 0.000071 mol). After 30 min of stirring at room temperature, benzyl bromide (0.0093 mL, 0.000077 mol) was added. After 2 hrs the DMF was evaporated and the residue was treated with 4N HCl in dioxane (15 mL) and stirred overnight. The reaction mixture was concentrated and the crude product was purified by prep HPLC (RP, gradient 5-50% acetonitrile in 0.1% TFA). The purified TFA salt was coevaporated with 1N HCl and lyophilized from water to give 0.009 g (29.4%) of Ex-242 as a pale yellow solid.

HRMS: (M+H)⁺655.3351 found for C₃₅H₄₂N₈O₅; 655.3278 calc'd.

¹H-NMR, 300 mHz, CD₃OD δ 7.45-7.28 (m, 5H), 7.19 (br s, 1H), 7.09 (br s, 1H), 6.93 (d, 1H), 6.87 (br s, 2 h), 6.61 (s, 1H), 5.17 (s, 2H), 4.52 (s, 2H), 4.45 (s, 2H), 4.05-3.94 (m, 2 h), 1.61-1.51 (m, 2 h), 1.38 (d, 6H, J=6.4 Hz), 1.19 (d, 3H, J=6.6 Hz), 0.93 (t, 3H, J=7.4 Hz).

### EXAMPLE 243

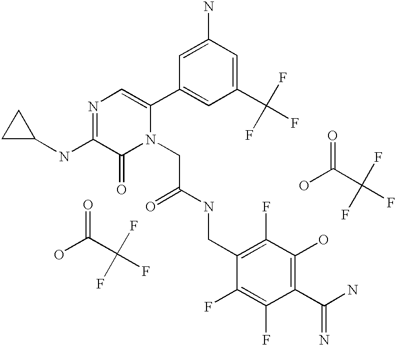

Prepared analogously to Example 185.

### EXAMPLE 244

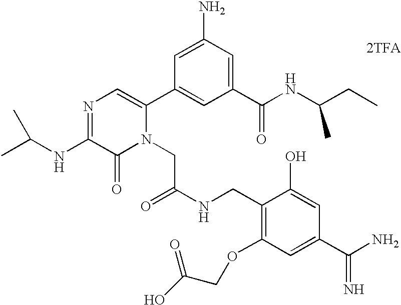

Ex-244a) Ex-244a was prepared analogously to example Ex-242a except that Ex-199 was used as the starting material.

HRMS: (M+H)+865.4454 found for C₄₃H₆₀N₈O₁₁; 865.4382 calc'd. Ex-244) The product from Ex-244a (0.031 g, 0.000035 mol) was dissolved in anhydrous DMF (0.30 mL). A DMF solution containing NaH (0.001 g, 0.000039 mol) was added. After 20 min t-butylbromoacetate (0.013 mL) was added and the reaction mixture was then stirred at room temperature overnight. The DMF was evaporated and the crude residue was treated with 4N HCl in dioxane (15 mL) overnight. The solvent was evaporated and the crude product was purified by prep HPLC (RP, gradient 5-50% acetonitrile in 0.1% TFA). Ex-244 was obtained as the TFA salt (0.010 g, 33%).

HRMS: (M+H)+623.2936 found for C₃₀H₃₈N₈O₇; 623.2863 calc'd.

¹H-NMR, 400 mHz, CD₃OD δ 7.12 (t, 1H), 7.02 (t, 1H), 6.88 (d, 1H, J=1.65 Hz), 6.80 (d, 2H, J=1.65 Hz), 6.6 (s, 1H), 4.80 (s, 2H), 4.53 (s, 2H), 4.46 (s, 2H), 3.99-3.95 (m, 1h), 4.06-4.00 (m, 1H), 1.61-1.51 (m, 2H), 1.37 (d, 6H, J=6.4 Hz), 1.20 (d, 3H, J=6.7 Hz), 0.95 (t, 3H, 7.4 Hz).

### EXAMPLE 245

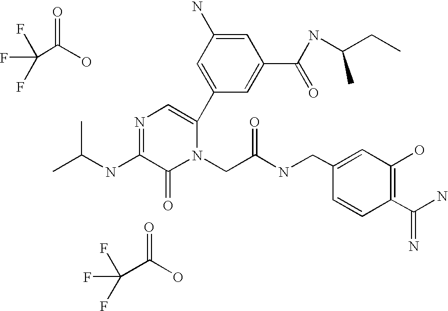

Prepared analogously to Example 181.

### EXAMPLE 246

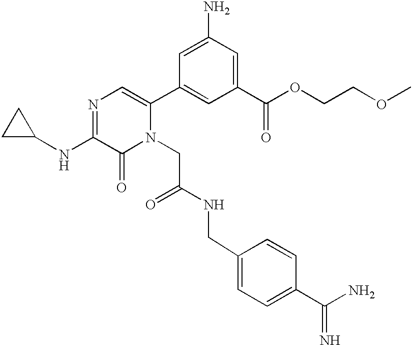

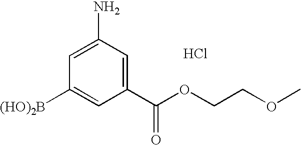

Ex-246a) ¹HNMR (300 MHz, DMSO-d₆) δ 3.30 (s, 3H), 3.62-3.68 (m, 2H), 4.36-4.42 (m, 2H), 7.56 (s, 1H), 7.69 (s, 1H), 7.88 (s, 1H). LCMS (ES+) m/z 240.

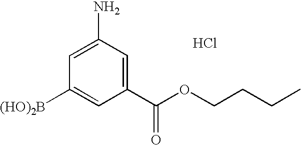

Ex-246b) Into a solution of (3-amino-5-carboxylphenyl) boronic acid (1.0 g, 5.5 mmol) in n-butanol (15 ml) was bubbled hydrogen chloride gas for 5 minutes. The reaction was sealed and heated at 85° C. for two hours. The reaction was diluted with diethyl ether and the resulting crystals collected by vacuum filtration to give 1.2 g of a colorless solid (ca. 40 mole % n-butanol). LCMS (ES+) m/z 238.

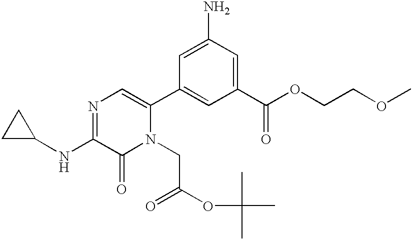

Ex-246c) LCMS (ES+) m/z 459.

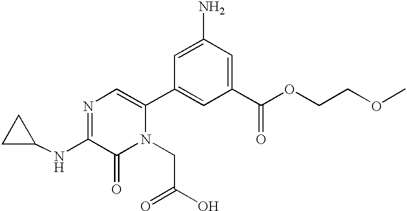

Ex-246d) LCMS (ES+) m/z 403.

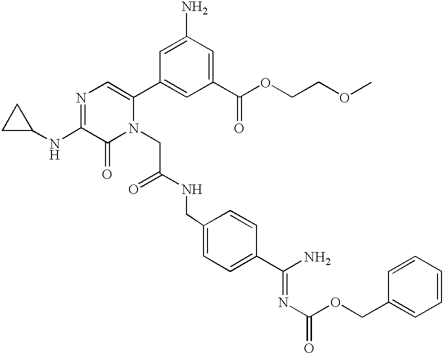

Ex-246e) To a stirred solution of Ex-246c (200 mg, 0.46 mmol), Ex-246d (204 mg, 0.64 mmol), and N-methylmorpholine (0.3 ml, 2.73 mmol) in N,N-dimethylacetamide (4 ml) cooled in an ice bath was added TBTU (161 mg, 0.5 mmol). Stirring was continued at ambient temperature for 1.5 hours. Purification by reverse phase HPLC (10-55% acetonitrile/water) followed by lyophilization yielded 169 mg (38% yield) of an off-white solid. ¹HNMR (300 MHz, DMSO-d₆) δ 0.62-0.79 (m, 4H), 2.70-2.80 (m, 1H), 3.58-3.63 (m, 2H), 4.40-4.50 (m, 6H), 5.33 (s, 2H), 6.74 (s, 1H), 6.77 (s, 1H), 7.10 (s, 1H), 7.27 (s, 1H), 7.39 (d, J=8.4 Hz, 2H), 7.39-7.50 (m, 5H), 7.73 (d, J=8.4 Hz, 2H), 8.67 (t, J=6.0 Hz, 1H), 10.41 (br s, 1H). HRMS (ES) calcd for C₃₅H₃₈N₇O₇ (M+H): 668.2827. Found: 668.2805. Anal. Calcd for C₃₅H₃₇N₇O₇+2.45 TFA+0.75H₂O: C, 49.88; H, 4.29; N, 10.20. Found: C, 49.90; H, 4.32; N, 10.18.

Ex-246) A solution of Ex-246e (25 mg) in ethanol (25 ml) was shaken with 10% palladium on carbon under hydrogen at 40 psi. The reaction was filtered and purification by reverse phase HPLC (5-50% acetonitrile/water) followed by lyophilization yielded 8 mg of an off-white solid. ¹HNMR (300 MHz, DMSO-d₆) δ 0.60-0.78 (m, 4H), 2.70-2.80 (m, 1H), 3.28 (s, 3H), 3.59-3.65 (m, 2H), 4.29-4.39 (m, 6H), 6.73 (s, 1H), 6.77 (s, 1H), 7.10 (s, 1H), 7.25-7.29 (m, 1H), 7.39 (d, J=8.4 Hz, 2H), 7.71 (d, J=8.4 Hz, 2H), 8.54 (t, J=5.7 Hz, 1H), 8.90 (br s, 1H), 9.24 (br S, 2H). HRMS (ES) calcd for C₂₇H₃₂N₇05 (M+H): 534.2459. Found: 534.2417.

### EXAMPLE 247

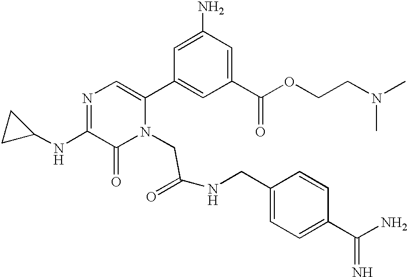

2-(dimethylamino)ethyl 3-amino-5-[1-[2-({4-[amino(imino)methyl]benzyl}amino)-2-oxoethyl]-5-(cyclopropylamino)-6-oxo-1,6-dihydropyrazin-2-yl]benzoate

HRMS calcd for C₂₈H₃₄N₈O₄ (M+H): 547.2776. Found: 547.2791.

¹H NMR (DMSO-d₆, 300 MHz) δ 0.65 (m, 2H), 0.75 (m, 2H), 2.86 (bs, 7H), 4.38 (m, 5H), 4.53 (m, 3H), 6.74 (s, 1H), 6.82 (s, 1H), 7.19 (s, 1H), 7.30 (s, 1H), 7.42 (d, 2H), 7.73 (d, 2H), 7.96 (m, 1H), 8.73 (t, 1H), 9.12 (bs, 2H), 9.26 (bs, 2H), 9.77 (bs, 1H).

### EXAMPLE 248

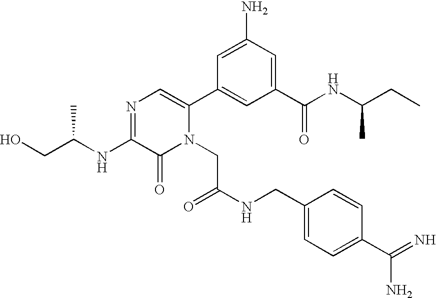

HPLC/LRMS: >97%, 550 (M+H)+; HRMS(ES+) calcd. for C₂₈H₃₇N₈O₄ 549.2932, found 549.2912.

### EXAMPLE 249

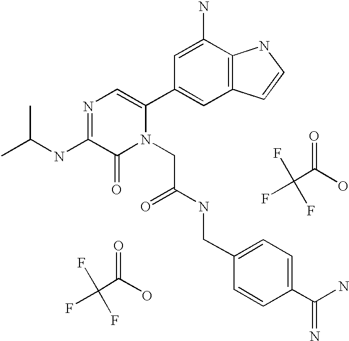

Prepared analogously to Example 172.

### EXAMPLE 250

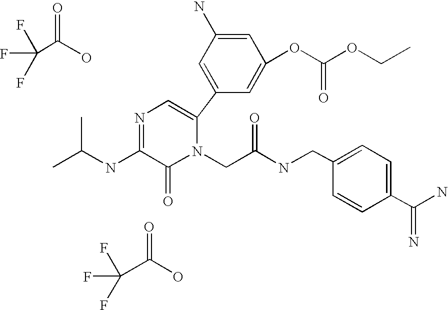

Prepared analogously to Example 55.

### EXAMPLE 251

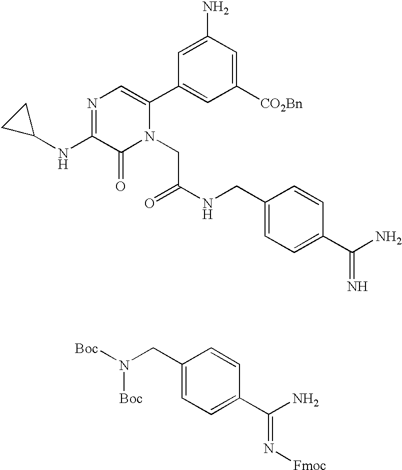

Ex-251a) To a solution of di(tert-butyl)-4-[amino(imino)methyl]benzylimidodicarbonate (1.07 g, 2.71 mmol) in 48 ml of tetrahydrofuran and 5.3 ml of water at 0° C. was added sodium carbonate (1.44 g, 13.6 mmol) and 9-fluorenylmethyl chloroformate (1.54 g, 5.97 mmol). The reaction mixture was stirred overnight while warming to room temperature. The mixture was treated with brine and extracted with ethyl acetate. The organic layer was dried over sodium sulfate, filtered and evaporated in vacuo to give a white solid. The solid was purified by silica gel chromatography with 10-50% EA/Hex to give 0.63 g (41% yield) of a white solid. m/z(M+H)+572

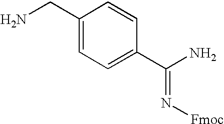

Ex-251b) The product from Ex-251a (0.626 g, 1.10 mmol) was dissolved in 1.5 ml of dioxane and treated with 4M hydrogen chloride in dioxane (4 ml, 16 mmol) and stirred overnight. The mixture was diluted with 20 ml of ethyl ether and the resulting precipitate was collected by vacuum filtration to give 0.475 g (quantitative yield) of a white solid. m/z(M+H)⁺372

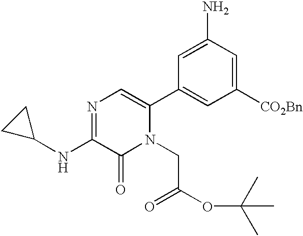

Ex-251c) m/z(M+H)⁺491

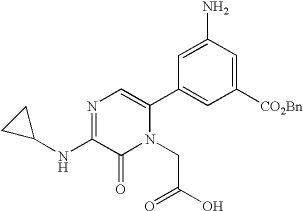

Ex-251d) To the product from Ex-251c (0.216 g, 0.745 mmol) was added 4M hydrogen chloride in dioxane (7 ml, 28 mmol). The solution was heated at 65° C. for 2 hours. The mixture was diluted with ethyl ether and the resulting precipitate was collected by vacuum filtration and dried over phosphorous pentoxide under high vacuum to give 0.194 g (94% yield) of a yellow solid. m/z(M+H)+435

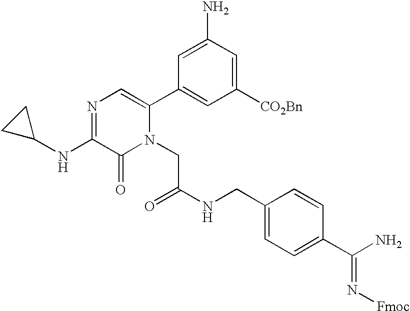

Ex-251e) To the product from Ex-251d (0.190 g, 0.404 mmol) and the product from Ex-251b (197 mg, 0.485 mmol) in 2.5 ml of N,N-dimethylformamide at 0° C. was added N,N-diisopropylethylamine (0.28 ml, 1.62 mmol) and then benzotriazol-1-yl tetramethyluronium tetrafluoroborate (0.156 g, 0.485 mmol). The solution was stirred for 2 hours at room temperature and then added to a solution of brine. The precipitate was collected by vacuum filtration and dried over phosphorous pentoxide under high vacuum to give 288 mg (91% yield) of an off-white solid m/z(M+H)+788

Ex-251) To the product from Ex-251e (0.282 g, 0.358 mmol) in 4 ml of dichlormethane was added diethylamine (0.4 ml, 3.87 mmol) and the solution was stirred for 2 hours. Another portion of diethylamine (0.8 ml, 7.74) was added and the solution was stirred an additional 1.5 hours. The solvent was removed in vacuo and the residue was dissolved in methyl sulfoxide, acetonitrile and acidified with trifluoroacetic acid. The solution was purified by reverse phase chromatography to give 120 mg (43% yield) of a yellow solid m/z(M+H)⁺566

Analysis: C₃₁H₃₁N₇O₄+1.60 TFA+2.35H₂O calcd: C, 51.97; H, 4.76; N, 12.40; found: C, 51.92; H, 4.69; N, 12.46.

HRMS calcd: 566.2510; Found: 566.2471

¹H NMR(400 MHz, DMSO): 0.66 (2H, br s), 0.75 (2H, m), 2.71 (1H, m), 4.22 (2H, d), 4.31 (2H, s), 5.23 (2H, s), 6.69 (1H, s), 6.74 (1H, s), 7.10 (1H, s), 7.28-7.39 (8H, m), 7.67 (2H, d), 8.66 (1H, t), 9.08 (2H, s), 9.26 (2H, s).

### EXAMPLE 252

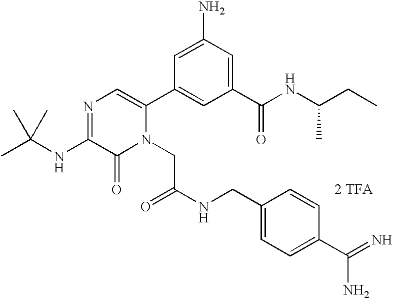

¹H NMR (400 MHz, DMF-d₇) δ 10.02 (s, 2H), 9.42 (br s, 6H), 8.70 (t, J=6.0 Hz, 1H), 8.14 (d, J=8.3 Hz, 1H), 7.90 (d, J=8.3 Hz, 2H), 7.44 (d, J=8.3 Hz, 3H), 7.26 (s, 1H), 7.01 (s, 1H), 6.76 (s, 1H), 6.37 (br s, 1H), 4.64 (s, 2H), 4.49 (d, J=5.9 Hz, 2H), 4.04-3.94 (m, 1H), 1.63-1.43 (m, 11H), 1.16 (d, J=6.7 Hz, 3H), 0.88 (t, J=7.4 Hz, 3H); ¹³C NMR (100 MHz, DMF-d₇) δ 167.50, 167.41, 166.7, 152.5, 149.8, 147.4, 146.4, 137.4, 134.1, 129.5, 128.77, 128.02, 127.6, 51.7, 48.6, 47.5, 42.7, 29.7, 28.3, 20.4, 10.9; HRMS (ES) calcd for C₂₉H₃₉N₈O₃ 547.3140, found 547.3147.

### EXAMPLE 253

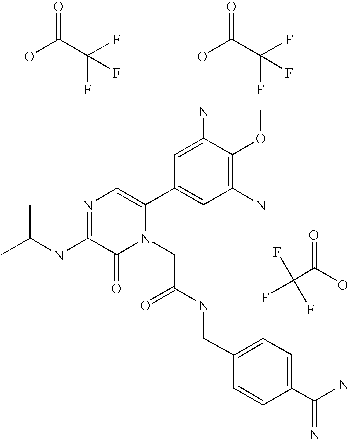

Prepared analogously to Example 172.

### EXAMPLE 254

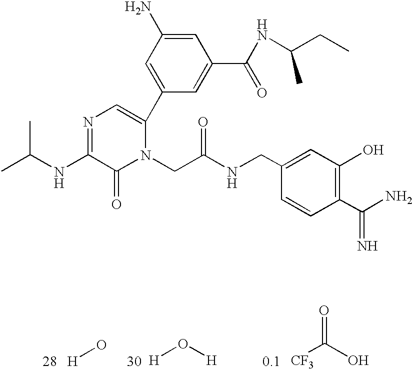

Prepared analogously to Example 181.

### EXAMPLE 255

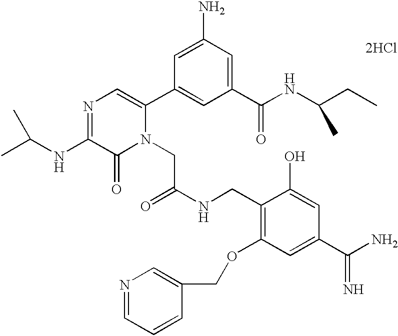

The product from Ex-244a (0.030 g, 0.000035 mol) was dissolved in anhydrous DMF (0.300 mL). To this solution was added NaH (0.0010 g in 0.5 mL DMF). After 30 min a solution of pyridyl bromide (0.0087 g, 0.000035 mol) neutralized with DIEA was added (0.0062 mL) in DMF (0.20 mL). The solution was stirred at room temperature overnight. The DMF was evaporated and the crude product was treated with 4N HCl in dioxane overnight. The solvent was evaporated and the crude product was purified by prep HPLC (RP, gradient 5-35% acetonitrile in 0.1% TFA). The purified TFA salt was coevaporated with 1N HCl and lyophilized from water to give 2.6 mg (10.2%) of the product Ex-255 as a pale yellow solid.

HRMS: (M+H)+656.3303 found for C₃₄H₄₁N₉O₅; 656.3231.

¹H-NMR, 400 mHz, CD₃OD δ 8.88 (s, 1H), 8.78 (d, 1H, J=5.6 Hz), 8.66 (d, 1H, J=8.0 Hz), 8.08 (t, 1H, J=6.9 Hz), 7.16 (s, 1H), 7.08 (s, 1HO, 6.79 (s, 1 h), 6.78-6.68 (m, 2H), 6.58 (s, 1H), 4.63 (s, 2H), 4.49 (s, 2H), 4.39 (s, 2H), 4.04-3.95 (m, 2H), 1.60-1.56 (m, 2H), 1.39 (d, 6H, J=6.4 Hz), 1.20 (d, 3H, J=6.6 Hz), 0.93 (t, 3H, J=7.4 Hz).

### EXAMPLE 256

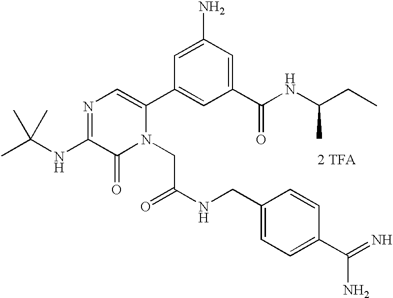

Ex-256a) tert-butyl [3-(tert-butylamino)-6-[3-({[(1R)-1-methylpropyl]amino}carbonyl)-5-nitrophenyl]-2-oxopyrazin-1 (2H)-yl]acetate

¹H NMR (400 MHz, CDCl₃) δ 8.67-8.66 (m, 1H), 8.35-8.34 (m, 1H), 8.17-8.16 (m, 1H), 6.82 (s, 1H), 6.55 (br d, J=8.3 Hz, 1H), 6.23 (s, 1H), 4.34 (s, 2H), 4.19-4.09 (m, 1H), 1.66-1.56 (m, 2H), 1.47-1.44 (m, 18H), 1.26 (d, J=6.6 Hz, 3H), 0.98 (t, J=7.4 Hz, 3H); ¹³C NMR (100 MHz, CDCl₃) δ 166.5, 163.6, 151.8, 149.8, 148.3, 137.1, 134.8, 133.6, 126.2, 124.6, 123.8, 122.1, 83.2, 51.5, 48.2, 47.8, 29.5, 28.1, 27.8, 20.2, 10.4; HRMS (ES) calcd for C₂₅H₃₆N₅O₆ 502.2660, found 502.2636.

Ex-256b) A solution of EX-256a (5.61 g, 11.19 mmol) in a solution 112.0 mL of ethyl acetate and ethanol (1:1, 0.1 M) was added 326.6 mg 10% Pd-C (wet) in one portion. The resulting suspension was flushed with hydrogen and then allowed to stir under an atmosphere of hydrogen (balloon) over night (approximately 18 hours). The reaction mixture is filtered through a pad of Celite 545 and the filtrate is concentrated under reduced pressure. Purification of the crude product by trituration with ethyl ether to afforded pure tert-butyl [6-[3-amino-5-({[(1R)-1-methylpropyl]amino}carbonyl)phenyl]-3-(tert-butylamino)-2-oxopyrazin-1 (2H)-yl]acetate in 80% yield: ¹H NMR (400 MHz, DMF-d₇) δ 8.03-8.01 (br m, 1H), 7.2-7.28 (m, 1H), 7.03 (s, 1H), 6.76-6.75 (m, 2H), 6.24 (s, 1H), 5.52 (s, 2H), 4.44 (s, 2H), 4.05-3.95 (m, 1H), 1.65-1.48 (m, 11H), 1.39 (s, 9H), 1.17 (d, J=6.6 Hz, 3H), 0.90 (t, J=7.4 Hz, 3H); ¹³C NMR (100 MHz, DMF-d₇) δ 167.4, 166.9, 152.3, 150.04, 149.93, 137.7, 133.7, 129.3, 121.6, 117.8, 116.4, 114.3, 82.5, 51.6, 48.1, 47.4, 29.8, 28.46, 27.99, 20.5, 11.1; HRMS (ES) calcd for C₂₅H₃₈N₅O₄ 472.2918, found 472.2923.

Ex-256c) A solution of EX-256b (4.0056 g, 8.494 mmol) in chloroform (85.0 mL, 0.15 M) was added trifluoroacetic acid (16.00 mL, 207.7 mmol) in one portion at room temperature. The solution was allowed to stir over night (approximately 18 hours). The reaction was concentrated to an oil and then diluted with aqueous hydrochloric acid (25.0 mL, 1N) and the solvent was removed under reduced pressure. This process was repeated two more times to afford pure [6-[3-amino-5-({[(lR)-1-methylpropyl]amino}carbonyl)phenyl]-3-(tert-butylamino)-2-oxopyrazin-1 (2H)-yl]acetic acid dihydrochloride in 69% yield: ¹H NMR (400 MHz, DMF-d₇) δ 10.96 (br s, 5H), 8.57 (d, J=8.1 Hz, 1H), 8.16 (s, 1H), 7.95 (s, 1H), 7.75 (s, 1H), 6.91 (s, 1H), 4.58 (s, 2H), 4.05-3.94 (m, 1H), 1.67-1.48 (m, 11H), 1.18 (d, J=6.6 Hz, 3H), 0.88 (t, J=7.4 Hz, 3H); ¹³C NMR (100 MHz, DMF-d₇) δ 168.9, 165.0, 152.7, 147.6, 138.0, 135.6, 133.3, 128.2, 127.2, 126.6, 123.4, 53.3, 48.2, 47.7, 29.6, 27.9, 20.3, 10.9; HRMS (ES) calcd for C₂₁H₃₀N₅O₄ 416.2292, found 416.2320.

Ex-256d) benzyl (1Z)-amino{4-[({[6-[3-amino-5-({[(1R)-1-methylpropyl]amino}carbonyl)phenyl]-3-(tert-butylamino)-2-oxopyrazin-1 (2H)-yl]acetyl}amino)methyl]phenyl}methylidenecarbamate

¹H NMR (400 MHz, DMF-d₇) δ 9.50 (br s, 1H), 9.12 (br s, 1H), 8.56 (t, J=5.8 Hz, 1H), 8.07-8.01 (m, 4H), 7.48-7.31 (m, 7H), 7.13 (s, 1H), 6.85 (s, 1H), 6.75 (s, 1H), 6.25 (s, 1H), 5.53 (s, 2H), 5.21 (s, 2H), 4.60 (s, 2H), 4.46 (d, J=5.8 Hz, 2H), 4.06-3.96 (m, 1H), 1.64-1.44 (m, 11H), 1.17 (d, J=6.7 Hz, 3H), 0.89 (t, J=7.5 Hz, 3H); ¹³C NMR (100 MHz, DMF-d₇) δ 168.0, 167.45, 166.95, 165.2, 152.5, 150.0, 149.9, 144.3, 138.3, 137.4, 134.1, 133.9, 129.81, 129.01, 128.58, 128.51, 128.37, 127.7, 121.6, 117.9, 116.6, 114.2, 66.9, 51.5, 48.8, 47.4, 43.0, 29.8, 28.5, 20.5, 11.0; HRMS (ES) calcd for C₃₇H₄₅N₈O₅ 681.3507, found 681.3498.

Ex-256) A solution of EX-256d (2.2515 g, 3.307 mmol) in methanol/HCl (34.0 mL, 3:1 methanol:4 M HCl in methanol, 0.1M) was added 378.8 g 10% Pd—C (wet) in one portion. The resulting suspension was flushed with hydrogen and then allowed to stir under an atmosphere of hydrogen (balloon) for approximately 4 hours. The reaction mixture is filtered through a pad of Celite 545 and the filtrate is concentrated under reduced pressure. Purification by reverse phase HPLC (5% acetonitrile to 50% acetonitrile/water/0.1%trifluoroacetic acid) afford pure 3-amino-5-[1-[2-({4-[amino(imino)methyl]benzyl}amino)-2-oxoethyl]-5-(tert-butylamino)-6-oxo-1,6-dihydropyrazin-2-yl]-N-[(lR)-1-methylpropyl]benzamide bis(trifluoroacetate) in 81% yield: ¹H NMR (400 MHz, DMF-d₇) δ 9.98-9.81 (br m, 8H), 9.44 (s, 2H), 8.720 (t, J=5.9 Hz, 1H), 8.17 (d, J=8.2 Hz, 1H), 7.90 (d, J=8.3 Hz, 2H), 7.51-7.50 (m, 1H), 7.43 (d, J=8.3 Hz, 2H), 7.31 (s, 1H), 7.07-7.06 (m, 1H), 6.77 (s, 1H), 6.41 (br s, 1H), 4.66 (s, 2H), 4.48 (d, J=5.9 Hz, 2H), 4.04-3.94 (m, 1H), 1.63-1.44 (m, 11H), 1.16 (d, J=6.6 Hz, 3H), 0.88 (t, J=7.4 Hz, 3H); ¹³C NMR (100 MHz, DMF-d₇) δ 167.46, 167.39, 166.6, 152.5, 149.7, 146.48, 146.43, 137.4, 134.1, 129.3, 128.77, 128.01, 127.5, 121.0, 119.88, 119.11, 118.1, 116.2, 115.2, 112.3, 51.7, 48.6, 47.5, 42.7, 29.7, 28.3, 20.4, 10.9; HRMS (ES) calcd for C₂₉H₃₉N₈O₃ 547.3140, found 547.3127.

### EXAMPLE 257

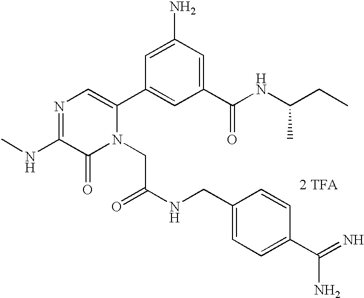

HPLC/LRMS: >98%, 505 (M+H)+; HRMS cacld for C₂₆H₃₃N₈O₃ 505.2670, found 505.2661.

### EXAMPLE 258

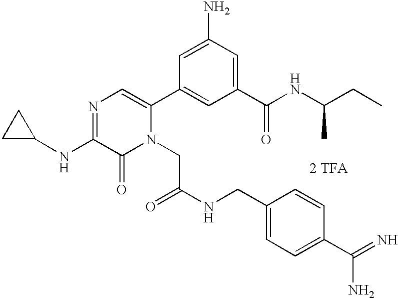

¹H NMR (400 MHz, DMF-d₇) δ 10.20 (br m, 7H), 9.44-9.36 (m, 3H), 8.81-8.78 (m, 1H), 8.14 (d, J=8.2 Hz, 1H), 7.91 (d, J=8.3 Hz, 2H), 7.44-7.42 (m, 3H), 7.20 (s, 1H), 6.94-6.92 (m, 2H), 4.66 (s, 2H), 4.49 (d, J=5.8 Hz, 2H), 4.05-3.94 (m, 1H), 2.99-2.95 (m, 1H), 1.64-1.47 (m, 2H), 1.17 (d, J=6.6 Hz, 3H), 1.00-0.87 (m, 7H); ¹³C NMR (100 MHz, DMF-d₇) δ 167.5, 166.87, 166.71, 160.3, 159.9, 152.7, 150.3, 149.0, 146.2, 137.6, 132.7, 131.1, 128.79, 128.07, 127.7, 118.3, 117.4, 115.3, 48.7, 47.5, 42.8, 29.7, 24.9, 20.4, 10.9, 7.2; HRMS (ES) calcd for C₂₈H₃₅N₈O₃ 531.2827, found 531.2794.

### EXAMPLE 259

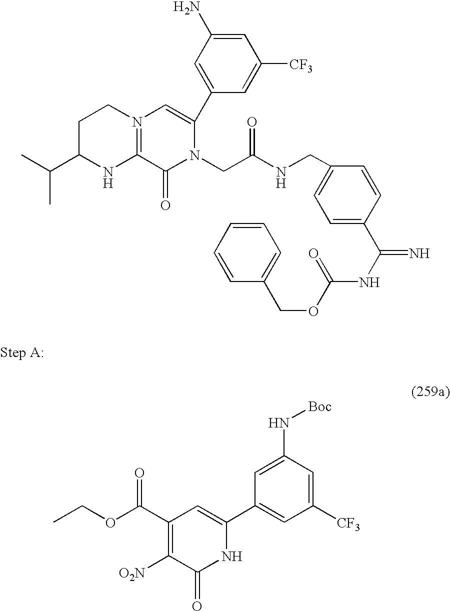

To a slurry of 500 mmol of the ammonium salt of 2-nitroacetamide in 400 grams of water is added 600 mmol of ethyl 2,4-dioxo-4-(3-(t-butoxycarbonylamino)-5-trifluoromethylphenyl)butanoate (prepared by standard methods from diethyl oxalate and 1-acetyl-3-(t-butoxycarbonylamino)-5-trifluoromethylbenzene). A solution of piperidinium acetate (prepared by adding 72 mL of piperidine to 42 mL of acetic acid in 200 mL of water) is then added. The resulting reaction mixture is stirred at 40° C. for about 24 hours. The reaction product 259a is then separated, dried and used in the next step.

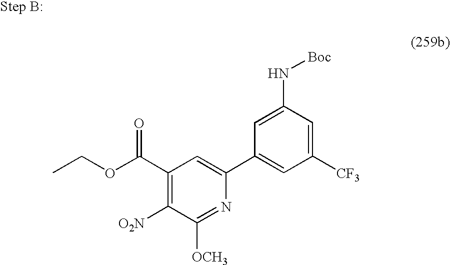

A solution of the pyridone 259a from Step A (400 mmol) in 500 mL of methylene chloride is treated with 500 mmol of solid trimethyloxonium tetrafluoroborate and the mixture stirred at 40° C. until the reaction is complete as monitored by liquid chromatography. The reaction mixture is concentrated about 70% and chromatographed on silica gel to afford the methoxy pyridine 259b.

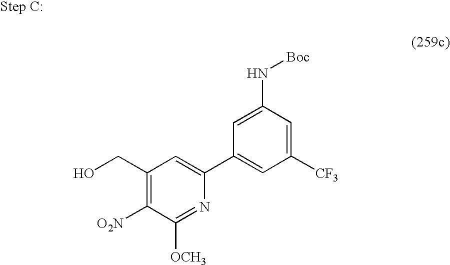

To a solution of the pyridine 259b from Step B (350 mmol) in 1000 mL of methylene chloride at −70° C. is added with 700 mmol of DIBAL (1 molar in hexane) using a dropping funnel. The resulting solution is stirred for 1 hour and then warmed to room temperature over an additional hour. The reaction mixture is quenched by the careful addition of saturated sodium potassium tartrate. After stirring for 30 additional minutes, the solid is filtered and washed with 500 mL methylene chloride. The filtrate is washed twice with 500 mL of saturated sodium potassium tartrate and then 500 mL of brine. The solution is concentrated and then chromatographed to afford the desired alcohol 259c.

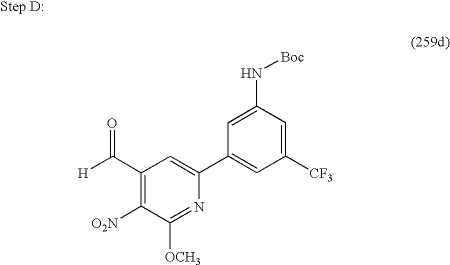

To a solution of phosgene (350 mmol) in 1000 mL of methylene chloride at −70° C. is added 700 mmol of DMSO in 100 mL methylene chloride using a dropping funnel. Then, the resulting solution is treated with the pyridone alcohol 259c from Step C (300 mmol) in 500 mL of methylene chloride, stirred for an additional 15-30 minutes, treated with 225 mL of triethylamine, and then warmed to room temperature over an additional 1.5 hours. The reaction mixture is quenched by the addition of 1000 mL water and the two phases separated. The aqueous is extracted twice with 1000 mL of methylene chloride and the combined organic extracts is washed with 500 mL of brine. The methylene chloride solution is dried over MgSO4₁ concentrated, and then chromatographed to afford the desired aldehyde 259d.

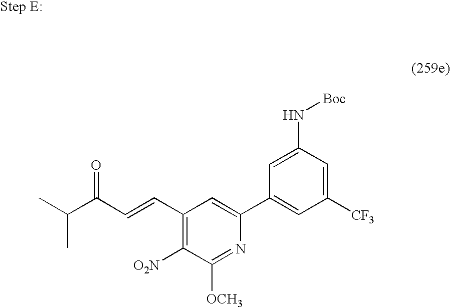

To a solution of diethyl 2-(3-methyl-2-oxo-butyl)phosphonate (250 mmol; obtainable through a standard Arbuzov reaction between 1-bromo-3-methyl-2-butanone and triethyl phosphite) in 1000 mL of THF at 0° C. is added 250 mmol of NaH. Then, the resulting solution is stirred until hydrogen evolution ceased and then treated with the pyridine aldehyde 259d from Step D (250 mmol) in 800 mL of THF. The solution is heated at 50° C. for 180 minutes, cooled, and evaporated. The residue is redissolved in 2000 mL of ethyl acetate and quenched to a pH of 7 with saturated ammonium chloride. The organic phase is washed with brine, dried over MgSO₄, concentrated, and then chromatographed to afford the desired nitro ketone 259e.

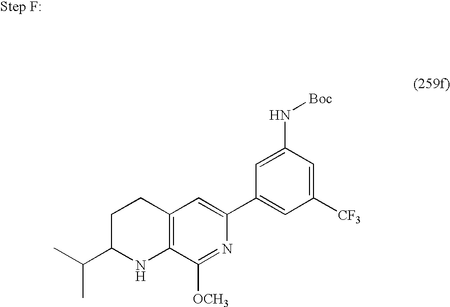

To a solution of nitro ketone 259e from Step E (225 mmol) in 1000 mL of ethyl acetate is added 20 grams of 10% Pd/C. Hydrogen gas is added until uptake of hydrogen stopped. The reaction mixture is filtered through Celite and the filtrate evaporated. The residue is then chromatographed to afford the desired bicyclic methoxy pyridine 259f.

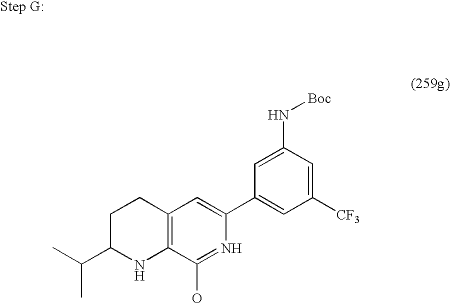

To a solution of methoxy pyridine 259f from Step F (200 mmol) in 1000 mL of dichloroethane at ambient temperature is added 400 mmol of boron tribromide in 400 mL methylene chloride. After stirring for about two hours, the reaction mixture is quenched to a pH of 8 with saturated sodium bicarbonate. The mixture was diluted with 2000 mL of ethyl acetate and 200 mL of THF. The aqueous phase is discarded and the organic solution washed with 200 mL water followed by 200 mL of brine. The reaction mixture evaporated to afford the desired bicyclic pyridone 259 g.

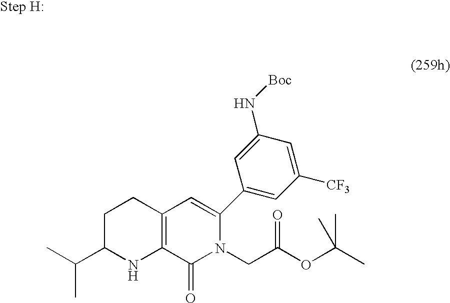

The bicyclic pyridone 259 g from Step G (150 mmol) is alkylated with tert-butyl bromoacetate using procedures previously disclosed to afford the desired bicyclic pyridone acetate 259 h.

Step I:

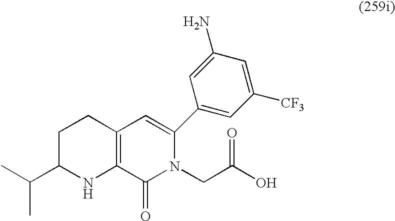

The bicyclic pyridone acetate 259 h from Step H (100 mmol) is deprotected with trifluoroacetic acid as described in Example 1g to afford the desired bicyclic pyridone acetic acid 259i.

A solution of compound bicyclic pyridone acetic acid 259i (50 mmol) in DMF (250 mL) is treated with N-hydroxybenzotriazole (60 mmol) and EDC hydrochloride (60 mmol). The mixture is stirred at room temperature for 30 min and treated with 4-(N-Cbz-amidinobenzylamine (50 mmol). The resulting mixture is allowed to stir overnight. Typical aqueous workup is followed by chromatographic purification to afford pure Example 259 product.

### EXAMPLE 260

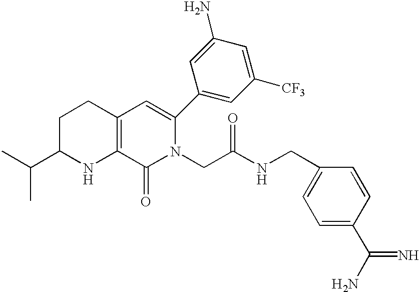

Compound of Example 259 (10 mmol) and 10% Pd on activated carbon (0.100 g) are mixed with 100 mL methanol. The mixture is stirred for 2 hours under an atmosphere of hydrogen that is introduced through a rubber balloon. After filtering off the catalyst and removing the methanol, the remaining residue is obtained as Example 260.

### EXAMPLE 261

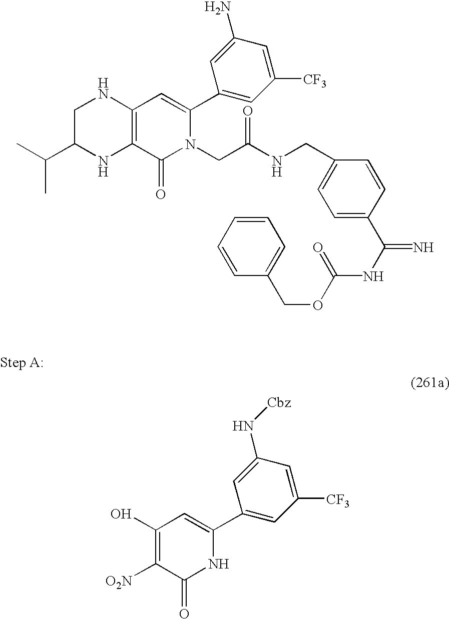

To a slurry of 500 mmol of the ammonium salt of 2-nitroacetamide in 400 grams of water is added 600 mmol of ethyl 3-oxo-3-(3-(t-butoxycarbonylamino)-5-trifluoromethylphenyl)propanoate (prepared by standard methods from diethyl carbonate and 1-acetyl-3-(t-butoxycarbonylamino)-5-trifluoromethylbenzene). A solution of piperidinium acetate (prepared by adding 36 mL of piperidine to 21 mL of acetic acid in 100 mL of water) is then added. The resulting reaction mixture is stirred at 40° C. for about 24 hours. The reaction product 261a is then separated, dried and used in the next step.

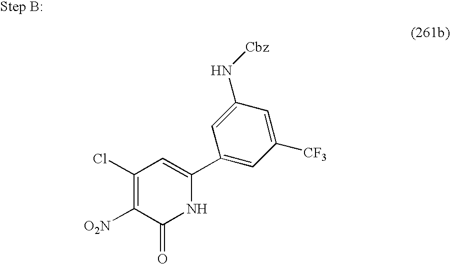

A solution of the pyridone 261a from Step A (400 mmol) in 2000 mL of acetonitrile is treated with 1.6 moles of phosphorusoxychloride and 1.5 moles N-benzyl-N,N,N-triethylammonium chloride. The mixture is stirred at 40° C. and then heated at reflux until the reaction is complete as monitored by liquid chromatography. The reaction mixture is concentrated to remove solvent, and the residue is slurried with water (1000 mL). The product is separated to afford the chloro pyridone 261b.

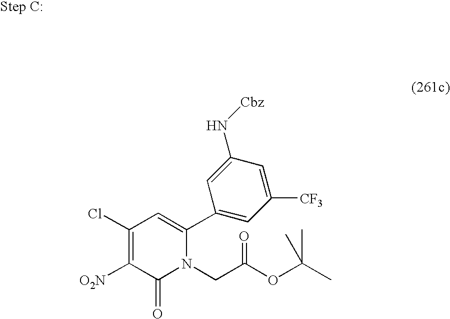

The chloro pyridone 261b from Step B (350 mmol) is alkylated with tert-butyl bromoacetate using the procedure of Example 1d to afford the desired bicyclic pyridone acetate 261c.

Step D:

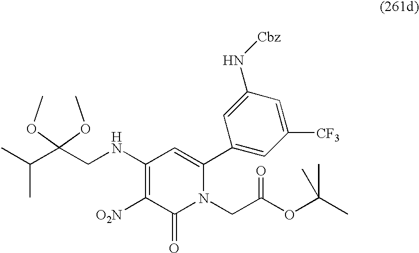

To a solution of bicyclic pyridone acetate 261c from Step C (300 mmol) in 1500 mL of ethanol is added 2,2-dimethoxy-3-methylbutanamine (300 mmol) and 600 mmol of triethylamine. The solution is stirred at 70° C. for 16 hours or until the reaction is complete. The reaction mixture is cooled and evaporated to remove all of the ethanol. The residue is partitioned between ethyl acetate and water, and the organic phase is washed with brine, dried over MgSO₄, concentrated, and then chromatographed to afford the desired nitro ketal 261d.

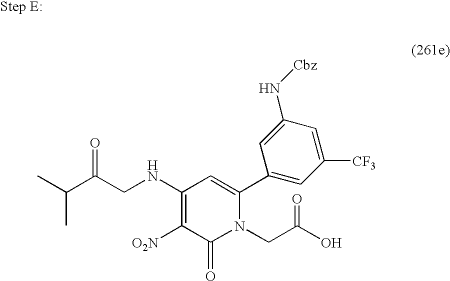

(261e)

The nitro ketal 261d from Step D (250 mmol) is hydrolyzed and the tert-butyl ester removed by stirring with trifluoroacetic acid (50 mL), water (200 mL) and THF (500 mL) until completion as monitored by chromatography. The reaction mixture is concentrated at ambient temperature to give the trifluroacetic acid salt of unpurified nitro ketone 261e and used as is in the next step.

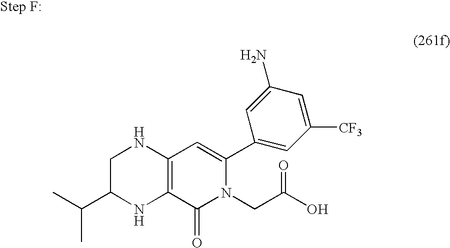

To the nitro ketone 261e from Step E (225 mmol) in 1000 mL of ethyl acetate is added 20 grams of 10% Pd/C. Hydrogen gas is added until uptake of hydrogen stopped. The reaction mixture is filtered through Celite and the filtrate evaporated. The residue is then chromatographed to afford the desired bicyclic pyridone acetic acid 19f.

A solution of compound bicyclic pyridone acetic acid 261f (50 mmol) in DMF (250 mL) is treated with N-hydroxybenzotriazole (60 mmol) and EDC hydrochloride (60 mmol). The mixture is stirred at room temperature for 30 min and treated with 4-(N-Cbz-amidinobenzylamine (50 mmol). The resulting mixture is allowed to stir overnight. Typical aqueous workup is followed by chromatographic purification to afford pure Example 261 product.

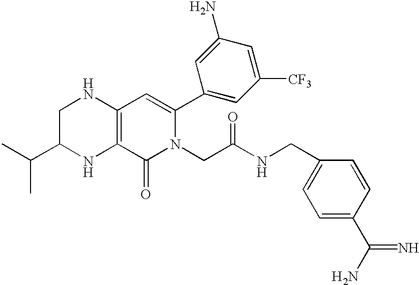

Compound of Example 261 (10 mmol) and 10% Pd on activated carbon (0.100 g) are mixed with 100 mL methanol. The mixture is stirred for 2 hours under an atmosphere of hydrogen that is introduced through a rubber balloon. After filtering off the catalyst and removing the methanol, the remaining residue is obtained as Example 262.

Using these methods and ordinary skill in the art numerous novel compounds of the present invention have been or can be prepared.

### Biological Data

Effect of Compounds of the Invention in Combination with Aspirin for the Treatment of Thrombus in Mammals

Deep Vein Thrombosis Model

This model measures the accumulation of thrombus on a set of four threads (4-0 surgical silk) that are introduced into the vena cava of a non-human primate. The device used to introduce the threads consists of a copper wire (12 cm) contained in a sheath of polyethylene tubing (9 cm). Four 4-0 surgical silk threads (3 cm) are tied to the end of the wire and retracted into the tubing. When the device is inserted into the iliac vein, the wire is advanced introducing the threads into the vena cava between the bifurcation and the left renal vein.

Non-human primates (Macaca fascicularis) are lightly anesthetized with ketamine to facilitate placement of an endotracheal tube and a femoral venous catheter. Animals are put into and maintained in a deep plane of anesthesia using pentobarbital via the femoral vein cannula. A jugular vein is cannulated for infusion of drug and a carotid artery is cannulated for blood sampling and for monitoring hemodynamic parameters.

The abdominal cavity is opened and an ultrasonic flow probe placed on the left iliac vein. The right iliac vein (RIV) is dissected free of surrounding tissue for a length of approximately 4 cm. The RIV is ligated approximately 4 cm distal to the bifurcation of the abdominal vena cava and a second ligature is placed, loosely, approximately 2 cm distal to the bifurcation. An incision is made in the RIV between the two ligatures, the device described above introduced into the iliac vein and the threads advanced into the vena cava. Every study consists of three thrombus measurements:

Control—Saline infusion. Threads are introduced and left in for 30 min, removed and thrombus weighed.

Treatment 1—Aspirin administered as a bolus or compound as an infusion. For aspirin treatment, aspirin is administered as a bolus and a 30 min equilibration period is allowed to assure the platelets are inhibited. For compound treatment, infusion is started and allowed to run for 30 min to reach steady-state blood levels of compound. Threads are introduced and left in for 30 min, removed and thrombus weighed;

Treatment 2—Compound administered as an infusion. Compound infusion is started and allowed to run for 30 min to reach steady-state blood levels of compound. Threads are introduced and left in for 30 min, removed and thrombus weighed.

During the 30 min equilibration/infusion periods of Treatment 1 and Treatment 2, a piece of tubing containing a copper wire, but no threads, is introduced into the vein to maintain the integrity of the vessel. After the threads have been in the vessel for 30 min, the entire device is removed from the vessel and the threads are cut from the device and weighed. The weight of the threads prior to insertion in the vessel is subtracted to get the net thrombus weight

To measure bleeding time, a blood pressure cuff is placed on the upper arm and inflated to 40 mm Hg. Two uniform cuts are made in the volar surface of the forearm, the blood from the cuts is collected onto a gauze pad every 30 sec. The time from when the cuts are made until the bleeding stops is considered the bleeding time. All gauze used to collect blood is placed in Drabkin's solution and the amount of blood lost from the bleeding time site is calculated based on the amount of hemoglobin measured in the Drabkin's solution.

The table below depicts the effects of Aspirin coadministered with a compound of the present invention (compounds 54 and 55 are salts of the compound tested) on thrombus formation, prothrombin time and bleeding time in a non-human primate model of deep vein thrombosis.

| | | | | Thrombus | | Pro- | | | |
|---|---|---|---|---|---|---|---|---|---|
| | | Net | | Wt: % Δ | | thrombin | | Bleeding | |
| | | Thrombus | | from | | Time | | Time | |
| n= | Treatment* | Wt (mg) | SD | Control | SD | (sec) | SD | (min) | SD |
| 4 | Control | 34.8 | 5.5 | — | — | 10.3 | 0.5 | 3.5 | 0.2 |
| 4 | 100 | 15.3 | 1.5 | −56 | 3.8 | 22.7 | 2.0 | 0.1 | |
| | μg/kg/min | | | | | | | | |
| 4 | 200 | 5.1 | 0.7 | −85 | 0.6 | 46.0 | 4.0 | 3.9 | 0.1 |
| | μg/kg/min | | | | | | | | |
| 4 | Control | 34.5 | 9.3 | — | — | 10.2 | 0.1 | 3.3 | 0.3 |
| 4 | Aspirin | 30.9 | 9.9 | −12 | 5.3 | 11.8 | 1.2 | 4.6 | 0.5 |
| 4 | 100 | 3.6 | 1.7 | −89 | 5.2 | 22.4 | 1.5 | 4.7 | 0.4 |
| | μg/kg/min | | | | | | | | |
| 4 | Control | 27.8 | 7.5 | — | — | 10.5 | 0.6 | 3.0 | 0.0 |
| 4 | Aspirin | 26.7 | 7.0 | −4 | 6.6 | 11.5 | 0.8 | 4.3 | 0.5 |
| 4 | 50 | 19.5 | 16.8 | −34 | 39.2 | 17.3 | 3.5 | 4.1 | 0.1 |
| | μg/kg/min | | | | | | | | |

*All animals treated with Compounds 54 and 55

1. A compound having the structure: 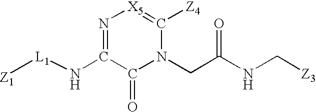
wherein
X₅ is CH, C(F), or C(Br);
L₁ is a linker, linking Z₁ to the heterocyclic ring;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy, or alkoxycarbonyl;
Z₃ comprises a substituted phenyl, thienyl, or furanyl ring, the phenyl, thienyl or furanyl ring being substituted with an amidine or a derivatized amidine group, and optionally further substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂₁ Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅;
R₄₂ is amino; and
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen;
provided, however, one of the following conditions exist: (a) Z₁ is other than unsubstituted cyclobutyl when X₅ is CH; (b) Z₁ is other than unsubstituted isopropyl when (i) X₅ is CH and (ii) Z₄ is 3,5-diaminophenyl or 3-amino-5-(2,2,2-trifluoroacetamide)phenyl; or (c) Z₃ is other than 4-amidinobenzyl, 4-amidino-2-fluorobenzyl, or 4-amidino-3-fluorobenzyl.

2. The compound of claim 1 wherein Z₁ is C₁-C₈ alkyl, C₂-C₈ alkenyl, or C₂-C₈ alkynyl, the alkyl, alkenyl, or alkynyl being optionally substituted at any substitutable position with fluorine;
Z₃ comprises a substituted phenyl or substituted thienyl ring, the phenyl or thienyl ring being substituted with an amidine or derivatized amidine, and optionally further substituted with fluorine or hydroxy;
R₄₄ is hydrocarbyl, substituted hydrocarbyl, heterocyclo, halogen, or a substituted or unsubstituted heteroatom selected from nitrogen, oxygen, sulfur and phosphorus; and
X₅, L₁, L₃, Z₄ and R₄₂ are as defined in claim 1;
provided, however, one of the following conditions exist: (a) Z₁ is other than cyclobutyl when X₅ is CH; (b) Z₁ is other than isopropyl when (i) X₅ is CH and (ii) Z₄ is 3,5-diaminophenyl or 3-amino-5-(2,2,2-trifluoroacetamide)phenyl; or (c) Z₃ is other than 4-amidinobenzyl, 4-amidino-2-fluorobenzyl, or 4-amidino-3-fluorobenzyl.

3. The compound of claims 1 or 2 wherein L₁ is a bond.

4. The compound of claims 1 or 2 wherein Z₁ is C₁-C₅ alkyl optionally substituted at any substitutable position with fluorine.

5. The compound of claim 3 wherein Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl.

6. The compound of claim 3 wherein Z₁ is selected from the group consisting of cyclopropyl, isopropyl, methyl, ethyl, cyclobutyl, isobutyl, and sec-butyl.

7. The compound of claim 1 wherein Z₁ is isopropyl or cyclobutyl substituted with fluorine, hydroxy, carboxy, or alkoxycarbonyl.

8. The compound of claim 3 wherein Z₃ is a phenyl ring substituted with an amidine group.

9. The compound of claim 1 wherein Z₃ is a phenyl, thienyl, or furanyl ring substituted with a derivatized amidine which, upon hydrolysis, oxidation, reduction, or elimination, or any combination thereof, under physiological conditions yields an amidine group.

10. The compound of claim 2 wherein Z₃ is a phenyl or thienyl ring substituted with a derivatized amidine which, upon hydrolysis, oxidation, reduction or elimination under physiological conditions yields an amidine group.

11. The compound of any of claims 8-10 wherein Z₃ is further substituted at any position with fluorine or hydroxy.

12. The compound of claim 1 wherein Z₃ is 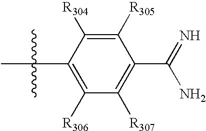
wherein
R₃₀₄ and R₃₀₆ are independently selected from the group consisting of hydrogen, fluorine, hydroxy, carboxy, hydrocarbyloxy and alkoxycarbonyl; and
R₃₀₅ and R₃₀₇ are independently selected from the group consisting of hydrogen, fluorine, methoxy, hydroxy and carboxy.

13. The compound of claim 1 or 2 wherein Z₄ is 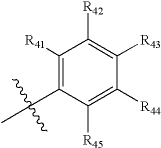
wherein:
R₄₂ is as defined in claim 1;
R₄₄ is hydrocarbyl, substituted hydrocarbyl, halogen or an optionally substituted heteroatom selected from the group consisting of oxygen, nitrogen, and sulfur; and
R₄₁, R₄₃ and R₄₅ are independently hydrogen, hydrocarbyl, substituted hydrocarbyl, halogen or an optionally substituted heteroatom selected from the group consisting of oxygen, nitrogen, and sulfur.

14. The compound of claim 13 wherein R₄₂ is as defined in claim 1, R₄₄ is as defined in claim 13, and R₄₁, R₄₃ and R₄₅ are independently hydrogen, halogen, alkoxy, or alkyl, optionally substituted with halogen or alkoxy.

15. The compound claim 13 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

16. The compound of claim 15 wherein R₄₄ is selected from the group consisting of hydroxy, carboxy, carboxamido, alkoxy, alkylsulfonyl, sulfonamido, or alkoxycarbonyl.

17. The compound of claim 16 wherein R₄₄ is sec-butylamide, carboxy, ethoxycarbonyl, isopropoxycarbonyl, butoxycarbonyl, isopropylamide or hydroxy.

18. The compound of claim 15 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine.

19. The compound of claim 16 wherein L₁ is a bond, Z₁ is isopropyl or cyclopropyl, Z₃ is phenyl substituted with an amidine group, and R₄₄ is as defined in claim 16.

20. The compound of claim 13 wherein Z₄₁, Z₄₃ or Z₄₅ is substituted with fluorine or chlorine.

21. The compound of claim 1 wherein Z₄ is 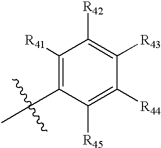
wherein
R₄₂ is as defined in claim 1;
R₄₃ is hydrocarbyl, substituted hydrocarbyl, halogen or an optionally substituted heteroatom selected from the group consisting of oxygen, nitrogen, and sulfur; and
R₄₁, R₄₄ and R₄₅ are independently hydrogen, halogen or alkoxy.

22. The compound of claim 1 wherein Z₄ is 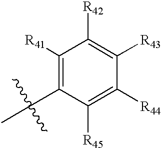
wherein
R₄₂ is as defined in claim 1;
R₄₅ is hydrocarbyl, substituted hydrocarbyl, halogen or an optionally substituted heteroatom selected from the group consisting of oxygen, nitrogen, and sulfur; and
R₄₁, R₄₃ and R₄₄ are independently hydrogen, halogen or alkoxy.

23. The compound of claim 1 wherein Z₄ is 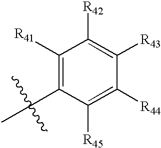
wherein
R₄₂ is as defined in claim 1;
R₄₁ is hydrocarbyl, substituted hydrocarbyl, halogen or an optionally substituted heteroatom selected from the group consisting of oxygen, nitrogen, and sulfur; and
R₄₃, R₄₄ and R₄₅ are independently hydrogen, halogen or alkoxy.

24. The compound of claim 1 wherein L₁ is a bond; Z₁ is selected from the group consisting of cyclopropyl, isopropyl, methyl, ethyl, cyclobutyl, isobutyl, tert-butyl, and sec-butyl optionally substituted at any substitutable position with fluorine, hydroxy, carboxy, or alkoxycarbonyl; Z₃ is phenyl substituted with an amidine group and optionally substituted by hydrogen, fluorine, hydroxy, carboxy, alkoxycarbonyl, or hydrocarbyloxy; and one of R₄₁, R₄₃, R₄₄ or R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

25. The compound of claim 2 wherein L₁ is a bond; Z₁ is selected from the group consisting of cyclopropyl, isopropyl, methyl, ethyl, cyclobutyl, isobutyl, and sec-butyl optionally substituted at any substitutable position with fluorine; and R₄₄ is selected from the group consisting of hydroxy, alkylsulfonyl, haloalkyl, haloalkoxy, haloalkylthio, carboxamidoalkyl, and carboxamidoalkylaryl.

26. The compound of claim 1 having the structure: 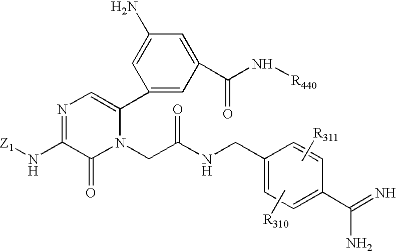
wherein
Z₁ is isopropyl or cyclopropyl optionally substituted with fluorine, hydroxy, carboxy, or alkoxycarbonyl;
R₄₄₀ is C₁-C₆ alkyl, aryl, aralkyl, carboxy, or carboxyalkyl, wherein said alkyl, aryl, aralkyl, carboxy, or carboxyalkyl is optionally further substituted by fluorine; and
R₃₁₀ and R₃₁₁ are independently selected from the group consisting of hydrogen, fluorine, hydroxy, alkoxy, and carboxy.

27. The compound of claim 1 having the structure: 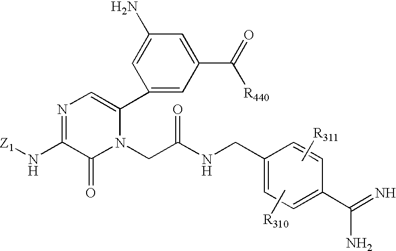
wherein
Z₁ is isopropyl or cyclopropyl optionally substituted with fluorine, hydroxy, carboxy, or alkoxycarbonyl;
R₄₄₀ is C₁-C₆ alkyl, aryl, aralkyl, carboxy, hydroxy or carboxyalkyl, wherein said alkyl, aryl, aralkyl, carboxy, hydroxy or carboxyalkyl is optionally further substituted by fluorine; and
R₃₁₀ and R₃₁₁ are independently selected from the group consisting of hydrogen, fluorine, hydroxy, alkoxy, and carboxy.

28. The compound of claim 2 having the structure: 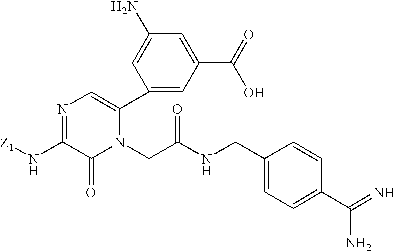
wherein Z₁ is isopropyl or cyclopropyl.

29. The compound of claim 2 having the structure: 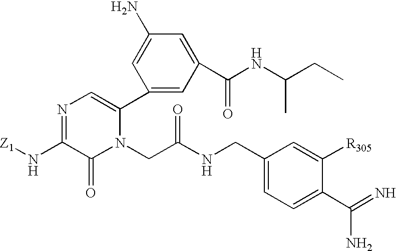
wherein R₃₀₅ is hydrogen or hydroxy; and Z₁ is isopropyl or cyclopropyl.

30. The compound of claim 2 having the structure: 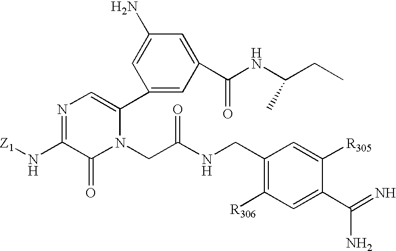
wherein one of R₃₀₅ and R₃₀₆ is hydroxy and the other is hydrogen; and Z₁ is isopropyl or cyclopropyl.

31. The compound of claim 2 having the structure: 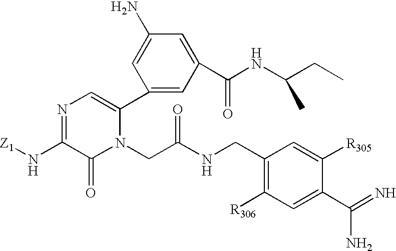
wherein one of R₃₀₅ and R₃₀₆ is hydroxy and the other is hydrogen; and Z₁ is isopropyl or cyclopropyl.

32. The compound of claim 1 having the structure: 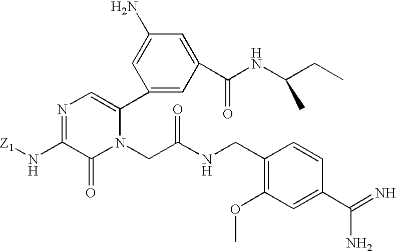
wherein Z₁ is isopropyl or cyclopropyl.

33. The compound of claim 2 having the structure: 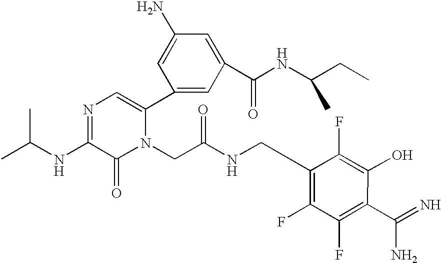
wherein Z₁ is isopropyl or cyclopropyl.

34. The compound of claim 2 having the structure: 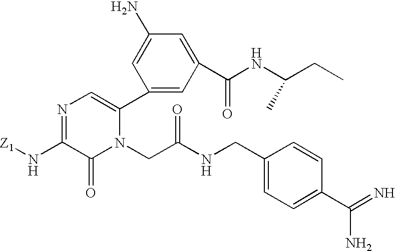
wherein Z₁ is isopropyl or cyclopropyl.

35. The compound of claim 2 having the structure: 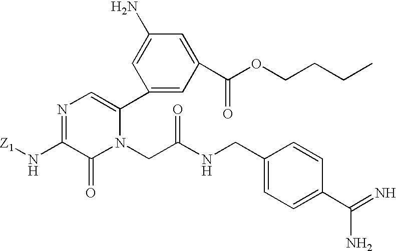
wherein Z₁ is isopropyl or cyclopropyl.

36. The compound of claim 2 having the structure: 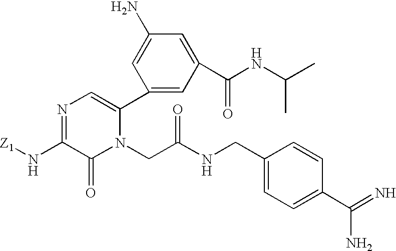
wherein Z₁ is isopropyl or cyclopropyl.

37. The compound of claim 2 having the structure: 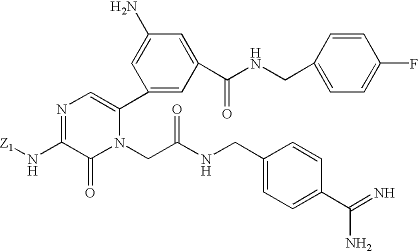
wherein Z₁ is isopropyl or cyclopropyl.

38. The compound of claim 2 having the structure: 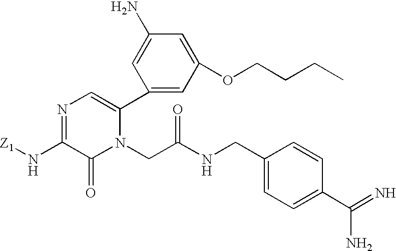
wherein Z₁ is isopropyl or cyclopropyl.

39. The compound of claim 2 having the structure: 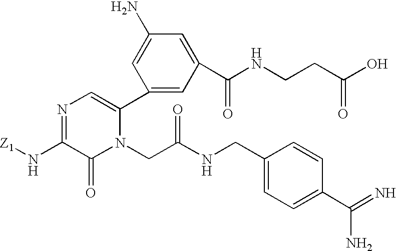
wherein Z₁ is isopropyl or cyclopropyl.

40. The compound of claim 2 having the structure: 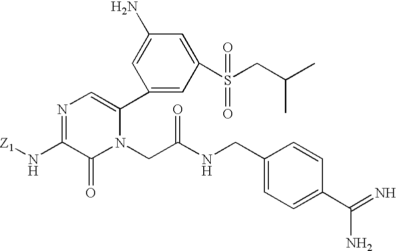
wherein Z₁ is isopropyl or cyclopropyl.

41. The compound of claim 2 having the structure: 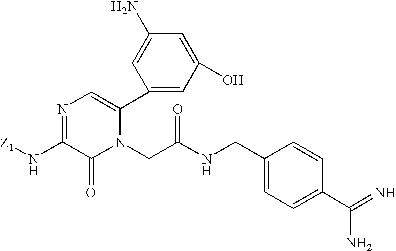
wherein Z₁ is isopropyl or cyclopropyl.

42. A compound having the structure: 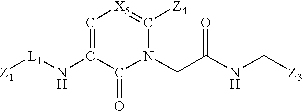
wherein
X₅ is CH, C(Br), C(Cl), or C(F)
L₁ is a linker, linking Z₁ to the heterocyclic ring and optionally containing a bond to the carbon of the heterocyclic ring that is gamma to the substituted nitrogen of the heterocyclic ring to form a fused ring with the heterocyclic ring;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy or alkoxycarbonyl;
Z₃ comprises a substituted phenyl, thienyl, or furanyl ring, the phenyl, thienyl or furanyl ring being substituted with an amidine or a derivatized amidine group, and optionally further substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅;
R₄₂ is amino; and
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁₁ R₄₃, R₄₄ or R₄₅ is other than hydrogen;
provided, however, one of the following conditions exist: (a) Z₃ is other than 4-amidinobenzyl, 4-amidino-2-fluorobenzyl, and 4-amidino-3-fluorobenzyl; or (b) (i) Z₁ is other than unsubstituted cyclobutyl and unsubstituted isopropyl when X₅ is CH or C(Cl) and (ii) neither Z₄₁ nor Z₄₅ is sulfur when Z₄ is thienyl;

43. The compound of claim 42 wherein Z₁ is C₁-C₈ alkyl, C₂-C₈ alkenyl, or C₂-C₈ alkynyl, the alkyl, alkenyl, or alkynyl being optionally substituted at any substitutable position with fluorine;
Z₃ comprises a substituted phenyl or substituted thienyl ring, the phenyl or thienyl ring being substituted with an amidine or derivatized amidine, and optionally further substituted with fluorine or hydroxy;
R₄₄ is hydrocarbyl, substituted hydrocarbyl, heterocyclo, halogen, or a substituted or unsubstituted heteroatom selected from nitrogen, oxygen, sulfur and phosphorus; and
X₅, L₁, L₃, Z₄ and R₄₂ are as defined in claim 39;
provided, however, one of the following conditions exist: (a) Z₃ is other than 4-amidinobenzyl, 4-amidino-2-fluorobenzyl, and 4-amidino-3-fluorobenzyl; or (b) (i) Z₁ is other than cyclobutyl and isopropyl when X₅ is CH or C(Cl) and (ii) neither Z₄₁ nor Z₄₅ is sulfur when Z₄ is thienyl;

44. The compound of claims 42 or 43 wherein L₁ is a bond.

45. The compound of claims 42 or 43 wherein Z₁ is C₁-C₅ alkyl optionally substituted at any substitutable position with fluorine.

46. The compound of claim 44 wherein Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl.

47. The compound of claim 44 wherein Z₁ is selected from the group consisting of cyclopropyl, isopropyl, methyl, ethyl, cyclobutyl, isobutyl, and sec-butyl.

48. The compound of claim 42 wherein Z₁ is isopropyl or cyclobutyl substituted with fluorine, hydroxy, carboxy, or alkoxycarbonyl.

49. The compound of claim 44 wherein Z₃ is a phenyl ring substituted with an amidine group.

50. The compound of claim 42 wherein Z₃ is a phenyl, thienyl, or furanyl ring substituted with a derivatized amidine which, upon hydrolysis, oxidation, reduction, or elimination, or any combination thereof, under physiological conditions yields an amidine group.

51. The compound of claim 43 wherein Z₃ is a phenyl or thienyl ring substituted with a derivatized amidine which, upon hydrolysis, oxidation, reduction or elimination under physiological conditions yields an amidine group.

52. The compound of any of claims 49-51 wherein Z₃ is further substituted at any position with fluorine or hydroxy.

53. The compound of claim 42 wherein Z₃ is 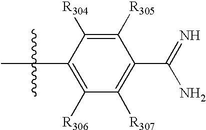
wherein
R₃₀₄ and R₃₀₆ are independently selected from the group consisting of hydrogen, fluorine, hydroxy, carboxy, hydrocarbyloxy and alkoxycarbonyl; and
R₃₀₅ and R₃₀₇ are independently selected from the group consisting of hydrogen, fluorine, methoxy, hydroxy and carboxy.

54. The compound of claim 42 or 43 wherein Z₄ is 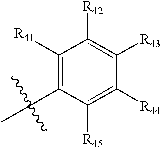
wherein:
R₄₂ is as defined in claim 1;
R₄₄ is hydrocarbyl, substituted hydrocarbyl, halogen or an optionally substituted heteroatom selected from the group consisting of oxygen, nitrogen, and sulfur; and
R₄₁₁ R₄₃ and R₄₅ are independently hydrogen, hydrocarbyl, substituted hydrocarbyl, halogen or an optionally substituted heteroatom selected from the group consisting of oxygen, nitrogen, and sulfur.

55. The compound of claim 54 wherein R₄₂ is as defined in claim 42, R₄₄ is as defined in claim 54, and R₄₁, R₄₃ and R₄₅ are independently hydrogen, halogen, alkoxy, or alkyl, optionally substituted with halogen or alkoxy.

56. The compound claim 54 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

57. The compound of claim 56 wherein R₄₄ is selected from the group consisting of hydroxy, carboxy, carboxamido, alkoxy, alkylsulfonyl, sulfonamido, or alkoxycarbonyl.

58. The compound of claim 57 wherein R₄₄ is sec-butylamide, carboxy, ethoxycarbonyl, isopropoxycarbonyl, butoxycarbonyl, isopropylamide or hydroxy.

59. The compound of claim 56 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine.

60. The compound of claim 57 wherein L₁ is a bond, Z₁ is isopropyl or cyclopropyl, Z₃ is phenyl substituted with an amidine group, and R₄₄ is as defined in claim 57.

61. The compound of claim 54 wherein Z₄₁, Z₄₃ or Z₄₅ is substituted with fluorine or chlorine.

62. The compound of claim 42 wherein Z₄ is 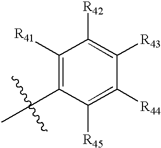
wherein
R₄₂ is as defined in claim 42;
R₄₃ is hydrocarbyl, substituted hydrocarbyl, halogen or an optionally substituted heteroatom selected from the group consisting of oxygen, nitrogen, and sulfur; and
R₄₁, R₄₄ and R₄₅ are independently hydrogen, halogen or alkoxy.

63. The compound of claim 42 wherein Z₄ is 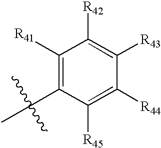
wherein
R₄₂ is as defined in claim 42;
R₄₅ is hydrocarbyl, substituted hydrocarbyl, halogen or an optionally substituted heteroatom selected from the group consisting of oxygen, nitrogen, and sulfur; and
R₄₁, R₄₃ and R₄₄ are independently hydrogen, halogen or alkoxy.

64. The compound of claim 42 wherein Z₄ is 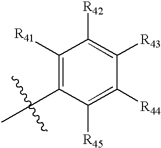
wherein
R₄₂ is as defined in claim 42;
R₄₁ is hydrocarbyl, substituted hydrocarbyl, halogen or an optionally substituted heteroatom selected from the group consisting of oxygen, nitrogen, and sulfur; and
R₄₃, R₄₄ and R₄₅ are independently hydrogen, halogen or alkoxy.

65. The compound of claim 42 wherein L₁ is a bond; Z₁ is selected from the group consisting of cyclopropyl, isopropyl, methyl, ethyl, cyclobutyl, isobutyl, tert-butyl, and sec-butyl optionally substituted at any substitutable position with fluorine, hydroxy, carboxy, or alkoxycarbonyl; Z₃ is phenyl substituted with an amidine group and optionally substituted by hydrogen, fluorine, hydroxy, carboxy, alkoxycarbonyl, or hydrocarbyloxy; and one of R₄₁, R₄₃, R₄₄ or R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

66. The compound of claim 43 wherein L₁ is a bond; Z₁ is selected from the group consisting of cyclopropyl, isopropyl, methyl, ethyl, cyclobutyl, isobutyl, and sec-butyl optionally substituted at any substitutable position with fluorine; and R₄₄ is selected from the group consisting of hydroxy, alkylsulfonyl, haloalkyl, haloalkoxy, haloalkylthio, carboxamidoalkyl, and carboxamidoalkylaryl.

67. A compound having the structure: 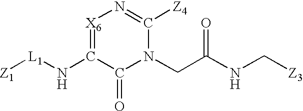
wherein
X₆ is CH, C(Br), C(Cl), or C(F);
L₁ is a linker, linking Z₁ to the heterocyclic ring and optionally containing a bond to the carbon of the heterocyclic ring that is gamma to the substituted nitrogen of the heterocyclic ring to form a fused ring with the heterocyclic ring;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy, or alkoxycarbonyl provided that Z₁ is other than unsubstituted cyclobutyl or unsubstituted isopropyl.
Z₃ comprises a substituted phenyl, thienyl, or furanyl ring, the phenyl, thienyl or furanyl ring being substituted with an amidine or a derivatized amidine group, and optionally further substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅, provided neither Z₄₁ nor Z₄₅ is sulfur when Z₄ is thienyl;
R₄₂ is amino; and
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen.

68. The compound of claim 67 wherein X₆ is CH;
Z₁ is C₁-C₈ alkyl, C₂-C₈ alkenyl, or C₂-C₈ alkynyl, the alkyl, alkenyl, or alkynyl being optionally substituted at any substitutable position with fluorine, provided that Z₁ is other than cyclobutyl or isopropyl;
Z₃ comprises a substituted phenyl or substituted thienyl ring, the phenyl or thienyl ring being substituted with an amidine or a derivatized amidine group, and optionally further substituted with fluorine or hydroxy;
R₄₄ is hydrocarbyl, substituted hydrocarbyl, heterocyclo, halogen, or a substituted or unsubstituted heteroatom selected from nitrogen, oxygen, sulfur and phosphorus; and
L₁, L₃, Z₄ and R₄₂ are as defined in claim 62.

69. The compound of claims 67 or 68 wherein L₁ is a bond.

70. The compound of claims 67 or 68 wherein Z₁ is C₁-C₅ alkyl optionally substituted at any substitutable position with fluorine.

71. The compound of claim 69 wherein Z₁ is selected from the group consisting of cyclopropyl, methyl, ethyl, isobutyl, tert-butyl and sec-butyl optionally substituted at any substitutable position with fluorine, hydroxy, carboxy or alkoxycarbonyl.

72. The compound of claim 69 wherein Z₁ is selected from the group consisting of cyclopropyl, methyl, ethyl, isobutyl, and sec-butyl.

73. The compound of claim 67 wherein Z₁ is isopropyl or cyclobutyl substituted with fluorine, hydroxy, carboxy or alkoxycarbonyl.

74. The compound of claim 69 wherein Z₃ is a phenyl ring substituted with an amidine group.

75. The compound of claim 67 wherein Z₃ is a phenyl, thienyl, or furanyl ring substituted with a derivatized amidine which, upon hydrolysis, oxidation, reduction, or elimination, or any combination thereof, under physiological conditions yields an amidine group.

76. The compound of claim 68 wherein Z₃ is a phenyl or thienyl ring substituted with a derivatized amidine which, upon hydrolysis, oxidation, reduction or elimination under physiological conditions yields an amidine group.

77. The compound of any of claims 74-76 wherein Z₃ is further substituted at any position with fluorine or hydroxy.

78. The compound of claim 67 wherein Z₃ is 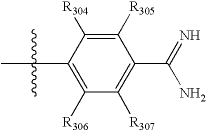
wherein
R₃₀₄ and R₃₀₆ are independently selected from the group consisting of hydrogen, fluorine, hydroxy, carboxy, hydrocarbyloxy and alkoxycarbonyl; and
R₃₀₅ and R₃₀₇ are independently selected from the group consisting of hydrogen, fluorine, methoxy, hydroxy and carboxy.

79. The compound of claim 67 or 68 wherein Z₄ is 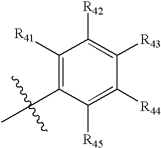
wherein:
R₄₂ is as defined in claim 67;
R₄₄ is hydrocarbyl, substituted hydrocarbyl, halogen or an optionally substituted heteroatom selected from the group consisting of oxygen, nitrogen, and sulfur; and
R₄₁, R₄₃ and R₄₅ are independently hydrogen, hydrocarbyl, substituted hydrocarbyl, halogen or an optionally substituted heteroatom selected from the group consisting of oxygen, nitrogen, and sulfur.

80. The compound of claim 79 wherein R₄₂ is as defined in claim 67, R₄₄ is as defined in claim 79, and R₄₁, R₄₃ and R₄₅ are independently hydrogen, halogen, alkoxy, or alkyl, optionally substituted with halogen or alkoxy.

81. The compound claim 79 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

82. The compound of claim 81 wherein R₄₄ is selected from the group consisting of hydroxy, carboxy, carboxamido, alkoxy, alkylsulfonyl, sulfonamido, or alkoxycarbonyl.

83. The compound of claim 82 wherein R₄₄ is sec-butylamide, carboxy, ethoxycarbonyl, isopropoxycarbonyl, butoxycarbonyl, isopropylamide or hydroxy.

84. The compound of claim 81 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine.

85. The compound of claim 82 wherein L₁ is a bond, Z₁ is isopropyl or cyclopropyl, Z₃ is phenyl substituted with an amidine group, and R₄₄ is as defined in claim 82.

86. The compound of claim 79 wherein Z₄₁, Z₄₃ or Z₄₅ is substituted with fluorine or chlorine.

87. The compound of claim 67 wherein Z₄ is 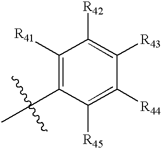
wherein
R₄₂ is as defined in claim 67;
R₄₃ is hydrocarbyl, substituted hydrocarbyl, halogen or an optionally substituted heteroatom selected from the group consisting of oxygen, nitrogen, and sulfur; and
R₄₁, R₄₄ and R₄₅ are independently hydrogen, halogen or alkoxy.

88. The compound of claim 67 wherein Z₄ is 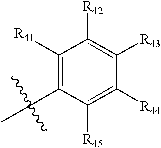
wherein
R₄₂ is as defined in claim 67;
R₄₅ is hydrocarbyl, substituted hydrocarbyl, halogen or an optionally substituted heteroatom selected from the group consisting of oxygen, nitrogen, and sulfur; and
R₄₁, R₄₃ and R₄₄ are independently hydrogen, halogen or alkoxy.

89. The compound of claim 67 wherein Z₄ is 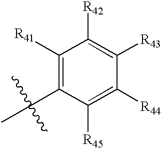
wherein
R₄₂ is as defined in claim 67;
R₄₁ is hydrocarbyl, substituted hydrocarbyl, halogen or an optionally substituted heteroatom selected from the group consisting of oxygen, nitrogen, and sulfur; and
R₄₃, R₄₄ and R₄₅ are independently hydrogen, halogen or alkoxy.

90. The compound of claim 67 wherein L₁ is a bond; Z₁ is selected from the group consisting of cyclopropyl, methyl, ethyl, isobutyl, tert-butyl, and sec-butyl optionally substituted at any substitutable position with fluorine, hydroxy, carboxy, or alkoxycarbonyl; Z₃ is phenyl substituted with an amidine group and optionally substituted by hydrogen, fluorine, hydroxy, carboxy, alkoxycarbonyl, or hydrocarbyloxy; and one of R₄₁, R₄₃, R₄₄ or R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

91. The compound of claim 68 wherein L₁ is a bond; Z₁ is selected from the group consisting of cyclopropyl, methyl, ethyl, isobutyl, and sec-butyl optionally substituted at any substitutable position with fluorine; and R₄₄ is selected from the group consisting of hydroxy, alkylsulfonyl, haloalkyl, haloalkoxy, haloalkylthio, carboxamidoalkyl, and carboxamidoalkylaryl.

92. A compound having the structure: 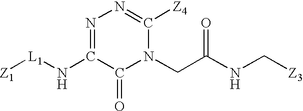
wherein
L₁ is a linker, linking Z₁ to the heterocyclic ring and optionally containing a bond to the carbon of the heterocyclic ring that is gamma to the substituted nitrogen of the heterocyclic ring to form a fused ring with the heterocyclic ring;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy, or alkoxycarbonyl provided that Z₁ is other than unsubstituted cyclobutyl or unsubstituted isopropyl.
Z₃ comprises a substituted phenyl, thienyl, or furanyl ring, the phenyl, thienyl or furanyl ring being substituted with an amidine or a derivatized amidine group, and optionally further substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀₁ Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅, provided neither Z₄₁ nor Z₄₅ is sulfur when Z₄ is thienyl;
R₄₂ is amino; and
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen.

93. The compound of claim 92 wherein Z₁ is C₁-C₈ alkyl, C₂-C₈ alkenyl, or C₂-C₈ alkynyl, the alkyl, alkenyl, or alkynyl being optionally substituted at any substitutable position with fluorine, provided that Z₁ is other than cyclobutyl or isopropyl;
Z₃ comprises a substituted phenyl or substituted thienyl ring, the phenyl or thienyl ring being substituted with an amidine or a derivatized amidine group, and optionally further substituted with fluorine or hydroxy;
R₄₄ is hydrocarbyl, substituted hydrocarbyl, heterocyclo, halogen, or a substituted or unsubstituted heteroatom selected from nitrogen, oxygen, sulfur and phosphorus; and
L₁, L₃, Z₄ and R₄₂ are as defined in claim 62.

94. The compound of claims 92 or 93 wherein L₁ is a bond.

95. The compound of claims 92 or 93 wherein Z₁ is C₁-C₅ alkyl optionally substituted at any substitutable position with fluorine.

96. The compound of claim 94 wherein Z₁ is selected from the group consisting of cyclopropyl, methyl, ethyl, isobutyl, tert-butyl and sec-butyl optionally substituted at any substitutable position with fluorine, hydroxy, carboxy or alkoxycarbonyl.

97. The compound of claim 94 wherein Z₁ is selected from the group consisting of cyclopropyl, methyl, ethyl, isobutyl, and sec-butyl.

98. The compound of claim 92 wherein Z₁ is isopropyl or cyclobutyl substituted with fluorine, hydroxy, carboxy or alkoxycarbonyl.

99. The compound of claim 94 wherein Z₃ is a phenyl ring substituted with an amidine group.

100. The compound of claim 92 wherein Z₃ is a phenyl, thienyl, or furanyl ring substituted with a derivatized amidine which, upon hydrolysis, oxidation, reduction, or elimination, or any combination thereof, under physiological conditions yields an amidine group.

101. The compound of claim 93 wherein Z₃ is a phenyl or thienyl ring substituted with a derivatized amidine which, upon hydrolysis, oxidation, reduction or elimination under physiological conditions yields an amidine group.

102. The compound of any of claims 99-101 wherein Z₃ is further substituted at any position with fluorine or hydroxy.

103. The compound of claim 92 wherein Z₃ is 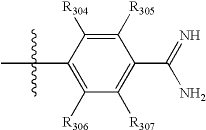
wherein
R₃₀₄ and R₃₀₆ are independently selected from the group consisting of hydrogen, fluorine, hydroxy, carboxy, hydrocarbyloxy and alkoxycarbonyl; and
R₃₀₅ and R₃₀₇ are independently selected from the group consisting of hydrogen, fluorine, methoxy, hydroxy and carboxy.

104. The compound of claim 92 or 93 wherein Z₄ is 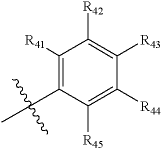
wherein:
R₄₂ is as defined in claim 92;
R₄₄ is hydrocarbyl, substituted hydrocarbyl, halogen or an optionally substituted heteroatom selected from the group consisting of oxygen, nitrogen, and sulfur; and
R₄₁, R₄₃ and R₄₅ are independently hydrogen, hydrocarbyl, substituted hydrocarbyl, halogen or an optionally substituted heteroatom selected from the group consisting of oxygen, nitrogen, and sulfur.

105. The compound of claim 104 wherein R₄₂ is as defined in claim 1, R₄₄ is as defined in claim 106, and R₄₁₁ R₄₃ and R₄₅ are independently hydrogen, halogen, alkoxy, or alkyl, optionally substituted with halogen or alkoxy.

106. The compound claim 104 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

107. The compound of claim 106 wherein R₄₄ is selected from the group consisting of hydroxy, carboxy, carboxamido, alkoxy, alkylsulfonyl, sulfonamido, or alkoxycarbonyl.

108. The compound of claim 107 wherein R₄₄ is sec-butylamide, carboxy, ethoxycarbonyl, isopropoxycarbonyl, butoxycarbonyl, isopropylamide or hydroxy.

109. The compound of claim 106 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine.

110. The compound of claim 107 wherein L₁ is a bond, Z₁ is isopropyl or cyclopropyl, Z₃ is phenyl substituted with an amidine group, and R₄₄ is as defined in claim 107.

111. The compound of claim 92 wherein Z₄₁, Z₄₃ or Z₄S is substituted with fluorine or chlorine.

112. The compound of claim 92 wherein Z₄ is 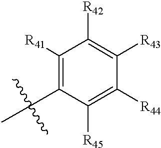
wherein
R₄₂ is as defined in claim 92;
R₄₃ is hydrocarbyl, substituted hydrocarbyl, halogen or an optionally substituted heteroatom selected from the group consisting of oxygen, nitrogen, and sulfur; and
R₄₁, R₄₄ and R₄₅ are independently hydrogen, halogen or alkoxy.

113. The compound of claim 92 wherein Z₄ is 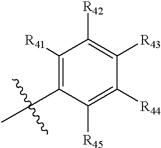
wherein
R₄₂ is as defined in claim 92;
R₄₅ is hydrocarbyl, substituted hydrocarbyl, halogen or an optionally substituted heteroatom selected from the group consisting of oxygen, nitrogen, and sulfur; and
R₄₁, R₄₃ and R₄₄ are independently hydrogen, halogen or alkoxy.

114. The compound of claim 92 wherein Z₄ is 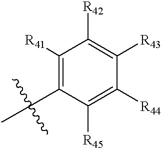
wherein
R₄₂ is as defined in claim 92;
R₄₁ is hydrocarbyl, substituted hydrocarbyl, halogen or an optionally substituted heteroatom selected from the group consisting of oxygen, nitrogen, and sulfur; and
R₄₃, R₄₄ and R₄₅ are independently hydrogen, halogen or alkoxy.

115. The compound of claim 92 wherein L₁ is a bond; Z₁ is selected from the group consisting of cyclopropyl, methyl, ethyl, isobutyl, tert-butyl, and sec-butyl optionally substituted at any substitutable position with fluorine, hydroxy, carboxy, or alkoxycarbonyl; Z₃ is phenyl substituted with an amidine group and optionally substituted by hydrogen, fluorine, hydroxy, carboxy, alkoxycarbonyl, or hydrocarbyloxy; and one of R₄₁, R₄₃, R₄₄ or R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine.

116. The compound of claim 93 wherein L₁ is a bond; Z₁ is selected from the group consisting of cyclopropyl, methyl, ethyl, isobutyl, and sec-butyl optionally substituted at any substitutable position with fluorine; and R₄₄ is selected from the group consisting of hydroxy, alkylsulfonyl, haloalkyl, haloalkoxy, haloalkylthio, carboxamidoalkyl, and carboxamidoalkylaryl

117. A compound having the structure: 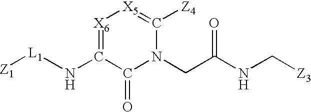
wherein
X₅ and X₆ are independently nitrogen, CH, C(F), C(Cl), or C(Br);
L₁ is a linker, linking Z₁ to the heterocyclic core ring;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy, or alkoxycarbonyl;
Z₃ comprises a substituted phenyl, thienyl, or furanyl ring, the phenyl, thienyl or furanyl ring being substituted with an amidine or a derivatized amidine group, and optionally substituted with fluorine, provided, however, when Z₃ is phenyl or thienyl, the phenyl or thienyl ring is further substituted by at least one of hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀₁ Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁₁ the substituent being R₄₃ when bonded to Z₄₃, the substituent being R₄₄ when bonded to Z₄₄, and the substituent being R₄₅ when bonded to Z₄₅;
R₄₂ is amino; and
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen.

118. A compound having the structure: 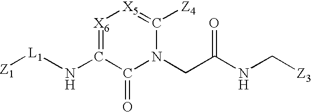
wherein
X₅ and X₆ are independently nitrogen, CH, C(F), C(Cl), or C(Br);
L₁ is a linker, linking Z₁ to the heterocyclic core ring;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy, or alkoxycarbonyl;
Z₃ comprises a substituted phenyl, thienyl, or furanyl ring, the phenyl, thienyl or furanyl ring being substituted with an amidine or a derivatized amidine group and optionally substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₁ when bonded to Z₄₅;
R₄₂ is amino; and
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₁ is other than hydrogen;
provided, however, (a) one of Z₁, Z₃ and Z₄ is hydroxy substituted or (b) one of Z₁ and Z₃ is carboxy substituted.

119. The compound of claims 117 or 118 wherein L₁ is a bond.

120. The compound of claim 119 wherein Z₁ is C₁-C₅ alkyl optionally substituted at any substitutable position with fluorine, hydroxy, carboxy, or alkoxycarbonyl.

121. The compound of claim 119 wherein Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl.

122. The compound of claim 119 wherein Z₃ is 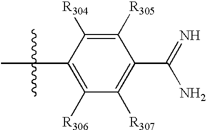
wherein
R₃₀₄ and R₃₀₆ are independently selected from the group consisting of hydrogen, fluorine, hydroxy, carboxy, hydrocarbyloxy, and alkoxycarbonyl; and
R₃₀₅ and R₃₀₇ are independently selected from the group consisting of hydrogen, fluorine, methoxy, hydroxy, and carboxy;
provided, however, at least one of R₃₀₄, R₃₀₅, R₃₀₆, and R₃₀₇ is other than hydrogen and fluorine.

123. The compound of claim 118 wherein Z₃ is 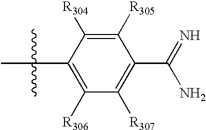
wherein
R₃₀₄ and R₃₀₆ are independently selected from the group consisting of hydrogen, fluorine, hydroxy, carboxy, hydrocarbyloxy, and alkoxycarbonyl; and
R₃₀₅ and R₃₀₇ are independently selected from the group consisting of hydrogen, fluorine, methoxy, hydroxy, and carboxy.

124. The compound of claim 123 wherein at least one of R₃₀₄, R₃₀₅ R₃₀₆, and R₃₀₇ is hydroxy or carboxy.

125. The compound of claims 117 or 118 wherein Z₃ is a phenyl, thienyl or furanyl ring substituted with a derivatized amidine which, upon hydrolysis, oxidation, reduction, or elimination, or any combination thereof, under physiological conditions yields an amidine group.

126. The compound of claim 117 or 118 wherein Z₄ is 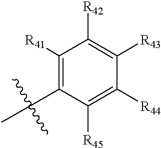
wherein:
R₄₂ is as defined in claim 117;
R₄₄ is hydrocarbyl, substituted hydrocarbyl, halogen or an optionally substituted heteroatom selected from the group consisting of oxygen, nitrogen, and sulfur; and
R₄₁, R₄₃ and R₄₅ are independently hydrogen, hydrocarbyl, substituted hydrocarbyl, halogen or an optionally substituted heteroatom selected from the group consisting of oxygen, nitrogen, and sulfur.

127. The compound of claim 126 wherein R₄₂ is as defined in claim 1, R₄₄ is as defined in claim 13, and R₄₁, R₄₃ and R₄₅ are independently hydrogen, halogen, alkoxy, or alkyl, optionally substituted with halogen or alkoxy.

128. The compound of claim 127 wherein R₄₄ is selected from the group consisting of hydroxy, carboxy, carboxamido, alkoxy, alkylsulfonyl, sulfonamido, or alkoxycarbonyl.

129. The compound of claim 128 wherein R₄₄ is sec-butylamide, carboxy, ethoxycarbonyl, isopropoxycarbonyl, butoxycarbonyl, isopropylamide or hydroxy.

130. The compound of claim 126 wherein R₄₄ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine.

131. The compound of claim 126 wherein Z₄₁, Z₄₃ or Z₄₅ is substituted with fluorine or chlorine.

132. The compound of claims 117 or 118 wherein Z₄ is 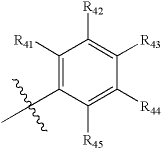
wherein
R₄₂ is as defined in claim 117;
R₄₃ is hydrocarbyl, substituted hydrocarbyl, halogen or an optionally substituted heteroatom selected from the group consisting of oxygen, nitrogen, and sulfur; and
R₄₁, R₄₄ and R₄₅ are independently hydrogen, halogen or alkoxy.

133. The compound of claims 117 or 118 wherein Z₄ is 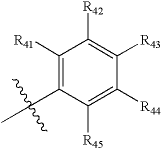
wherein
R₄₂ is as defined in claim 117;
R₄₅ is hydrocarbyl, substituted hydrocarbyl, halogen or an optionally substituted heteroatom selected from the group consisting of oxygen, nitrogen, and sulfur; and
R₄₁, R₄₃ and R₄₄ are independently hydrogen, halogen or alkoxy.

134. The compound of claims 117 or 118 wherein Z₄ is 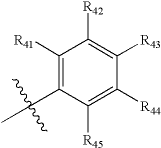
wherein
R₄₂ is as defined in claim 117;
R₄₁ is hydrocarbyl, substituted hydrocarbyl, halogen or an optionally substituted heteroatom selected from the group consisting of oxygen, nitrogen, and sulfur; and
R₄₃, R₄₄ and R₄₅ are independently hydrogen, halogen or alkoxy.

135. The compound of claim 117 having the structure: 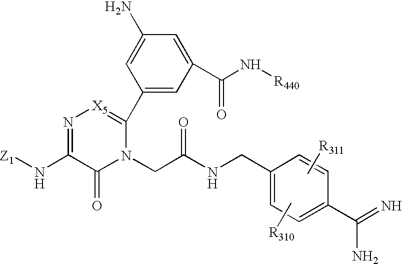
wherein
X₅ is CH, C(Cl) or C(F);
Z₁ is isopropyl, cyclopropyl, cyclobutyl or cycylopentyl optionally substituted by fluorine, hydroxy, carboxy, or alkoxycarbonyl;
R₃₁₀ and R₃₁₁ are independently selected from the group consisting of hydrogen, fluorine, hydroxy, alkoxy, and carboxy, provided at least one of R₃₁₀ and R₃₁₁ is other than fluorine and hydrogen; and
R₄₄₀ is C₁-C₆ alkyl, aryl, aralkyl, carboxy, or carboxyalkyl, wherein said alkyl, aryl, aralkyl, carboxy, or carboxyalkyl is optionally further substituted by fluorine.

136. A compound having the structure: 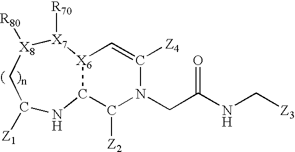
wherein
X₅ is nitrogen, CH, C(F), C(Cl), or C(Br);
X₆ is carbon or nitrogen, provided the dashed line represents a double bond when X₆ is carbon and the dashed line represents a single bond when X₆ is nitrogen;
X₇ and X₈ are independently carbon, nitrogen, oxygen or sulfur;
Z₁ is C₁-C₈ alkyl, C₂-C₈ alkenyl, or C₂-C₈ alkynyl, the alkyl, alkenyl, or alkynyl being optionally substituted at any substitutable position with fluorine, hydroxy, carboxy, or alkoxycarbonyl;
Z₂ is a hydrogen bond acceptor covalently or datively bonded to the carbon gamma to X₅.
Z₃ comprises a substituted phenyl, thienyl, or furanyl ring, the phenyl, thienyl or furanyl ring being substituted with an amidine or a derivatized amidine group and optionally substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅;
R₄₂ is amino;
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen;
R₇₀ and R₈₀ are independently selected from the group consisting of hydrogen, halogen, amino, hydrocarbyl, substituted hydrocarbyl, aryl, wherein aryl is phenyl optionally substituted by hydroxy, amino, C₁-C₈ alkyl, or halogen provided that R₇₀ is not present when X₇ is a bond and R₈₀ is not present when X₈ is a bond; or R₇₀ and R₈₀, along with the ring atoms to which each is attached, form a 5- or 6-membered saturated ring; and
n is 0 to 2.

137. The compound of claim 136 having the structure: 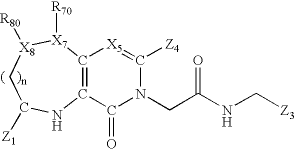
wherein
X₅, X₇, X₈, Z₁, Z₃, Z₄, R₇₀, R₈₀ and n are as defined in claim 136.

138. The compound of claim 137 wherein X₇ and X₈ are carbon.

139. The compound of claims 136 or 137 wherein Z₄ is 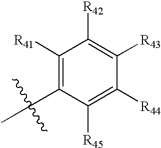
wherein:
R₄₂ is as defined in claim 136;
R₄₄ is hydrocarbyl, substituted hydrocarbyl, halogen or an optionally substituted heteroatom selected from the group consisting of oxygen, nitrogen, and sulfur; and
R₄₁, R₄₃ and R₄₅ are independently hydrogen, hydrocarbyl, substituted hydrocarbyl, halogen or an optionally substituted heteroatom selected from the group consisting of oxygen, nitrogen, and sulfur.

140. The compound of claim 137 wherein Z₁ is methyl, ethyl, isopropyl, cyclopropyl, sec-butyl, tert-butyl, and cyclobutyl optionally substituted at any substitutable position with fluorine, hydroxy, carboxy or alkoxycarbonyl.

141. The compound of claim 137 wherein Z₃ is 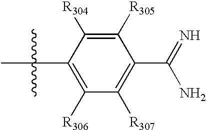
wherein
R₃₀₄ and R₃₀₆ are independently selected from the group consisting of hydrogen, fluorine, hydroxy, carboxy, hydrocarbyloxy, and alkoxycarbonyl; and
R₃₀₅ and R₃₀₇ are independently selected from the group consisting of hydrogen, fluorine, methoxy, hydroxy, and carboxy;

142. A compound having the structure: 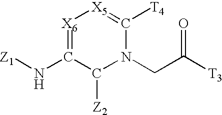
wherein
X₅ and X₆ are independently nitrogen, CH, C(F) or C(Br);
T₃ is hydroxy, alkoxy, substituted alkoxy, or substituted amino;
T₄ is Cl, Br, I, S(CH₃), or OSO₂ (CF₃);
Z₁ is C₁-C₈ alkyl, C₂-C₈ alkenyl, or C₂-C₈ alkynyl, the alkyl, alkenyl, or alkynyl being optionally substituted with fluorine, hydroxy, carboxy, or alkoxycarbonyl; and
Z₂ is a hydrogen bond acceptor covalently bonded to the carbon gamma to X₅.

143. A compound having the structure: 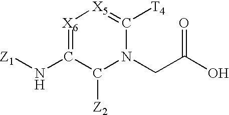
wherein
X₅ and X₆ are independently nitrogen, CH, C(F) or C(Br);
Z₁ is C₁-C₈ alkyl, C₂-C₈ alkenyl, or C₂-C₈ alkynyl, the alkyl, alkenyl, or alkynyl being optionally substituted with fluorine, hydroxy, carboxy, or alkoxycarbonyl;
Z₂ is a hydrogen bond acceptor covalently bonded to the carbon gamma to X₅; and
Z₄ is hydrocarbyl, substituted hydrocarbyl, or a 5- or 6-membered heterocyclic or carbocyclic ring, the ring atoms of the 5- or 6-membered heterocyclic or carboxylic ring of Z₄ being carbon, nitrogen, oxygen, or sulfur.

144. A composition for inhibiting thrombotic conditions in blood comprising a compound of any of claims 1, 42, 67, 92, 117, or 118 and a pharmaceutically acceptable carrier.

145. A method for inhibiting thrombotic conditions in blood comprising adding to blood a therapeutically effective amount of the composition of claim 144.

146. A method for inhibiting formation of blood platelet aggregates in blood comprising adding to blood a therapeutically effective amount of the composition of claim 144.

147. A method for inhibiting thrombus formation in blood comprising adding to blood a therapeutically effective amount of the composition of claim 144.

148. A method for treating or preventing venuous thromboembolism and pulmonary embolism in a mammal comprising administering to the mammal a therapeutically effective amount of the composition of claim 144.

149. A method for treating or preventing deep vein thrombosis in a mammal comprising administering to the mammal a therapeutically effective amount of the composition of claim 144.

150. A method for treating or preventing cardiogenic thromboembolism in a mammal comprising administering to the mammal a therapeutically effective amount of the composition of claim 144.

151. A method for treating or preventing thromboembolic stroke in mammals comprising administering to the mammal a therapeutically effective amount of the composition of claim 144.

152. A method for treating or preventing thrombosis associated with cancer and cancer chemotherapy in mammals comprising administering to the mammal a therapeutically effective amount of the composition of claim 144.

153. A method for treating or preventing unstable angina in mammals comprising administering to the mammal a therapeutically effective amount of the composition of claim 144.

154. A method for inhibiting thrombus formation in blood comprising adding to blood a therapeutically effective amount of the composition of claim 144 with a therapeutically effective amount of fibrinogen receptor antagonist.

155. A composition comprising a compound of each of claims 1, 42, 67, 92, 117 or 118 or a pharmaceutically acceptable salt or prodrug thereof and a thrombolytic agent.

156. The composition of claim 155 wherein the thrombolytic agent is selected from the group consisting of anti-platelet agents, anticoagulation agents and cardiovascular agents.

157. The composition of claim 155 wherein the thrombolytic agent is an anti-platelet agent.

158. The composition of claim 157 wherein the anti-platelet agent is selected from the group consisting of a salicylate compound, ticlopidine, clopidrogel, and a GP IIa/IIIa inhibitor.

159. The composition of claim 158 wherein the anti-platelet agent is a salicylate compound.

160. The composition of claim 159 wherein the salicylate compound is aspirin.

161. The composition of claim 157 wherein the anti-platelet agent substantially inhibits prostaglandin synthesis.

162. A method for the treatment or prevention of a thrombolytic condition in a subject, the method comprising administering to the subject a compound of each of claims 1, 42, 67, 92, 117 or 118 or a pharmaceutically acceptable salt or prodrug thereof and a thrombolytic agent.

163. The method of claim 162 wherein the thrombolytic agent is selected from the group consisting of anti-platelet agents, anticoagulation agents and cardiovascular agents.

164. The method of claim 163 wherein the thrombolytic agent is an anti-platelet agent.

165. The method of claim 164 wherein the anti-platelet agent is selected from the group consisting of a salicylate compound, ticlopidine, clopidrogel, and a GP IIa/IIIa inhibitor.

166. The method of claim 165 wherein the anti-platelet agent is a salicylate compound.

167. The method of claim 166 wherein the salicylate compound is aspirin.

168. The method of claim 164 wherein the anti-platelet agent substantially inhibits prostaglandin synthesis.

169. The method of claim 162 wherein the thrombolytic condition is selected from the group consisting of myocardial infarction, stroke, amaurosis fugax, aortic stenosis, cardiac stenosis, coronary stenosis and pulmonary stenosis.

170. The method of claim 162 wherein the compound of claim 1, 42, 67, 92, 117 or 118 and the thrombolytic agent are administered in a substantially simultaneous manner.

171. The method of claim 162 wherein the compound of claim 1, 42, 67, 92, 117 or 118 and the thrombolytic agent are administered sequentially.
